(19) United States
(12) Patent Application Publication
(10) Pub. No.: US 2023/0131195 A1
(43) Pub. Date: Apr. 27, 2023

(54) ORGANIC ELECTROLUMINESCENT COMPOUND AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Young-Kwang KIM, Gyeonggi-do (KR); Jeong-Eun YANG, Gyeonggi-do (KR); Hong-Se OH, Gyeonggi-do (KR); Hee-Ryong KANG, Gyeonggi-do (KR); Joon-Hyung KIL, Gyeonggi-do (KR); Eun-Joung CHOI, Gyeonggi-do (KR); YeJin JEON, Gyeonggi-do (KR); Dong-Hyung LEE, Gyeonggi-do (KR); Hyun-Woo KANG, Gyeonggi-do (KR)

(21) Appl. No.: 17/896,497

(22) Filed: Aug. 26, 2022

(30) Foreign Application Priority Data
Sep. 10, 2021 (KR) 10-2021-0121001
Aug. 8, 2022 (KR) 10-2022-0098680

(51) Int. Cl. C07C 211/54; C07D 209/82; C07D 333/76; C07D 307/91; C07D 213/74; C07D 405/12; C07D 239/42; C07D 407/12; C07D 345/00; H01L 51/00; H01L 51/50

(52) U.S. Cl. CPC C07C 211/54; C07D 209/82; C07D 333/76; C07D 307/91; C07D 213/74; C07D 405/12; C07D 239/42; C07D 407/12; C07D 345/00; H01L 51/0071; H01L 51/0072; H01L 51/0073; H01L 51/0074; H01L 51/0067; H01L 51/0059; H01L 51/0052; H01L 51/0054; H01L 51/5012

(57) ABSTRACT

The present disclosure relates to an organic electroluminescent compound, and an organic electroluminescent device comprising the same. By comprising the organic electroluminescent compound according to the present disclosure, it is possible to provide an organic electroluminescent device having improved current efficiency and/or lifetime properties.

### TECHNICAL FIELD

The present disclosure relates to an organic electroluminescent compound and an organic electroluminescent device comprising the same.

### BACKGROUND ART

An electroluminescent device (EL device) is a self-light-emitting display device which has advantages in that it provides a wider viewing angle, a greater contrast ratio, and a faster response time. An organic EL device was first developed by Eastman Kodak in 1987, by using small aromatic diamine molecules and aluminum complexes as materials for forming a light-emitting layer [Appl. Phys. Lett. 51, 913, 1987].

An organic electroluminescent device (OLED) consists of a multi-layer structure including a hole injection layer, a hole transport layer, a light-emitting layer, an electron transport layer, and an electron injection layer, etc., in order to improve its efficiency and stability. In this case, the selection of a compound included in the hole transport layer or the like is recognized as one of the means for improving device properties such as the hole transport efficiency to a light-emitting layer, the luminous efficiency, and the lifetime.

In this regard, copper phthalocyanine (CuPc), 4,4′-bis[N-(1-naphthyl)-N-phenylamino]biphenyl (NPB), N,N′-diphenyl-N,N′-bis(3-methylphenyl)-(1,1′-biphenyl)-4,4′-diamine (TPD), 4,4′,4″-tris(3-methylphenylphenylamino)triphenylamine (MTDATA), etc., were used as a hole injection and transport materials in an OLED. However, an OLED prepared using these materials have problems of reduction in quantum efficiency and lifetime. This is because, when an OLED is driven under high current, thermal stress occurs between an anode and a hole injection layer, thereby such thermal stress significantly reduces the lifetime of the device. Further, since the organic material used in the hole injection layer has very high hole mobility, there have been problems in that the hole-electron charge balance is broken and the quantum efficiency (cd/A) is lowered.

Meanwhile, Korean Patent Application Laying-Open No. 10-2020-0034638 discloses an organic electroluminescent device comprising a compound with a symmetric structure containing an amino group. However, the aforementioned reference does not specifically disclose a compound claimed in the present disclosure. In addition, there has been a need to develop hole transport materials for improving performances of OELDs.

### DISCLOSURE OF INVENTION

### Technical Problem

The objective of the present disclosure is firstly, to provide an organic electroluminescent compound effective for manufacturing an organic electroluminescent device having improved current efficiency and/or lifetime properties, and secondly, to provide an organic electroluminescent device comprising the organic electroluminescent compound.

### Solution to Problem

As a result of intensive studies to solve the technical problems, the present inventors found that the above objective can be achieved by an organic electroluminescent compound represented by the following formula 1.

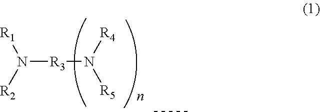

In formula 1,

R₁, R₂, R₄, and R₅, each independently, represent a substituted or unsubstituted (C6-C30)aryl, or a substituted or unsubstituted (3- to 30-membered)heteroaryl;

R₃ represents a substituted or unsubstituted (C6-C30)aryl(ene), or a substituted or unsubstituted (3- to 30-membered)heteroaryl(ene);

with the proviso that when n represents 0, at least one of R₁ to R₃ is substituted with the following formula 1′; and when n represents 1, at least one of R₁ to R₅ is substituted with the following formula 1′;

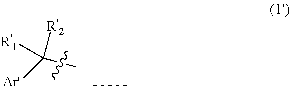

in formula 1′,

R′₁ and R′₂ represent a (C1-C5)alkyl unsubstituted or substituted with deuterium;

Ar′ represents a substituted or unsubstituted (C6-C30)aryl, or a substituted or unsubstituted (3- to 30-membered)heteroaryl, with the proviso that Ar′ does not comprise an amine group; and

n represents an integer of 0 or 1;

with the proviso that formula 1 does not comprise an acridine structure in a spiro form.

### Advantageous Effects of Invention

An organic electroluminescent device having improved current efficiency and/or lifetime properties is provided by using the organic electroluminescent compound according to the present disclosure.

### MODE FOR THE INVENTION

Hereinafter, the present disclosure will be described in detail. However, the following description is intended to explain the present disclosure and is not meant in any way to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc. A hole transport zone material may be at least one selected from the group of a hole transport material, a hole injection material, an electron blocking material, a hole auxiliary material, and a light-emitting auxiliary material.

The organic electroluminescent material of the present disclosure may comprise at least one compound represented by formula 1. The compound of formula 1 may be included in at least one layer of the layers constituting the organic electroluminescent device, and may be included in at least one layer of the layers constituting the hole transport zone, but is not limited thereto. When the compound of formula 1 is included in a hole transport layer, a hole auxiliary layer, a light-emitting layer or a light-emitting auxiliary layer, it may be included as a hole transport material, a hole auxiliary material, a host material, or a light-emitting auxiliary material.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, etc. The term “(C2-C30)alkenyl” in the present disclosure is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” in the present disclosure is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl. etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered)heterocycloalkyl” is meant to be a cycloalkyl having 3 to 7 ring backbone atoms, preferably 5 to 7 ring backbone atoms, and including at least one heteroatom selected from the group consisting of B, N, O, S, Si, and P, preferably the group consisting of O, S, and N. The above heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms, and may be partially saturated. The number of ring backbone carbon atoms is preferably 6 to 25 and more preferably 6 to 18. The above aryl may comprise a spiro structure. The above aryl may include phenyl, biphenyl, terphenyl, naphthyl, binaphthyl, phenylnaphthyl, naphthylphenyl, phenylterphenyl, fluorenyl, phenylfluorenyl, diphenylfluorenyl, dimethylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, etc. Specifically, the above aryl may include phenyl, 1-naphthyl, 2-naphthyl, 1-anthryl, 2-anthryl, 9-anthryl, benzanthryl, 1-phenanthryl, 2-phenanthryl. 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenyl, 3-biphenyl, 4-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-guaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11 ,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11, 11-dimethyl-1-benzo[b]fluorenyl, 11,11-dimethyl- 2-benzo[b]fluorenyl, 11,11-dimethyl-3-benzo[b]fluorenyl, 11,11-dimethyl-4-benzo[b]fluorenyl, 11,11-dirhethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11.11-diphenyl-7-benzo[a]fluorenyl, 11 ,11-diphenyl-8-benzo[a]fluorenyl, 11,11-diphenyl-9-benzo[a]fluorenyl, 11,11-diphenyl-10-benzo[b]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11 ,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” is meant to be an aryl(ene) having 3 to 30 ring backbone atoms, and including at least one heteroatoms selected from the group consisting of B, N, O, S, Si, and P. The number of heteroatoms is preferably 1 to 4. The above heteroaryl(ene) may be a monocyclic ring, or a fused ring condensed with at least one benzene ring; may be partially saturated. In addition, the above heteroaryl(ene) may be one formed by linking at least one heteroaryl or aryl group to a heteroaryl(ene) group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, benzophenanthrofuranyl, dibenzothiophenyl, benzimidazolyl, benzothiozolyl, benzoisothiozolyl, benzophenanthrothiophenyl, benzoisoxazolyl, benzoxazolyl, phenanthroxazolyl, phenanthrothiazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, etc. More specifically, the above heteroaryl may include 1-pyrrolyl, 2-pyrrolyl, 3-pyrrolyl, pyrazinyl, 2-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridinyl, 3-imidazopyridinyl, 5-imidazopyridinyl, 6-imidazopyridinyl, 7-imidazopyridinyl, 8-imidazopyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoguinolyl, 4-isoquinolyl, 5-isoguinolyl, 6-isoguinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtha-[1,2-b]-benzofuranyl, 6-naphtho-[1,2-b]-benzofuranyl, 7-naphtha-[1,2-b]-benzofuranyl, 8-naphtho-[1,2-b]-benzofuranyl, 9-naphtho-[1,2-b]-benzofuranyl, 10-naphtha-[1,2-b]-benzofuranyl, 1-naphtho-[2,3-b]-benzofuranyl, 2-naphtha-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho[2,3-b]-benzofuranyl, 7-naphtho-[2,3-b]-benzofuranyl, 8-naphtha-[2,3-b]-benzofuranyl, 9-naphtho-[2,3-b]-benzofuranyl, 10-naphtha-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtha-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho[1,2-b]-benzothiophenyl, 2-naphtha-[1,2-b]-benzothiophenyl, 3-naphtho-[1,2-b]-benzothiophenyl, 4-naphtho[1,2-b]-benzothiophenyl, 5-naphtho[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiopheny, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtha-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silarluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Furthermore, “halogen” includes F, Cl, Br, and I.

In addition, “ortho (o-),” “meta (m-),” and “para (p-)” are prefixes, which represent the relative positions of substituents respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a pars position.

Herein, “substituted” in the expression “substituted or unsubstituted” means that a hydrogen atom in a certain functional group is replaced with another atom or another functional group, i.e., a substituent, and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as a heteroaryl substituent, or as substituents in which two heteroaryl substituents are linked. Herein, the substituent(s) of the substituted aryl(ene) and the substituted heteroaryl(ene), each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of a (C1-C30)alkyl(s) and a (3- to 30-membered)heteroaryl(s); a tri(C1-C30)alkylsilyl; a tri(C6-C30)arylsilyl; a di(C1-C30)alkyl(C6-C30)arylsilyl: a (C1-C30)alkyldi(C6-C30)arylsilyl: a fused ring group of a (C3-C30) aliphatic ring(s) and a (C6-C30) aromatic ring(s); an amino; a mono- or di- (C1-C30)alkylamino; a mono- or di- (C2-C30)alkenylamino; a mono- or di- (C6-C30)arylamino unsubstituted or substituted with a (C1-C30)alkyl(s); a mono- or di- (3- to 30-membered)heteroarylamino; a (C1-C30)alkyl(C2-C30)alkenylamino; a (C1-C30)alkyl(C6-C30)arylamino: a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)alylboronyl; a (C6-C30)aryl(C1-C30)alkyl; and a (C1-C30)alkyl(C6-C30)aryl. According to one embodiment of the present disclosure, the substituent(s), each independently, are at least one selected from the group consisting of deuterium; a (C1-C30)alkyl; a (C6-C30)aryl(C1-C30)alkyl; a (C6-C30)aryl; and a (5- to 30-membered)heteroaryl. According to another embodiment of the present disclosure, the substituent(s), each independently, are at least one selected from the group consisting of deuterium; a (C1-C10)alkyl; a (C6-C30)aryl(C1-C20)alkyl; a (C6-C20)aryl; and a (5- to 20-membered)heteroaryl. For example, the substituent(s), each independently, may be at least one selected from the group consisting of deuterium; a methyl; a phenyl; a biphenyl; a naphthyl; a pyridyl; a carbazolyl; and a tert-butyl substituted with at least one of a phenyl(s), a naphthyl(s), a phenanthrenyl(s) and a triphenylene(s).

Hereinafter, the compound represented by formula 1 will be described in more detail.

In formula 1, R₁, R₂, R₄, and R₅, each independently, represent a substituted or unsubstituted (C6-C30)aryl, or a substituted or unsubstituted (3- to 30-membered)heteroaryl. According to one embodiment of the present disclosure, R₁, R₂, R₄, and R₅, each independently, represent a (C6-C30)aryl unsubstituted or substituted with at least one of a (C1-C30)alkyl(s), a (C6-C30)aryl(s), a (3- to 30-membered)heteroaryl(s), and formula 1′; or a (3- to 30-membered)heteroaryl unsubstituted or substituted with at least one of a (C6-C30)aryl(s) and formula 1′. According to another embodiment of the present disclosure, R₁, R₂, R₄, and R₅, each independently, represent a (C6-C30)aryl unsubstituted or substituted with at least one of a (C1-C10)alkyl(s), a (C6-C20)aryl(s), a (5- to 18-membered)heteroaryl(s), and formula 1′; or a (6- to 28-membered)heteroaryl unsubstituted or substituted with at least one of a (C6-C18)aryl(s) and formula 1′. For example, R₁, R₂, R₄, and R₅, each independently, may be a phenyl, a biphenyl, a terphenyl, a naphthyl, a phenylnaphthyl, a naphthylphenyl, a phenanthrenyl, a benzophenanthrenyl, a triphenylenyl, a chrysenyl, a dimethylfluorenyl, a diphenylfluorenyl unsubstituted or substituted with a phenyl(s), a dimethylbenzofluorenyl, a diphenylbenzofluorenyl, a spirobifluorenyl unsubstituted or substituted with a phenyl(s), a spiro[fluorene-benzofluoren]yl, a biphenyl substituted with a carbazolyl(s), a terphenyl substituted with a carbazolyl(s), a phenyl substituted with a pyridyl(s), a dibenzofuranyl unsubstituted or substituted with a phenyl(s), a benzonaphthofuranyl, a dibenzothiophenyl unsubstituted or substituted with a phenyl(s), a benzonaphthothiophenyl, a phenylcarbazolyl unsubstituted or substituted with a phenyl(s), a biphenylcarbazolyl unsubstituted or substituted with a phenyl(s), a phenylbenzocarbazolyl, a pyridyl substituted with a phenyl(s), or a pyrimidinyl substituted with a phenyl(s), etc., and may be further substituted with formula 1′.

In formula 1, R₃ represents a substituted or unsubstituted (C6-C30)aryl(ene), or a substituted or unsubstituted (3- to 30-membered)heteroaryl(ene). According to one embodiment of the present disclosure, R₃ may represent a (C6-C30)aryl(ene) unsubstituted or substituted with at least one of a (C1-C30)alkyl(s), a (C6-C30)aryl(s), a (3- to 30-membered)heteroaryl(s), and formula 1′; or a (3- to 30-membered)heteroaryl(ene) unsubstituted or substituted with at least one of a (C6-C30)aryl(s) and formula 1′. According to another embodiment of the present disclosure, R₃ may represent a (C6-C30)aryl(ene) unsubstituted or substituted with at least one of a (C1-C10)alkyl(s), a (C6-C20)aryl(s), a (5-to 18-membered)heteroaryl(s), and formula 1′; or a (6- to 28-membered)heteroaryl(ene) unsubstituted or substituted with at least one of a (C6-C18)aryl(s) and formula 1′. For example, R₃ may be a phenyl, a biphenyl, a terphenyl, a naphthyl, a phenylnaphthyl, a naphthylphenyl, a phenanthrenyl, a benzophenanthrenyl, a triphenylenyl, a chrysenyl, a dimethylfluorenyl, a diphenylbenzofluorenyl, a diphenylfluorenyl unsubstituted or substituted with a phenyl(s), a dimethylbenzofluorenyl, a spirobifluorenyl unsubstituted or substituted with a phenyl(s), a spiro[fluorene-benzofluoren]yl, a biphenyl substituted with a carbazolyl(s), a terphenyl substituted with a carbazolyl(s), a phenyl substituted with a pyridyl(s), a dibenzofuranyl unsubstituted or substituted with a phenyl(s), a benzonaphthofuranyl, a dibenzothiophenyl unsubstituted or substituted with a phenyl(s), a benzonaphthothiophenyl, a phenylcarbazolyl unsubstituted or substituted with a phenyl(s), a biphenylcarbazolyl unsubstituted or substituted with a phenyl(s), a phenylbenzocarbazolyl, a pyridyl substituted with a phenyl(s), a pyrimidinyl substituted with a phenyl(s), a phenylene unsubstituted or substituted with a phenyl(s) or biphenyl(s), a biphenylene, a terphenylene, a dimethylfluorenylene, a dimethylbenzofluorenylene, a diphenylfluorenylene, a spirobifluorenylene, a phenylcarbazolylene, a dibenzofuranylene, a dibenzothiophenylene, or a dibenzoselenophenylene, etc., and may be further substituted with formula 1′.

In formula 1, when n represents 0, at least one of R₁ to R₃ is substituted with the following formula 1′; and when n represents 1, at least one of R₁ to R₅ is substituted with the following formula 1′.

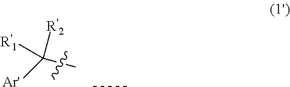

In formula 1′, R′₁ and R′₂ represent a (C1-C5)alkyl unsubstituted or substituted with deuterium. For example, R′₁ and R′₂ may be a methyl, etc.

In formula 1′, Ar′ represents a substituted or unsubstituted (C6-C30)aryl, or a substituted or unsubstituted (3- to 30-membered)heteroaryl, with the proviso that Ar′ does not comprise an amine group. According to one embodiment of the present disclosure, Ar′ represents an unsubstituted (C6-C20)aryl. For example, Ar′ may be a phenyl, a naphthyl, a phenanthrenyl, a triphenylenyl, etc.

In formula 1, n represents an integer of 0 or 1; with the proviso that formula 1 does not comprise an acridine structure in a spino form.

According to one embodiment of the present disclosure, formula 1 is represented by any one of the following formulas 1-1 to 1-3:

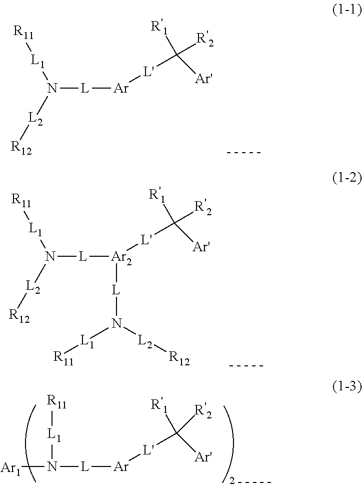

In formulas 1-1 to 1-3, R₁₁ and R₁₂, each independently, represent a substituted or unsubstituted (C6-C30)aryl, or a substituted or unsubstituted (3- to 30-membered)heteroaryl. According to one embodiment of the present disclosure, R₁₁ and R₁₂, each independently, represent a (C6-C30)aryl unsubstituted or substituted with at least one of a (C1-C30)alkyl(s), a (C6-C30)aryl(s), and a (3- to 30-membered)heteroaryl(s); or a (3- to 30-membered)heteroaryl unsubstituted or substituted with a (C6-C30)aryl(s). According to another embodiment of the present disclosure, R₁₁ and R₁₂, each independently, represent a (C6-C30)aryl unsubstituted or substituted with at least one of a (C1-C10)alkyl(s) and a (C6-C20)aryl(s); or a (5- to 20-membered)heteroaryl unsubstituted or substituted with a (C6-C18)aryl(s). For example, R₁₁ and R₁₂, each independently, may be a phenyl, a biphenyl, a terphenyl, a naphthyl, a phenylnaphthyl, a phenanthrenyl, a benzophenanthrenyl, a triphenylenyl, a chrysenyl, a dimethylfluorenyl, a diphenylfluorenyl unsubstituted or substituted with a phenyl(s), a dimethylbenzofluorenyl, a diphenylbenzofluorenyl, a spirobifluorenyl, a spiropuorene-benzofluorenlyl, a dibenzofuranyl, a benzonaphthofuranyl, a dibenzothiophenyl, a benzonaphthothiophenyl, a dibenzoselenophenyl, a carbazolyl substituted with a phenyl(s), a phenylcarbazolyl, or a phenylbenzocarbazolyl, etc.

In formulas 1-1 to 1-3, L, L₁, L₂ and L′, each independently, represent a single bond, a substituted or unsubstituted (C6-C30)arylene, or a substituted or unsubstituted (3- to 30-membered)heteroarylene. According to one embodiment of the present disclosure. L, L₁, L₂ and L′, each independently, represent a single bond; a (C6-C30)arylene unsubstituted or substituted with at least one of a (C6-C30)aryl(s) and a (3- to 30-membered)heteroaryl(s); or an unsubstituted (3- to 30-membered)heteroarylene, According to another embodiment of the present disclosure, L, L₁, L₂ and L′, each independently, represent a single bond; a (C6-C28)arylene unsubstituted or substituted with at least one of a (C6-C18)aryl(s) and a (6- to 20-membered)heteroaryl(s); or an unsubstituted (6- to 28-membered)heteroarylene. For example, L, L₁, L₂ and L′. each independently, may be a single bond, a phenylene, a biphenylene, a naphthylene, a phenylene unsubstituted or substituted with a carbazolyl(s), a biphenylene unsubstituted or substituted with a carbazolyl(s), a dibenzofuranylene, a dibenzothiophenylene, a pyridylene, a pyrimidinylene, a diphenylfluorenylene, a spirobifluorenylene, a carbazolylene, a phenylcarbazolylene, or a biphenylcarbazolylene, etc.

In formulas 1-1 to 1-3, Ar and Ar₁, each independently, represent a substituted or unsubstituted (C6-C30)arylene, or a substituted or unsubstituted (3- to 30-membered)heteroarylene. According to one embodiment of the present disclosure, Ar and Ar₁, each independently, represent a (C6-C30)arylene unsubstituted or substituted with at least one of a (C1-C30)alkyl(s), a (C6-C30)aryl(s) and a (3- to 30-membered)heteroaryl(s); or a (3- to 30-membered)heteroarylene unsubstituted or substituted with a (C6-C30)aryl(s). According to another embodiment of the present disclosure, Ar and Ar₁, each independently, represent a (C6-C30)arylene unsubstituted or substituted with at least one of a (C1-C10)alkyl(s), a (C6-C18)aryl(s) and a (6- to 20-membered)heteroaryl(s); or a (6- to 28-membered)heteroarylene unsubstituted or substituted with a (C6-C15)aryl(s). For example, Ar and Ar₁, each independently, may be a phenylene, a biphenylene, a terphenylene, a naphthylene, a phenylene-naphthylene, a naphthylene-phenylene, a dimethylfluorenylene, a diphenylfluorenylene unsubstituted or substituted with a phenyl(s), a dimethylbenzofluorenylene, a diphenylbenzofluorenylene, a spirobifluorenylene unsubstituted or substituted with a phenyl(s), a spiro[fluorene-benzofluoren]ylene, a biphenylene substituted with a carbazolyl(s), a terphenylene substituted with a carbazolyl(s), a phenylene substituted with a pyridyl(s), a pyridylene, a dibenzofuranylene unsubstituted or substituted with a phenyl(s), a dibenzothiophenylene unsubstituted or substituted with a phenyl(s), a dibenzoselenophenylene, a phenylcarbazolylene unsubstituted or substituted with a phenyl(s), a biphenylcarbazolylene unsubstituted or substituted with a phenyl(s), a pyridylene unsubstituted or substituted with a phenyl(s), or a pyrimidinylene substituted with a phenyl(s), etc.

In formulas 1-1 to 1-3, Ar₂ represents a trivalent group of a substituted or unsubstituted (C6-C30)aryl ring or a substituted or unsubstituted (3- to 30-membered)heteroaryl ring. According to one embodiment of the present disclosure, Ar₂ represents a trivalent group of a substituted or unsubstituted (C6-C25)aryl ring. According to another embodiment of the present disclosure, Ar₂ represents a trivalent group of an unsubstituted (C6-C20)aryl ring. For example, Ar₂ may be a trivalent group of a phenyl ring, a biphenyl ring, or a terphenyl ring.

When a plurality of R₁₁, R₁₂, L, L₁, L₂, Ar, R′₁, R′₂, and Ar′ are present, each of R₁₁, each of R₁₂, each of L, each of L₁, each of L₂, each of L′, each of Ar, each of R′₁, each of R′₂, and each of Ar′ may be the same as or different from each other; and R′₁, R′₂ and Ar′ are as defined in formula 1.

According to one embodiment of the present disclosure, formula 1 is represented by any one of the following formulas 1-1-1 to 1-1-6:

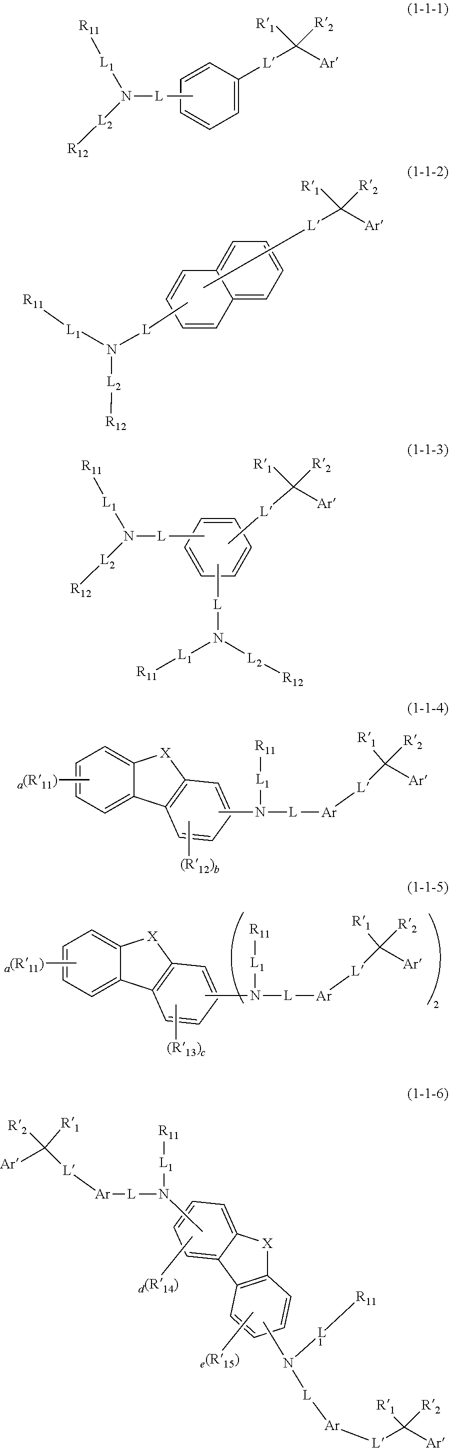

In formulas 1-1-1 to 1-1-6, X represents —CR′ₐR′_{b}—, —NR′_{c}—, —O—, —S— or —Se—; R′ₐ to R′_{c}, each independently, represent hydrogen, deuterium, an unsubstituted (C1-C30)alkyl, an unsubstituted (C6-C30)aryl, or an unsubstituted (3- to 30-membered)heteroaryl; or may be linked to an adjacent substituent(s) to form a spiro ring(s); R′₁₁ to R′₁₅, each independently, represent hydrogen, an unsubstituted (C6-C30)aryl, or an unsubstituted (3- to 30-membered)heteroaryl; or may be linked to an adjacent substituent(s) to form a ring(s); a represents an integer of 1 to 4; b d, and e, each independently, represent an integer of 1 to 3, and c represents an integer of 1 or 2; where if a to e are each an integer of 2 or more, each of R′₁₁ to each of R′₁₅ may be the same as or different from each other; and R′₁, R′₂, Ar′, R₁₁, R₁₂, L, L₁, L₂, L′, and Ar are as defined in formulas 1-1 to 1-3.

According to one embodiment of the present disclosure, R′ₐ to R′_{c}, each independently, represent hydrogen, deuterium, an unsubstituted (C1-C20)alkyl, an unsubstituted (C6-C20)aryl, or an unsubstituted (6- to 30-membered)heteroaryl; or may be linked to an adjacent substituent(s) to form a spiro ring(s); According to another embodiment of the present disclosure, R′ₐ to R′_{c}, each independently, represent an unsubstituted (C1-C10)alkyl, or an unsubstituted (C6-C15)aryl; or may be linked to an adjacent substituent(s) to form a spiro ring(s). For example, R′ₐ to R′_{c}, each independently, may be a methyl, a phenyl, or a biphenyl; or R′ₐ and R′_{b} may be linked to each other to form a spiro fluorene ring.

According to one embodiment of the present disclosure, R′₁₁ to R′₁₅, each independently, represent hydrogen, an unsubstituted (C6-C25)aryl, or an unsubstituted (6- to 30-membered)heteroaryl; or may be linked to an adjacent substituent(s) to form a ring(s). According to another embodiment of the present disclosure, R′₁₁ to R′₁₅, each independently, represent hydrogen, or an unsubstituted (C6-C20)aryl; or may be linked to an adjacent substituent(s) to form a (C6-C18) aromatic ring(s). For example, R′₁₁ to R′₁₅, each independently, may be a phenyl or a biphenyl, or may be linked to an adjacent substituent to form a fused benzene ring.

The compound represented by formula 1 may be selected from the group consisting of the following compounds, but is not limited thereto.

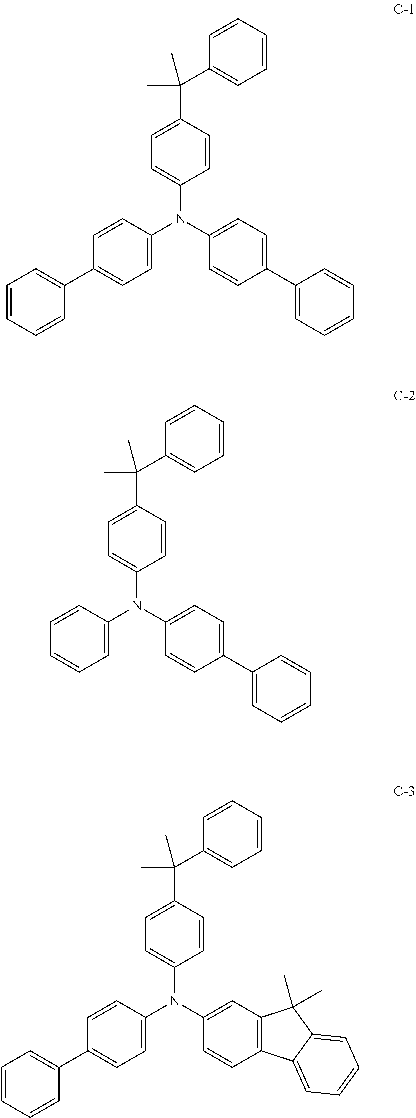 [SMILES: CC(C)(c1ccccc1)c1ccc(N(c2ccc(-c3ccccc3)cc2)c2ccc(-c3ccccc3)cc2)cc1 ; CC(C)(c1ccccc1)c1ccc(N(c2ccccc2)c2ccc(-c3ccccc3)cc2)cc1 ; CC(C)(c1ccccc1)c1ccc(N(c2ccc(-c3ccccc3)cc2)c2ccc3c(c2)C(C)(C)c2ccccc2-3)cc1]

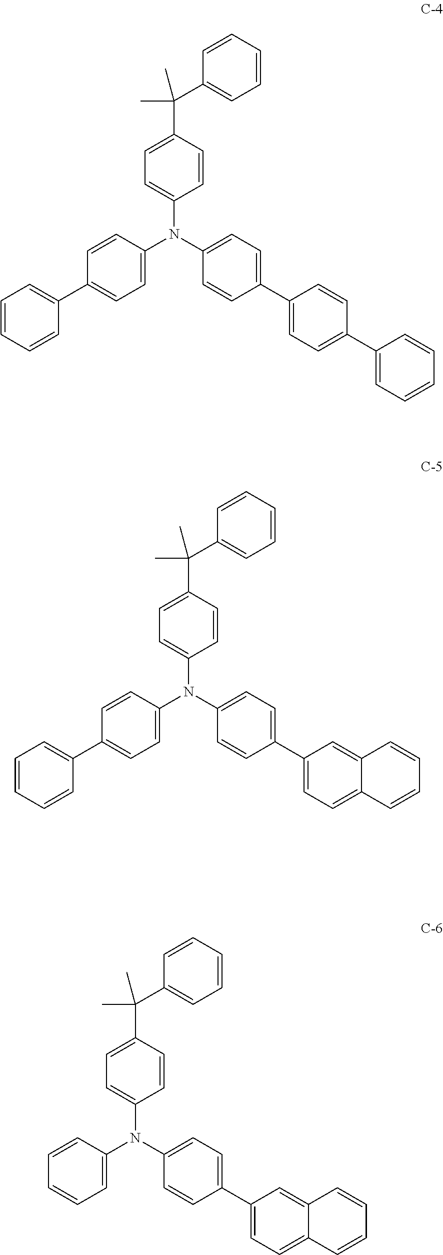 [SMILES: CC(C)(c1ccccc1)c1ccc(N(c2ccc(-c3ccccc3)cc2)c2ccc(-c3ccc(-c4ccccc4)cc3)cc2)cc1 ; CC(C)(c1ccccc1)c1ccc(N(c2ccc(-c3ccccc3)cc2)c2ccc(-c3ccc4ccccc4c3)cc2)cc1 ; CC(C)(c1ccccc1)c1ccc(N(c2ccccc2)c2ccc(-c3ccc4ccccc4c3)cc2)cc1]

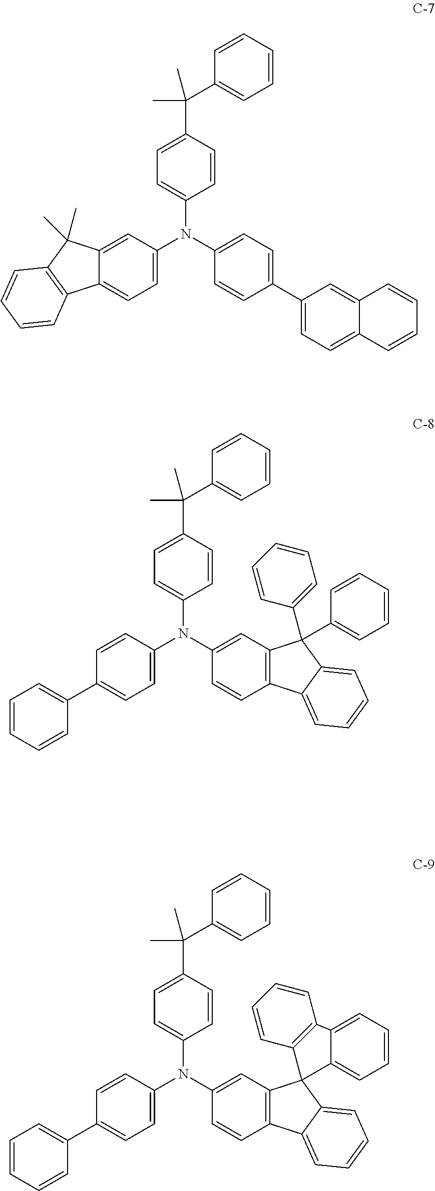 [SMILES: CC(C)(c1ccccc1)c1ccc(N(c2ccc(-c3ccc4ccccc4c3)cc2)c2ccc3c(c2)C(C)(C)c2ccccc2-3)cc1 ; CC(C)(c1ccccc1)c1ccc(N(c2ccc(-c3ccccc3)cc2)c2ccc3c(c2)C(c2ccccc2)(c2ccccc2)c2ccccc2-3)cc1 ; CC(C)(c1ccccc1)c1ccc(N(c2ccc(-c3ccccc3)cc2)c2ccc3c(c2)C2(c4ccccc4-c4ccccc42)c2ccccc2-3)cc1]

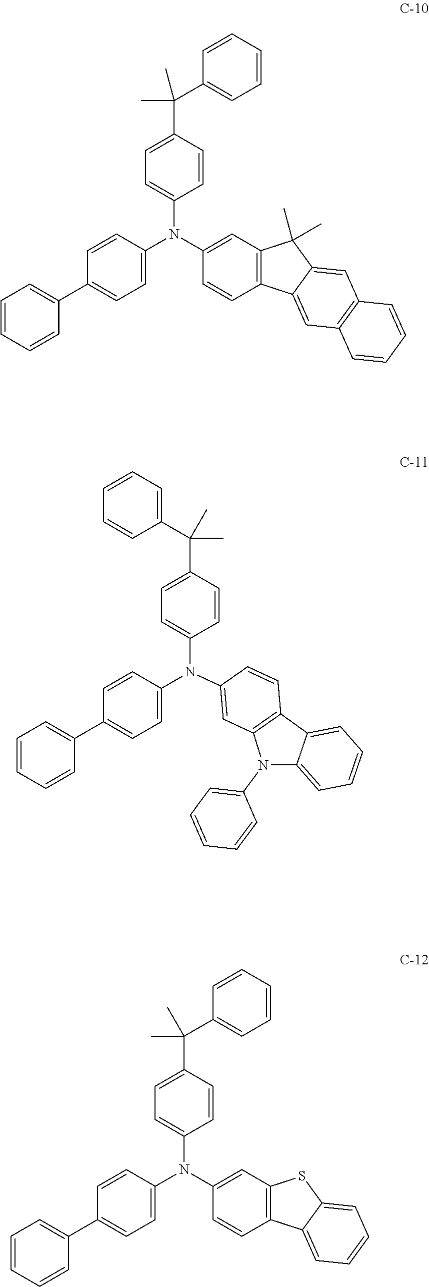 [SMILES: CC(C)(c1ccccc1)c1ccc(N(c2ccc(-c3ccccc3)cc2)c2ccc3c(c2)C(C)(C)c2cc4ccccc4cc2-3)cc1 ; CC(C)(c1ccccc1)c1ccc(N(c2ccc(-c3ccccc3)cc2)c2ccc3c4ccccc4n(-c4ccccc4)c3c2)cc1 ; CC(C)(c1ccccc1)c1ccc(N(c2ccc(-c3ccccc3)cc2)c2ccc3c(c2)sc2ccccc23)cc1]

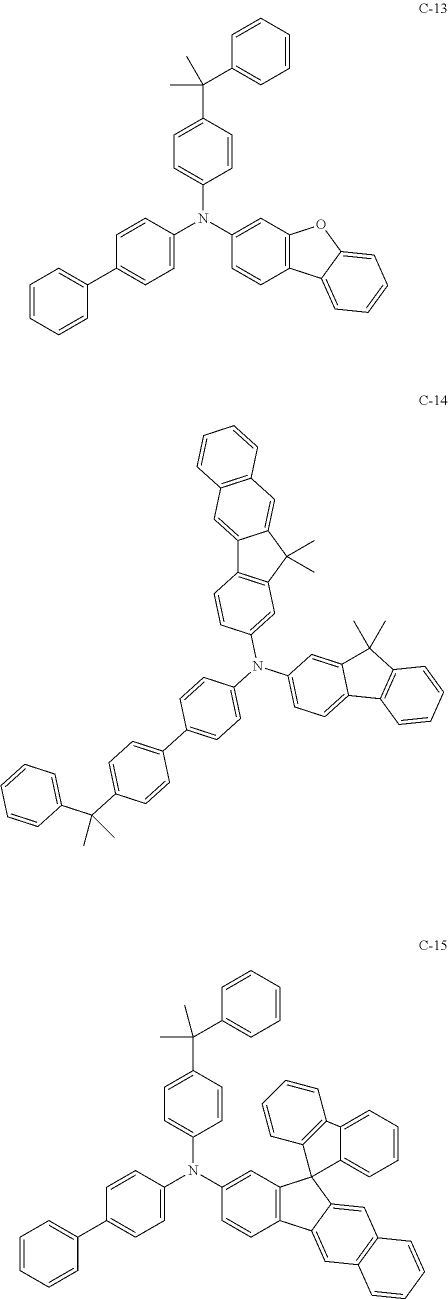 [SMILES: CC(C)(c1ccccc1)c1ccc(N(c2ccc(-c3ccccc3)cc2)c2ccc3c(c2)oc2ccccc23)cc1 ; CC(C)(c1ccccc1)c1ccc(-c2ccc(N(c3ccc4c(c3)C(C)(C)c3ccccc3-4)c3ccc4c(c3)C(C)(C)c3cc5ccccc5cc3-4)cc2)cc1 ; CC(C)(c1ccccc1)c1ccc(N(c2ccc(-c3ccccc3)cc2)c2ccc3c(c2)C2(c4ccccc4-c4ccccc42)c2cc4ccccc4cc2-3)cc1]

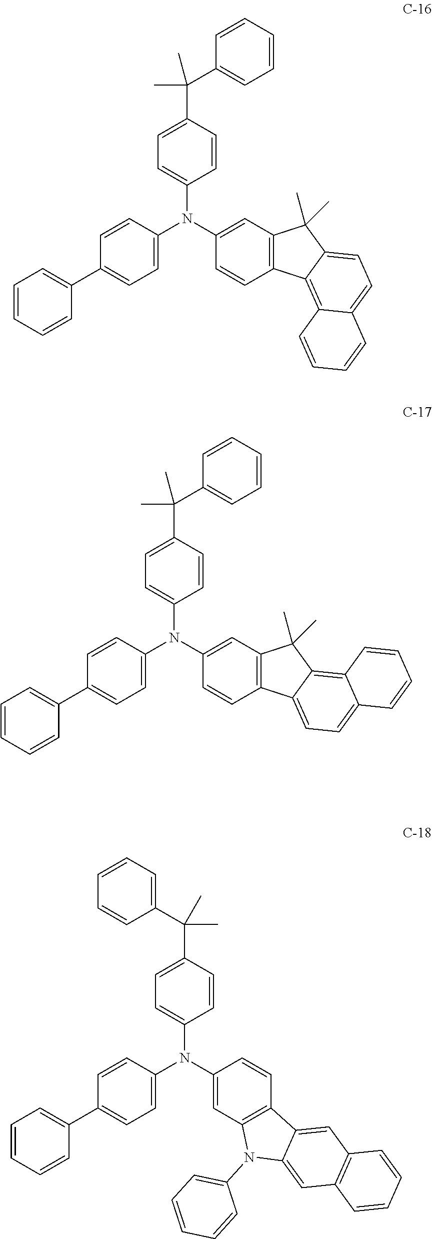 [SMILES: CC(C)(c1ccccc1)c1ccc(N(c2ccc(-c3ccccc3)cc2)c2ccc3c(c2)C(C)(C)c2ccc4ccccc4c2-3)cc1 ; CC(C)(c1ccccc1)c1ccc(N(c2ccc(-c3ccccc3)cc2)c2ccc3c(c2)C(C)(C)c2c-3ccc3ccccc23)cc1 ; CC(C)(c1ccccc1)c1ccc(N(c2ccc(-c3ccccc3)cc2)c2ccc3c4cc5ccccc5cc4n(-c4ccccc4)c3c2)cc1]

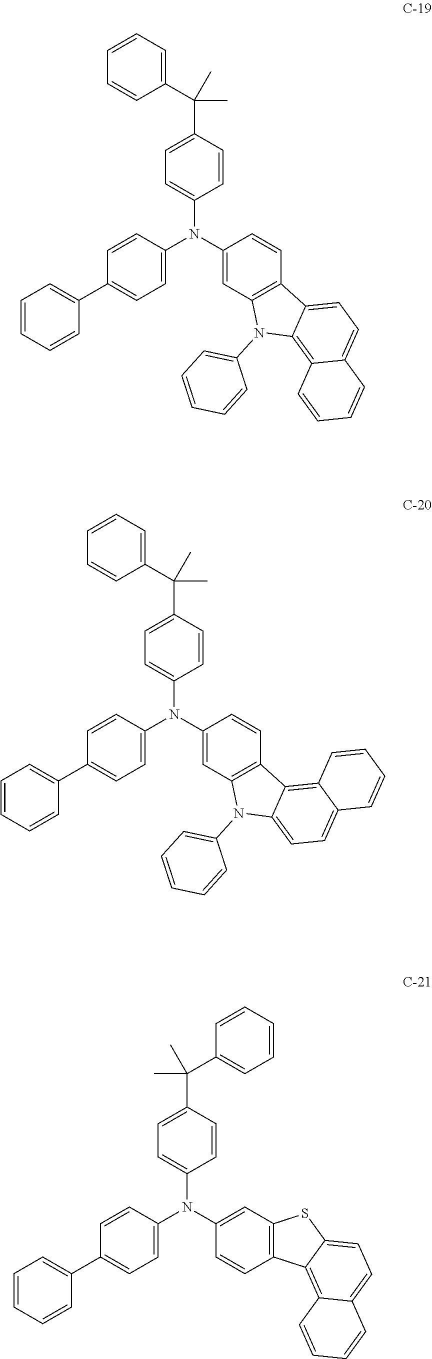 [SMILES: CC(C)(c1ccccc1)c1ccc(N(c2ccc(-c3ccccc3)cc2)c2ccc3c4ccc5ccccc5c4n(-c4ccccc4)c3c2)cc1 ; CC(C)(c1ccccc1)c1ccc(N(c2ccc(-c3ccccc3)cc2)c2ccc3c4c5ccccc5ccc4n(-c4ccccc4)c3c2)cc1 ; CC(C)(c1ccccc1)c1ccc(N(c2ccc(-c3ccccc3)cc2)c2ccc3c(c2)sc2ccc4ccccc4c23)cc1]

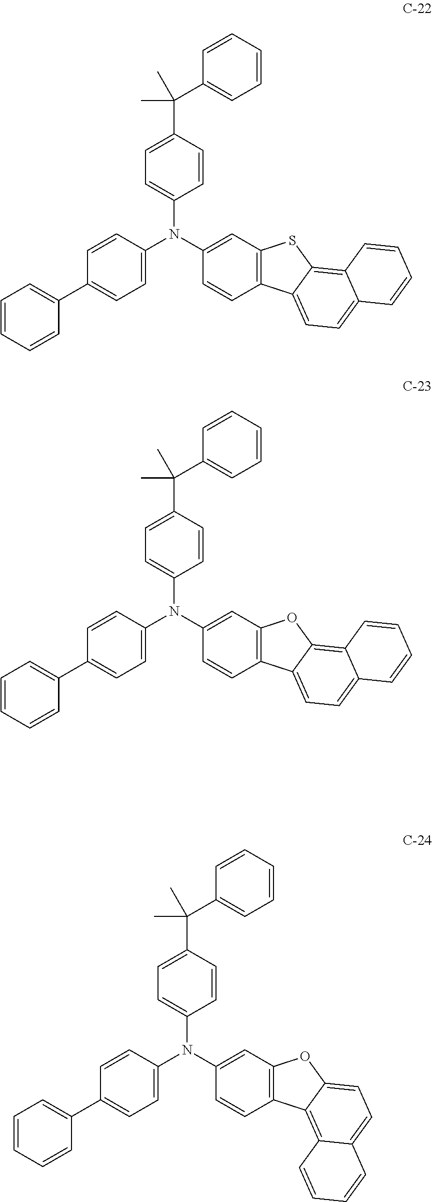 [SMILES: CC(C)(c1ccccc1)c1ccc(N(c2ccc(-c3ccccc3)cc2)c2ccc3c(c2)sc2c4ccccc4ccc32)cc1 ; CC(C)(c1ccccc1)c1ccc(N(c2ccc(-c3ccccc3)cc2)c2ccc3c(c2)oc2c4ccccc4ccc32)cc1 ; CC(C)(c1ccccc1)c1ccc(N(c2ccc(-c3ccccc3)cc2)c2ccc3c(c2)oc2ccc4ccccc4c23)cc1]

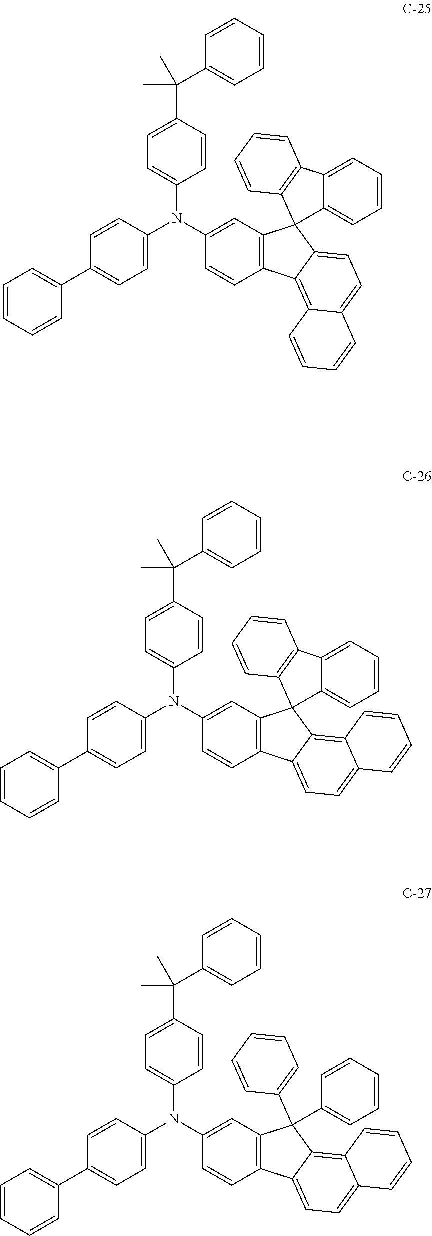 [SMILES: CC(C)(c1ccccc1)c1ccc(N(c2ccc(-c3ccccc3)cc2)c2ccc3c(c2)C2(c4ccccc4-c4ccccc42)c2ccc4ccccc4c2-3)cc1 ; CC(C)(c1ccccc1)c1ccc(N(c2ccc(-c3ccccc3)cc2)c2ccc3c(c2)C2(c4ccccc4-c4ccccc42)c2c-3ccc3ccccc23)cc1 ; CC(C)(c1ccccc1)c1ccc(N(c2ccc(-c3ccccc3)cc2)c2ccc3c(c2)C(c2ccccc2)(c2ccccc2)c2c-3ccc3ccccc23)cc1]

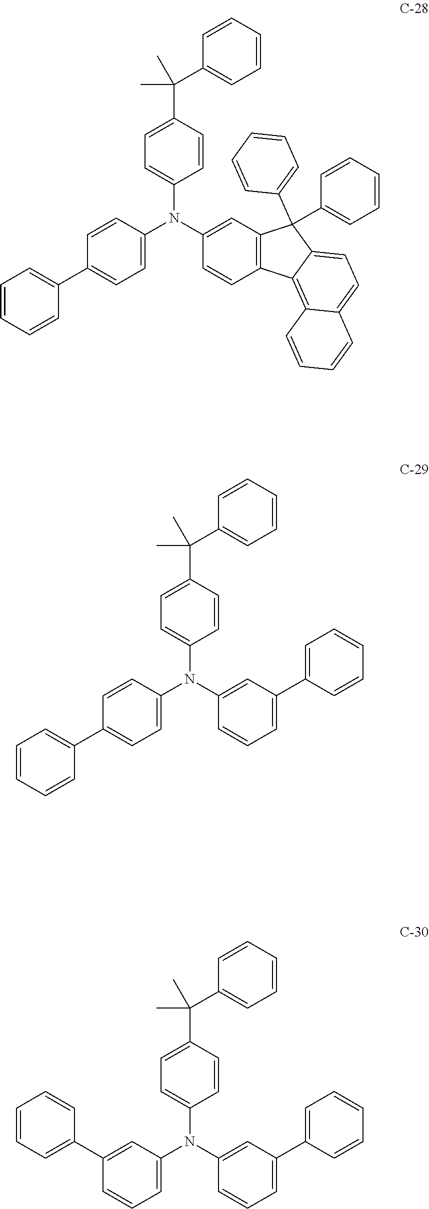 [SMILES: CC(C)(c1ccccc1)c1ccc(N(c2ccc(-c3ccccc3)cc2)c2ccc3c(c2)C(c2ccccc2)(c2ccccc2)c2ccc4ccccc4c2-3)cc1 ; CC(C)(c1ccccc1)c1ccc(N(c2ccc(-c3ccccc3)cc2)c2cccc(-c3ccccc3)c2)cc1 ; CC(C)(c1ccccc1)c1ccc(N(c2cccc(-c3ccccc3)c2)c2cccc(-c3ccccc3)c2)cc1]

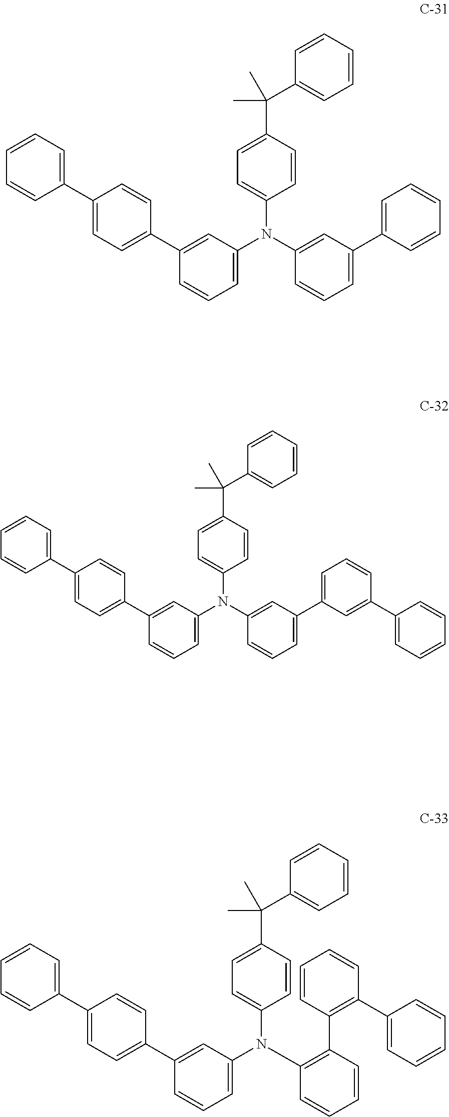 [SMILES: CC(C)(c1ccccc1)c1ccc(N(c2cccc(-c3ccccc3)c2)c2cccc(-c3ccc(-c4ccccc4)cc3)c2)cc1 ; CC(C)(c1ccccc1)c1ccc(N(c2cccc(-c3ccc(-c4ccccc4)cc3)c2)c2cccc(-c3cccc(-c4ccccc4)c3)c2)cc1 ; CC(C)(c1ccccc1)c1ccc(N(c2cccc(-c3ccc(-c4ccccc4)cc3)c2)c2ccccc2-c2ccccc2-c2ccccc2)cc1]

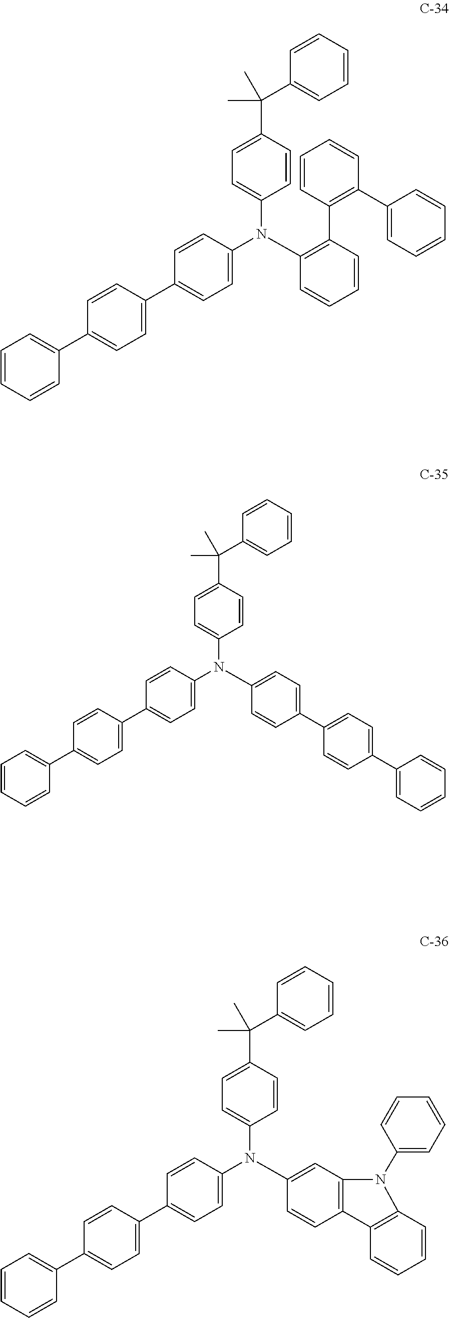 [SMILES: CC(C)(c1ccccc1)c1ccc(N(c2ccc(-c3ccc(-c4ccccc4)cc3)cc2)c2ccccc2-c2ccccc2-c2ccccc2)cc1 ; CC(C)(c1ccccc1)c1ccc(N(c2ccc(-c3ccc(-c4ccccc4)cc3)cc2)c2ccc(-c3ccc(-c4ccccc4)cc3)cc2)cc1 ; CC(C)(c1ccccc1)c1ccc(N(c2ccc(-c3ccc(-c4ccccc4)cc3)cc2)c2ccc3c4ccccc4n(-c4ccccc4)c3c2)cc1]

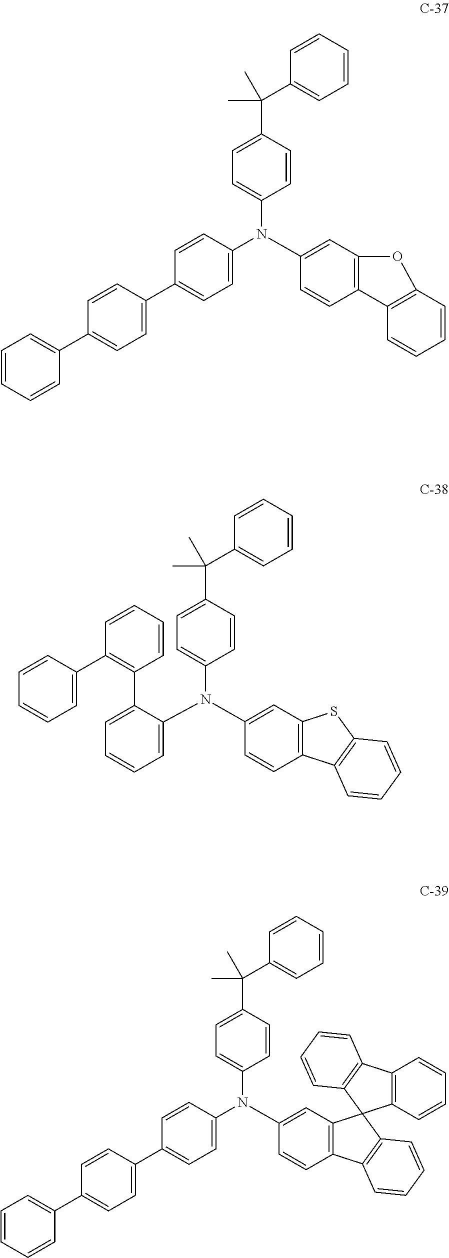 [SMILES: CC(C)(c1ccccc1)c1ccc(N(c2ccc(-c3ccc(-c4ccccc4)cc3)cc2)c2ccc3c(c2)oc2ccccc23)cc1 ; CC(C)(c1ccccc1)c1ccc(N(c2ccc3c(c2)sc2ccccc23)c2ccccc2-c2ccccc2-c2ccccc2)cc1 ; CC(C)(c1ccccc1)c1ccc(N(c2ccc(-c3ccc(-c4ccccc4)cc3)cc2)c2ccc3c(c2)C2(c4ccccc4-c4ccccc42)c2ccccc2-3)cc1]

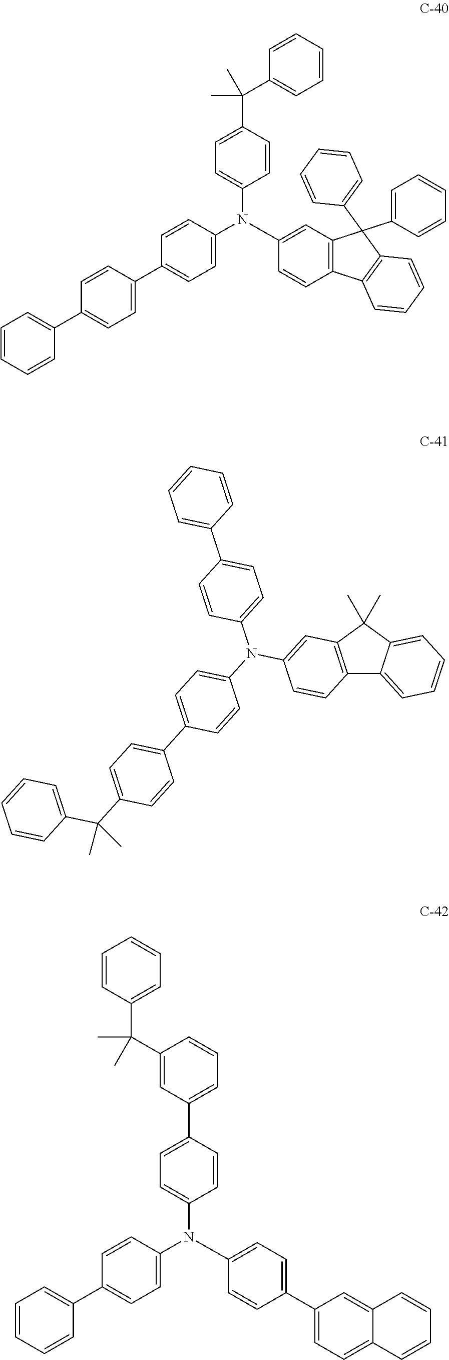 [SMILES: CC(C)(c1ccccc1)c1ccc(N(c2ccc(-c3ccc(-c4ccccc4)cc3)cc2)c2ccc3c(c2)C(c2ccccc2)(c2ccccc2)c2ccccc2-3)cc1 ; CC(C)(c1ccccc1)c1ccc(-c2ccc(N(c3ccc(-c4ccccc4)cc3)c3ccc4c(c3)C(C)(C)c3ccccc3-4)cc2)cc1 ; CC(C)(c1ccccc1)c1cccc(-c2ccc(N(c3ccc(-c4ccccc4)cc3)c3ccc(-c4ccc5ccccc5c4)cc3)cc2)c1]

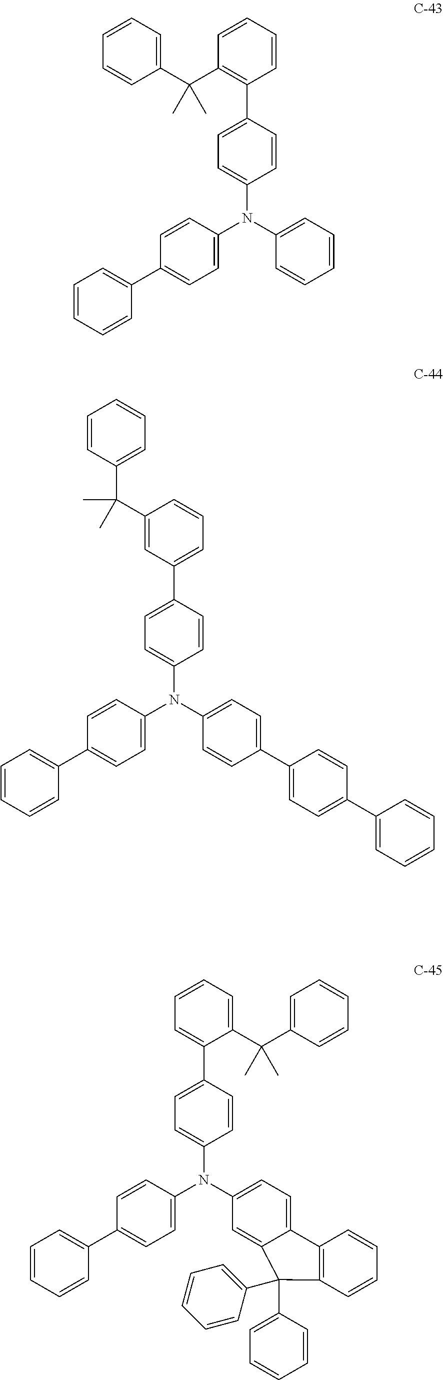 [SMILES: CC(C)(c1ccccc1)c1ccccc1-c1ccc(N(c2ccccc2)c2ccc(-c3ccccc3)cc2)cc1 ; CC(C)(c1ccccc1)c1cccc(-c2ccc(N(c3ccc(-c4ccccc4)cc3)c3ccc(-c4ccc(-c5ccccc5)cc4)cc3)cc2)c1 ; CC(C)(c1ccccc1)c1ccccc1-c1ccc(N(c2ccc(-c3ccccc3)cc2)c2ccc3c(c2)C(c2ccccc2)(c2ccccc2)c2ccccc2-3)cc1]

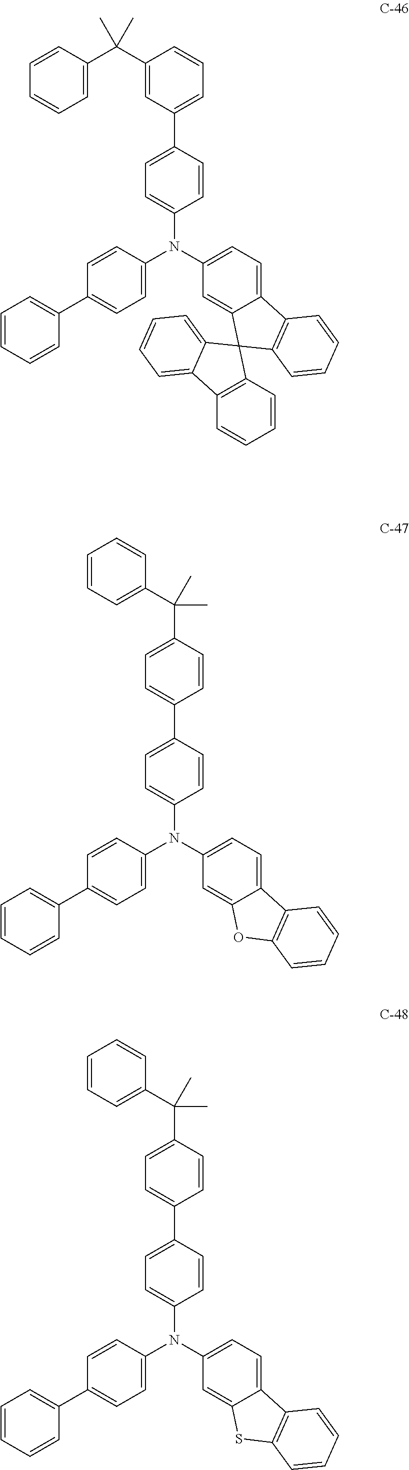 [SMILES: CC(C)(c1ccccc1)c1cccc(-c2ccc(N(c3ccc(-c4ccccc4)cc3)c3ccc4c(c3)C3(c5ccccc5-c5ccccc53)c3ccccc3-4)cc2)c1 ; CC(C)(c1ccccc1)c1ccc(-c2ccc(N(c3ccc(-c4ccccc4)cc3)c3ccc4c(c3)oc3ccccc34)cc2)cc1 ; CC(C)(c1ccccc1)c1ccc(-c2ccc(N(c3ccc(-c4ccccc4)cc3)c3ccc4c(c3)sc3ccccc34)cc2)cc1]

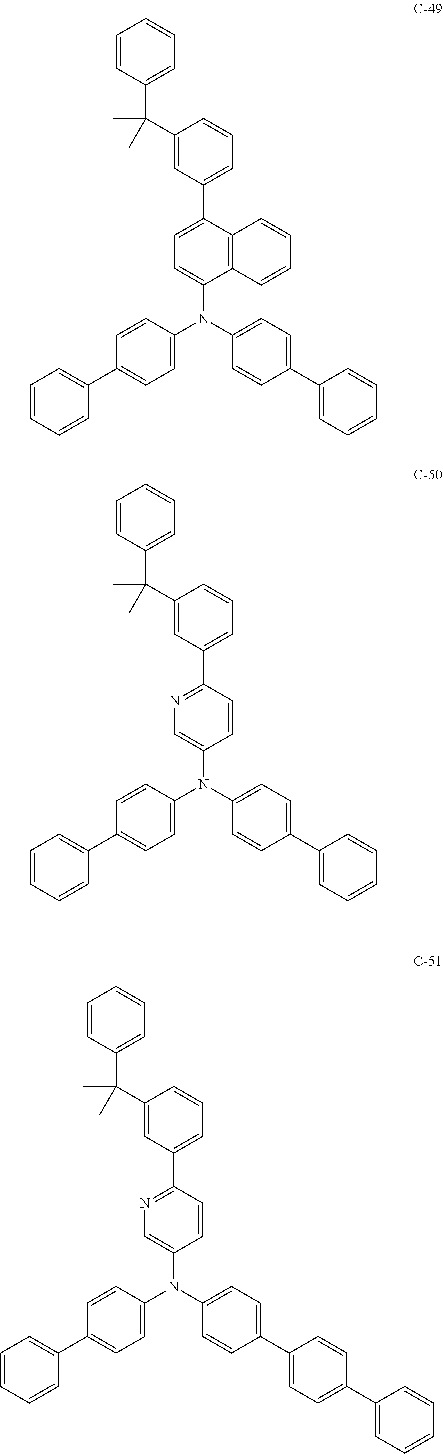 [SMILES: CC(C)(c1ccccc1)c1cccc(-c2ccc(N(c3ccc(-c4ccccc4)cc3)c3ccc(-c4ccccc4)cc3)c3ccccc23)c1 ; CC(C)(c1ccccc1)c1cccc(-c2ccc(N(c3ccc(-c4ccccc4)cc3)c3ccc(-c4ccccc4)cc3)cn2)c1 ; CC(C)(c1ccccc1)c1cccc(-c2ccc(N(c3ccc(-c4ccccc4)cc3)c3ccc(-c4ccc(-c5ccccc5)cc4)cc3)cn2)c1]

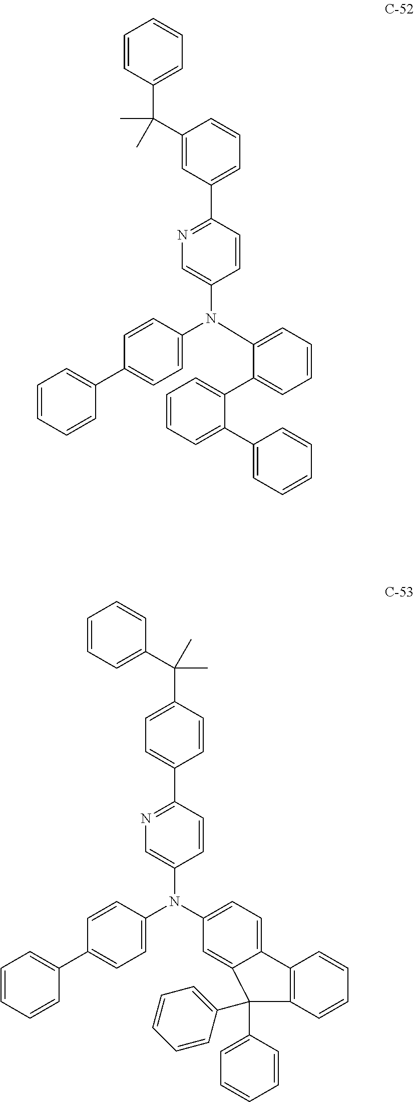 [SMILES: CC(C)(c1ccccc1)c1cccc(-c2ccc(N(c3ccc(-c4ccccc4)cc3)c3ccccc3-c3ccccc3-c3ccccc3)cn2)c1 ; CC(C)(c1ccccc1)c1ccc(-c2ccc(N(c3ccc(-c4ccccc4)cc3)c3ccc4c(c3)C(c3ccccc3)(c3ccccc3)c3ccccc3-4)cn2)cc1]

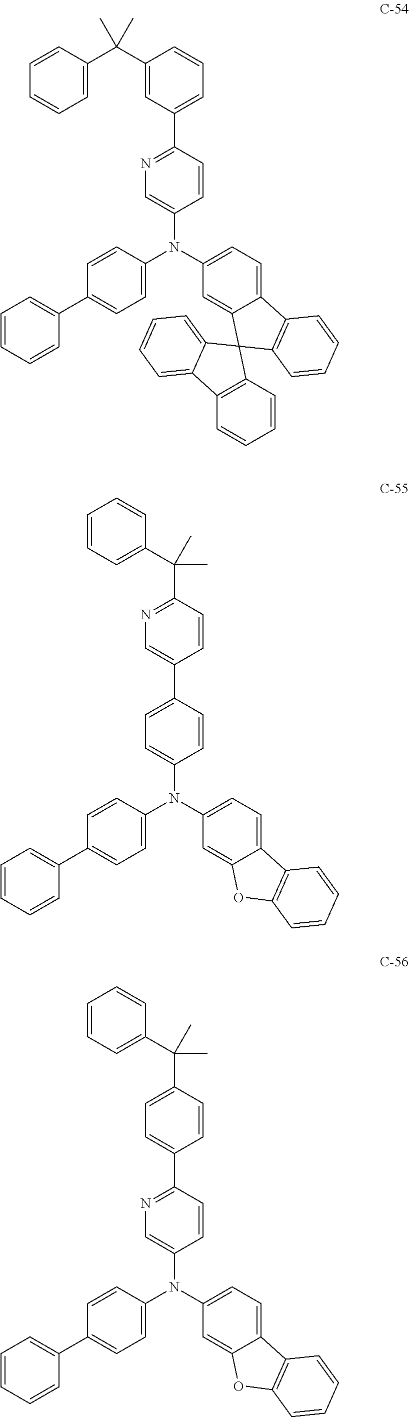 [SMILES: CC(C)(c1ccccc1)c1cccc(-c2ccc(N(c3ccc(-c4ccccc4)cc3)c3ccc4c(c3)C3(c5ccccc5-c5ccccc53)c3ccccc3-4)cn2)c1 ; CC(C)(c1ccccc1)c1ccc(-c2ccc(N(c3ccc(-c4ccccc4)cc3)c3ccc4c(c3)oc3ccccc34)cc2)cn1 ; CC(C)(c1ccccc1)c1ccc(-c2ccc(N(c3ccc(-c4ccccc4)cc3)c3ccc4c(c3)oc3ccccc34)cn2)cc1]

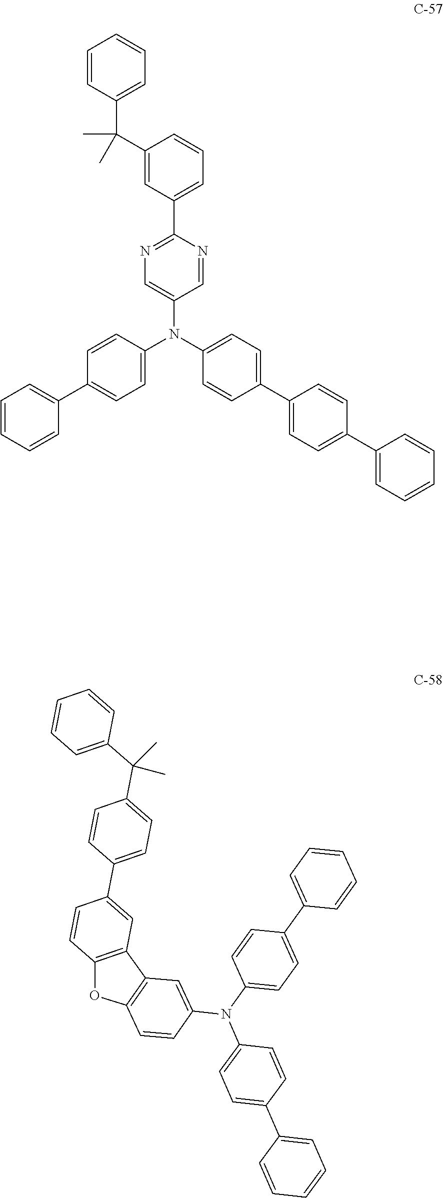 [SMILES: CC(C)(c1ccccc1)c1cccc(-c2ncc(N(c3ccc(-c4ccccc4)cc3)c3ccc(-c4ccc(-c5ccccc5)cc4)cc3)cn2)c1 ; CC(C)(c1ccccc1)c1ccc(-c2ccc3oc4ccc(N(c5ccc(-c6ccccc6)cc5)c5ccc(-c6ccccc6)cc5)cc4c3c2)cc1]

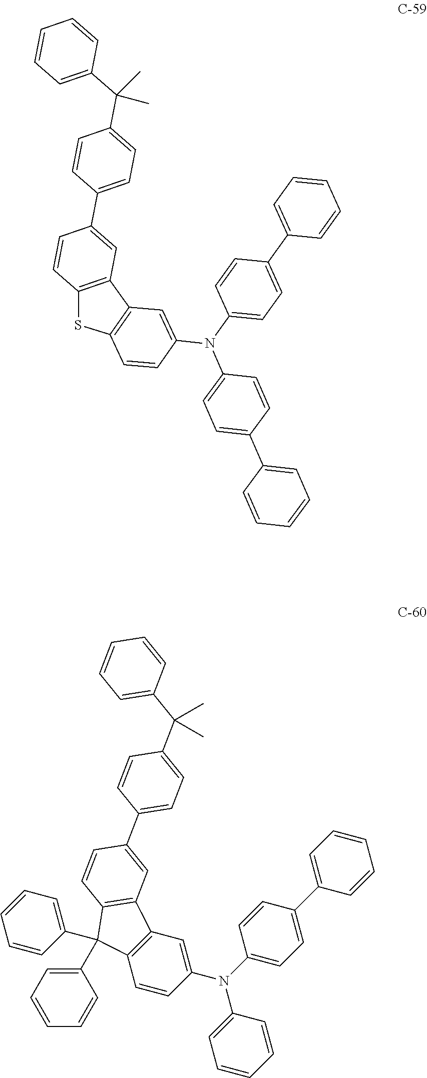 [SMILES: CC(C)(c1ccccc1)c1ccc(-c2ccc3sc4ccc(N(c5ccc(-c6ccccc6)cc5)c5ccc(-c6ccccc6)cc5)cc4c3c2)cc1 ; CC(C)(c1ccccc1)c1ccc(-c2ccc3c(c2)-c2cc(N(c4ccccc4)c4ccc(-c5ccccc5)cc4)ccc2C3(c2ccccc2)c2ccccc2)cc1]

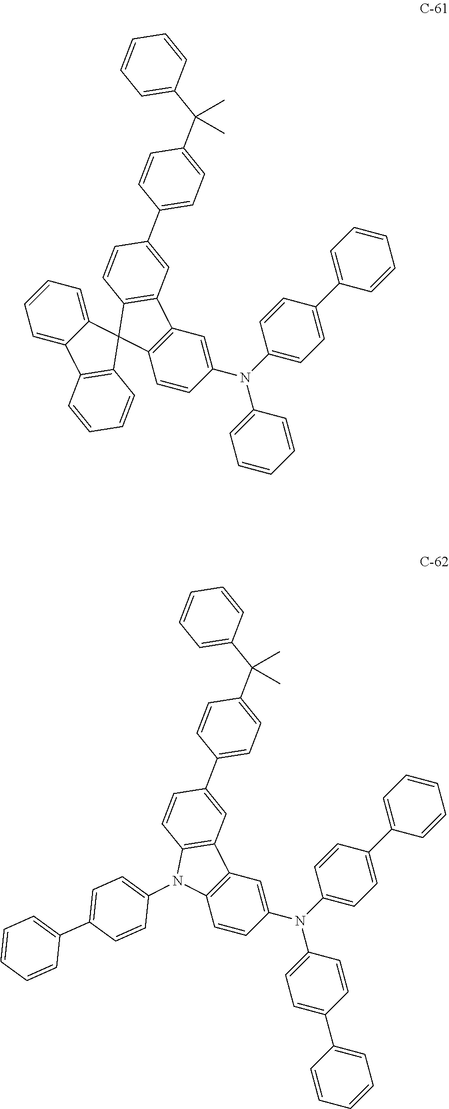 [SMILES: CC(C)(c1ccccc1)c1ccc(-c2ccc3c(c2)-c2cc(N(c4ccccc4)c4ccc(-c5ccccc5)cc4)ccc2C32c3ccccc3-c3ccccc32)cc1 ; CC(C)(c1ccccc1)c1ccc(-c2ccc3c(c2)c2cc(N(c4ccc(-c5ccccc5)cc4)c4ccc(-c5ccccc5)cc4)ccc2n3-c2ccc(-c3ccccc3)cc2)cc1]

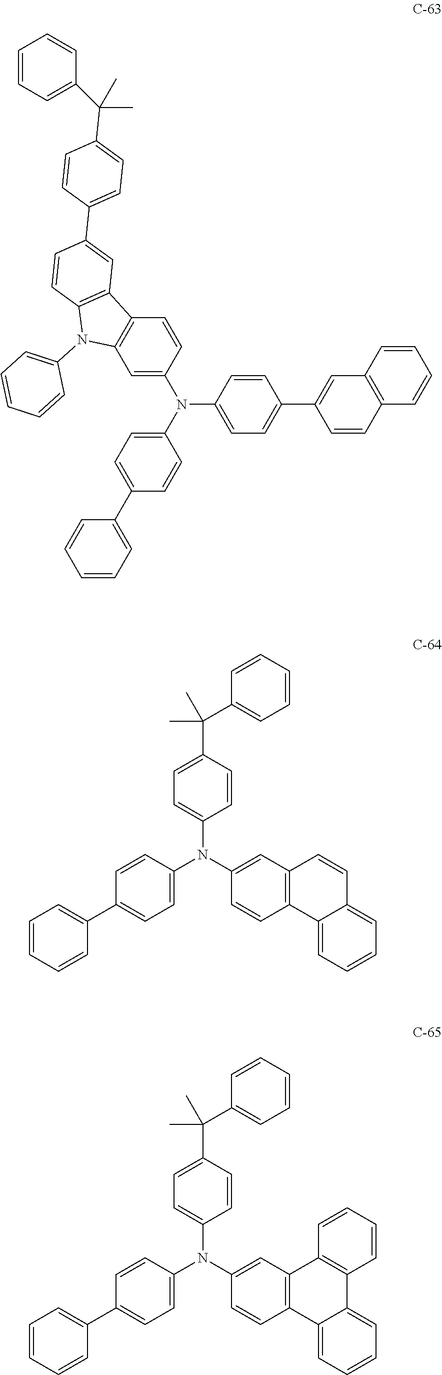 [SMILES: CC(C)(c1ccccc1)c1ccc(-c2ccc3c(c2)c2ccc(N(c4ccc(-c5ccccc5)cc4)c4ccc(-c5ccc6ccccc6c5)cc4)cc2n3-c2ccccc2)cc1 ; CC(C)(c1ccccc1)c1ccc(N(c2ccc(-c3ccccc3)cc2)c2ccc3c(ccc4ccccc43)c2)cc1 ; CC(C)(c1ccccc1)c1ccc(N(c2ccc(-c3ccccc3)cc2)c2ccc3c4ccccc4c4ccccc4c3c2)cc1]

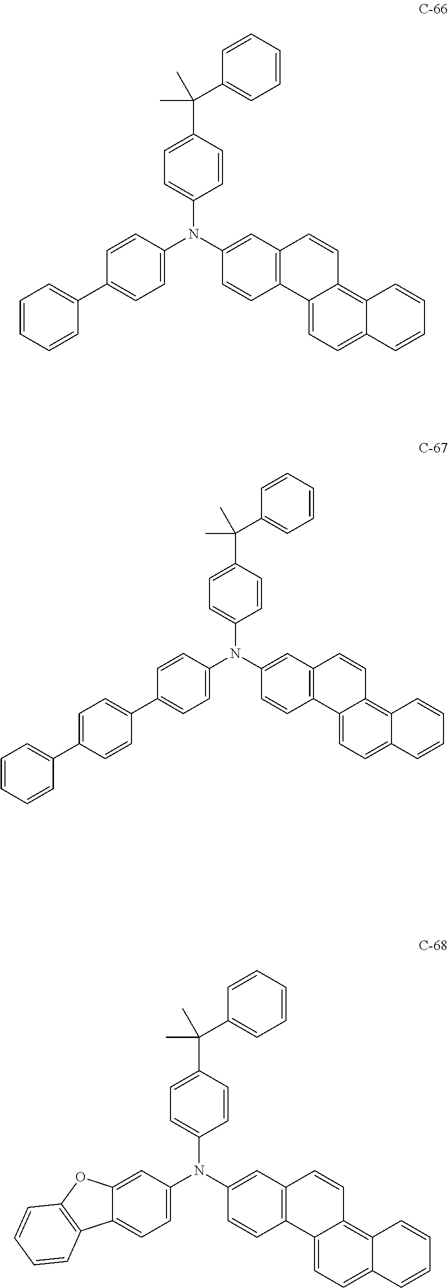 [SMILES: CC(C)(c1ccccc1)c1ccc(N(c2ccc(-c3ccccc3)cc2)c2ccc3c(ccc4c5ccccc5ccc34)c2)cc1 ; CC(C)(c1ccccc1)c1ccc(N(c2ccc(-c3ccc(-c4ccccc4)cc3)cc2)c2ccc3c(ccc4c5ccccc5ccc34)c2)cc1 ; CC(C)(c1ccccc1)c1ccc(N(c2ccc3c(ccc4c5ccccc5ccc34)c2)c2ccc3c(c2)oc2ccccc23)cc1]

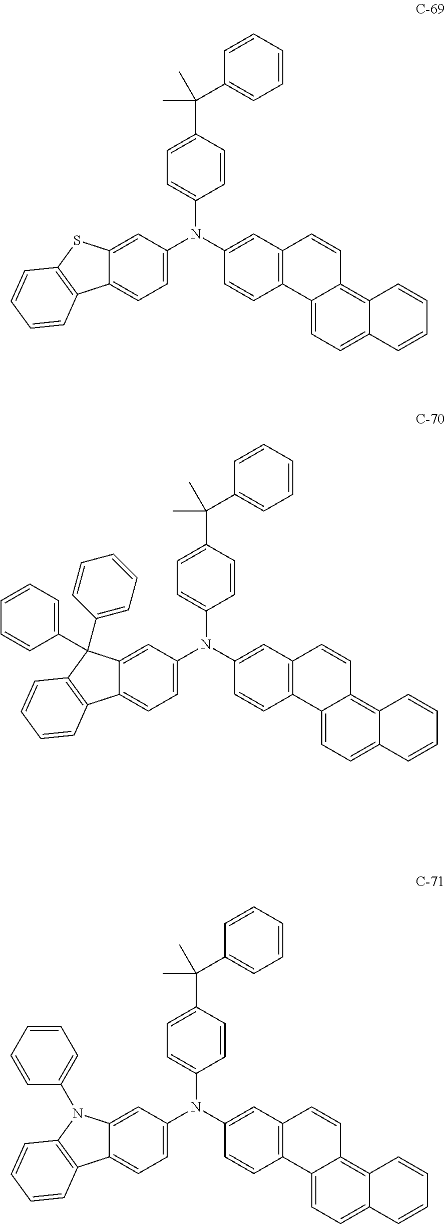 [SMILES: CC(C)(c1ccccc1)c1ccc(N(c2ccc3c(ccc4c5ccccc5ccc34)c2)c2ccc3c(c2)sc2ccccc23)cc1 ; CC(C)(c1ccccc1)c1ccc(N(c2ccc3c(c2)C(c2ccccc2)(c2ccccc2)c2ccccc2-3)c2ccc3c(ccc4c5ccccc5ccc34)c2)cc1 ; CC(C)(c1ccccc1)c1ccc(N(c2ccc3c(ccc4c5ccccc5ccc34)c2)c2ccc3c4ccccc4n(-c4ccccc4)c3c2)cc1]

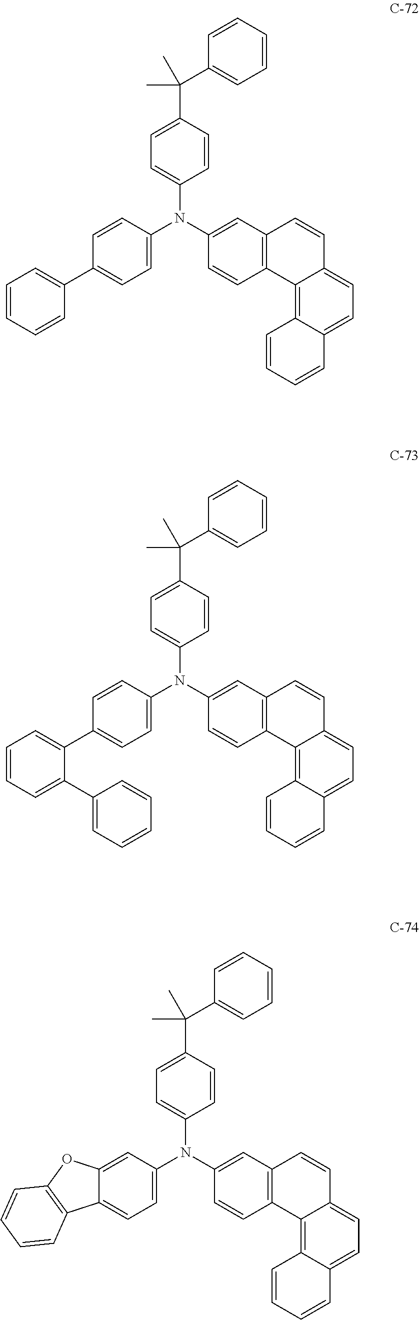 [SMILES: CC(C)(c1ccccc1)c1ccc(N(c2ccc(-c3ccccc3)cc2)c2ccc3c(ccc4ccc5ccccc5c43)c2)cc1 ; CC(C)(c1ccccc1)c1ccc(N(c2ccc(-c3ccccc3-c3ccccc3)cc2)c2ccc3c(ccc4ccc5ccccc5c43)c2)cc1 ; CC(C)(c1ccccc1)c1ccc(N(c2ccc3c(ccc4ccc5ccccc5c43)c2)c2ccc3c(c2)oc2ccccc23)cc1]

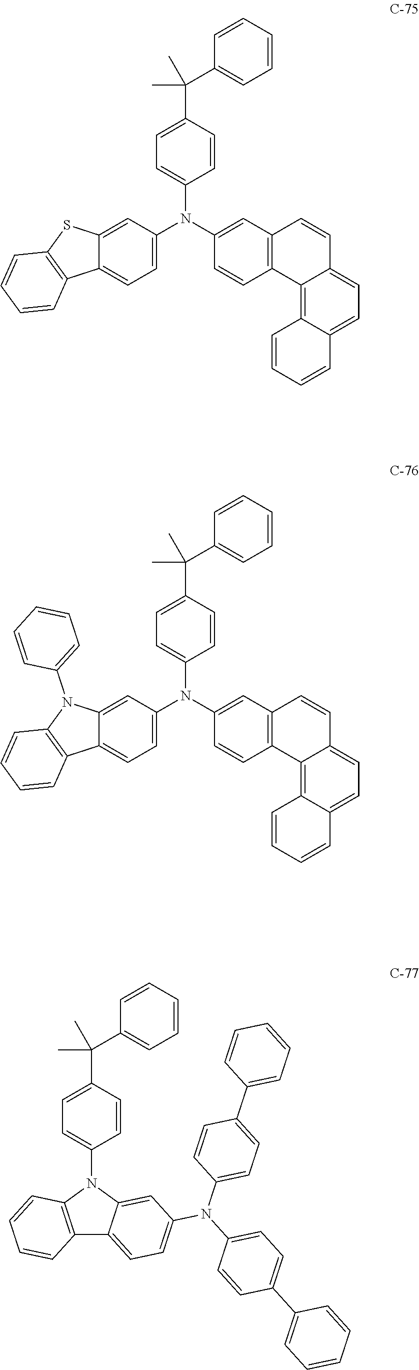 [SMILES: CC(C)(c1ccccc1)c1ccc(N(c2ccc3c(ccc4ccc5ccccc5c43)c2)c2ccc3c(c2)sc2ccccc23)cc1 ; CC(C)(c1ccccc1)c1ccc(N(c2ccc3c(ccc4ccc5ccccc5c43)c2)c2ccc3c4ccccc4n(-c4ccccc4)c3c2)cc1 ; CC(C)(c1ccccc1)c1ccc(-n2c3ccccc3c3ccc(N(c4ccc(-c5ccccc5)cc4)c4ccc(-c5ccccc5)cc4)cc32)cc1]

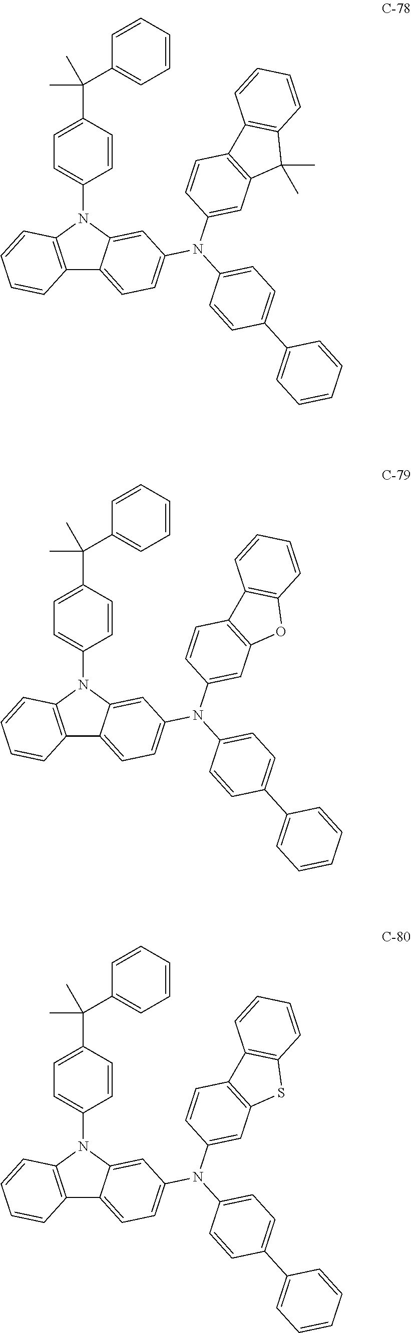 [SMILES: CC(C)(c1ccccc1)c1ccc(-n2c3ccccc3c3ccc(N(c4ccc(-c5ccccc5)cc4)c4ccc5c(c4)C(C)(C)c4ccccc4-5)cc32)cc1 ; CC(C)(c1ccccc1)c1ccc(-n2c3ccccc3c3ccc(N(c4ccc(-c5ccccc5)cc4)c4ccc5c(c4)oc4ccccc45)cc32)cc1 ; CC(C)(c1ccccc1)c1ccc(-n2c3ccccc3c3ccc(N(c4ccc(-c5ccccc5)cc4)c4ccc5c(c4)sc4ccccc45)cc32)cc1]

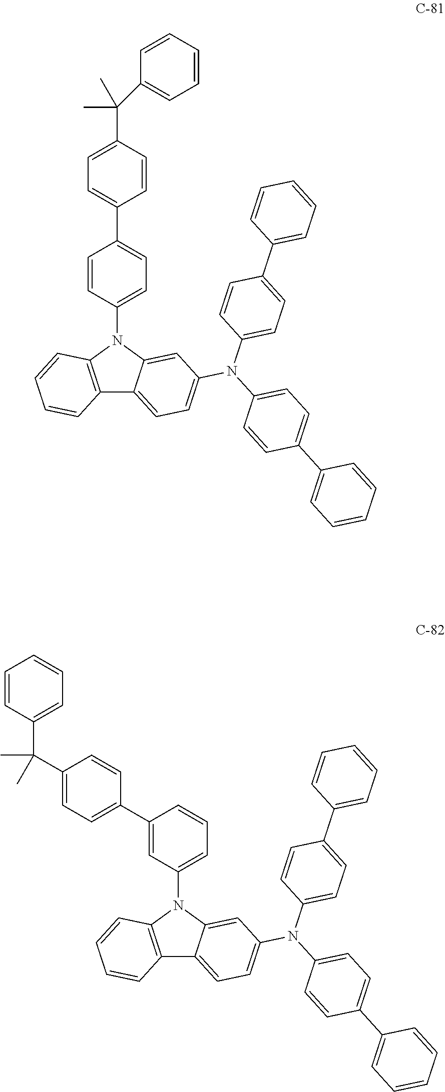 [SMILES: CC(C)(c1ccccc1)c1ccc(-c2ccc(-n3c4ccccc4c4ccc(N(c5ccc(-c6ccccc6)cc5)c5ccc(-c6ccccc6)cc5)cc43)cc2)cc1 ; CC(C)(c1ccccc1)c1ccc(-c2cccc(-n3c4ccccc4c4ccc(N(c5ccc(-c6ccccc6)cc5)c5ccc(-c6ccccc6)cc5)cc43)c2)cc1]

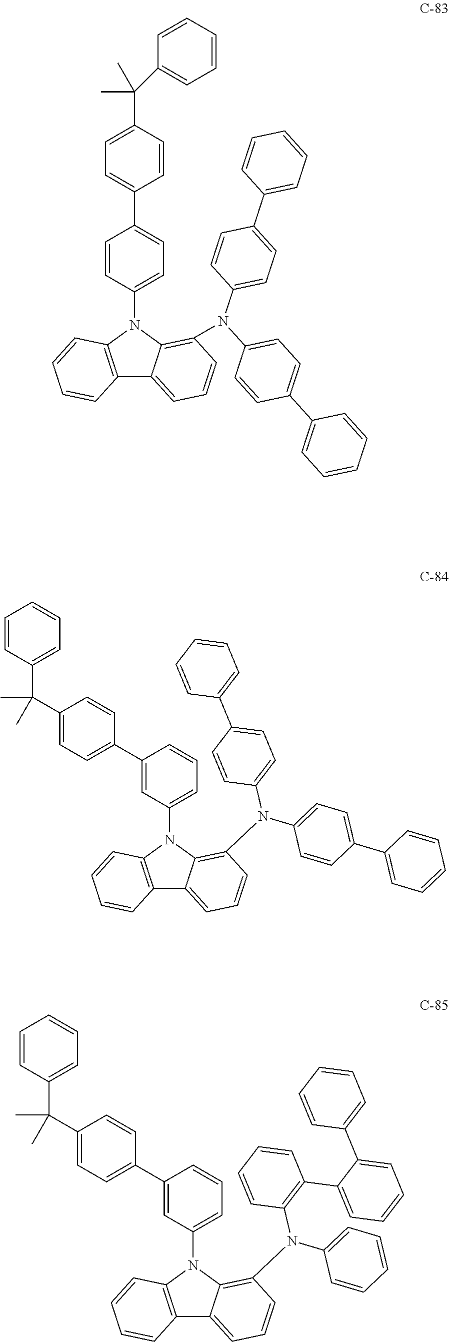 [SMILES: CC(C)(c1ccccc1)c1ccc(-c2ccc(-n3c4ccccc4c4cccc(N(c5ccc(-c6ccccc6)cc5)c5ccc(-c6ccccc6)cc5)c43)cc2)cc1 ; CC(C)(c1ccccc1)c1ccc(-c2cccc(-n3c4ccccc4c4cccc(N(c5ccc(-c6ccccc6)cc5)c5ccc(-c6ccccc6)cc5)c43)c2)cc1 ; CC(C)(c1ccccc1)c1ccc(-c2cccc(-n3c4ccccc4c4cccc(N(c5ccccc5)c5ccccc5-c5ccccc5-c5ccccc5)c43)c2)cc1]

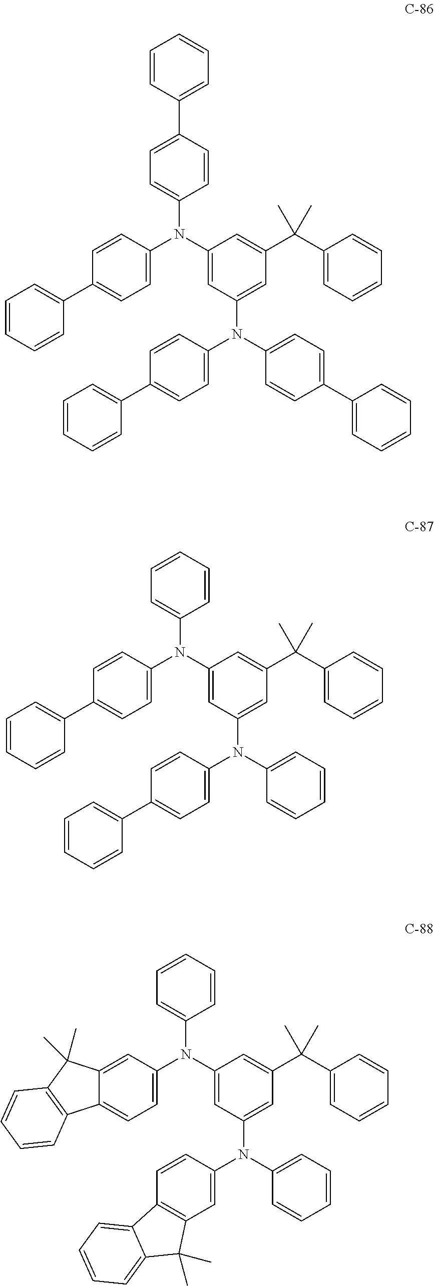 [SMILES: CC(C)(c1ccccc1)c1cc(N(c2ccc(-c3ccccc3)cc2)c2ccc(-c3ccccc3)cc2)cc(N(c2ccc(-c3ccccc3)cc2)c2ccc(-c3ccccc3)cc2)c1 ; CC(C)(c1ccccc1)c1cc(N(c2ccccc2)c2ccc(-c3ccccc3)cc2)cc(N(c2ccccc2)c2ccc(-c3ccccc3)cc2)c1 ; CC(C)(c1ccccc1)c1cc(N(c2ccccc2)c2ccc3c(c2)C(C)(C)c2ccccc2-3)cc(N(c2ccccc2)c2ccc3c(c2)C(C)(C)c2ccccc2-3)c1]

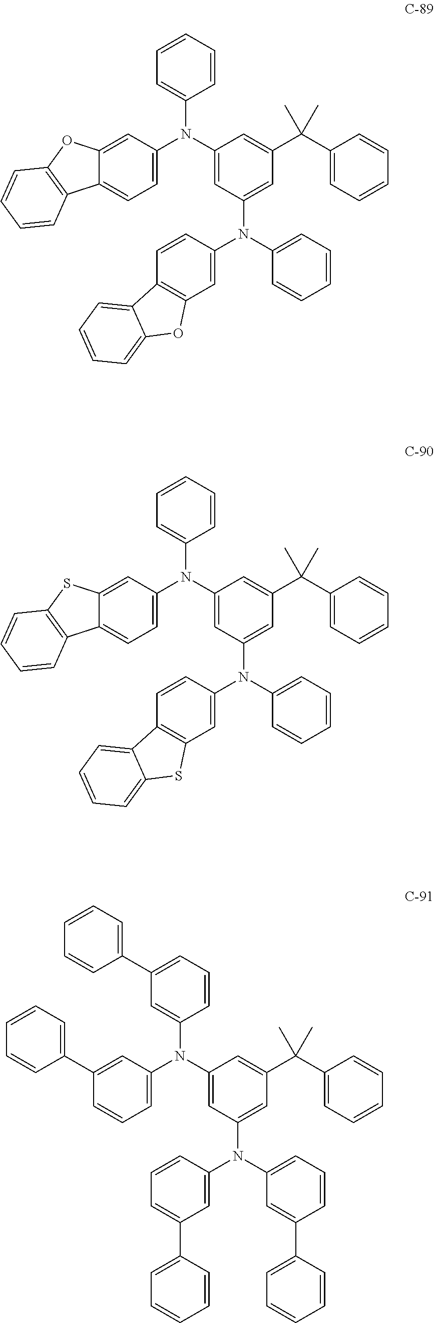 [SMILES: CC(C)(c1ccccc1)c1cc(N(c2ccccc2)c2ccc3c(c2)oc2ccccc23)cc(N(c2ccccc2)c2ccc3c(c2)oc2ccccc23)c1 ; CC(C)(c1ccccc1)c1cc(N(c2ccccc2)c2ccc3c(c2)sc2ccccc23)cc(N(c2ccccc2)c2ccc3c(c2)sc2ccccc23)c1 ; CC(C)(c1ccccc1)c1cc(N(c2cccc(-c3ccccc3)c2)c2cccc(-c3ccccc3)c2)cc(N(c2cccc(-c3ccccc3)c2)c2cccc(-c3ccccc3)c2)c1]

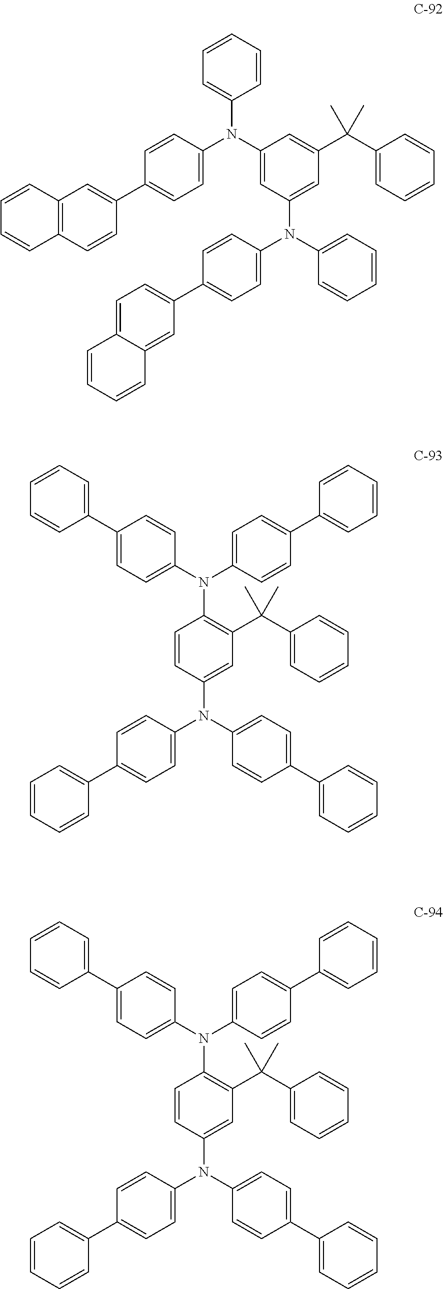 [SMILES: CC(C)(c1ccccc1)c1cc(N(c2ccccc2)c2ccc(-c3ccc4ccccc4c3)cc2)cc(N(c2ccccc2)c2ccc(-c3ccc4ccccc4c3)cc2)c1 ; CC(C)(c1ccccc1)c1cc(N(c2ccc(-c3ccccc3)cc2)c2ccc(-c3ccccc3)cc2)ccc1N(c1ccc(-c2ccccc2)cc1)c1ccc(-c2ccccc2)cc1 ; CC(C)(c1ccccc1)c1cc(N(c2ccc(-c3ccccc3)cc2)c2ccc(-c3ccccc3)cc2)ccc1N(c1ccc(-c2ccccc2)cc1)c1ccc(-c2ccccc2)cc1]

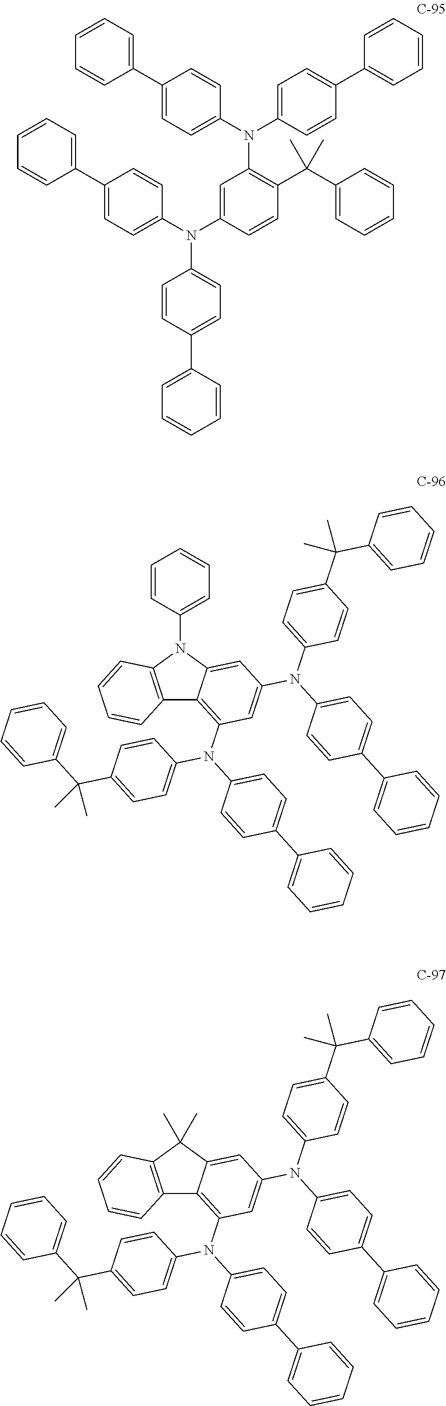 [SMILES: CC(C)(c1ccccc1)c1ccc(N(c2ccc(-c3ccccc3)cc2)c2ccc(-c3ccccc3)cc2)cc1N(c1ccc(-c2ccccc2)cc1)c1ccc(-c2ccccc2)cc1 ; CC(C)(c1ccccc1)c1ccc(N(c2ccc(-c3ccccc3)cc2)c2cc(N(c3ccc(-c4ccccc4)cc3)c3ccc(C(C)(C)c4ccccc4)cc3)c3c4ccccc4n(-c4ccccc4)c3c2)cc1 ; CC(C)(c1ccccc1)c1ccc(N(c2ccc(-c3ccccc3)cc2)c2cc(N(c3ccc(-c4ccccc4)cc3)c3ccc(C(C)(C)c4ccccc4)cc3)c3c(c2)C(C)(C)c2ccccc2-3)cc1]

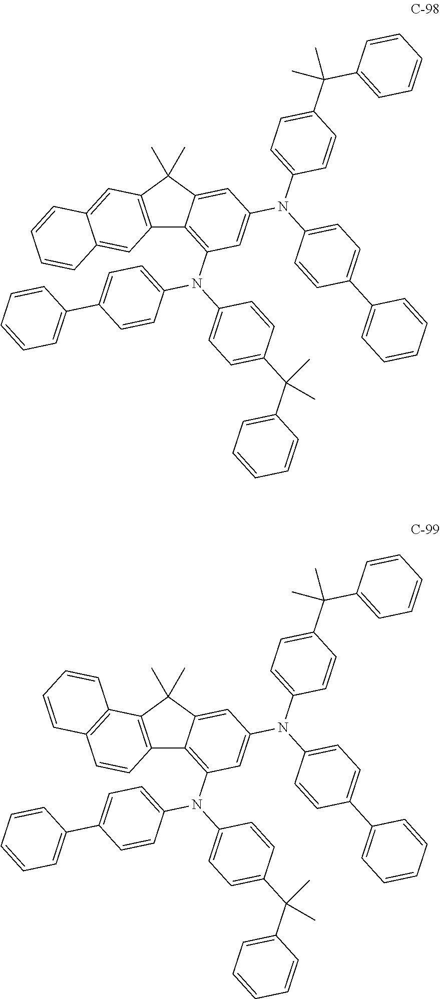 [SMILES: CC(C)(c1ccccc1)c1ccc(N(c2ccc(-c3ccccc3)cc2)c2cc(N(c3ccc(-c4ccccc4)cc3)c3ccc(C(C)(C)c4ccccc4)cc3)c3c(c2)C(C)(C)c2cc4ccccc4cc2-3)cc1 ; CC(C)(c1ccccc1)c1ccc(N(c2ccc(-c3ccccc3)cc2)c2cc(N(c3ccc(-c4ccccc4)cc3)c3ccc(C(C)(C)c4ccccc4)cc3)c3c(c2)C(C)(C)c2c-3ccc3ccccc23)cc1]

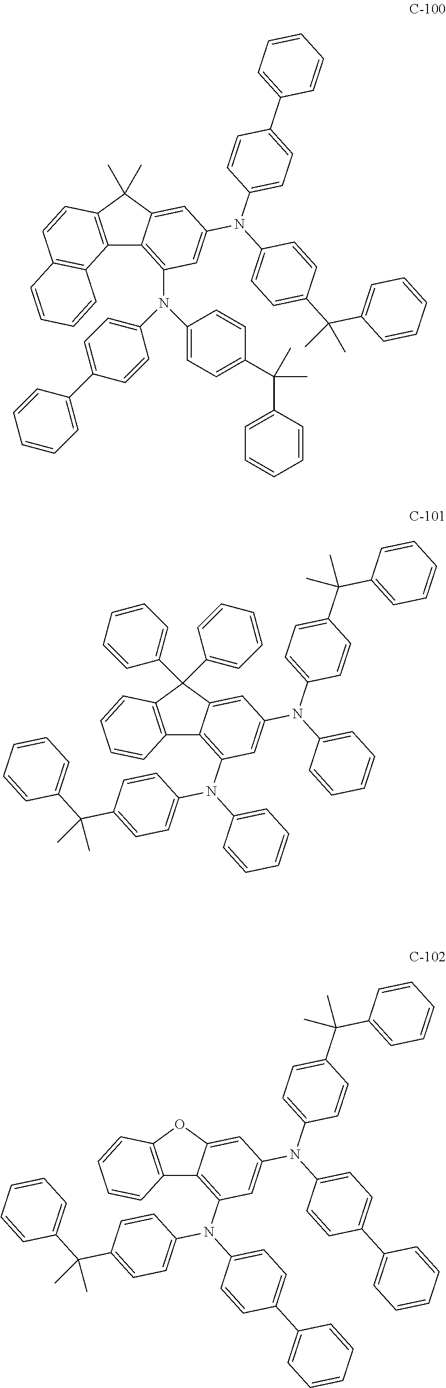 [SMILES: CC(C)(c1ccccc1)c1ccc(N(c2ccc(-c3ccccc3)cc2)c2cc(N(c3ccc(-c4ccccc4)cc3)c3ccc(C(C)(C)c4ccccc4)cc3)c3c(c2)C(C)(C)c2ccc4ccccc4c2-3)cc1 ; CC(C)(c1ccccc1)c1ccc(N(c2ccccc2)c2cc(N(c3ccccc3)c3ccc(C(C)(C)c4ccccc4)cc3)c3c(c2)C(c2ccccc2)(c2ccccc2)c2ccccc2-3)cc1 ; CC(C)(c1ccccc1)c1ccc(N(c2ccc(-c3ccccc3)cc2)c2cc(N(c3ccc(-c4ccccc4)cc3)c3ccc(C(C)(C)c4ccccc4)cc3)c3c(c2)oc2ccccc23)cc1]

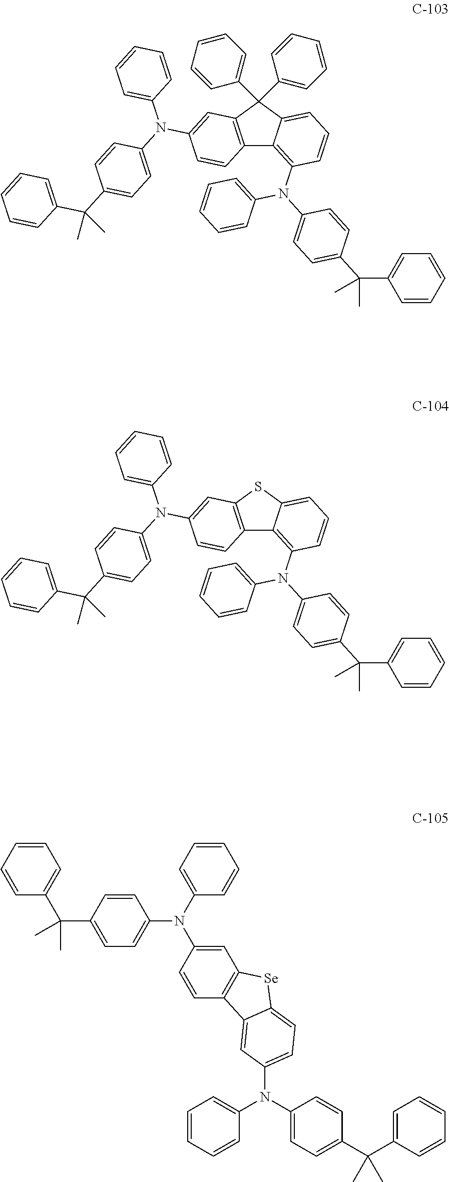 [SMILES: CC(C)(c1ccccc1)c1ccc(N(c2ccccc2)c2ccc3c(c2)C(c2ccccc2)(c2ccccc2)c2cccc(N(c4ccccc4)c4ccc(C(C)(C)c5ccccc5)cc4)c2-3)cc1 ; CC(C)(c1ccccc1)c1ccc(N(c2ccccc2)c2ccc3c(c2)sc2cccc(N(c4ccccc4)c4ccc(C(C)(C)c5ccccc5)cc4)c23)cc1 ; CC(C)(c1ccccc1)c1ccc(N(c2ccccc2)c2ccc3c(c2)[se]c2ccc(N(c4ccccc4)c4ccc(C(C)(C)c5ccccc5)cc4)cc23)cc1]

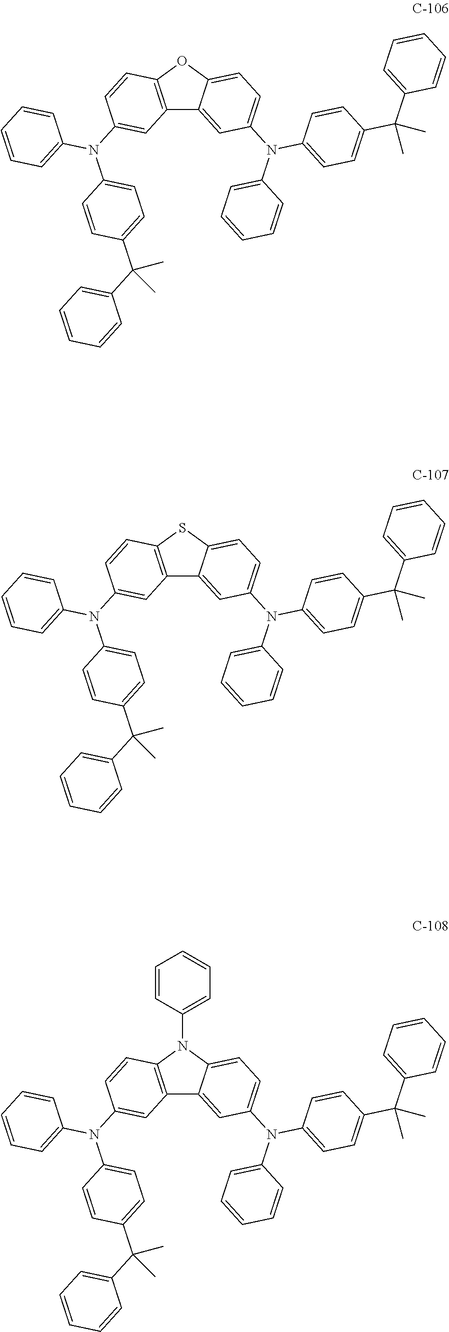 [SMILES: CC(C)(c1ccccc1)c1ccc(N(c2ccccc2)c2ccc3oc4ccc(N(c5ccccc5)c5ccc(C(C)(C)c6ccccc6)cc5)cc4c3c2)cc1 ; CC(C)(c1ccccc1)c1ccc(N(c2ccccc2)c2ccc3sc4ccc(N(c5ccccc5)c5ccc(C(C)(C)c6ccccc6)cc5)cc4c3c2)cc1 ; CC(C)(c1ccccc1)c1ccc(N(c2ccccc2)c2ccc3c(c2)c2cc(N(c4ccccc4)c4ccc(C(C)(C)c5ccccc5)cc4)ccc2n3-c2ccccc2)cc1]

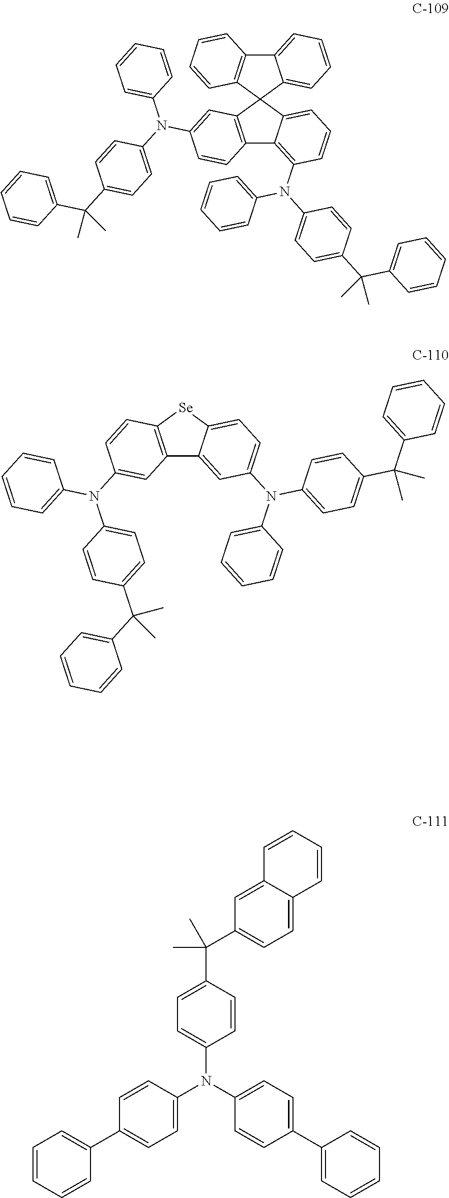 [SMILES: CC(C)(c1ccccc1)c1ccc(N(c2ccccc2)c2ccc3c(c2)C2(c4ccccc4-c4ccccc42)c2cccc(N(c4ccccc4)c4ccc(C(C)(C)c5ccccc5)cc4)c2-3)cc1 ; CC(C)(c1ccccc1)c1ccc(N(c2ccccc2)c2ccc3[se]c4ccc(N(c5ccccc5)c5ccc(C(C)(C)c6ccccc6)cc5)cc4c3c2)cc1 ; CC(C)(c1ccc(N(c2ccc(-c3ccccc3)cc2)c2ccc(-c3ccccc3)cc2)cc1)c1ccc2ccccc2c1]

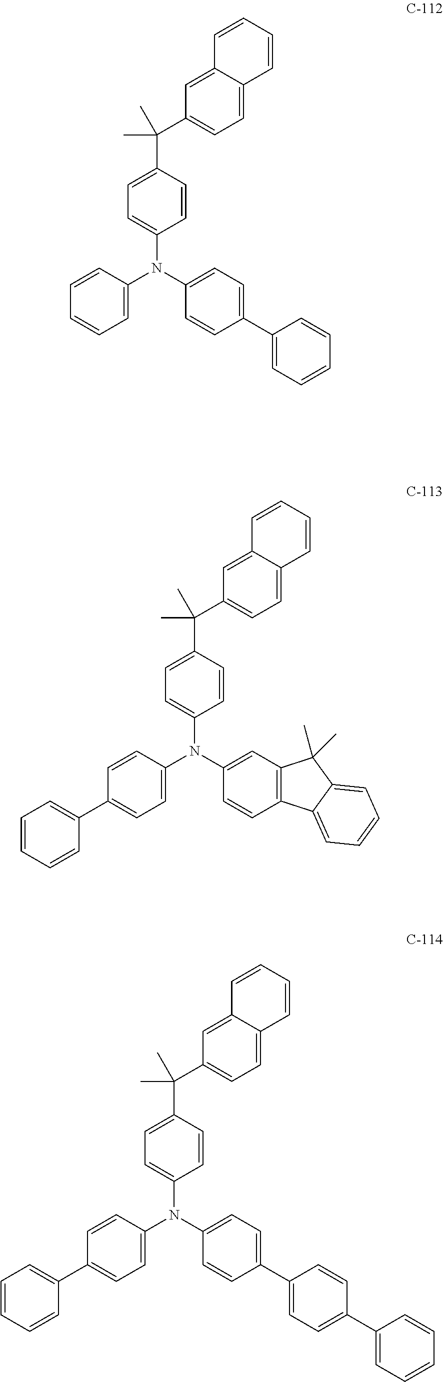 [SMILES: CC(C)(c1ccc(N(c2ccccc2)c2ccc(-c3ccccc3)cc2)cc1)c1ccc2ccccc2c1 ; CC(C)(c1ccc(N(c2ccc(-c3ccccc3)cc2)c2ccc3c(c2)C(C)(C)c2ccccc2-3)cc1)c1ccc2ccccc2c1 ; CC(C)(c1ccc(N(c2ccc(-c3ccccc3)cc2)c2ccc(-c3ccc(-c4ccccc4)cc3)cc2)cc1)c1ccc2ccccc2c1]

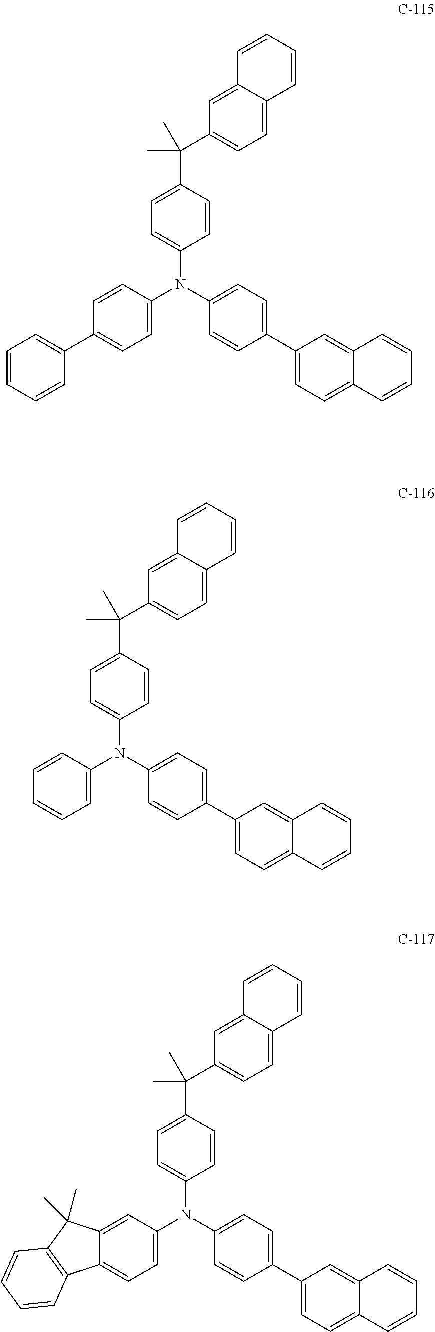 [SMILES: CC(C)(c1ccc(N(c2ccc(-c3ccccc3)cc2)c2ccc(-c3ccc4ccccc4c3)cc2)cc1)c1ccc2ccccc2c1 ; CC(C)(c1ccc(N(c2ccccc2)c2ccc(-c3ccc4ccccc4c3)cc2)cc1)c1ccc2ccccc2c1 ; CC(C)(c1ccc(N(c2ccc(-c3ccc4ccccc4c3)cc2)c2ccc3c(c2)C(C)(C)c2ccccc2-3)cc1)c1ccc2ccccc2c1]

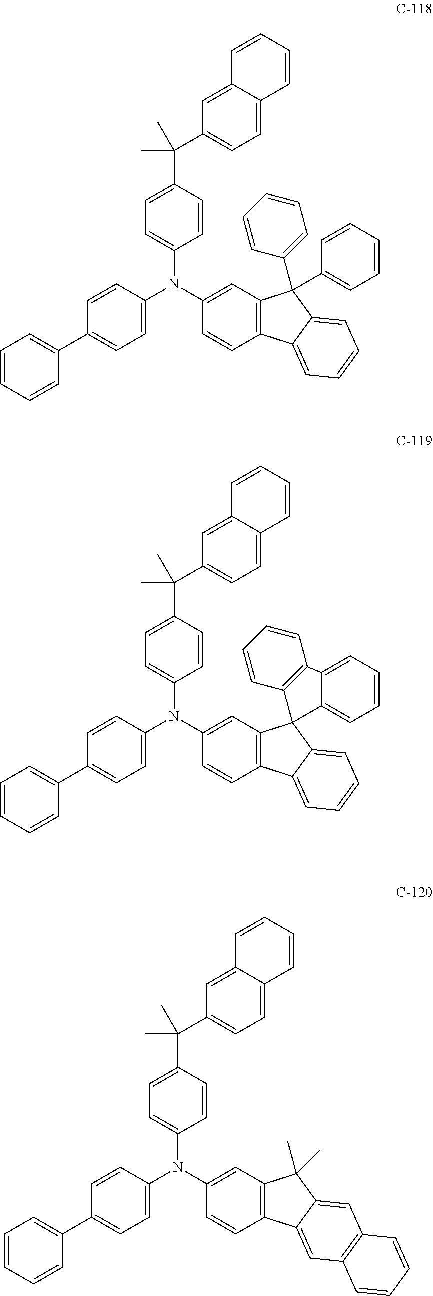 [SMILES: CC(C)(c1ccc(N(c2ccc(-c3ccccc3)cc2)c2ccc3c(c2)C(c2ccccc2)(c2ccccc2)c2ccccc2-3)cc1)c1ccc2ccccc2c1 ; CC(C)(c1ccc(N(c2ccc(-c3ccccc3)cc2)c2ccc3c(c2)C2(c4ccccc4-c4ccccc42)c2ccccc2-3)cc1)c1ccc2ccccc2c1 ; CC(C)(c1ccc(N(c2ccc(-c3ccccc3)cc2)c2ccc3c(c2)C(C)(C)c2cc4ccccc4cc2-3)cc1)c1ccc2ccccc2c1]

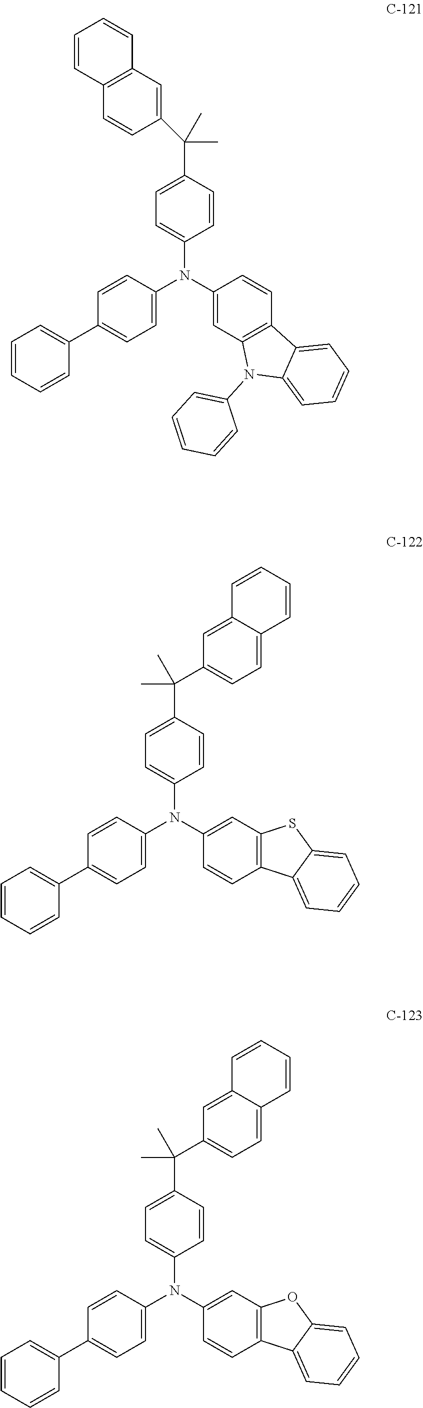 [SMILES: CC(C)(c1ccc(N(c2ccc(-c3ccccc3)cc2)c2ccc3c4ccccc4n(-c4ccccc4)c3c2)cc1)c1ccc2ccccc2c1 ; CC(C)(c1ccc(N(c2ccc(-c3ccccc3)cc2)c2ccc3c(c2)sc2ccccc23)cc1)c1ccc2ccccc2c1 ; CC(C)(c1ccc(N(c2ccc(-c3ccccc3)cc2)c2ccc3c(c2)oc2ccccc23)cc1)c1ccc2ccccc2c1]

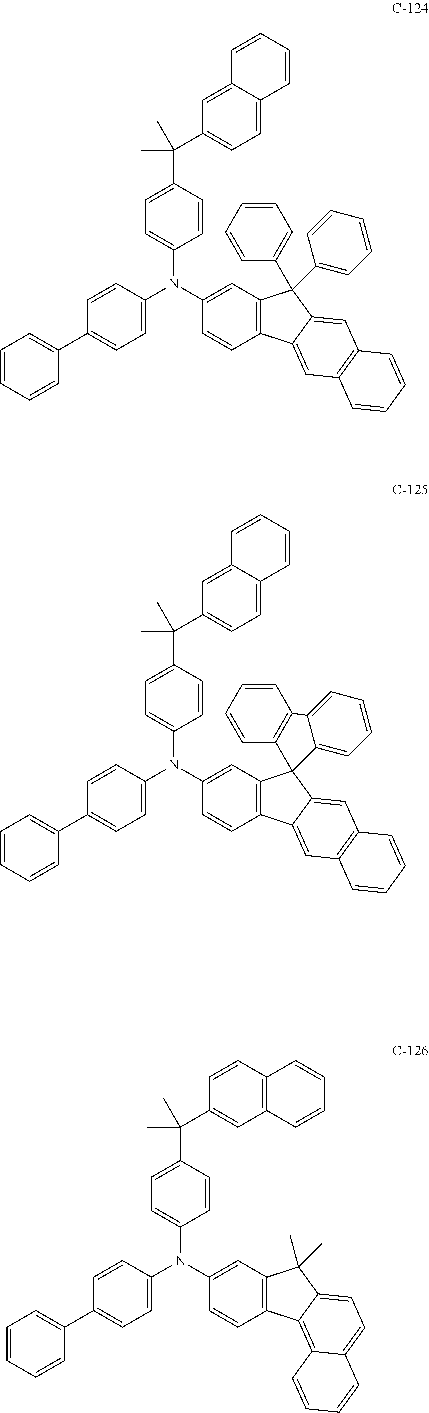 [SMILES: CC(C)(c1ccc(N(c2ccc(-c3ccccc3)cc2)c2ccc3c(c2)C(c2ccccc2)(c2ccccc2)c2cc4ccccc4cc2-3)cc1)c1ccc2ccccc2c1 ; CC(C)(c1ccc(N(c2ccc(-c3ccccc3)cc2)c2ccc3c(c2)C2(c4ccccc4-c4ccccc42)c2cc4ccccc4cc2-3)cc1)c1ccc2ccccc2c1 ; CC(C)(c1ccc(N(c2ccc(-c3ccccc3)cc2)c2ccc3c(c2)C(C)(C)c2ccc4ccccc4c2-3)cc1)c1ccc2ccccc2c1]

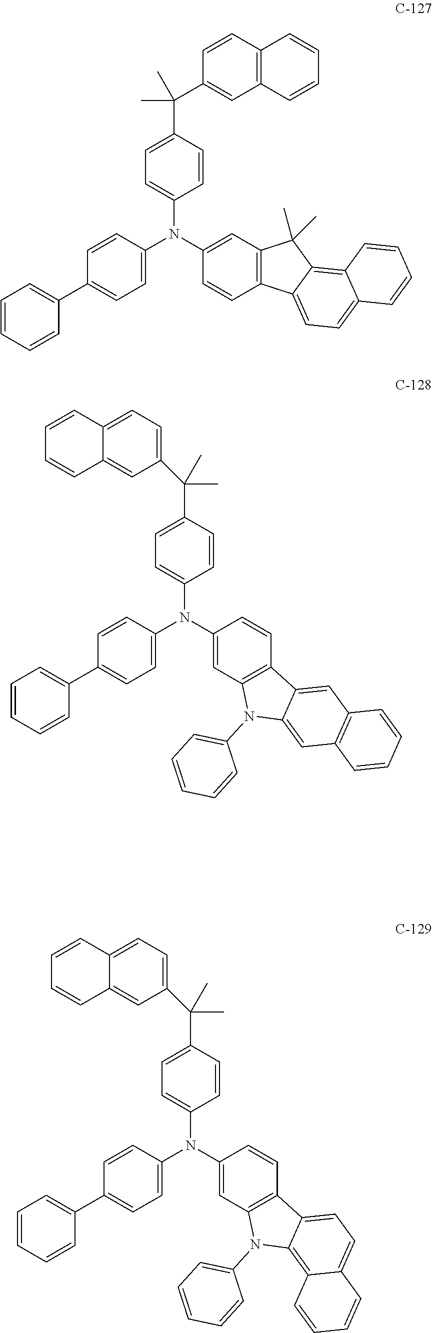 [SMILES: CC(C)(c1ccc(N(c2ccc(-c3ccccc3)cc2)c2ccc3c(c2)C(C)(C)c2c-3ccc3ccccc23)cc1)c1ccc2ccccc2c1 ; CC(C)(c1ccc(N(c2ccc(-c3ccccc3)cc2)c2ccc3c4cc5ccccc5cc4n(-c4ccccc4)c3c2)cc1)c1ccc2ccccc2c1 ; CC(C)(c1ccc(N(c2ccc(-c3ccccc3)cc2)c2ccc3c4ccc5ccccc5c4n(-c4ccccc4)c3c2)cc1)c1ccc2ccccc2c1]

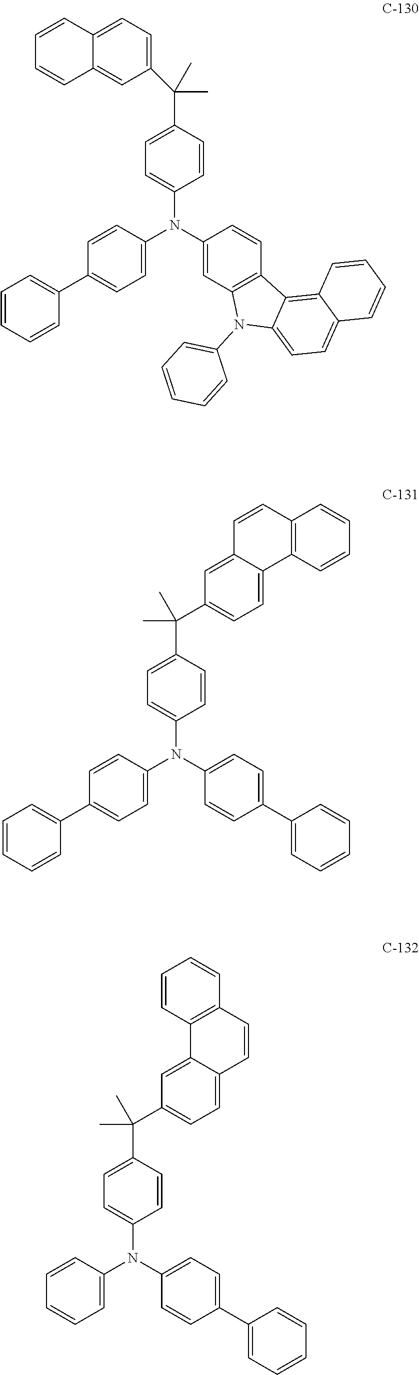 [SMILES: CC(C)(c1ccc(N(c2ccc(-c3ccccc3)cc2)c2ccc3c4c5ccccc5ccc4n(-c4ccccc4)c3c2)cc1)c1ccc2ccccc2c1 ; CC(C)(c1ccc(N(c2ccc(-c3ccccc3)cc2)c2ccc(-c3ccccc3)cc2)cc1)c1ccc2c(ccc3ccccc32)c1 ; CC(C)(c1ccc(N(c2ccccc2)c2ccc(-c3ccccc3)cc2)cc1)c1ccc2ccc3ccccc3c2c1]

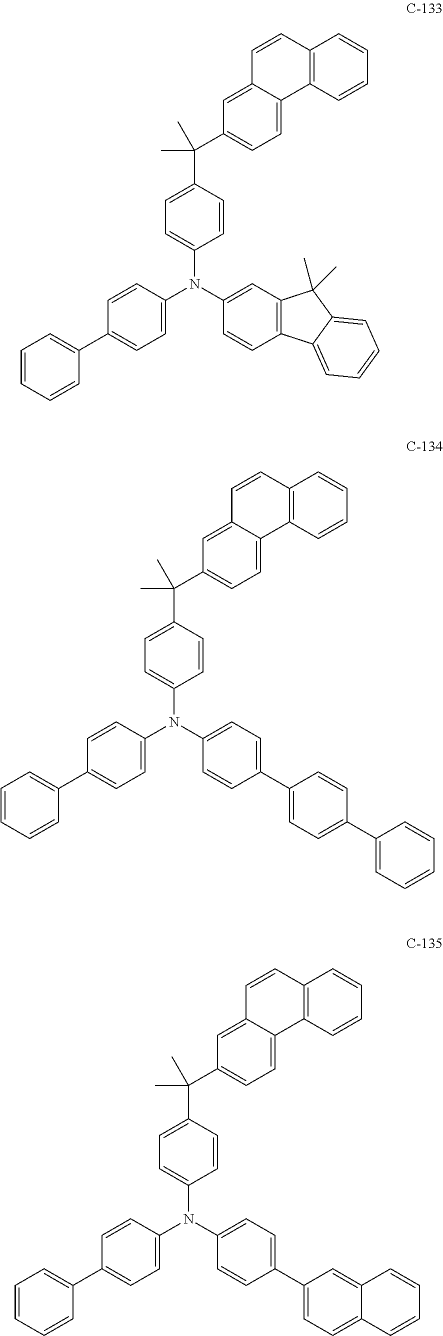 [SMILES: CC(C)(c1ccc(N(c2ccc(-c3ccccc3)cc2)c2ccc3c(c2)C(C)(C)c2ccccc2-3)cc1)c1ccc2c(ccc3ccccc32)c1 ; CC(C)(c1ccc(N(c2ccc(-c3ccccc3)cc2)c2ccc(-c3ccc(-c4ccccc4)cc3)cc2)cc1)c1ccc2c(ccc3ccccc32)c1 ; CC(C)(c1ccc(N(c2ccc(-c3ccccc3)cc2)c2ccc(-c3ccc4ccccc4c3)cc2)cc1)c1ccc2c(ccc3ccccc32)c1]

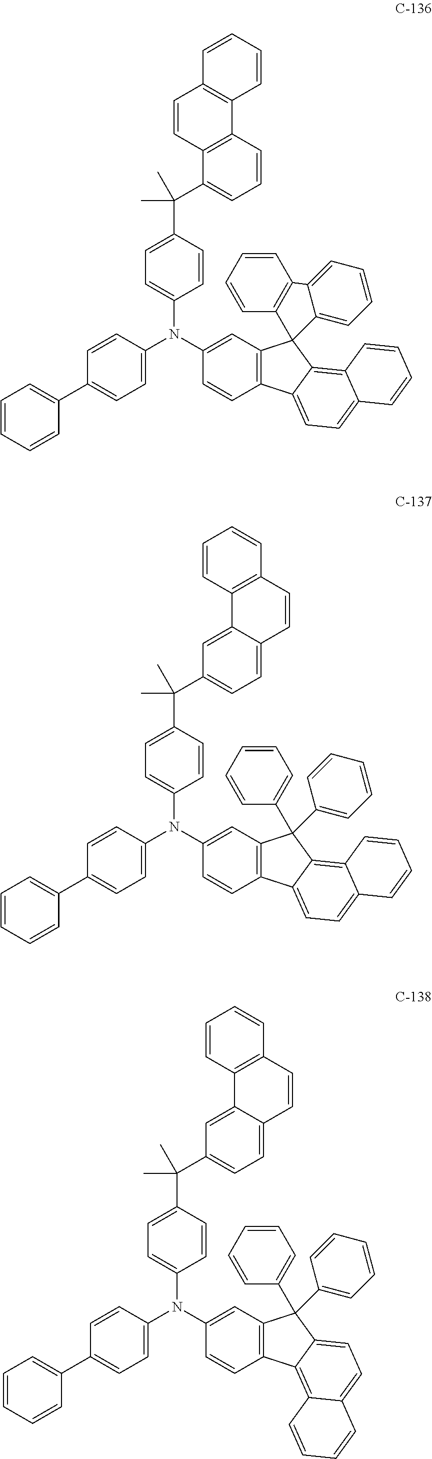 [SMILES: CC(C)(c1ccc(N(c2ccc(-c3ccccc3)cc2)c2ccc3c(c2)C2(c4ccccc4-c4ccccc42)c2c-3ccc3ccccc23)cc1)c1cccc2c1ccc1ccccc12 ; CC(C)(c1ccc(N(c2ccc(-c3ccccc3)cc2)c2ccc3c(c2)C(c2ccccc2)(c2ccccc2)c2c-3ccc3ccccc23)cc1)c1ccc2ccc3ccccc3c2c1 ; CC(C)(c1ccc(N(c2ccc(-c3ccccc3)cc2)c2ccc3c(c2)C(c2ccccc2)(c2ccccc2)c2ccc4ccccc4c2-3)cc1)c1ccc2ccc3ccccc3c2c1]

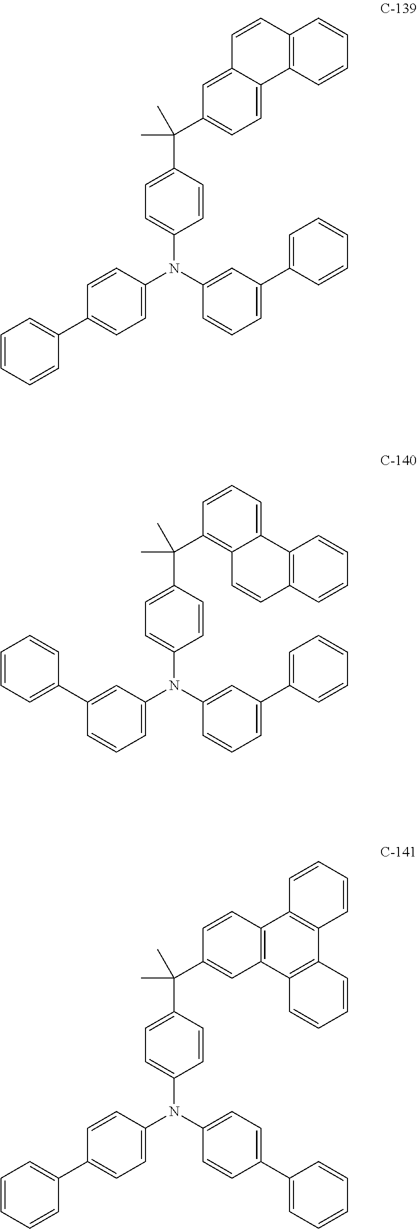 [SMILES: CC(C)(c1ccc(N(c2ccc(-c3ccccc3)cc2)c2cccc(-c3ccccc3)c2)cc1)c1ccc2c(ccc3ccccc32)c1 ; CC(C)(c1ccc(N(c2cccc(-c3ccccc3)c2)c2cccc(-c3ccccc3)c2)cc1)c1cccc2c1ccc1ccccc12 ; CC(C)(c1ccc(N(c2ccc(-c3ccccc3)cc2)c2ccc(-c3ccccc3)cc2)cc1)c1ccc2c3ccccc3c3ccccc3c2c1]

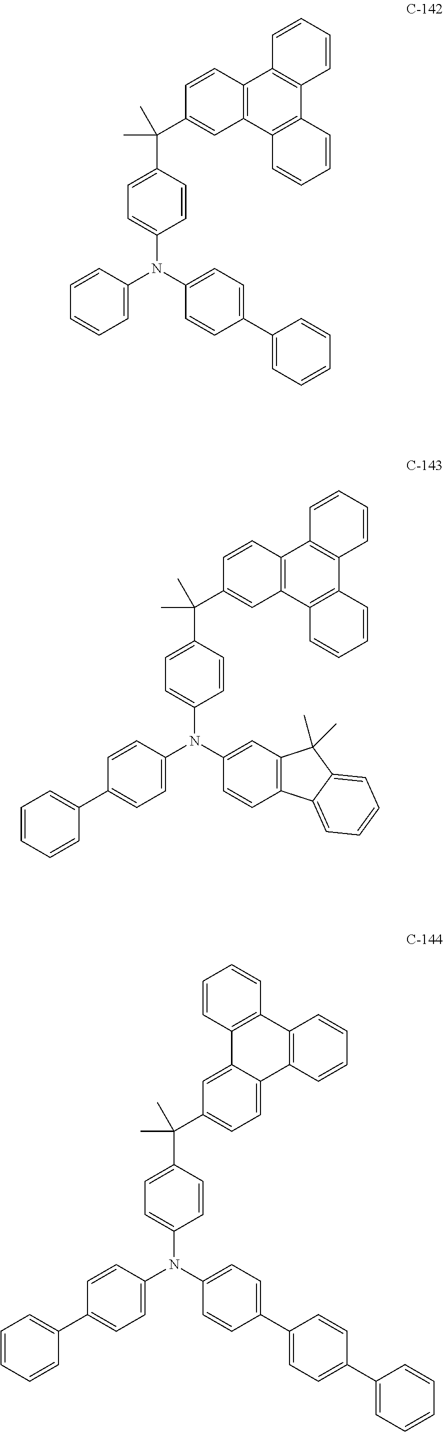 [SMILES: CC(C)(c1ccc(N(c2ccccc2)c2ccc(-c3ccccc3)cc2)cc1)c1ccc2c3ccccc3c3ccccc3c2c1 ; CC(C)(c1ccc(N(c2ccc(-c3ccccc3)cc2)c2ccc3c(c2)C(C)(C)c2ccccc2-3)cc1)c1ccc2c3ccccc3c3ccccc3c2c1 ; CC(C)(c1ccc(N(c2ccc(-c3ccccc3)cc2)c2ccc(-c3ccc(-c4ccccc4)cc3)cc2)cc1)c1ccc2c3ccccc3c3ccccc3c2c1]

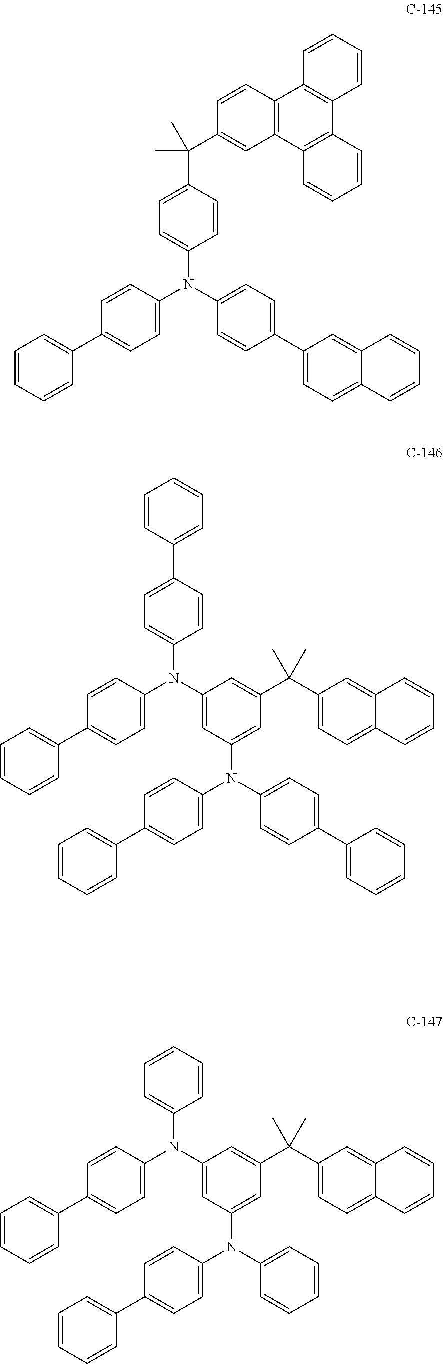 [SMILES: CC(C)(c1ccc(N(c2ccc(-c3ccccc3)cc2)c2ccc(-c3ccc4ccccc4c3)cc2)cc1)c1ccc2c3ccccc3c3ccccc3c2c1 ; CC(C)(c1cc(N(c2ccc(-c3ccccc3)cc2)c2ccc(-c3ccccc3)cc2)cc(N(c2ccc(-c3ccccc3)cc2)c2ccc(-c3ccccc3)cc2)c1)c1ccc2ccccc2c1 ; CC(C)(c1cc(N(c2ccccc2)c2ccc(-c3ccccc3)cc2)cc(N(c2ccccc2)c2ccc(-c3ccccc3)cc2)c1)c1ccc2ccccc2c1]

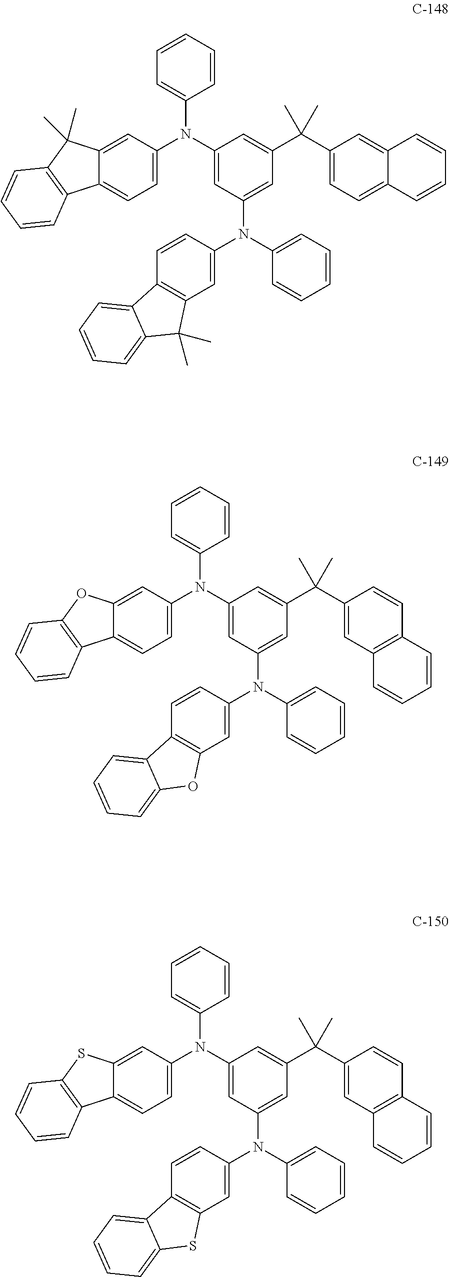 [SMILES: CC(C)(c1cc(N(c2ccccc2)c2ccc3c(c2)C(C)(C)c2ccccc2-3)cc(N(c2ccccc2)c2ccc3c(c2)C(C)(C)c2ccccc2-3)c1)c1ccc2ccccc2c1 ; CC(C)(c1cc(N(c2ccccc2)c2ccc3c(c2)oc2ccccc23)cc(N(c2ccccc2)c2ccc3c(c2)oc2ccccc23)c1)c1ccc2ccccc2c1 ; CC(C)(c1cc(N(c2ccccc2)c2ccc3c(c2)sc2ccccc23)cc(N(c2ccccc2)c2ccc3c(c2)sc2ccccc23)c1)c1ccc2ccccc2c1]

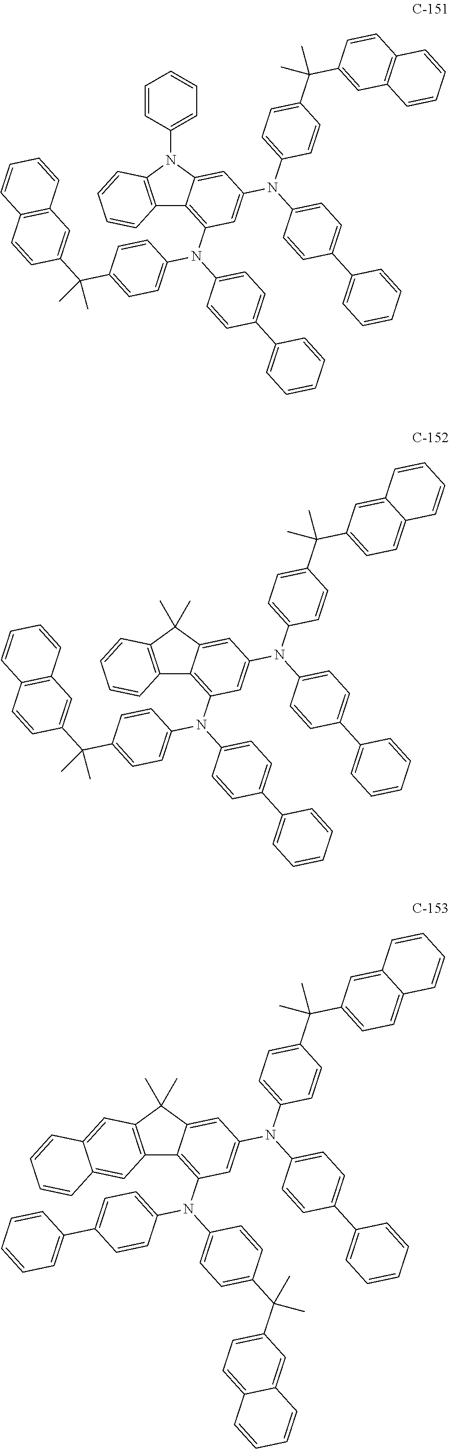 [SMILES: CC(C)(c1ccc(N(c2ccc(-c3ccccc3)cc2)c2cc(N(c3ccc(-c4ccccc4)cc3)c3ccc(C(C)(C)c4ccc5ccccc5c4)cc3)c3c4ccccc4n(-c4ccccc4)c3c2)cc1)c1ccc2ccccc2c1 ; CC(C)(c1ccc(N(c2ccc(-c3ccccc3)cc2)c2cc(N(c3ccc(-c4ccccc4)cc3)c3ccc(C(C)(C)c4ccc5ccccc5c4)cc3)c3c(c2)C(C)(C)c2ccccc2-3)cc1)c1ccc2ccccc2c1 ; CC(C)(c1ccc(N(c2ccc(-c3ccccc3)cc2)c2cc(N(c3ccc(-c4ccccc4)cc3)c3ccc(C(C)(C)c4ccc5ccccc5c4)cc3)c3c(c2)C(C)(C)c2cc4ccccc4cc2-3)cc1)c1ccc2ccccc2c1]

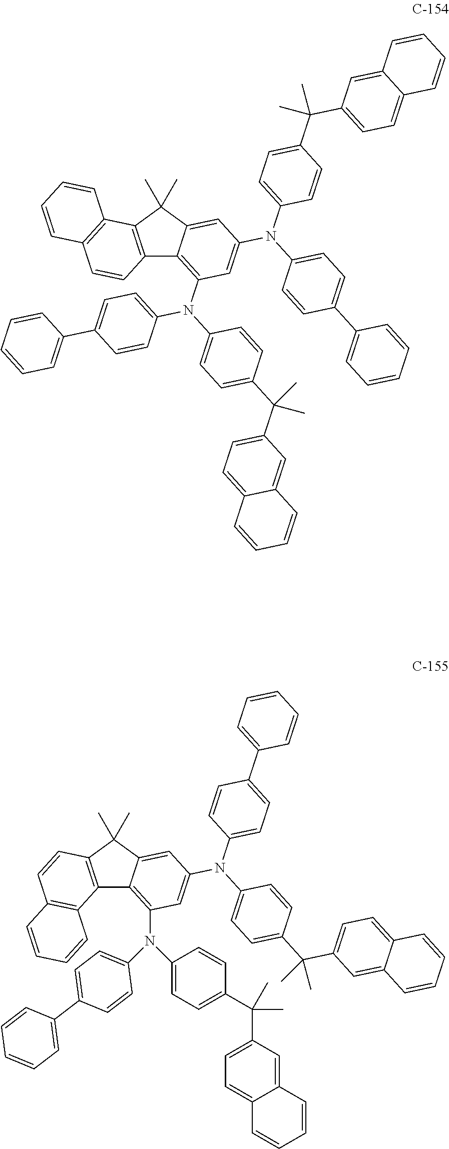 [SMILES: CC(C)(c1ccc(N(c2ccc(-c3ccccc3)cc2)c2cc(N(c3ccc(-c4ccccc4)cc3)c3ccc(C(C)(C)c4ccc5ccccc5c4)cc3)c3c(c2)C(C)(C)c2c-3ccc3ccccc23)cc1)c1ccc2ccccc2c1 ; CC(C)(c1ccc(N(c2ccc(-c3ccccc3)cc2)c2cc(N(c3ccc(-c4ccccc4)cc3)c3ccc(C(C)(C)c4ccc5ccccc5c4)cc3)c3c(c2)C(C)(C)c2ccc4ccccc4c2-3)cc1)c1ccc2ccccc2c1]

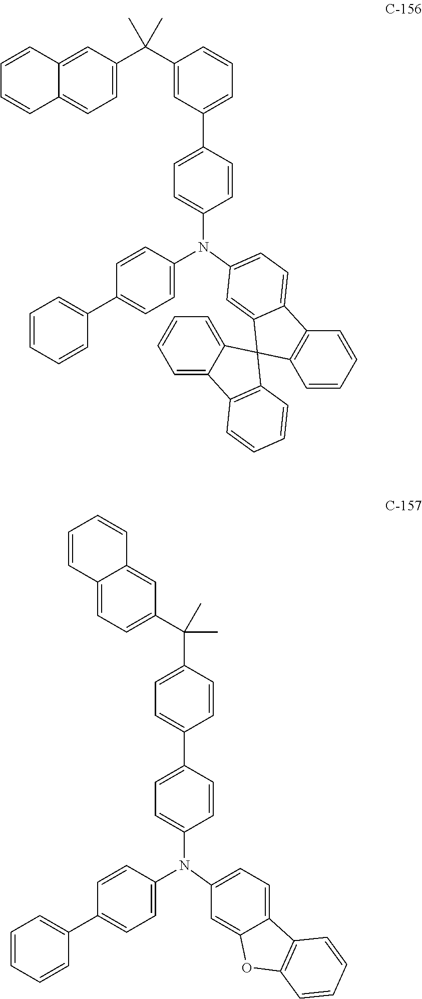 [SMILES: CC(C)(c1cccc(-c2ccc(N(c3ccc(-c4ccccc4)cc3)c3ccc4c(c3)C3(c5ccccc5-c5ccccc53)c3ccccc3-4)cc2)c1)c1ccc2ccccc2c1 ; CC(C)(c1ccc(-c2ccc(N(c3ccc(-c4ccccc4)cc3)c3ccc4c(c3)oc3ccccc34)cc2)cc1)c1ccc2ccccc2c1]

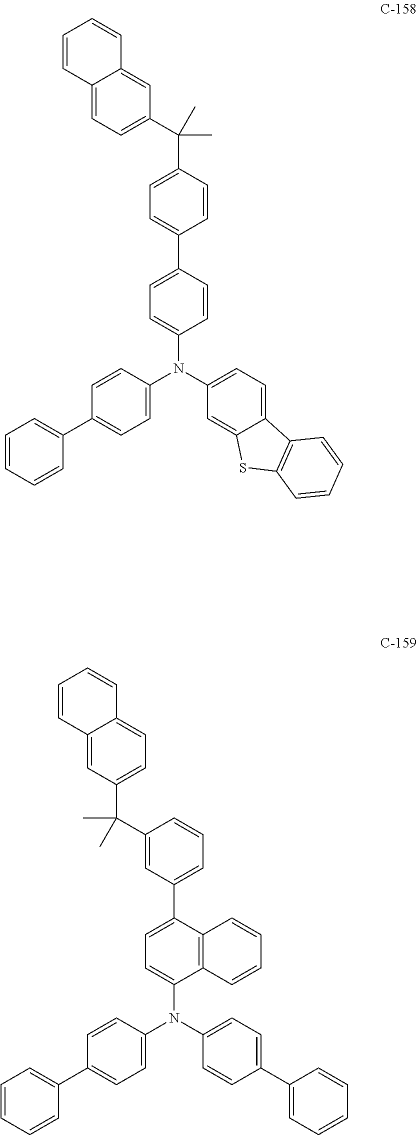 [SMILES: CC(C)(c1ccc(-c2ccc(N(c3ccc(-c4ccccc4)cc3)c3ccc4c(c3)sc3ccccc34)cc2)cc1)c1ccc2ccccc2c1 ; CC(C)(c1cccc(-c2ccc(N(c3ccc(-c4ccccc4)cc3)c3ccc(-c4ccccc4)cc3)c3ccccc23)c1)c1ccc2ccccc2c1]

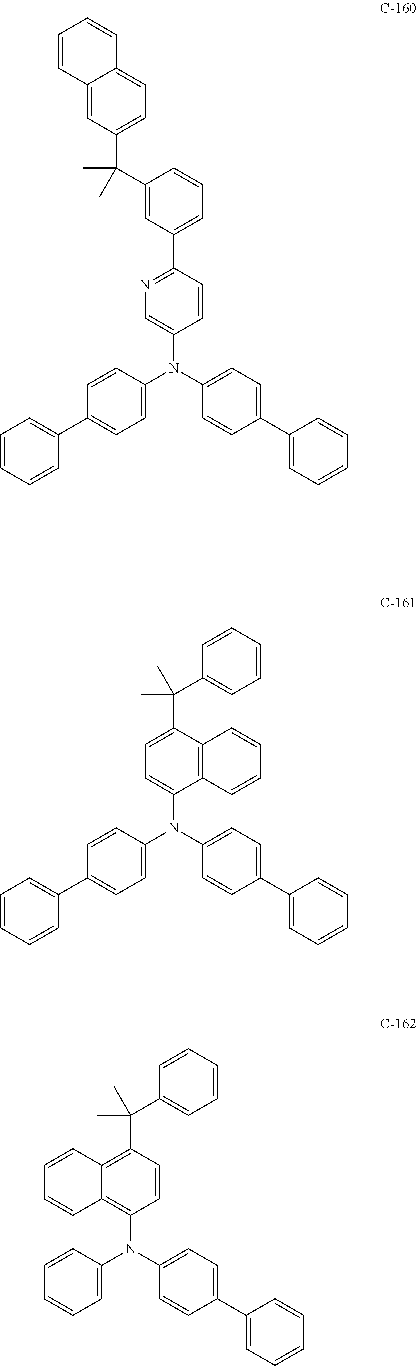 [SMILES: CC(C)(c1cccc(-c2ccc(N(c3ccc(-c4ccccc4)cc3)c3ccc(-c4ccccc4)cc3)cn2)c1)c1ccc2ccccc2c1 ; CC(C)(c1ccccc1)c1ccc(N(c2ccc(-c3ccccc3)cc2)c2ccc(-c3ccccc3)cc2)c2ccccc12 ; CC(C)(c1ccccc1)c1ccc(N(c2ccccc2)c2ccc(-c3ccccc3)cc2)c2ccccc12]

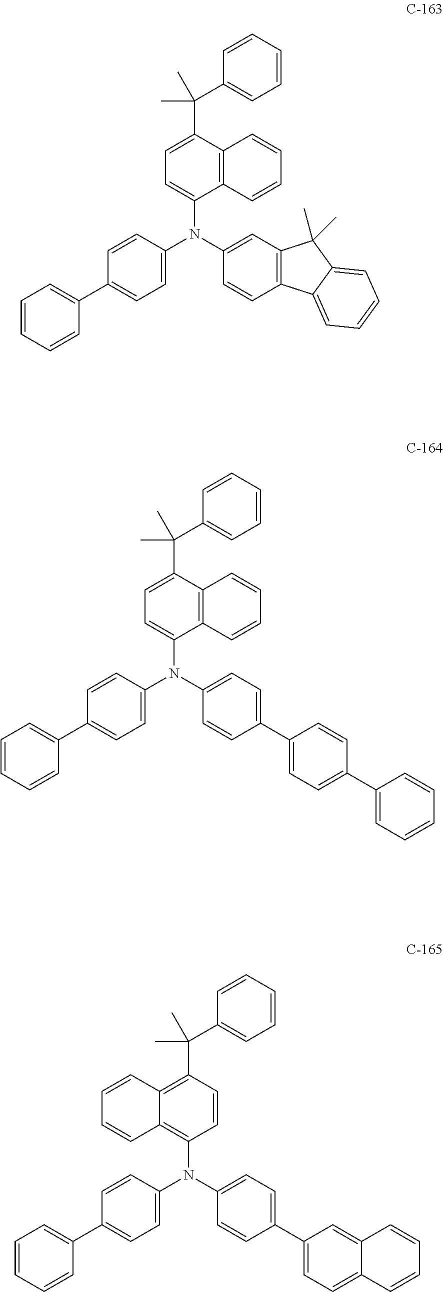 [SMILES: CC1(C)c2ccccc2-c2ccc(N(c3ccc(-c4ccccc4)cc3)c3ccc(C(C)(C)c4ccccc4)c4ccccc34)cc21 ; CC(C)(c1ccccc1)c1ccc(N(c2ccc(-c3ccccc3)cc2)c2ccc(-c3ccc(-c4ccccc4)cc3)cc2)c2ccccc12 ; CC(C)(c1ccccc1)c1ccc(N(c2ccc(-c3ccccc3)cc2)c2ccc(-c3ccc4ccccc4c3)cc2)c2ccccc12]

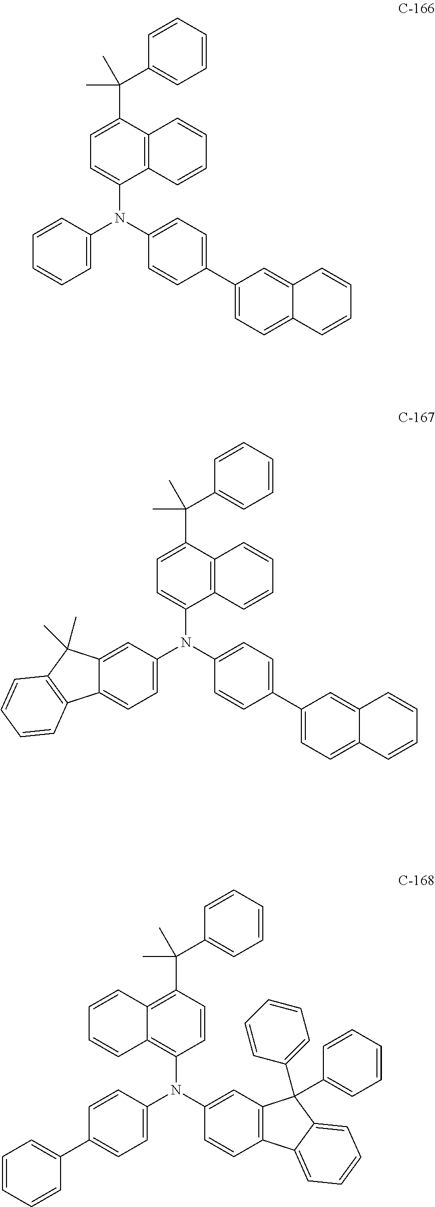 [SMILES: CC(C)(c1ccccc1)c1ccc(N(c2ccccc2)c2ccc(-c3ccc4ccccc4c3)cc2)c2ccccc12 ; CC1(C)c2ccccc2-c2ccc(N(c3ccc(-c4ccc5ccccc5c4)cc3)c3ccc(C(C)(C)c4ccccc4)c4ccccc34)cc21 ; CC(C)(c1ccccc1)c1ccc(N(c2ccc(-c3ccccc3)cc2)c2ccc3c(c2)C(c2ccccc2)(c2ccccc2)c2ccccc2-3)c2ccccc12]

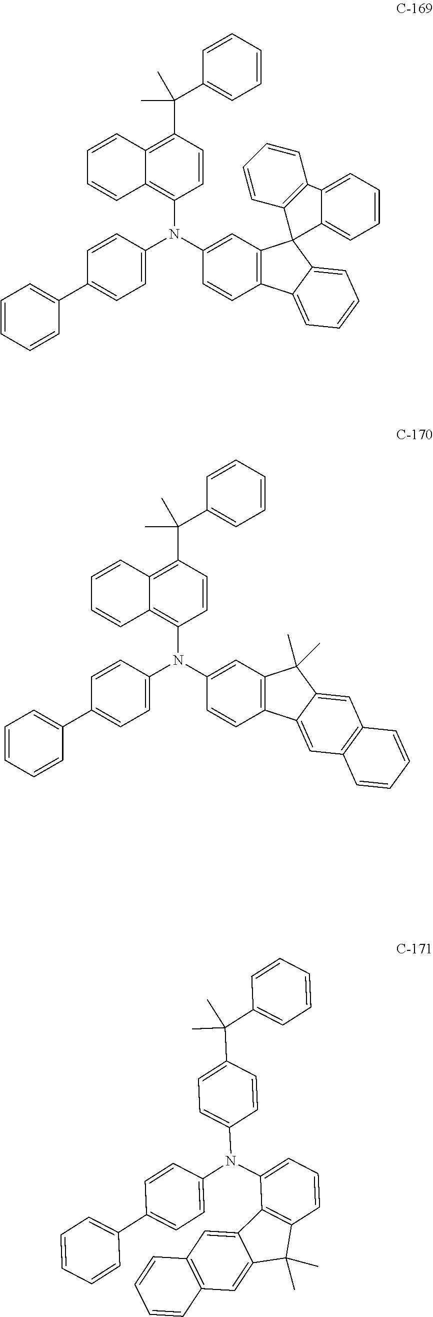 [SMILES: CC(C)(c1ccccc1)c1ccc(N(c2ccc(-c3ccccc3)cc2)c2ccc3c(c2)C2(c4ccccc4-c4ccccc42)c2ccccc2-3)c2ccccc12 ; CC1(C)c2cc(N(c3ccc(-c4ccccc4)cc3)c3ccc(C(C)(C)c4ccccc4)c4ccccc34)ccc2-c2cc3ccccc3cc21 ; CC(C)(c1ccccc1)c1ccc(N(c2ccc(-c3ccccc3)cc2)c2cccc3c2-c2cc4ccccc4cc2C3(C)C)cc1]

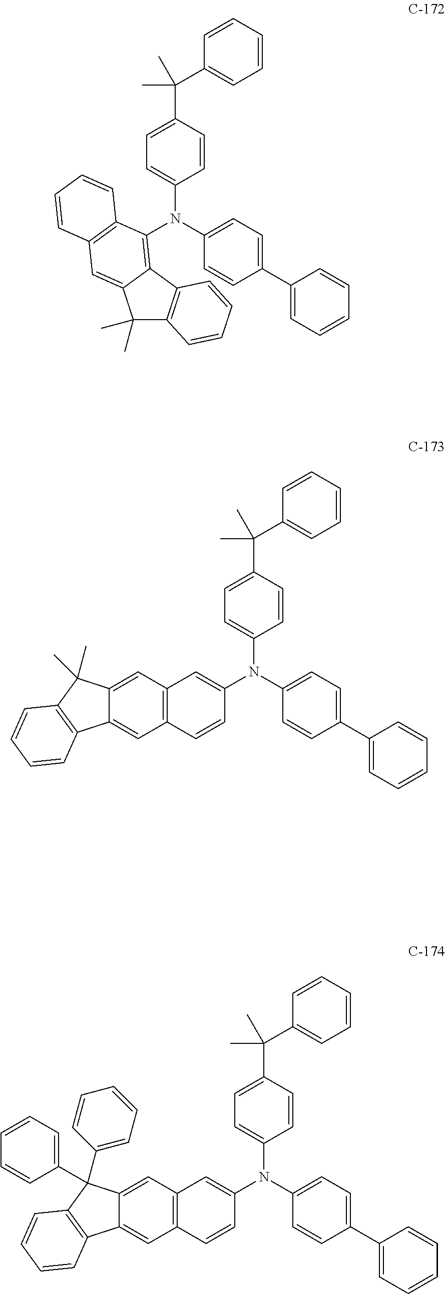 [SMILES: CC(C)(c1ccccc1)c1ccc(N(c2ccc(-c3ccccc3)cc2)c2c3c(cc4ccccc24)C(C)(C)c2ccccc2-3)cc1 ; CC(C)(c1ccccc1)c1ccc(N(c2ccc(-c3ccccc3)cc2)c2ccc3cc4c(cc3c2)C(C)(C)c2ccccc2-4)cc1 ; CC(C)(c1ccccc1)c1ccc(N(c2ccc(-c3ccccc3)cc2)c2ccc3cc4c(cc3c2)C(c2ccccc2)(c2ccccc2)c2ccccc2-4)cc1]

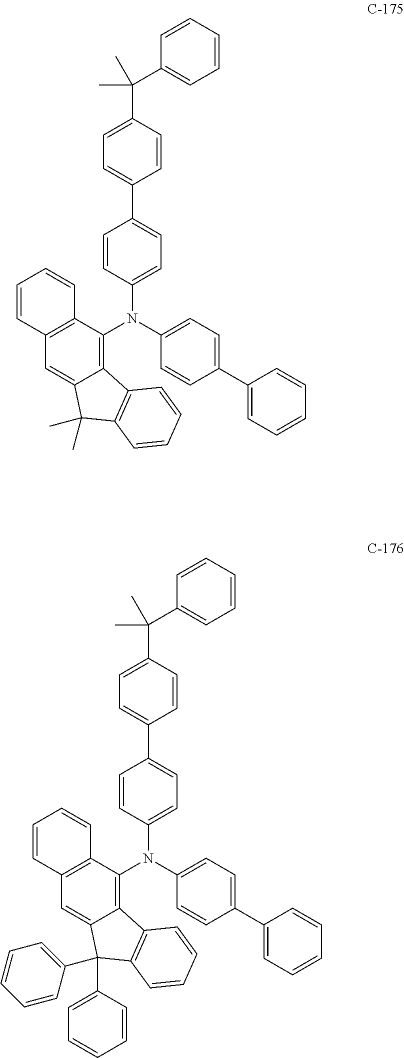 [SMILES: CC(C)(c1ccccc1)c1ccc(-c2ccc(N(c3ccc(-c4ccccc4)cc3)c3c4c(cc5ccccc35)C(C)(C)c3ccccc3-4)cc2)cc1 ; CC(C)(c1ccccc1)c1ccc(-c2ccc(N(c3ccc(-c4ccccc4)cc3)c3c4c(cc5ccccc35)C(c3ccccc3)(c3ccccc3)c3ccccc3-4)cc2)cc1]

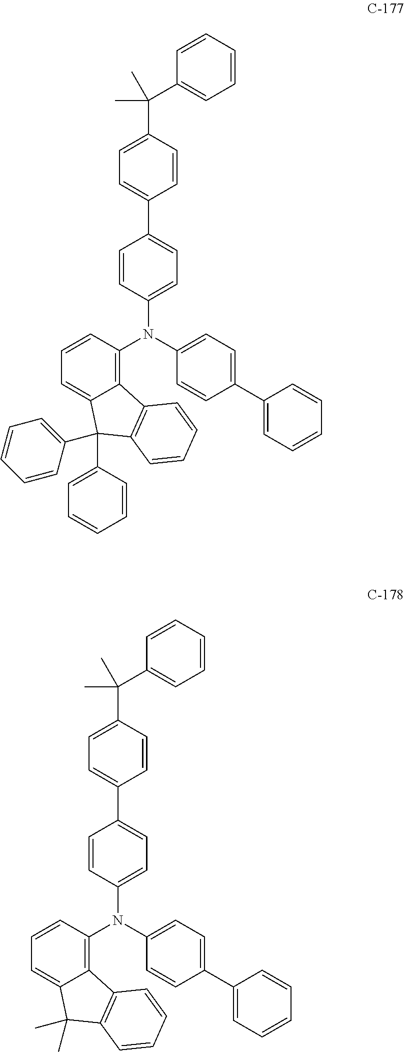 [SMILES: CC(C)(c1ccccc1)c1ccc(-c2ccc(N(c3ccc(-c4ccccc4)cc3)c3cccc4c3-c3ccccc3C4(c3ccccc3)c3ccccc3)cc2)cc1 ; CC(C)(c1ccccc1)c1ccc(-c2ccc(N(c3ccc(-c4ccccc4)cc3)c3cccc4c3-c3ccccc3C4(C)C)cc2)cc1]

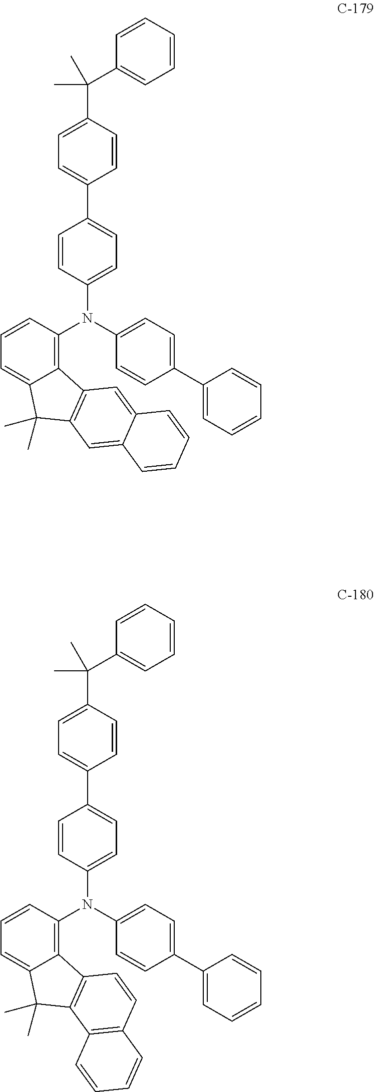 [SMILES: CC(C)(c1ccccc1)c1ccc(-c2ccc(N(c3ccc(-c4ccccc4)cc3)c3cccc4c3-c3cc5ccccc5cc3C4(C)C)cc2)cc1 ; CC(C)(c1ccccc1)c1ccc(-c2ccc(N(c3ccc(-c4ccccc4)cc3)c3cccc4c3-c3ccc5ccccc5c3C4(C)C)cc2)cc1]

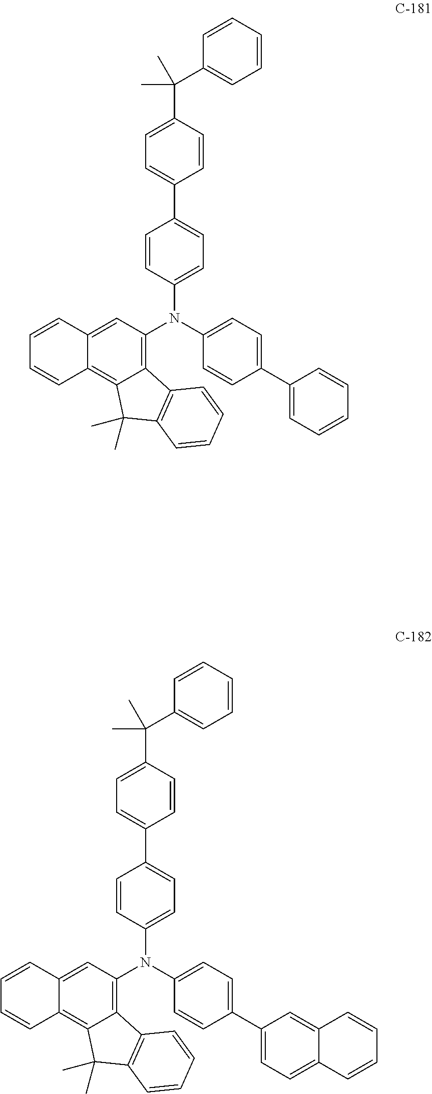 [SMILES: CC(C)(c1ccccc1)c1ccc(-c2ccc(N(c3ccc(-c4ccccc4)cc3)c3cc4ccccc4c4c3-c3ccccc3C4(C)C)cc2)cc1 ; CC(C)(c1ccccc1)c1ccc(-c2ccc(N(c3ccc(-c4ccc5ccccc5c4)cc3)c3cc4ccccc4c4c3-c3ccccc3C4(C)C)cc2)cc1]

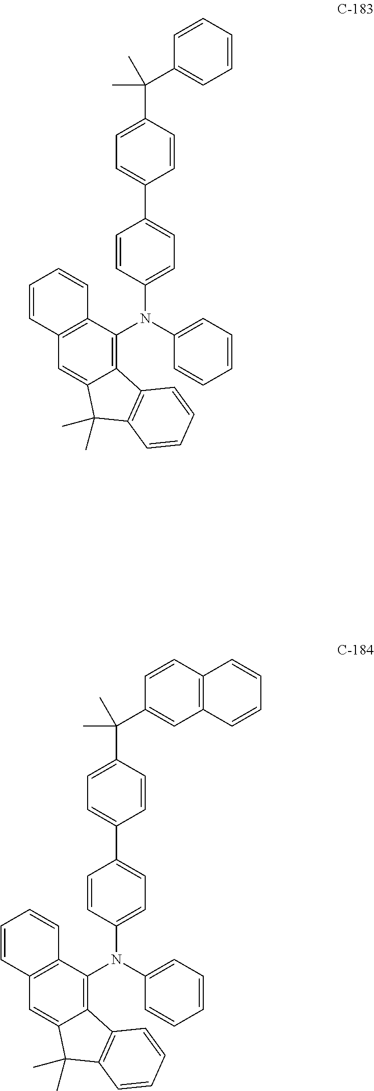 [SMILES: CC(C)(c1ccccc1)c1ccc(-c2ccc(N(c3ccccc3)c3c4c(cc5ccccc35)C(C)(C)c3ccccc3-4)cc2)cc1 ; CC(C)(c1ccc(-c2ccc(N(c3ccccc3)c3c4c(cc5ccccc35)C(C)(C)c3ccccc3-4)cc2)cc1)c1ccc2ccccc2c1]

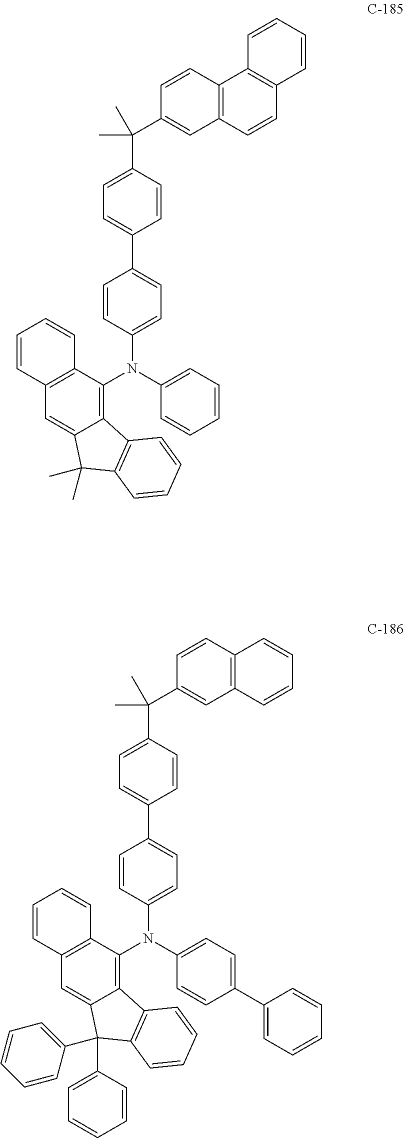 [SMILES: CC(C)(c1ccc(-c2ccc(N(c3ccccc3)c3c4c(cc5ccccc35)C(C)(C)c3ccccc3-4)cc2)cc1)c1ccc2c(ccc3ccccc32)c1 ; CC(C)(c1ccc(-c2ccc(N(c3ccc(-c4ccccc4)cc3)c3c4c(cc5ccccc35)C(c3ccccc3)(c3ccccc3)c3ccccc3-4)cc2)cc1)c1ccc2ccccc2c1]

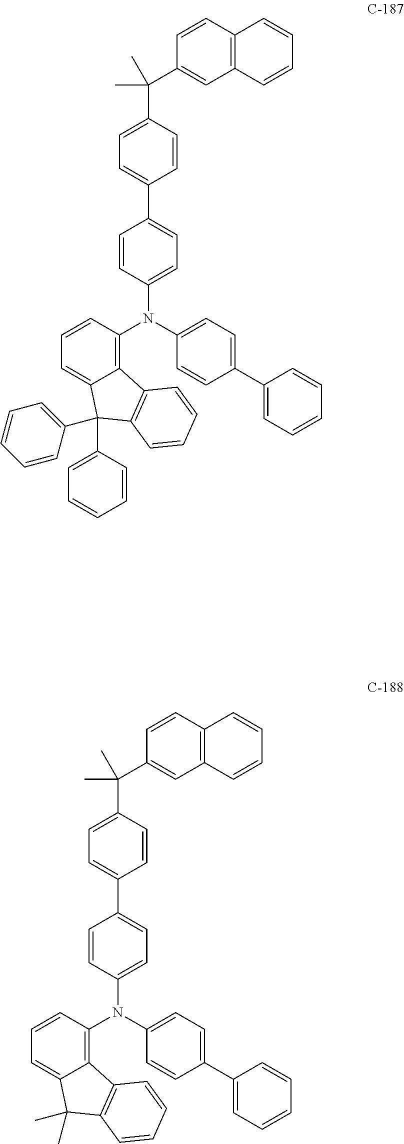 [SMILES: CC(C)(c1ccc(-c2ccc(N(c3ccc(-c4ccccc4)cc3)c3cccc4c3-c3ccccc3C4(c3ccccc3)c3ccccc3)cc2)cc1)c1ccc2ccccc2c1 ; CC(C)(c1ccc(-c2ccc(N(c3ccc(-c4ccccc4)cc3)c3cccc4c3-c3ccccc3C4(C)C)cc2)cc1)c1ccc2ccccc2c1]

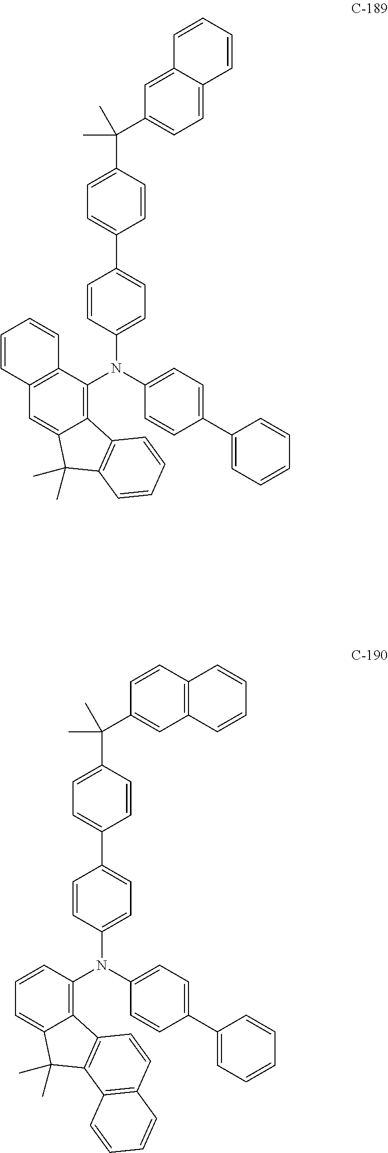 [SMILES: CC(C)(c1ccc(-c2ccc(N(c3ccc(-c4ccccc4)cc3)c3c4c(cc5ccccc35)C(C)(C)c3ccccc3-4)cc2)cc1)c1ccc2ccccc2c1 ; CC(C)(c1ccc(-c2ccc(N(c3ccc(-c4ccccc4)cc3)c3cccc4c3-c3ccc5ccccc5c3C4(C)C)cc2)cc1)c1ccc2ccccc2c1]

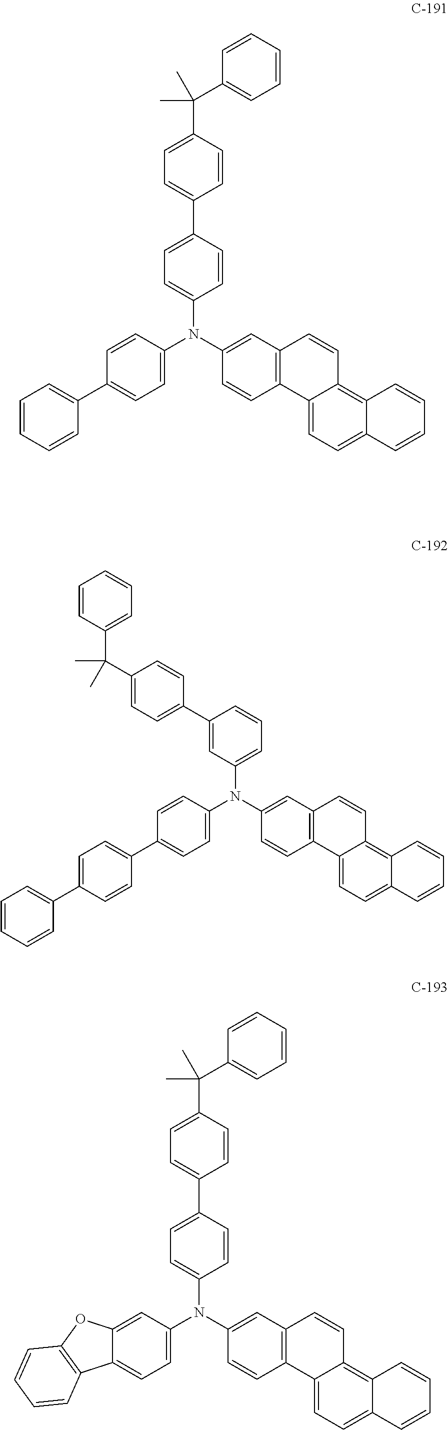 [SMILES: CC(C)(c1ccccc1)c1ccc(-c2ccc(N(c3ccc(-c4ccccc4)cc3)c3ccc4c(ccc5c6ccccc6ccc45)c3)cc2)cc1 ; CC(C)(c1ccccc1)c1ccc(-c2cccc(N(c3ccc(-c4ccc(-c5ccccc5)cc4)cc3)c3ccc4c(ccc5c6ccccc6ccc45)c3)c2)cc1 ; CC(C)(c1ccccc1)c1ccc(-c2ccc(N(c3ccc4c(ccc5c6ccccc6ccc45)c3)c3ccc4c(c3)oc3ccccc34)cc2)cc1]

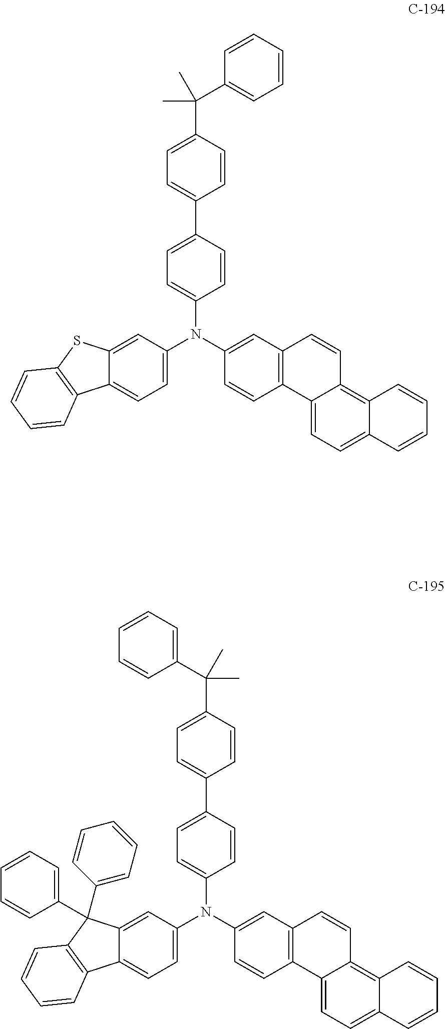 [SMILES: CC(C)(c1ccccc1)c1ccc(-c2ccc(N(c3ccc4c(ccc5c6ccccc6ccc45)c3)c3ccc4c(c3)sc3ccccc34)cc2)cc1 ; CC(C)(c1ccccc1)c1ccc(-c2ccc(N(c3ccc4c(c3)C(c3ccccc3)(c3ccccc3)c3ccccc3-4)c3ccc4c(ccc5c6ccccc6ccc45)c3)cc2)cc1]

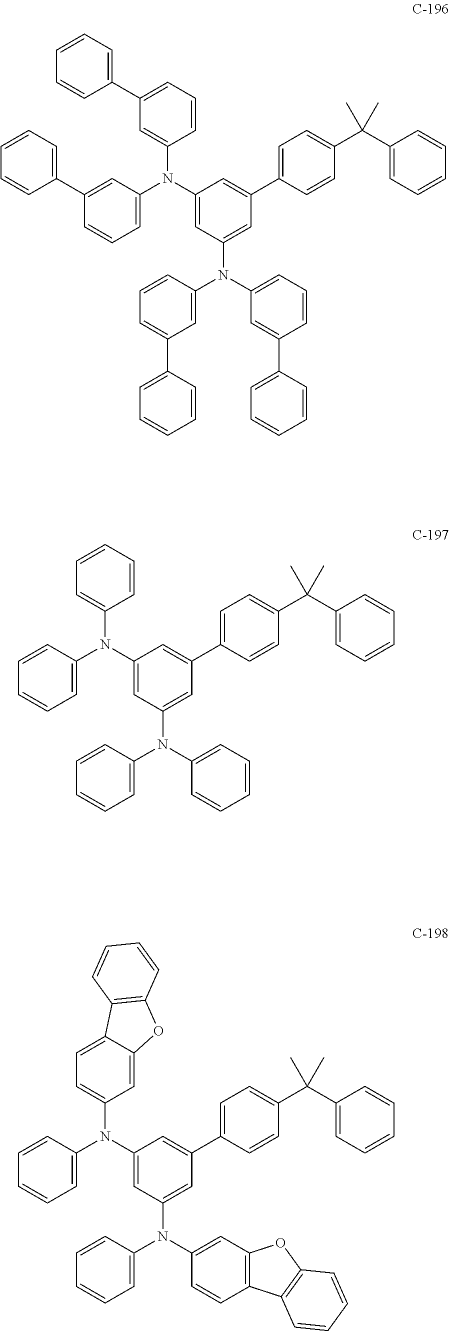 [SMILES: CC(C)(c1ccccc1)c1ccc(-c2cc(N(c3cccc(-c4ccccc4)c3)c3cccc(-c4ccccc4)c3)cc(N(c3cccc(-c4ccccc4)c3)c3cccc(-c4ccccc4)c3)c2)cc1 ; CC(C)(c1ccccc1)c1ccc(-c2cc(N(c3ccccc3)c3ccccc3)cc(N(c3ccccc3)c3ccccc3)c2)cc1 ; CC(C)(c1ccccc1)c1ccc(-c2cc(N(c3ccccc3)c3ccc4c(c3)oc3ccccc34)cc(N(c3ccccc3)c3ccc4c(c3)oc3ccccc34)c2)cc1]

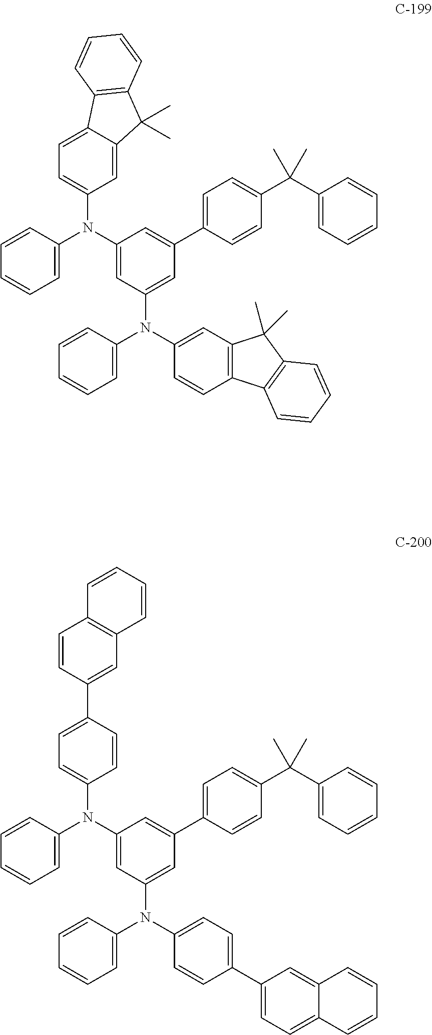 [SMILES: CC(C)(c1ccccc1)c1ccc(-c2cc(N(c3ccccc3)c3ccc4c(c3)C(C)(C)c3ccccc3-4)cc(N(c3ccccc3)c3ccc4c(c3)C(C)(C)c3ccccc3-4)c2)cc1 ; CC(C)(c1ccccc1)c1ccc(-c2cc(N(c3ccccc3)c3ccc(-c4ccc5ccccc5c4)cc3)cc(N(c3ccccc3)c3ccc(-c4ccc5ccccc5c4)cc3)c2)cc1]

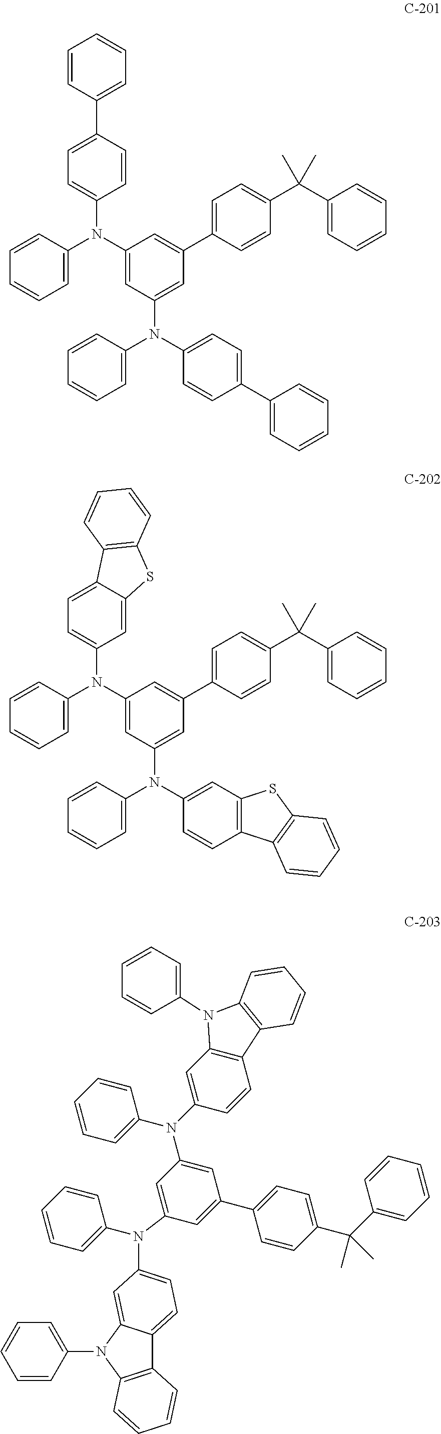 [SMILES: CC(C)(c1ccccc1)c1ccc(-c2cc(N(c3ccccc3)c3ccc(-c4ccccc4)cc3)cc(N(c3ccccc3)c3ccc(-c4ccccc4)cc3)c2)cc1 ; CC(C)(c1ccccc1)c1ccc(-c2cc(N(c3ccccc3)c3ccc4c(c3)sc3ccccc34)cc(N(c3ccccc3)c3ccc4c(c3)sc3ccccc34)c2)cc1 ; CC(C)(c1ccccc1)c1ccc(-c2cc(N(c3ccccc3)c3ccc4c5ccccc5n(-c5ccccc5)c4c3)cc(N(c3ccccc3)c3ccc4c5ccccc5n(-c5ccccc5)c4c3)c2)cc1]

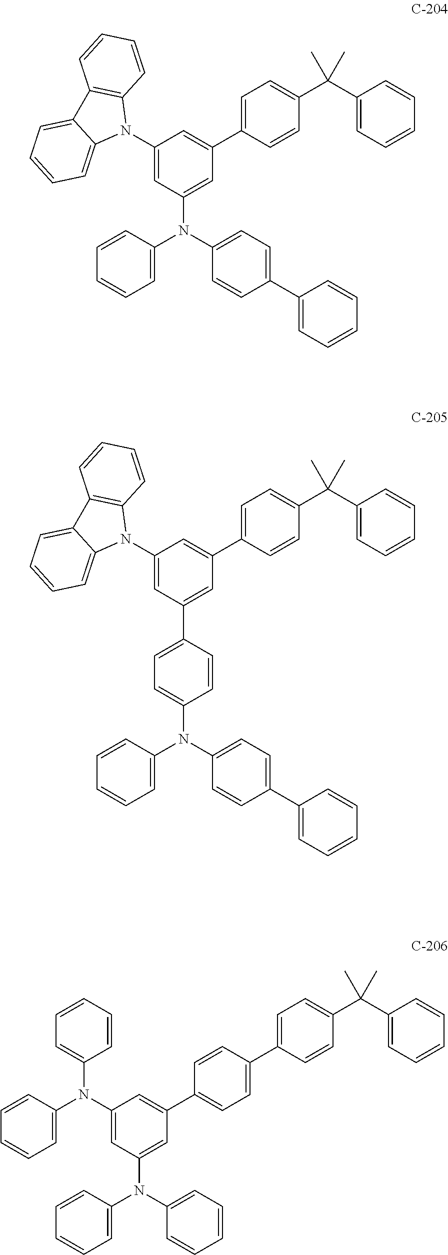 [SMILES: CC(C)(c1ccccc1)c1ccc(-c2cc(N(c3ccccc3)c3ccc(-c4ccccc4)cc3)cc(-n3c4ccccc4c4ccccc43)c2)cc1 ; CC(C)(c1ccccc1)c1ccc(-c2cc(-c3ccc(N(c4ccccc4)c4ccc(-c5ccccc5)cc4)cc3)cc(-n3c4ccccc4c4ccccc43)c2)cc1 ; CC(C)(c1ccccc1)c1ccc(-c2ccc(-c3cc(N(c4ccccc4)c4ccccc4)cc(N(c4ccccc4)c4ccccc4)c3)cc2)cc1]

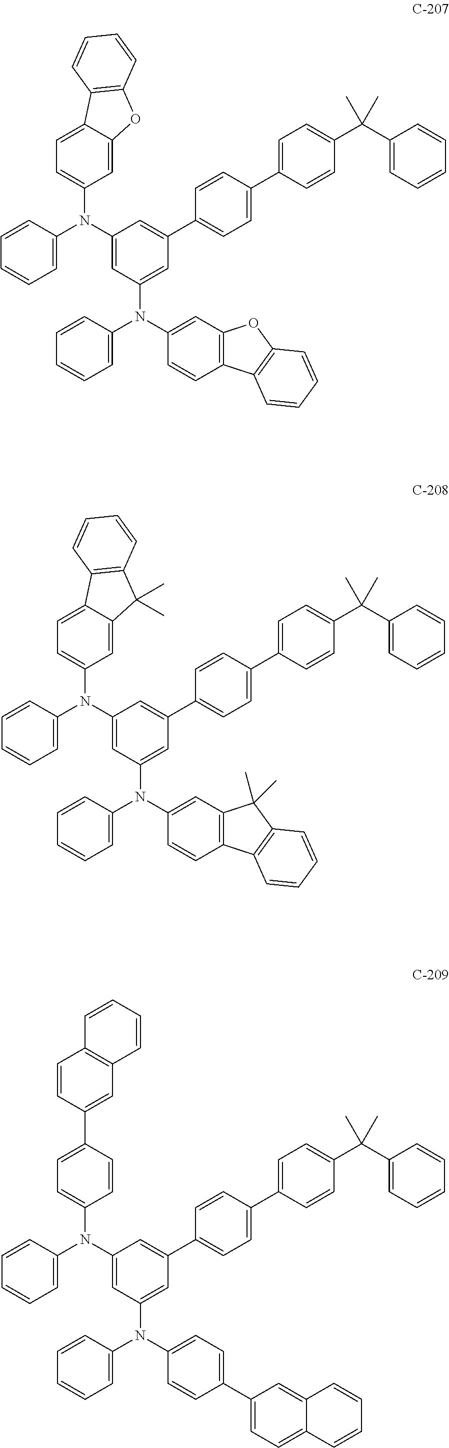 [SMILES: CC(C)(c1ccccc1)c1ccc(-c2ccc(-c3cc(N(c4ccccc4)c4ccc5c(c4)oc4ccccc45)cc(N(c4ccccc4)c4ccc5c(c4)oc4ccccc45)c3)cc2)cc1 ; CC(C)(c1ccccc1)c1ccc(-c2ccc(-c3cc(N(c4ccccc4)c4ccc5c(c4)C(C)(C)c4ccccc4-5)cc(N(c4ccccc4)c4ccc5c(c4)C(C)(C)c4ccccc4-5)c3)cc2)cc1 ; CC(C)(c1ccccc1)c1ccc(-c2ccc(-c3cc(N(c4ccccc4)c4ccc(-c5ccc6ccccc6c5)cc4)cc(N(c4ccccc4)c4ccc(-c5ccc6ccccc6c5)cc4)c3)cc2)cc1]

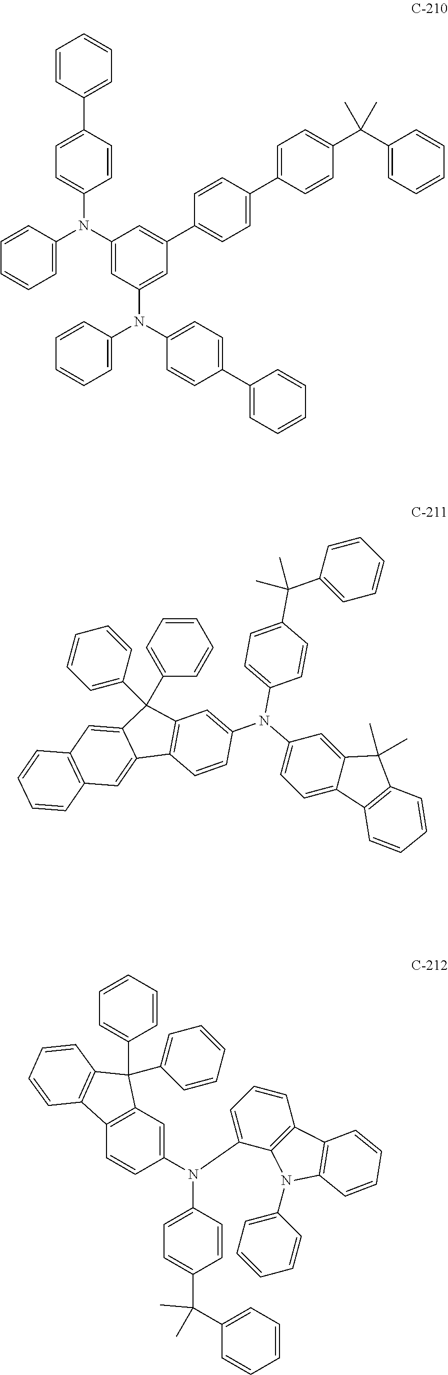 [SMILES: CC(C)(c1ccccc1)c1ccc(-c2ccc(-c3cc(N(c4ccccc4)c4ccc(-c5ccccc5)cc4)cc(N(c4ccccc4)c4ccc(-c5ccccc5)cc4)c3)cc2)cc1 ; CC(C)(c1ccccc1)c1ccc(N(c2ccc3c(c2)C(C)(C)c2ccccc2-3)c2ccc3c(c2)C(c2ccccc2)(c2ccccc2)c2cc4ccccc4cc2-3)cc1 ; CC(C)(c1ccccc1)c1ccc(N(c2ccc3c(c2)C(c2ccccc2)(c2ccccc2)c2ccccc2-3)c2cccc3c4ccccc4n(-c4ccccc4)c23)cc1]

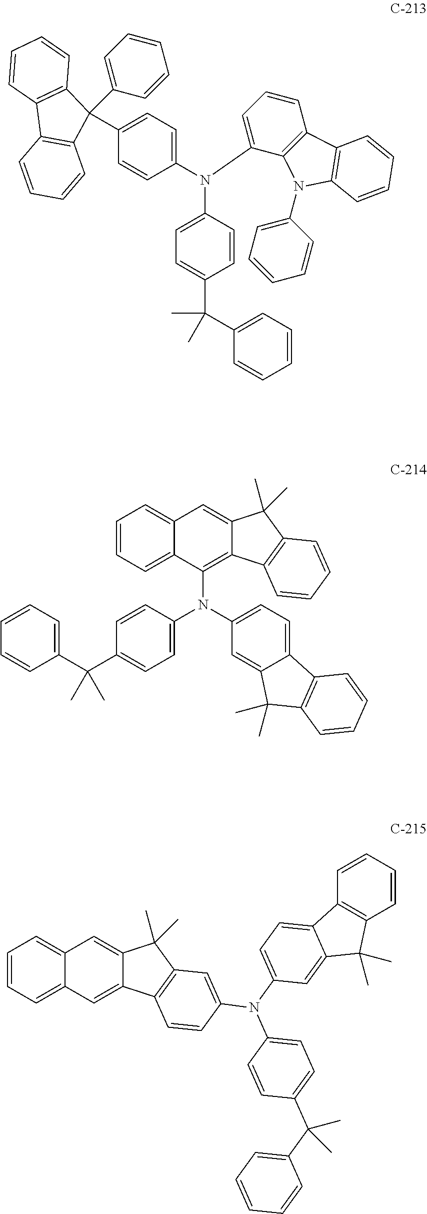 [SMILES: CC(C)(c1ccccc1)c1ccc(N(c2ccc(C3(c4ccccc4)c4ccccc4-c4ccccc43)cc2)c2cccc3c4ccccc4n(-c4ccccc4)c23)cc1 ; CC(C)(c1ccccc1)c1ccc(N(c2ccc3c(c2)C(C)(C)c2ccccc2-3)c2c3c(cc4ccccc24)C(C)(C)c2ccccc2-3)cc1 ; CC(C)(c1ccccc1)c1ccc(N(c2ccc3c(c2)C(C)(C)c2ccccc2-3)c2ccc3c(c2)C(C)(C)c2cc4ccccc4cc2-3)cc1]

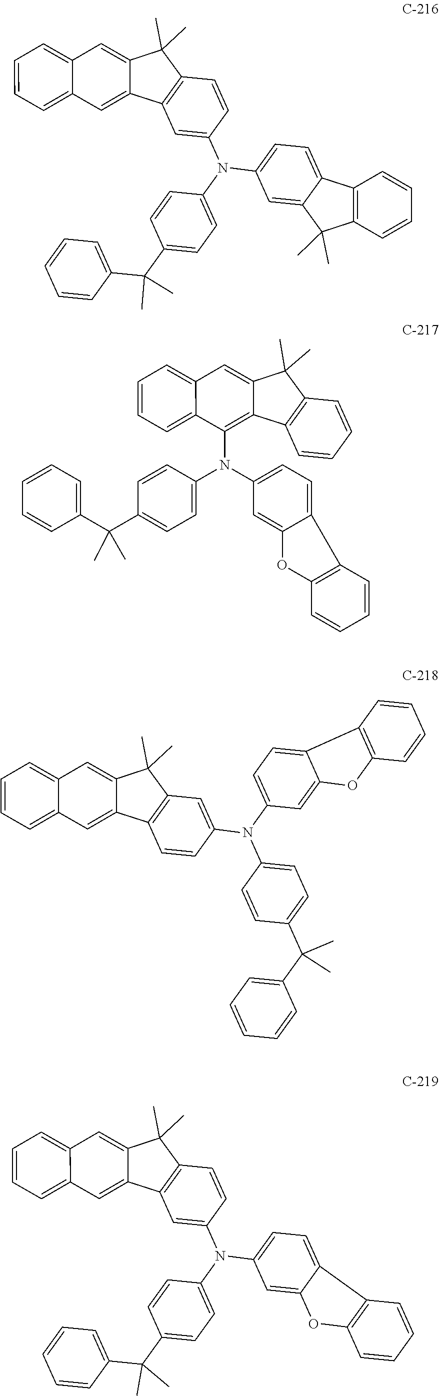 [SMILES: CC(C)(c1ccccc1)c1ccc(N(c2ccc3c(c2)-c2cc4ccccc4cc2C3(C)C)c2ccc3c(c2)C(C)(C)c2ccccc2-3)cc1 ; CC(C)(c1ccccc1)c1ccc(N(c2ccc3c(c2)oc2ccccc23)c2c3c(cc4ccccc24)C(C)(C)c2ccccc2-3)cc1 ; CC(C)(c1ccccc1)c1ccc(N(c2ccc3c(c2)C(C)(C)c2cc4ccccc4cc2-3)c2ccc3c(c2)oc2ccccc23)cc1 ; CC(C)(c1ccccc1)c1ccc(N(c2ccc3c(c2)-c2cc4ccccc4cc2C3(C)C)c2ccc3c(c2)oc2ccccc23)cc1]

The compound represented by formula 1 according to the present disclosure may be produced as shown in the following reaction schemes 1 to 3, but is not limited thereto.

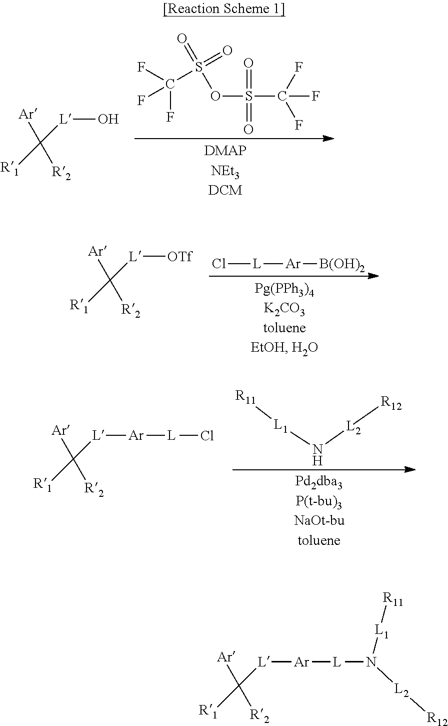

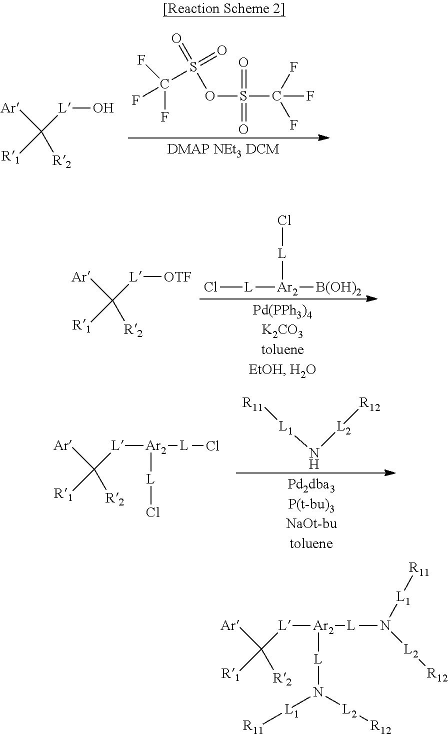

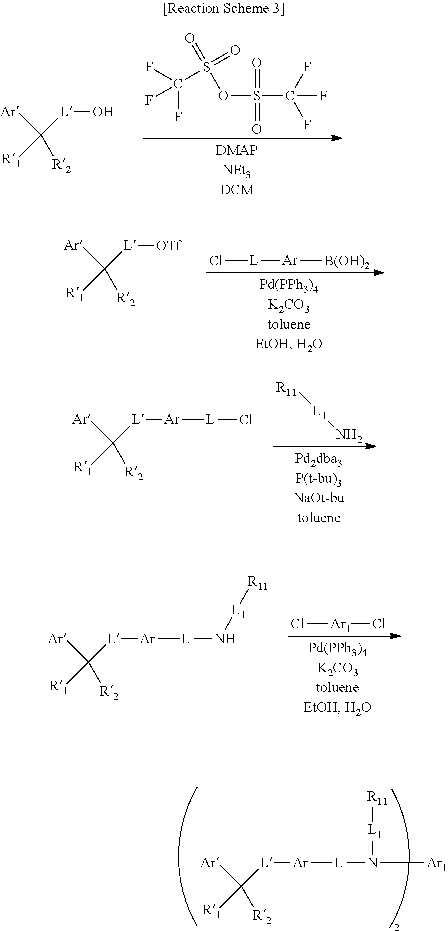

In reaction schemes 1 to 3, R′₁, R′₂, R₁₁, R₁₂, Ar′, Ar, Ar₂, L, L₁, L₂, and L′ are as defined in formula 1 and formulas 1-1 to 1-3.

Although illustrative synthesis examples of the compounds represented by formula 1 of the present disclosure are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, and a Phosphine-mediated reductive cyclization reaction, etc., and the reactions above proceed even when substituents which are defined in formula 1 above, but are not specified in the specific synthesis examples, are bonded.

As a host compound that can be used in combination with the organic electroluminescent compound of the present disclosure, a compound represented by any one of the following formulas 11 to 13 may be exemplified, but is not limited thereto.

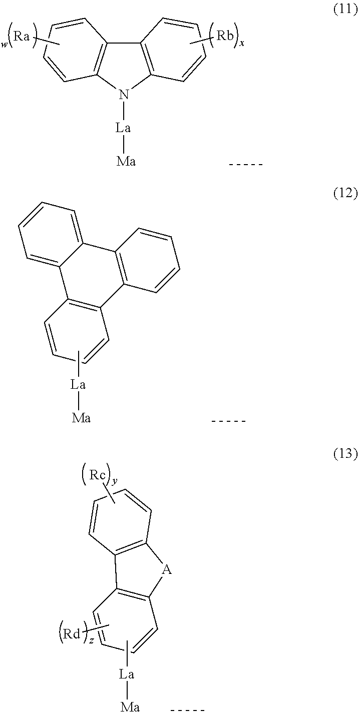

In formulas 11 to 13,

Ma represents a substituted or unsubstituted (C6-C30)aryl, a substituted or unsubstituted mono- or di- (C6-C30)arylamino, or a substituted or unsubstituted (3- to 30-membered)heteroaryl;

La represents a single bond, a substituted or unsubstituted (C6-C30)arylene, or a substituted or unsubstituted (3- to 30-membered)heteroarylene;

A represents S, O, N(Re) or C(Rf)(Rg);

Ra to Rd, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted (3- to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted (C1-C30)alkyl(C6-C30)arylamino, or a substituted or unsubstituted mono- or di- (C6-C30)arylamino; or may be linked to an adjacent substituent(s) to form a substituted or unsubstituted, mono- or polycyclic, (3- to 30-membered) alicyclic or aromatic ring, or the combination thereof, and the formed alicyclic or aromatic ring, or the combination thereof may contain at least one heteroatom selected from N, O, and S;

Re to Rg, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino; or Rf and Rg may be linked to each other to form a substituted or unsubstituted, mono- or polycyclic, (3- to 30-membered) alicyclic or aromatic ring, or the combination thereof, and the formed alicyclic or aromatic ring, or the combination thereof may contain at least one heteroatom selected from N, O, and S;

w to y, each independently, represent an integer of 1 to 4, and z represents an integer of 1 to 3; where if w to z are each an integer of 2 or more, each of Ra to each of Rd may be the same as or different from each other; and

the heteroaryl(ene) contains at least one heteroatom selected from B, N, O, S, Si and P.

The compound represented by any one of formulas 11 to 13 of the present disclosure may be prepared by a synthetic method known to one skilled in the art, but is not limited thereto.

The present disclosure provides an organic electroluminescent material comprising the organic electroluminescent compound of formula 1, and an organic electroluminescent device comprising the organic electroluminescent material.

The present disclosure provides an organic electroluminescent material comprising the organic electroluminescent compound represented by formula 1 and an organic electroluminescent compound represented by any one of formulas 11 to 13; and an organic electroluminescent device comprising the organic electroluminescent material.

The organic electroluminescent material may be a hole transport material, a hole auxiliary material or a light-emitting auxiliary material, and specifically, a hole transport material, a hole auxiliary material or a light-emitting auxiliary material of a blue light-emitting organic electroluminescent device. When the hole transport layer is two or more layers, the organic electroluminescent material may be a hole transport material (a hole auxiliary material) included in the hole transport layer adjacent to a light-emitting layer.

The organic electroluminescent material may be comprised solely of the organic electroluminescent compound of the present disclosure, or may further comprise conventional materials included in the organic electroluminescent material.

The hole transport zone of the present disclosure may be comprised of one or more layers from the group consisting of a hole transport layer, a hole injection layer, an electron blocking layer and a hole auxiliary layer, and each of the layers may consist of one or more layers.

According to one embodiment of the present disclosure, the hole transport zone includes a hole transport layer. In addition, the hole transport zone may include a hole transport layer, and further include at least one of a hole injection layer, an electron blocking layer, and a hole auxiliary layer.

The organic electroluminescent device according to the present disclosure includes a first electrode; a second electrode; and at least one organic layer interposed between the first electrode and the second electrode. The organic layer may comprise at least one organic electroluminescent compound represented by formula 1. One of the first electrode and the second electrode may be an anode and the other may be a cathode. The organic layer may comprise a light-emitting layer, and may further include at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer.

The organic electroluminescent compound represented by formula 1 of the present disclosure may be included in any one layer of the light-emitting layer, the hole injection layer, the hole transport layer, the hole auxiliary layer, the light-emitting auxiliary layer, the electron transport layer, the electron buffer layer, the electron injection layer, the interlayer, the hole blocking layer, and the electron blocking layer. In some cases, preferably, it may be included in at least one layer of the hole transport layer, the hole auxiliary layer, the light-emitting auxiliary layer, and the light-emitting layer. When the hole transport layer is two or more layers, the organic electroluminescent compound represented by formula 1 of the present disclosure may be used in at least one of the hole transport layers. For example, when used in the hole transport layer, the organic electroluminescent compound of the present disclosure may be comprised as a hole transport material. In addition, when used in the light-emitting layer, the organic electroluminescent compound of the present disclosure may be comprised as a host material.

The light-emitting layer may include at least one host and at least one dopant. If necessary, the light-emitting layer may include a co-host material, i.e., two or more host materials. The organic electroluminescent compound of the present disclosure may be used as a co-host material.

The host used in the present disclosure may be a phosphorescent host compound or a fluorescent host compound, and these host compounds are not particularly limited.

The dopant comprised in the organic electroluminescent device of the present disclosure may be at least one phosphorescent or fluorescent dopant, and is preferably a phosphorescent dopant. The phosphorescent dopant material applied to the present disclosure is not particularly limited, but may be a complex compound of a metal atom selected from iridium (Ir), osmium (Os), copper (Cu) and platinum (Pt), and preferably ortho-metallated complex compounds of a metal atom selected from iridium (Ir), osmium (Os), copper (Cu), and platinum (Pt), and more preferably ortho-metallated iridium complex compounds.

The dopant comprised in the organic electroluminescent device of the present disclosure may comprise a compound represented by the following formula 101, but is not limited thereto.

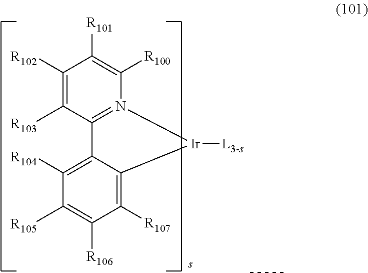

In formula 101,

L is selected from the following structures 1 to 3:

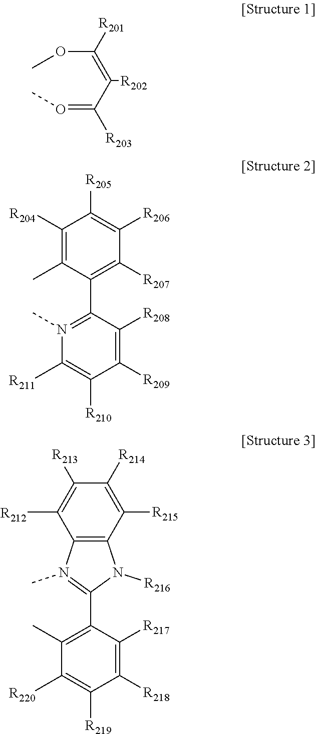

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent(s) to form a ring(s), e.g., a substituted or unsubstituted quinoline, a substituted or unsubstituted isoquinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline, together with pyridine;

R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent(s) to form a substituted or unsubstituted ring(s), e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine, together with benzene;

R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent(s) to form a substituted or unsubstituted ring(s); and

s represents an integer of 1 to 3,

The specific examples of the dopant compound are as follows, but are not limited thereto.

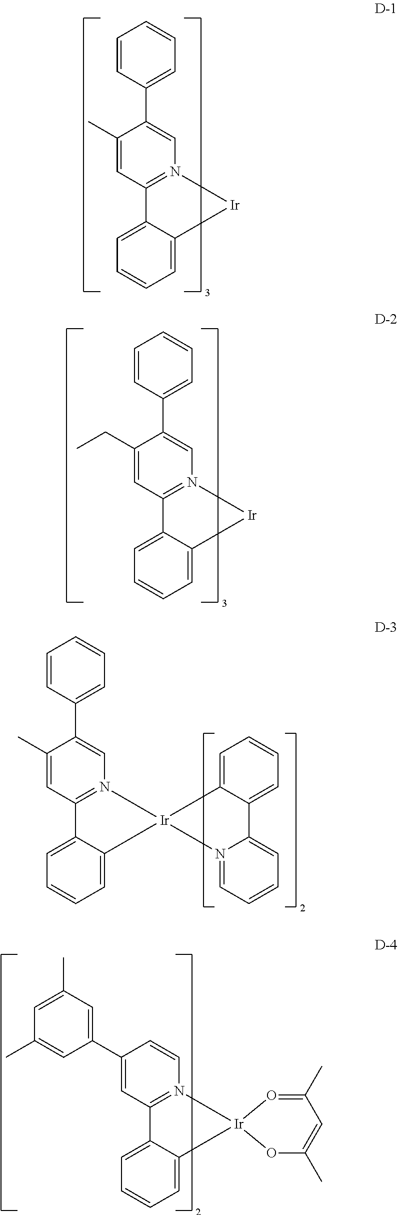

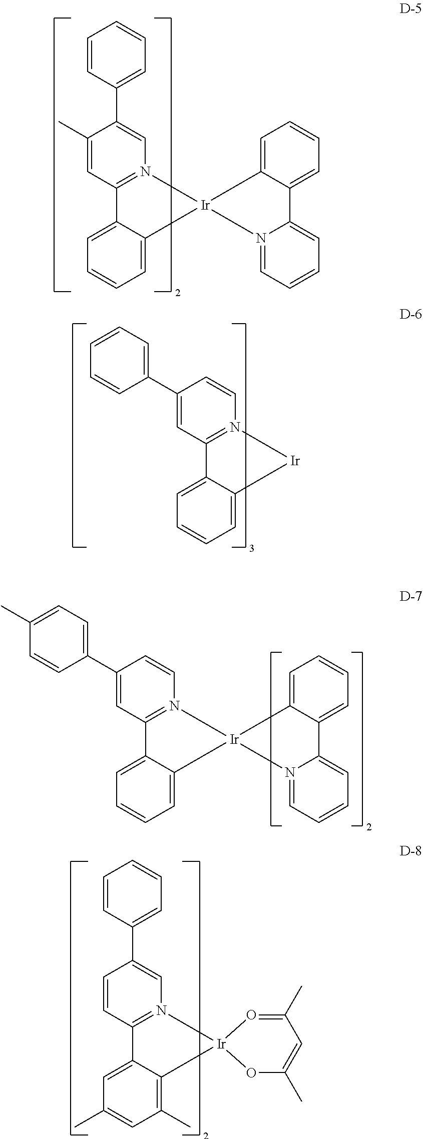

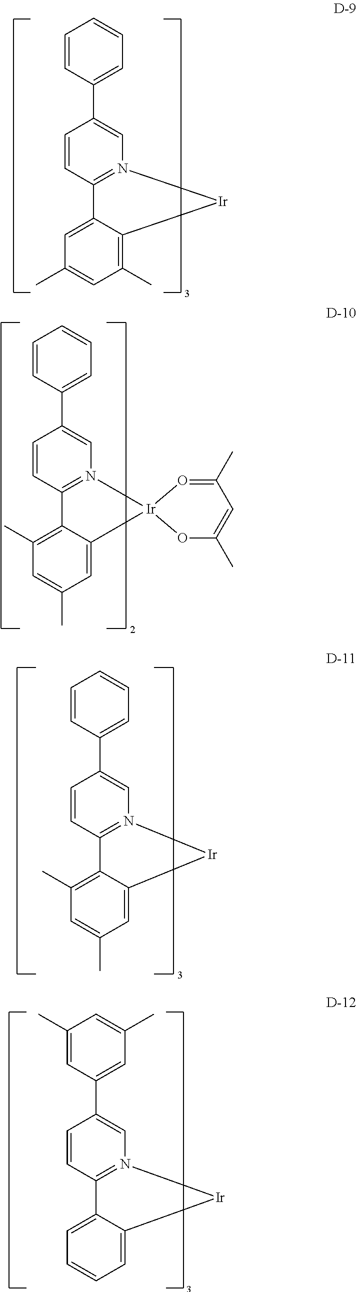

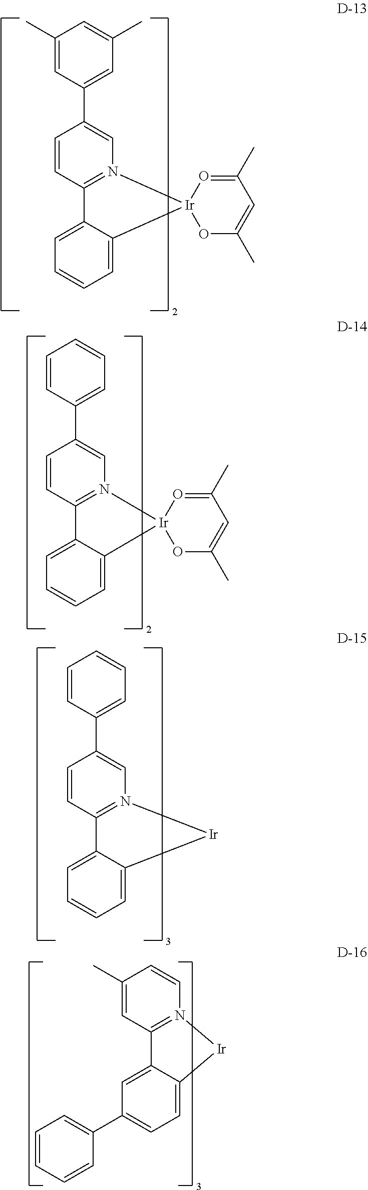

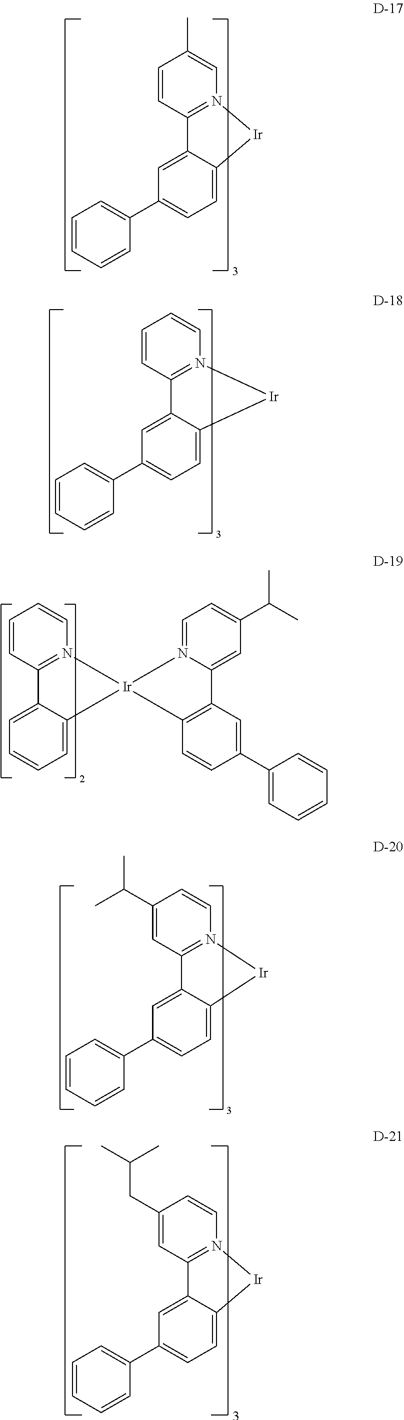

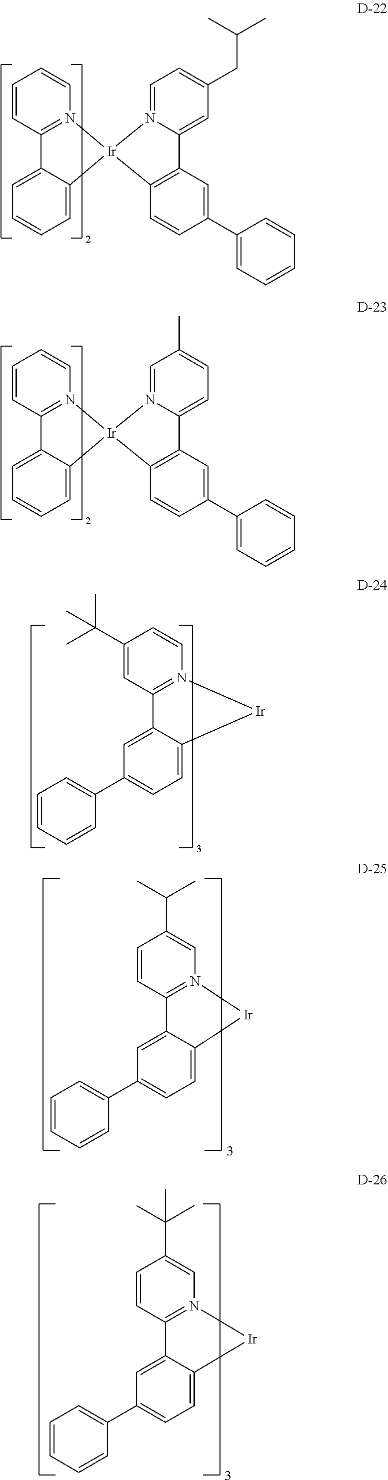

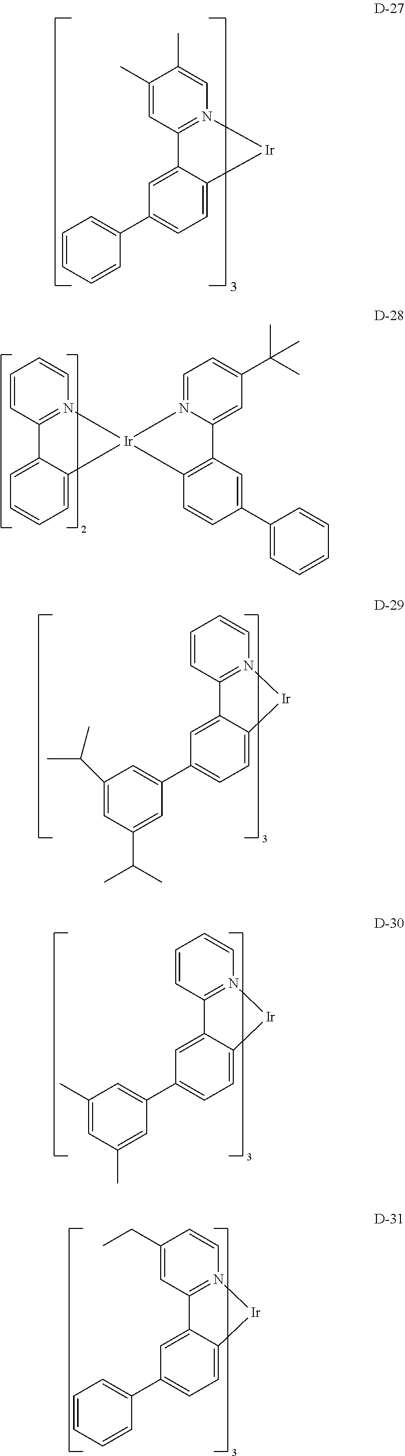

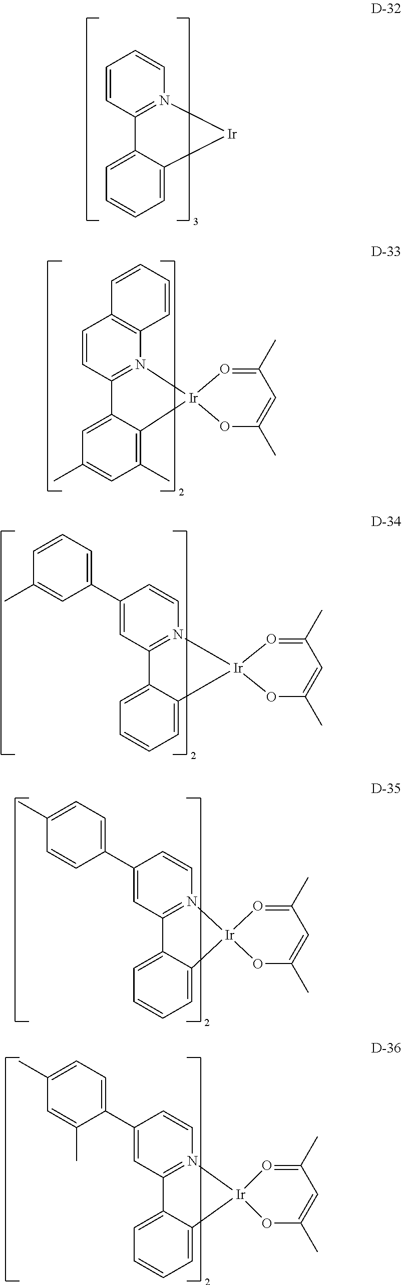

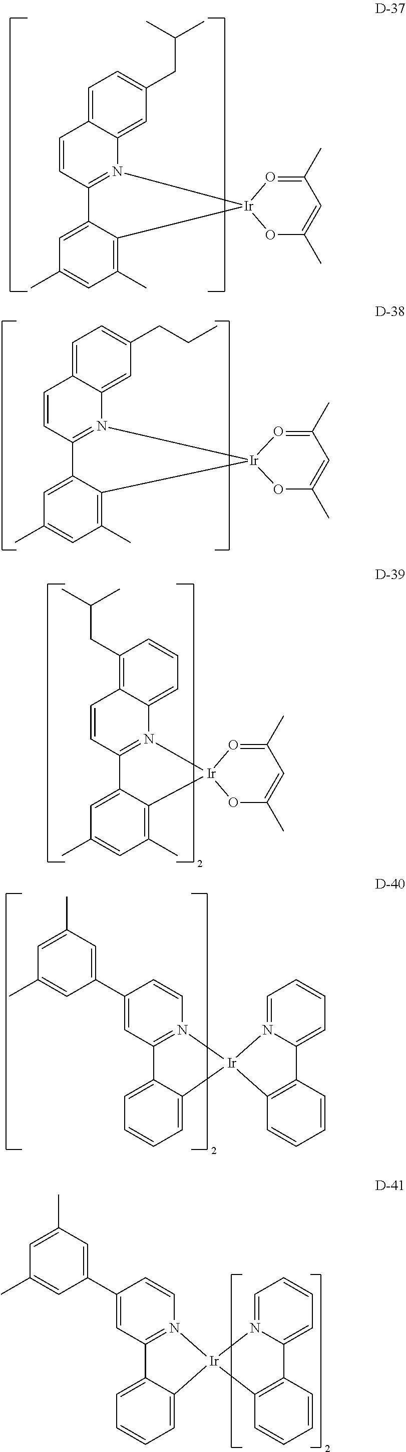

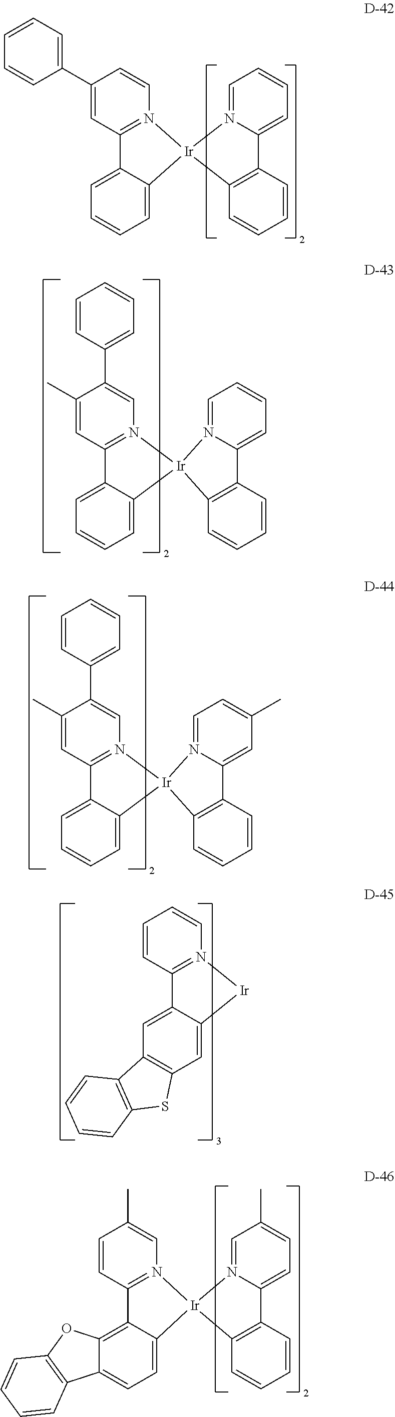

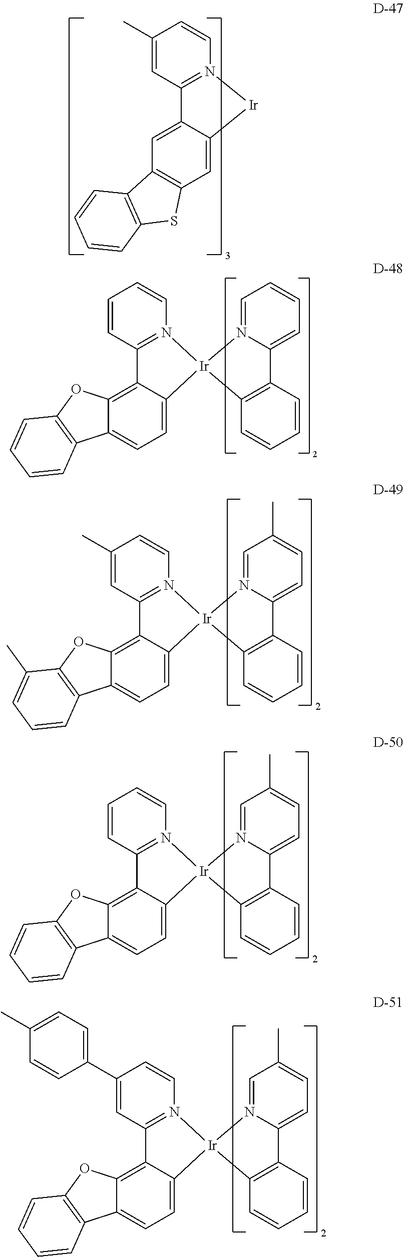

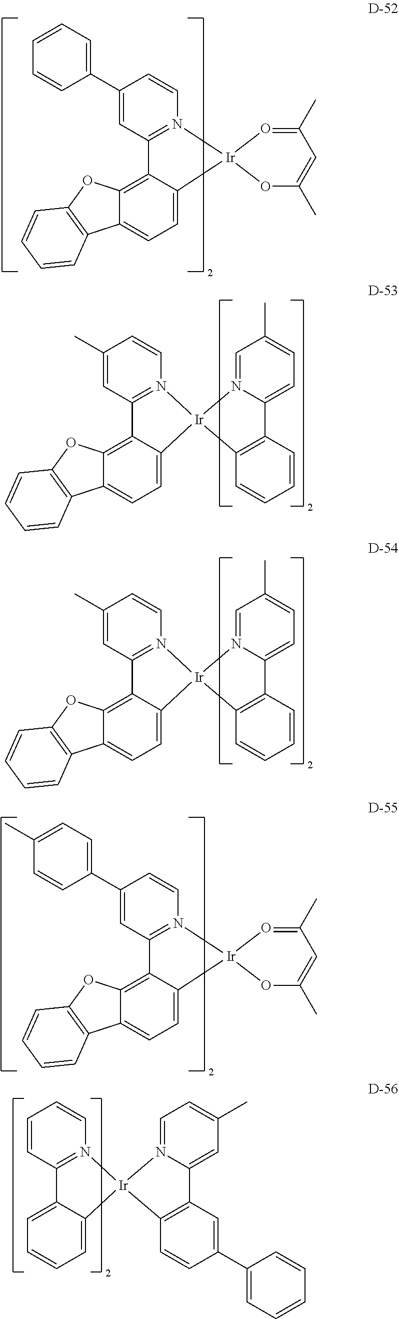

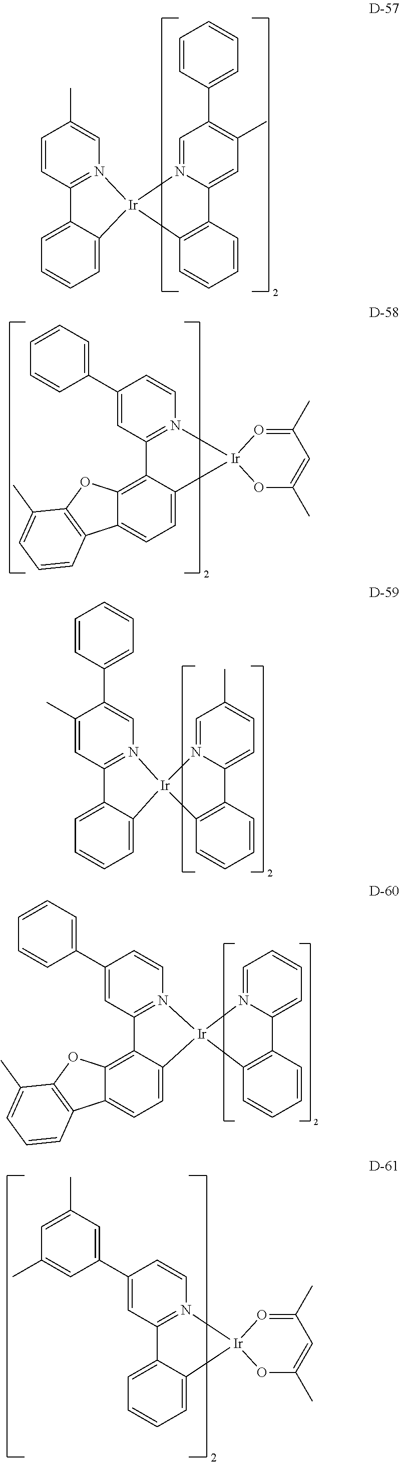

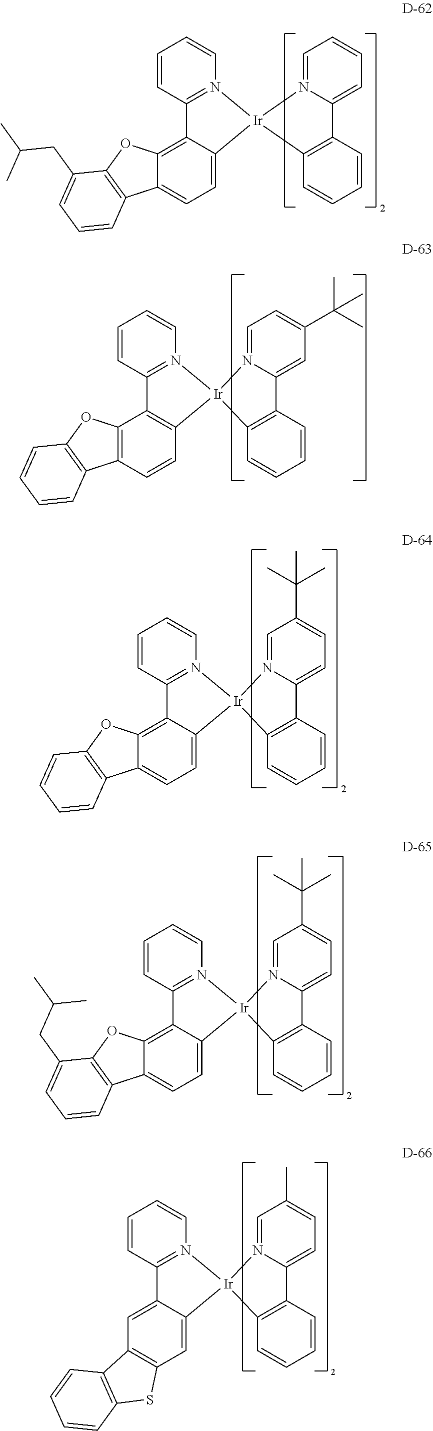

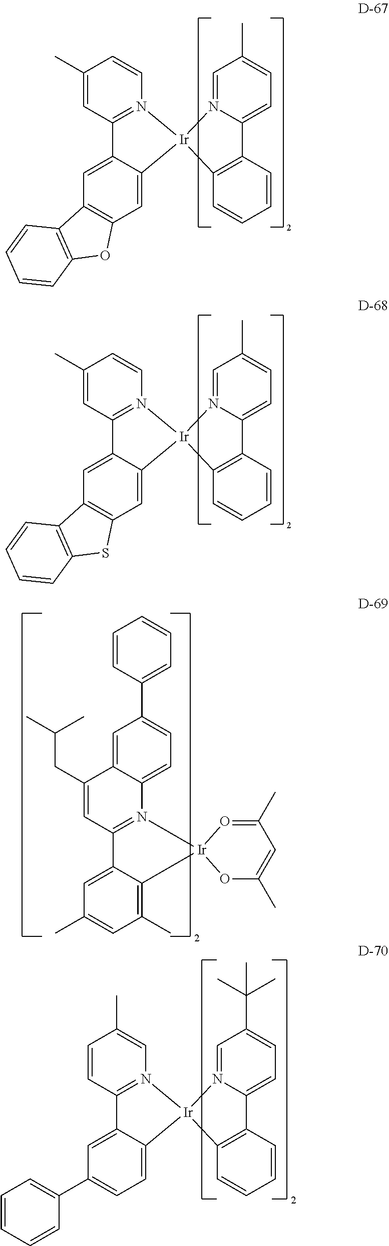

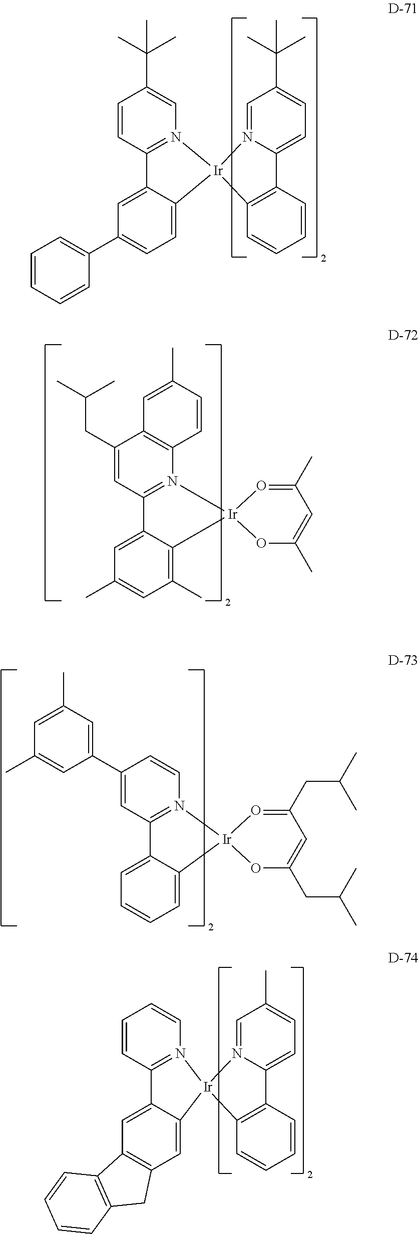

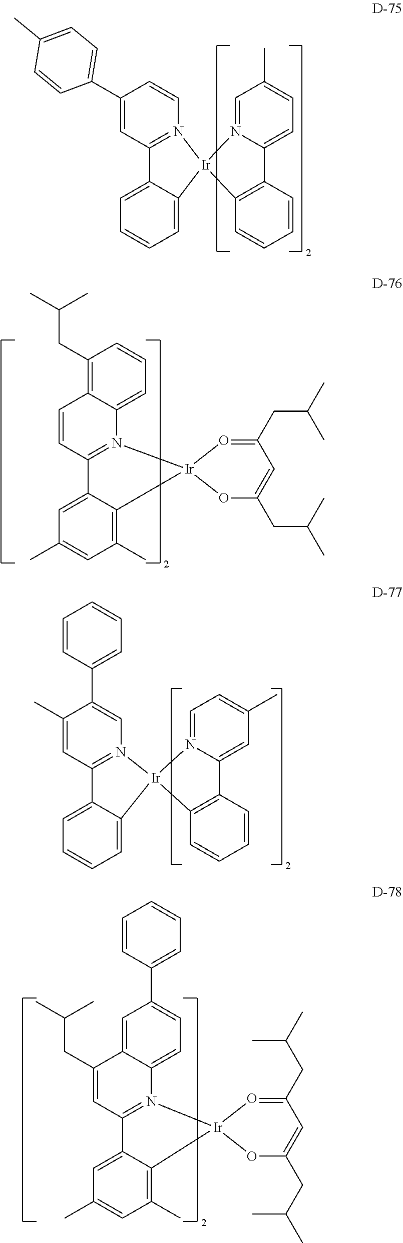

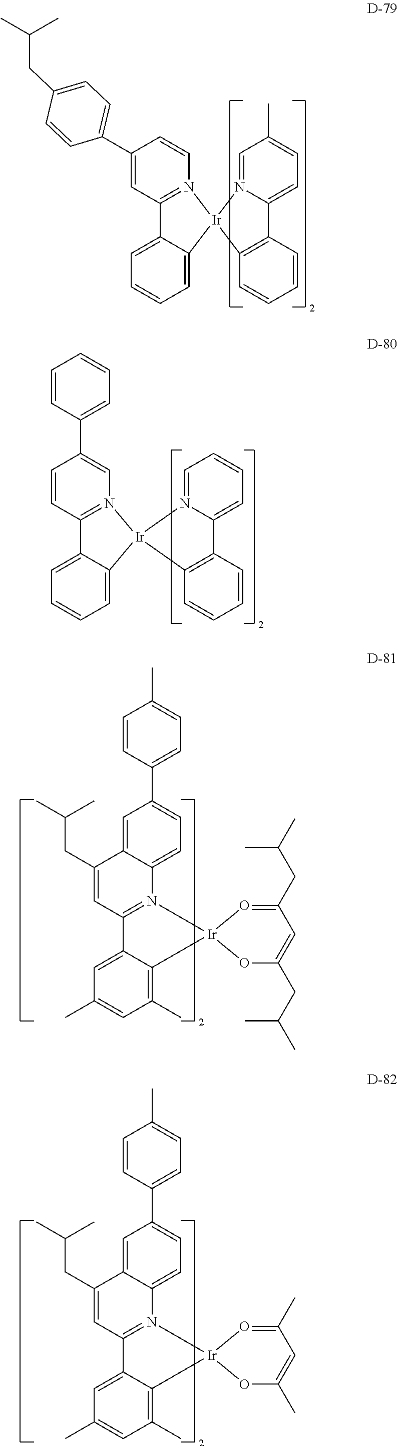

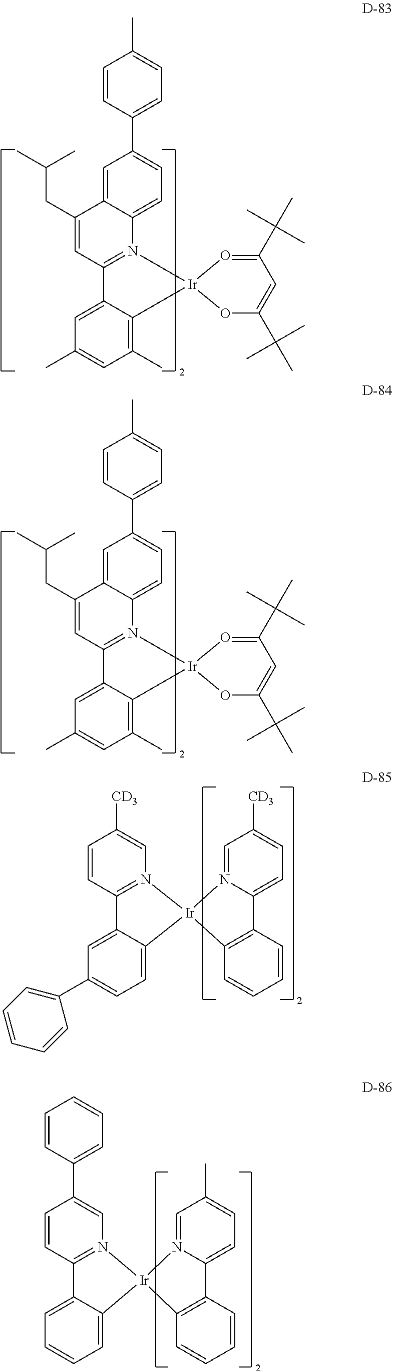

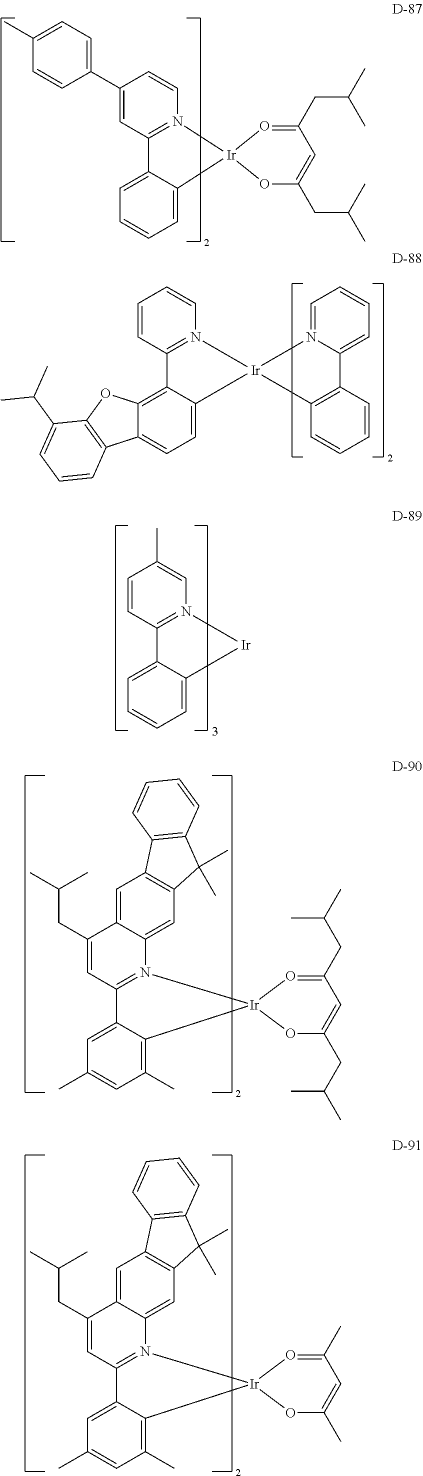

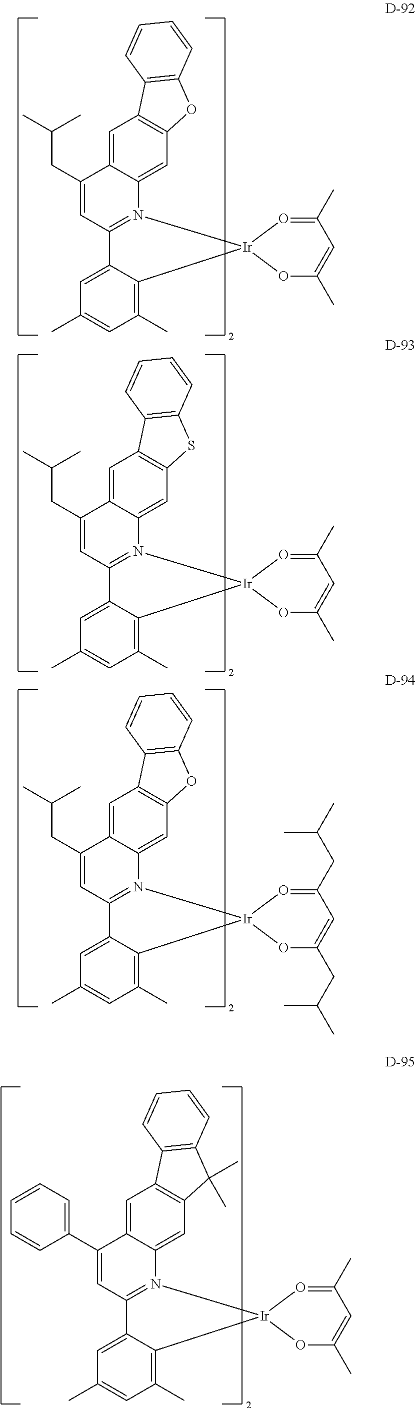

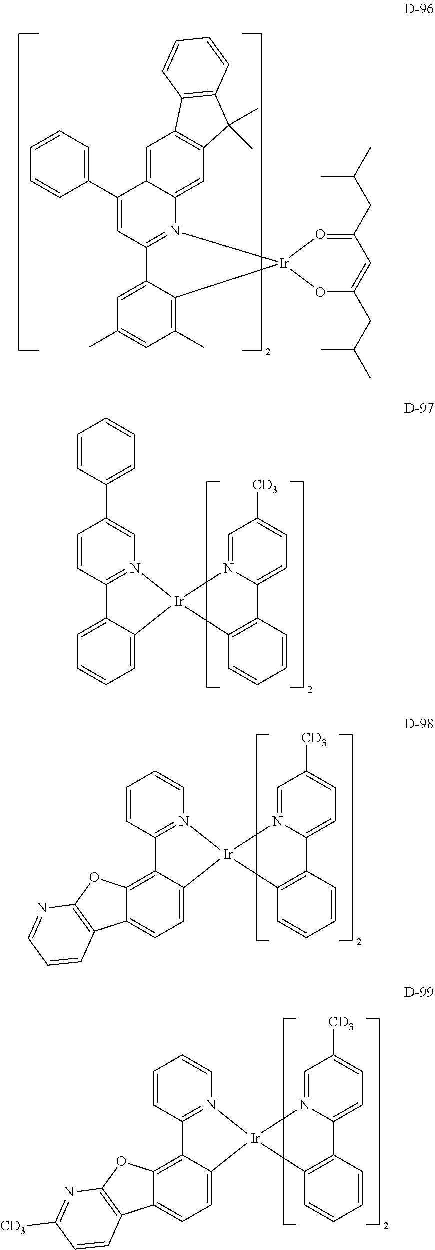

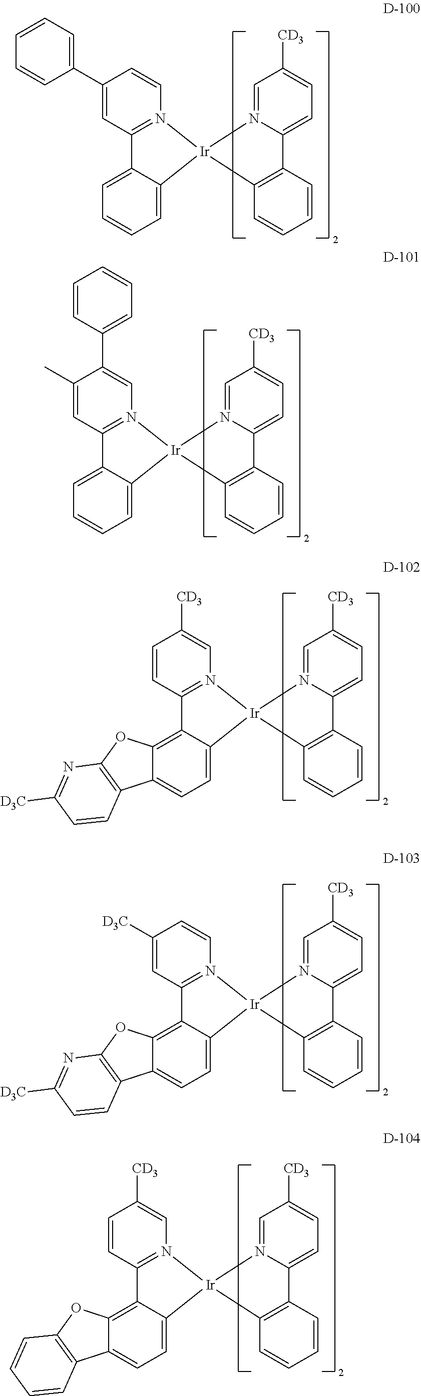

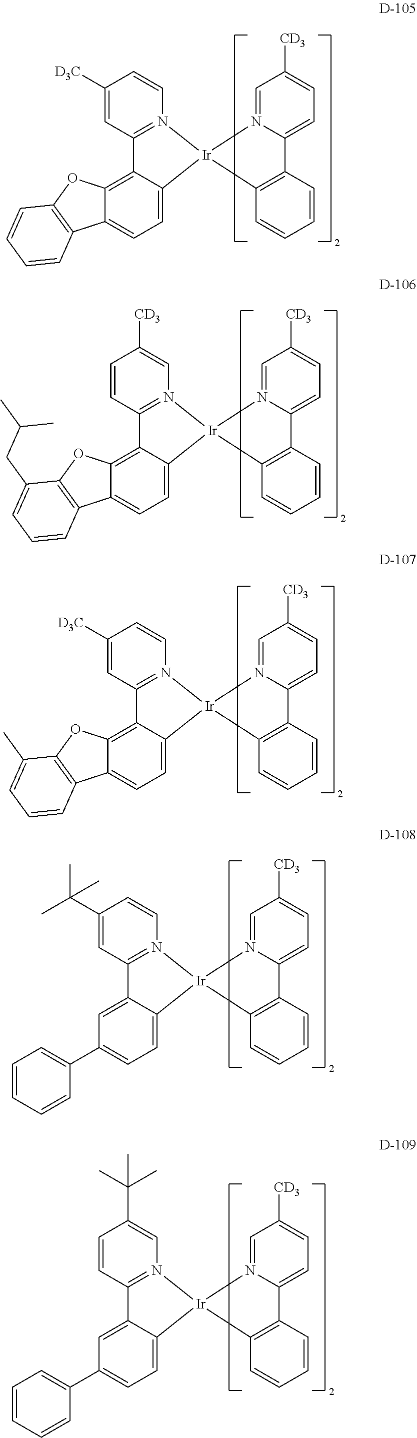

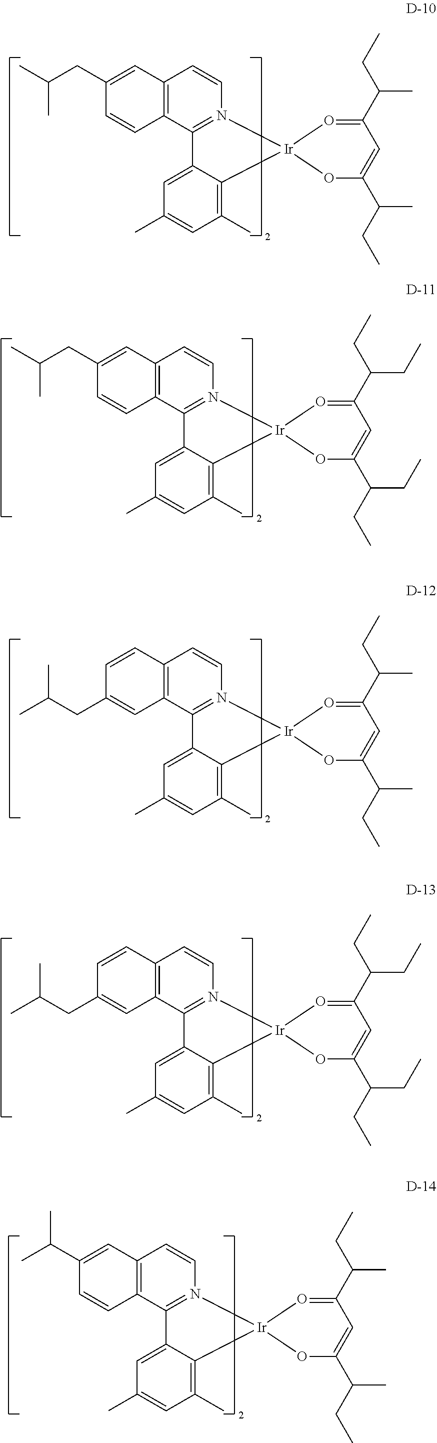

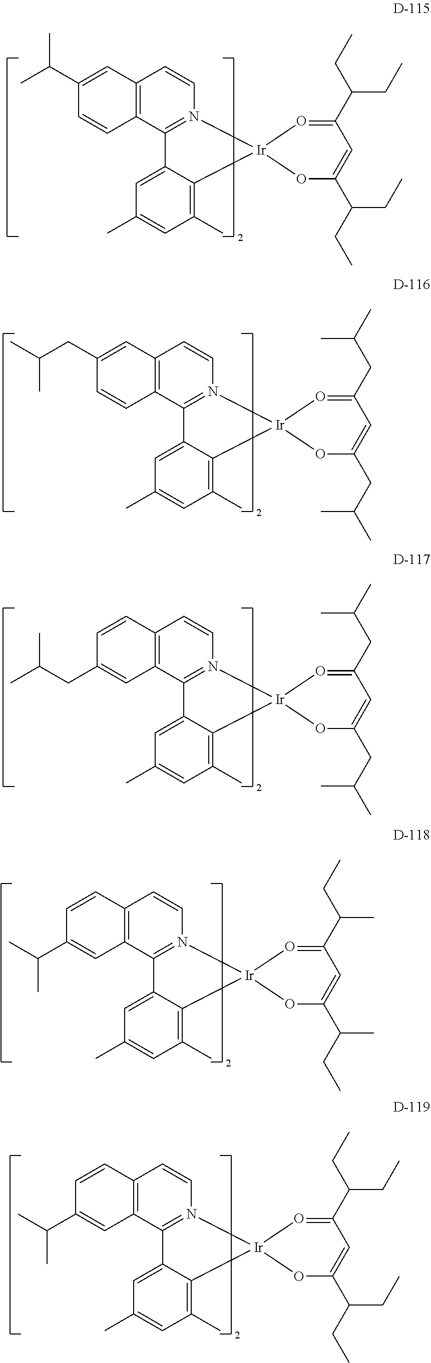

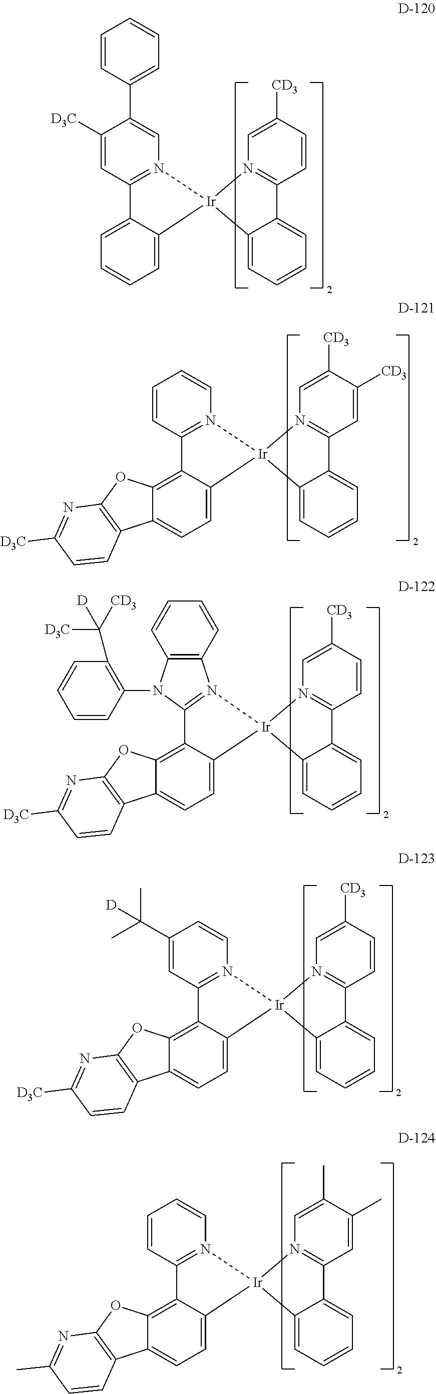

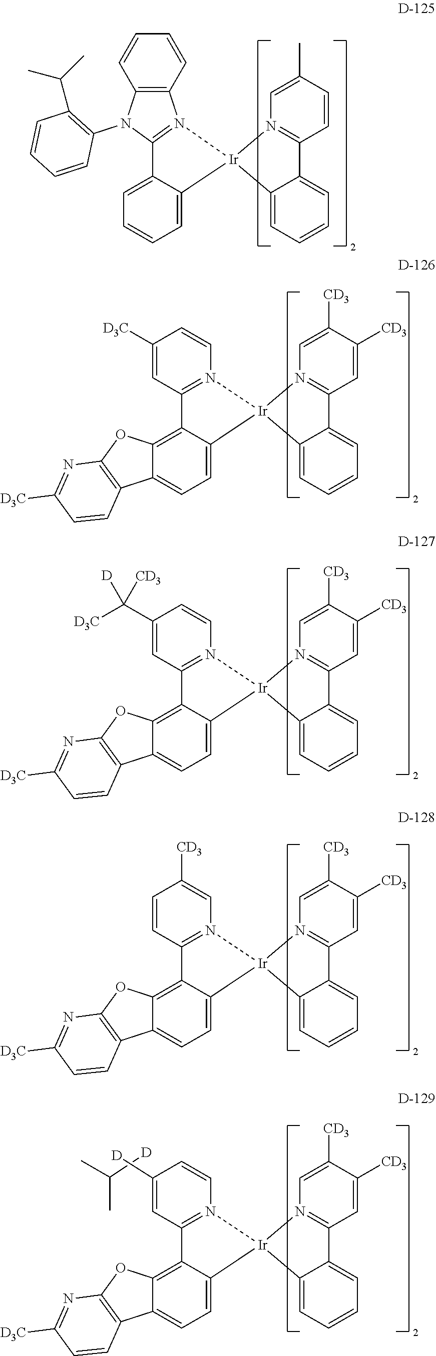

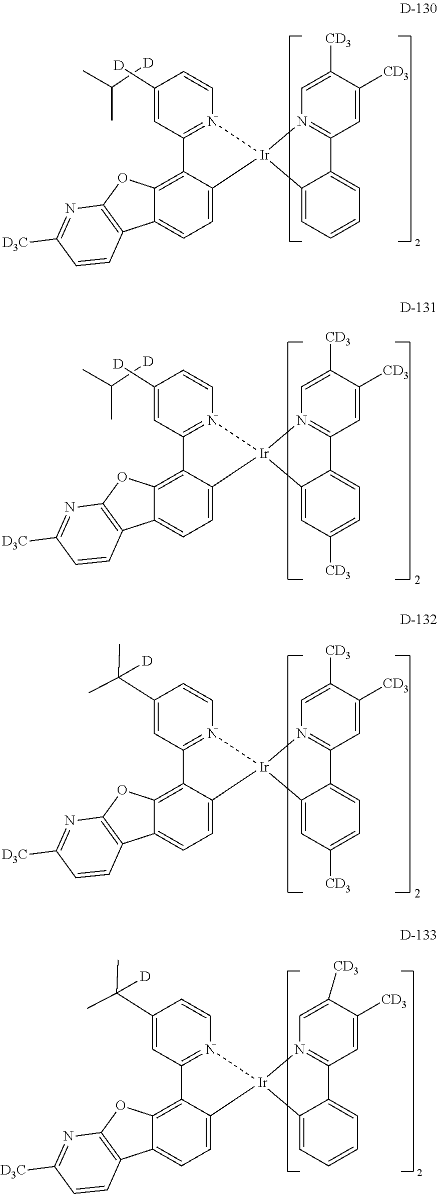

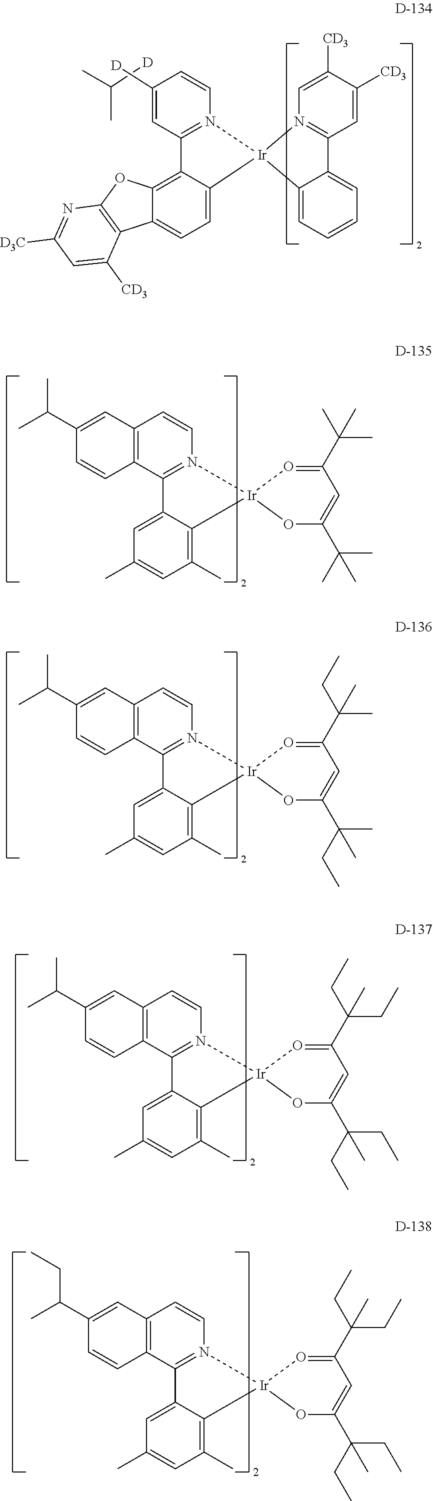

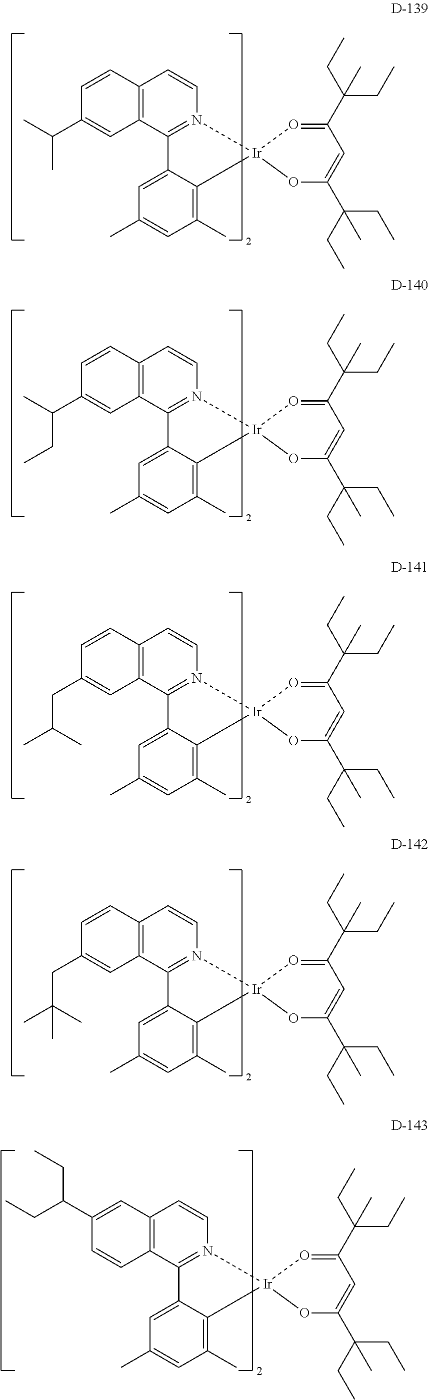

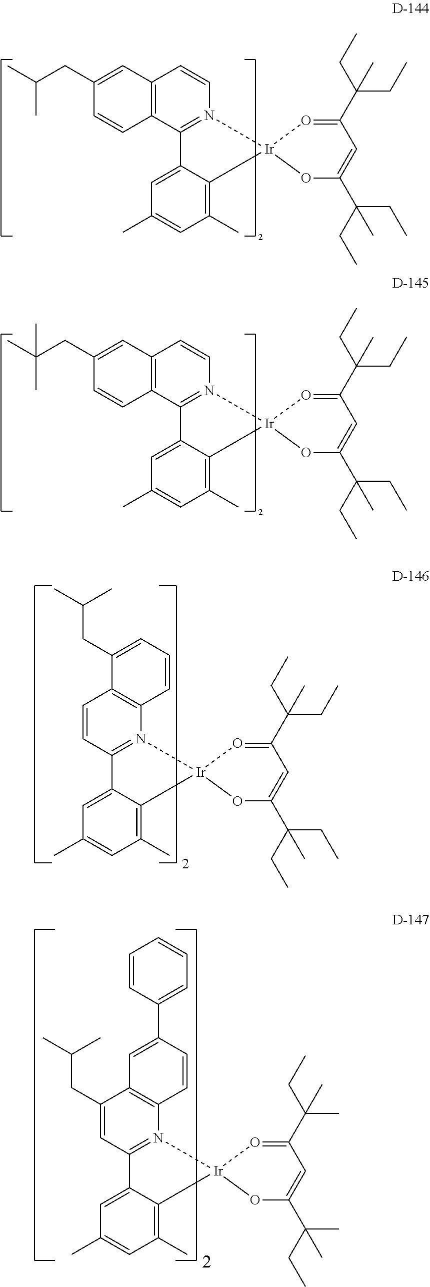

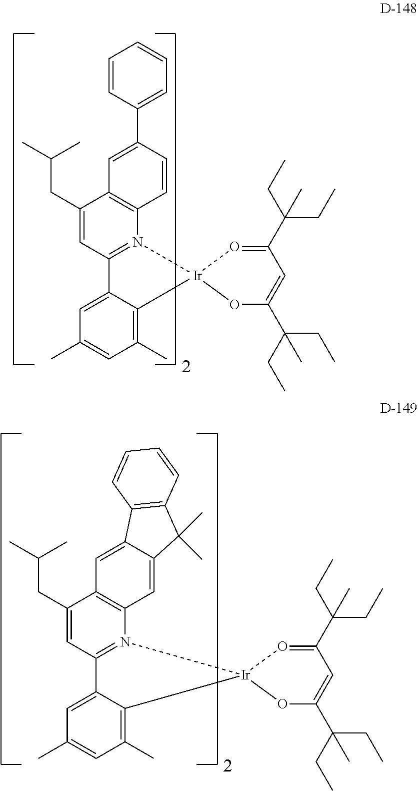

According to a further embodiment of the present disclosure, the present disclosure provides a composition for manufacturing an organic electroluminescent device. The composition is preferably a composition for manufacturing a hole transport layer, a hole auxiliary layer or a light-emitting auxiliary layer of an organic electroluminescent device, and includes the compound of the present disclosure. When the hole transport layer is two or more layers, the compound of the present disclosure may be included in the composition for manufacturing a hole transport layer (a hole auxiliary layer) adjacent to the light-emitting layer.

An organic electroluminescent device according to the present disclosure comprises an anode, a cathode, and at least one organic layer between the anode and the cathode. The organic layer comprises a light-emitting layer and may further comprise at least one layer selected from the group consisting of a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may further consist of a plurality of layers.

The anode and the cathode may be respectively formed with a transparent conductive material, or a transflective or reflective conductive material. The organic electroluminescent device may be a top emission type, a bottom emission type, or a both-sides emission type, depending on the materials forming the anode and the cathode. In addition, the hole injection layer may be further doped with a p-dopant, and the electron injection layer may be further doped with an n-dopant.

The organic layer may further comprise at least one compound selected from the group consisting of arylamine-based compounds and styrylarylamine-based compounds.

Further, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising the metal.

In addition, the organic electroluminescent device of the present disclosure may emit white light by further comprising at least one light-emitting layer, which comprises a blue, a red, or a green electroluminescent compound known in the field, besides the compound of the present disclosure. If necessary, it may further comprise a yellow or an orange light-emitting layer.

In the organic electroluminescent device of the present disclosure, preferably, at least one layer selected from the group consisting of a chalcogenide layer, a metal halide layer, and a metal oxide layer (hereinafter, “a surface layer”) may be placed on an inner surface(s) of one or both electrode(s). Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, the chalcogenide includes SiOₓ(1≤X≤2), AlOₓ(1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. The hole transport layer or the electron blocking layer may also be multi-layers.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each of the multi-layers may use a plurality of compounds.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or hole transport, or for preventing the overflow of electrons. When the light-emitting auxiliary layer is placed between the cathode and the light-emitting layer, it can be used for promoting the electron injection and/or electron transport, or for preventing the overflow of holes. Also, the hole auxiliary layer may be placed between the hole transport layer (or hole injection layer) and the light-emitting layer, and may be effective to promote or block the hole transport rate (or hole injection rate), thereby enabling the charge balance to be controlled. Further, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifetime of the organic electroluminescent device.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferably, the oxidative dopant includes various Lewis acids and acceptor compounds; and the reductive dopant includes alkali metals, alkali metal compounds, alkaline earth metals, rare-earth metals, and mixtures thereof. The reductive dopant layer may be employed as a charge-generating layer to produce an organic electroluminescent device having two or more light-emitting layers and emitting white light.

The organic electroluminescent material according to one embodiment of the present disclosure may be used as a light-emitting material for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. In addition, the organic electroluminescent material according to the one embodiment of the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film can be formed by dissolving or diffusing materials forming each layer into any suitable solvent such as ethanol, chloroform, tetrahydrofuran, dioxane, etc. The solvent can be any one where the materials forming each layer can be dissolved or diffused, and where there are no problems in film-formation capability.

In addition, it is possible to produce a display system, for example, a display system for smart phones, tablets, notebooks, PCs, TVs, or cars; or a lighting system, for example an outdoor or indoor lighting system, by using the organic electroluminescent device of the present disclosure.

Hereinafter, the preparation method of the organic electroluminescent compound according to the present disclosure, the properties thereof, and light-emitting characteristics of the organic electroluminescent device comprising the same will be explained in detail with reference to the representative compounds of the present disclosure. However, the present disclosure is not limited by the following examples.

### EXAMPLE 1: PREPARATION OF COMPOUND C-1

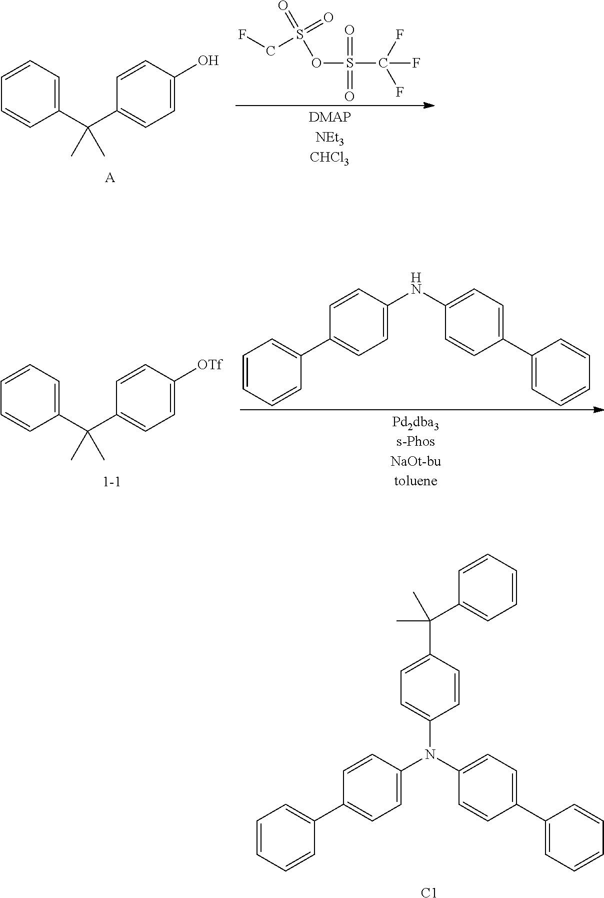

### Synthesis of Compound 1-1

In a flask, compound A (100 g, 471 mmol), triethylamine (99 mL, 706 mmol), 4-dimethylaminopyridine (DMAP) (5.7 g, 47.1 mmol), and chloroform (1.2 L) were added, the mixture was lowered to 0° C., and then stirred. Next, trifluoromethanesulfonic anhydride (120 mL) was slowly added thereto. After reaction for 3 hours, the resulting product was distilled under reduced pressure and separated by column chromatography to obtain compound 1-1 (155 g, yield: 95%).

### Synthesis of Compound C-1

In a flask, compound 1-1 (21.4 g, 62 mmol), diphenylamine (20 g, 62 mmol), tris(dibenzylideneacetone)dipalladium(0) (5.6 g, 6.2 mmol), s-phos (5.1 g, 12.4 mmol), sodium tert-butoxide (12 g, 124 mmol), and toluene (311 mL) were added, and stirred at 60° C. for 26 hours. After completion of the reaction, the organic layer was extracted with ethyl acetate, and the residual moisture was removed with magnesium sulfate. The resulting product was dried and separated by column chromatography to obtain compound C-1 (8.5 g, yield: 27%).

### EXAMPLE 2: PREPARATION OF COMPOUND C-41

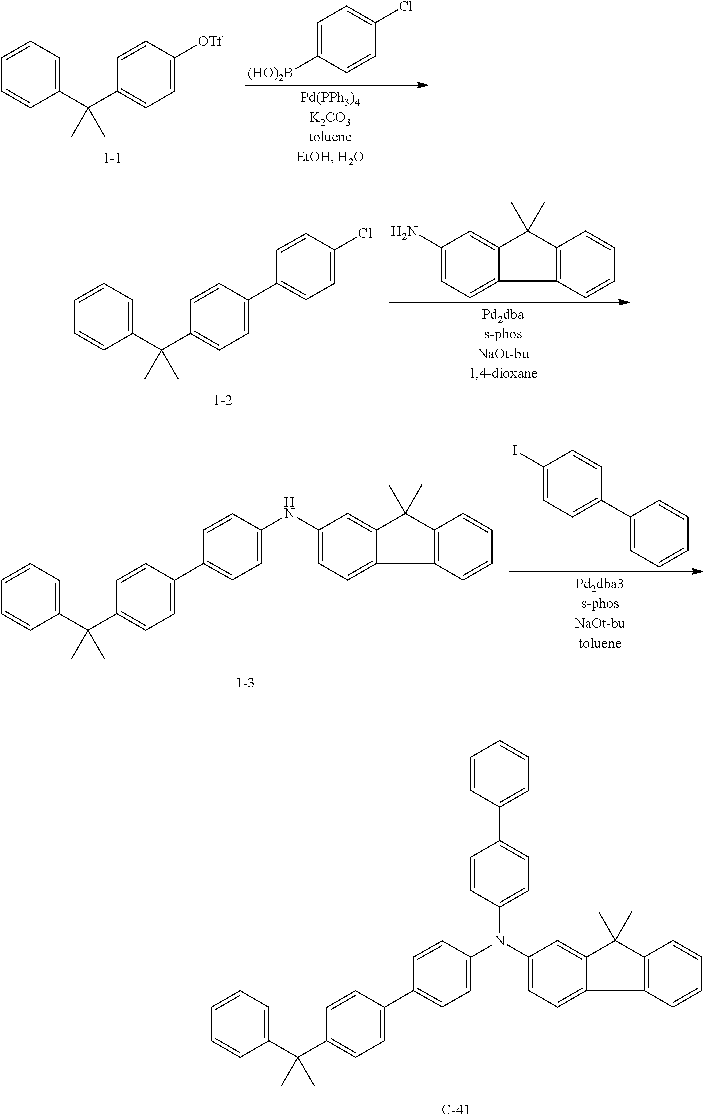

### Synthesis of Compound 1-2

In a flask, compound 1-1 (110 g, 320 mmol), 4-chlorophenylbonic acid (50 g, 320 mmol), tetrakis(triphenylphosphine)palladium(0) (Pd(PPh₃)₄ (18 g, 16 mmol), potassium carbonate (2 M, 320 mL), toluene (1000 mL), ethanol (320 mL), and water (320 mL) were added, dissolved, and stirred under reflux at 140° C. for 3 hours. After completion of the reaction, the organic layer was extracted with ethyl acetate, and the residual moisture was removed with magnesium sulfate. The resulting product was dried and separated by column chromatography to obtain compound 1-2 (64 g, yield: 65%).

### Synthesis of Compound 1-3

In a flask, compound 1-2 (30 g, 98 mmol), 9,9-dimethyl-9H-fluorene-2-amine (41 g, 195 mmol), tris(dibenzylideneacetone)dipalladium(0) (4.4 g, 4.9 mmol), s-phos (4 g, 9.8 mmol), sodium tert-butoxide (28.2 g, 294 mmol), and 1,4-dioxane (500 mL) were added and stirred under reflux at 150° C. for 3 hours. After completion of the reaction, the resulting product was filtered through celite, concentrated under reduced pressure, and separated by column chromatography to obtain compound 1-3 (41 g, yield: 87%).

### Synthesis of Compound C-41

In a flask, compound 1-3 (3 g, 6.2 mmol), 4-iodo-1,1-biphenyl (2.3 g, 8.1 mmol), tris(dibenzylideneacetone)dipalladium(0) (284 mg, 0.31 mmol), s-phos (254 mg, 0.62 mmol), sodium tert-butoxide (1.5 g, 15.5 mmol), and toluene (62 mL) were added and stirred at 140° C. for 25 hours. After completion of the reaction, the organic layer was extracted with ethyl acetate, and the residual moisture was removed with magnesium sulfate. The resulting product was dried and separated by column chromatography to obtain compound C-41 (2.1 g, yield: 55%).

### EXAMPLE 3: PREPARATION OF COMPOUND C-14

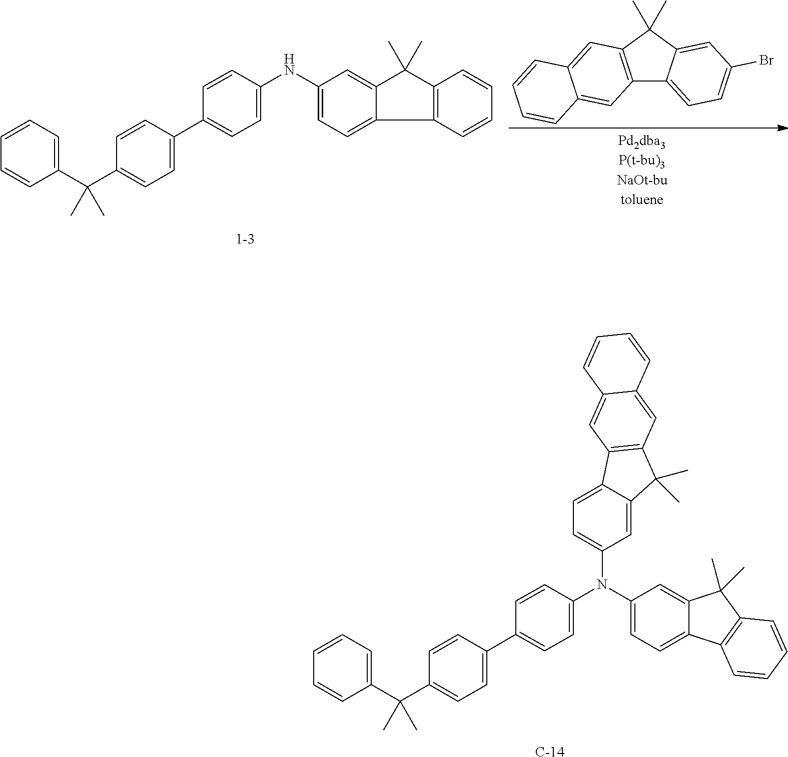

In a flask, compound 1-3 (15 g, 31 mmol), 2-bromo-11,11-dimethyl-11H-benzo[B]fluorene (12 g, 38 mmol), tris(dibenzylideneacetone)dipalladium(0) (1.4 g, 1.55 mmol), P(t-bu)₃ (50%, in o-xylene) (1.5 mL, 3.1 mmol), sodium tert-butoxide (8.9 g, 93 mmol), and toluene (160 mL) were added and stirred at 150° C. for 3 hours. After completion of the reaction, the organic layer was extracted with ethyl acetate, and the residual moisture was removed with magnesium sulfate. The resulting product was dried and separated by column chromatography to obtain compound C-14 (7.2 g, yield: 32%).

### EXAMPLE 4: PREPARATION OF COMPOUND C-211

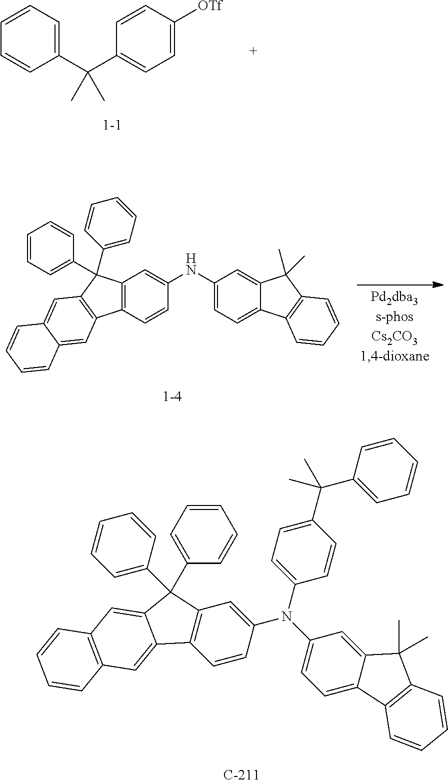 [SMILES: CC(C)(c1ccccc1)c1ccc(OS(=O)(=O)C(F)(F)F)cc1 ; CC1(C)c2ccccc2-c2ccc(Nc3ccc4c(c3)C(c3ccccc3)(c3ccccc3)c3cc5ccccc5cc3-4)cc21 ; CC(C)(c1ccccc1)c1ccc(N(c2ccc3c(c2)C(C)(C)c2ccccc2-3)c2ccc3c(c2)C(c2ccccc2)(c2ccccc2)c2cc4ccccc4cc2-3)cc1]

In a flask, compound 1-1 (9 g, 26 mmol), compound 1-4 (10 g, 17.4 mmol), tris(dibenzylideneacetone)dipalladium(0) (1.6 g, 1.74 mmol), s-phos (1.4 g, 3.5 mmol), cesium carbonate (17 g, 52.2 mmol), and 1,4-dioxane (150 mL) were added, and stirred at 140° C. for 27 hours. After completion of the reaction, the organic layer was extracted with ethyl acetate, and the residual moisture was removed with magnesium sulfate. The resulting product was dried and separated by column chromatography to obtain compound C-211 (2.1 g, yield: 10%).

### EXAMPLE 5: PREPARATION OF COMPOUND C-10

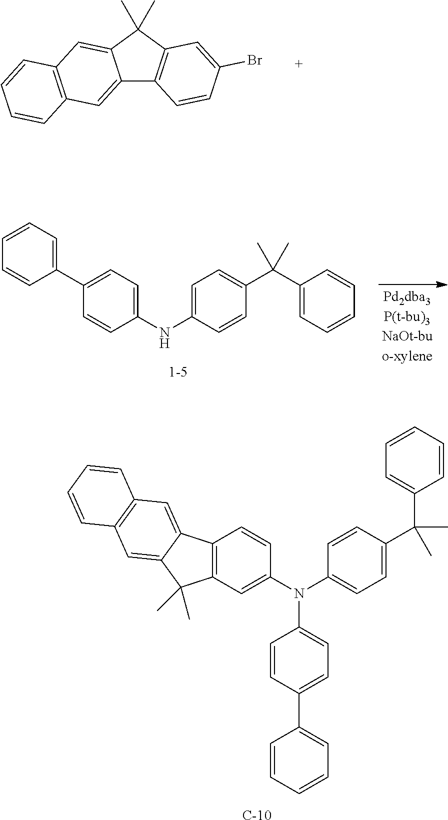

In a flask, 2-bromo-11,11-dimethyl-11H-benzo[B]fluorene (4.8 g, 14.8 mmol), compound 1-5 (4.5 g, 12.4 mmol), tris(dibenzylideneacetone)dipalladium(0) (568 mg, 0.62 mmol), P(t-bu)₃ (50%, in o-xylene) (0.6 mL, 1.24 mmol), sodium tert-butoxide (3.6 g, 37.2 mmol), and o-xylene (62 mL) were added and stirred at 150° C. for 1 hour. After completion of the reaction, the organic layer was extracted with ethyl acetate, and the residual moisture was removed with magnesium sulfate. The resulting product was dried and separated by column chromatography to obtain compound C-10 (1.5 g, yield: 17%).

### EXAMPLE 6: PREPARATION OF COMPOUND C-175

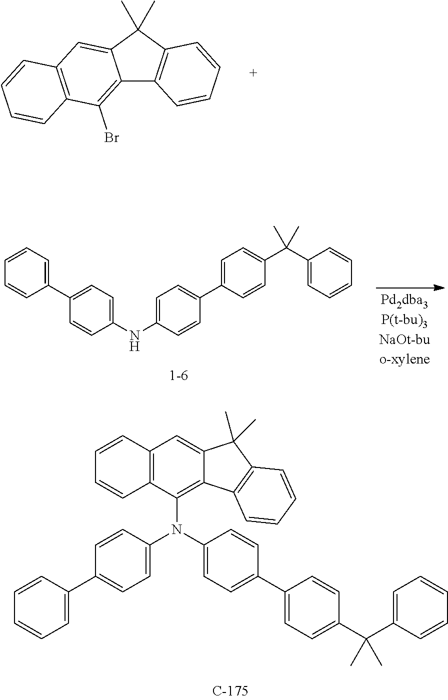 [SMILES: CC1(C)c2ccccc2-c2c1cc1ccccc1c2Br ; CC(C)(c1ccccc1)c1ccc(-c2ccc(Nc3ccc(-c4ccccc4)cc3)cc2)cc1 ; CC(C)(c1ccccc1)c1ccc(-c2ccc(N(c3ccc(-c4ccccc4)cc3)c3c4c(cc5ccccc35)C(C)(C)c3ccccc3-4)cc2)cc1]

In a flask, 5-bromo-11,11-dimethyl-11H-benzo[B]fluorene (10 g, 31 mmol), compound 1-6 (11.3 g, 25 mmol), tris(dibenzylideneacetone)dipalladium(0) (1.1 g, 1.25 mmol), P(t-bu)₃ (50%, in o-xylene) (1.2 mL, 2.5 mmol), sodium tert-butoxide (6 g, 62.5 mmol), and toluene (125 mL) were added and stirred at 140° C. for 3 hours. After completion of the reaction, the organic layer was extracted with ethyl acetate, and the residual moisture was removed with magnesium sulfate. The resulting product was dried and separated by column chromatography to obtain compound C-175 (1.3 g, yield: 6%).

### EXAMPLE 7: PREPARATION OF COMPOUND C-212

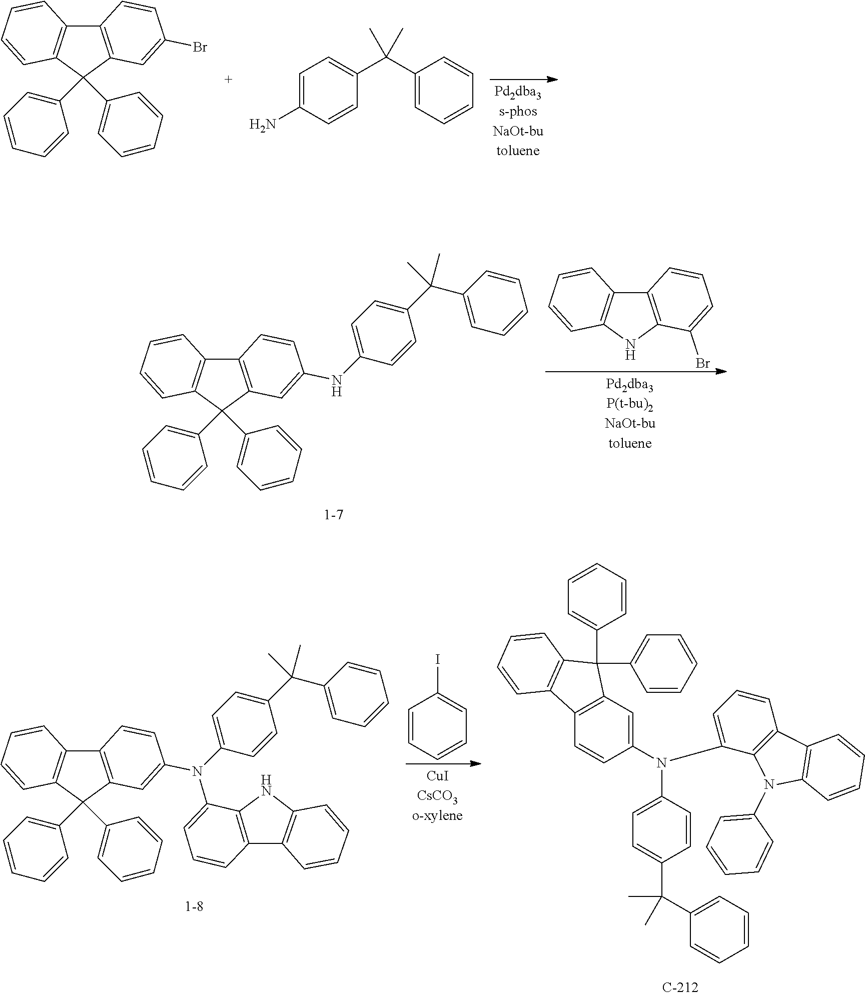

### Synthesis of Compound 1-7

In a flask, 2-bromo-9,9-diphenyl-9H-fluorene (25 g, 63 mmol), 4-(2-phenylpropan-2-yl)aniline (20 g, 95 mmol), tris(dibenzylideneacetone)dipalladium(0) (2.9 g, 3.15 mmol), s-phos (2.6 g, 6.3 mmol), sodium tert-butoxide (15.4 g, 158 mmol), and toluene (313 mL) were added, and stirred at 140° C. for 20 hours. After completion of the reaction, the organic layer was extracted with ethyl acetate, and the residual moisture was removed with magnesium sulfate. The resulting product was dried and separated by column chromatography to obtain compound 1-7 (18.7 g, yield: 43%).

### Synthesis of Compound 1-8

In a flask, compound 1-7 (18.7 g, 35.4 mmol), 1-bromo-9H-carbazole (8.7 g, 35.4 mmol), tris(dibenzylideneacetone)dipalladium(0) (1.6 g, 1.77 mmol) P(t-bu)₃ (50%, in o-xylene) (1.7 mL, 3.5 mmol), sodium tert-butoxide (8.5 g, 88.5 mmol), and o-xylene (350 mL) were added and stirred at 180° C. for 20 hours. After completion of the reaction, the organic layer was extracted with ethyl acetate, and the residual moisture was removed with magnesium sulfate. The resulting product was dried and separated by column chromatography to obtain compound 1-8 (20 g, yield: 82%).

### Synthesis of Compound C-212

In a flask, compound 1-8 (20 g, 29 mmol), 1-iodobenzene (18 g, 87 mmol), CuI (5.5 g, 29 mmol), cesium carbonate (28 g, 87 mmol), and o-xylene (290 mL) were added, and stirred at 190° C. for 52 hours. After completion of the reaction, the organic layer was extracted with ethyl acetate, and the residual moisture was removed with magnesium sulfate. The resulting product was dried and separated by column chromatography to obtain compound C-212 (2.1 g, yield: 9%).

### EXAMPLE 8: PREPARATION OF COMPOUND C-213

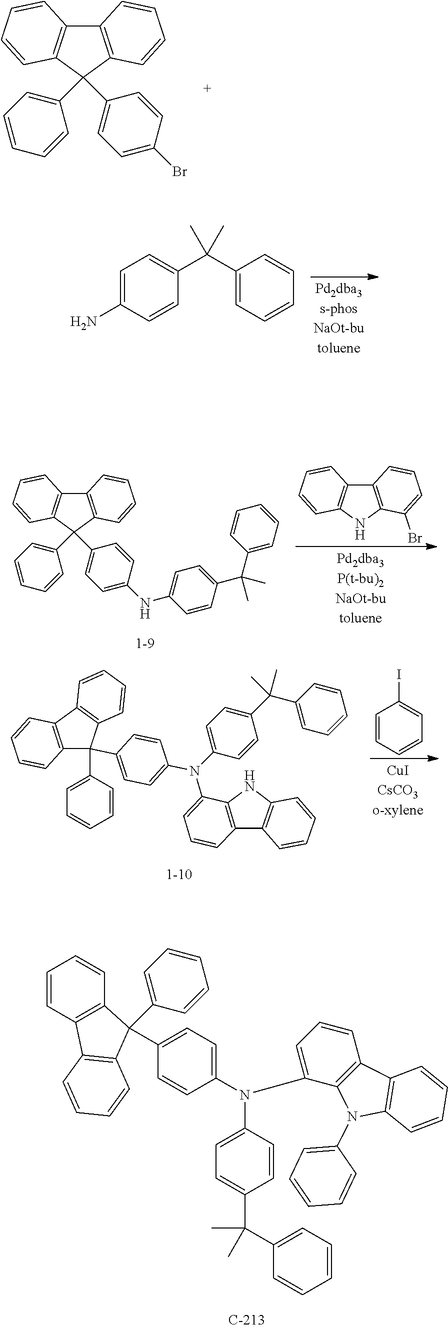

### Synthesis of Compound 1-9

In a flask, 9-(4-bromophenyl)-9-phenyl-9H-fluorene (25 g, 63 mmol), 4-(2-phenylpropan-2-yl)aniline (20 g, 95 mmol), tris(dibenzylideneacetone)dipalladium(0) (2.9 g, 3.15 mmol), s-phos (2.6 g, 6.3 mmol), sodium tert-butoxide (15.4 g, 158 mmol), and toluene (313 mL) were added, and stirred at 140° C. for 4 hours. After completion of the reaction, the organic layer was extracted with ethyl acetate, and the residual moisture was removed with magnesium sulfate. The resulting product was dried and separated by column chromatography to obtain compound C-9 (13 g, yield: 39%).

### Synthesis of Compound 1-10

In a flask, compound 1-9 (13 g, 25 mmol), 1-bromo-9H-carbazole (6 g, 25 mmol), tris(dibenzylideneacetone)dipalladium(0) (1.1 g, 1.25 mmol), P(t-bu)₃ (50%, in o-xylene) (1.2 mL, 2.5 mmol), sodium tert-butoxide (6 g, 62.5 mmol), and o-xylene (250 mL) were added, and stirred at 180° C. for 48 hours. After completion of the reaction, the organic layer was extracted with ethyl acetate, and the residual moisture was removed with magnesium sulfate. The resulting product was dried and separated by column chromatography to obtain compound C-10 (10.5 g, yield: 58%).

### Synthesis of Compound C-213

In a flask, compound 1-10 (10 g, 14.4 mmol), 1-iodobenzene (9 g, 43.2 mmol), CuI (3 g, 14.4 mmol), cesium carbonate (14 g, 43.2 mmol), and o-xylene (150 mL) were added, and stirred at 190° C. for 6 hours. After completion of the reaction, the organic layer was extracted with ethyl acetate, and the residual moisture was removed with magnesium sulfate. The resulting product was dried and separated by column chromatography to obtain compound C-213 (3.2 g, yield: 9%).

### EXAMPLE 9: PREPARATION OF COMPOUND C-214

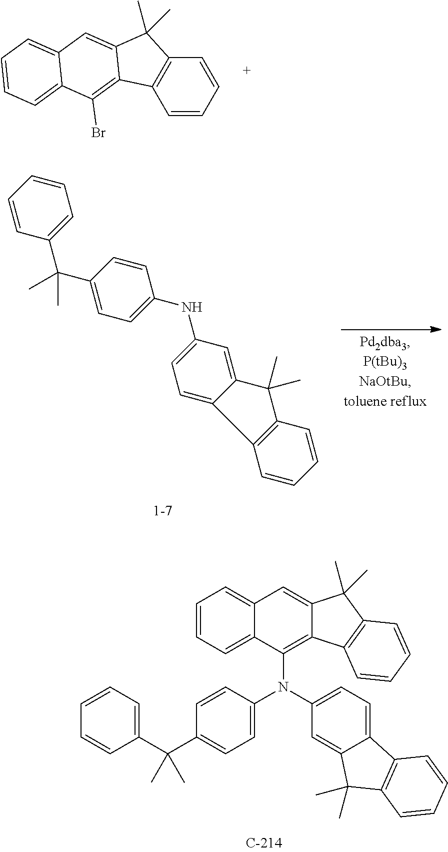 [SMILES: CC1(C)c2ccccc2-c2c1cc1ccccc1c2Br ; CC(C)(c1ccccc1)c1ccc(Nc2ccc3c(c2)C(C)(C)c2ccccc2-3)cc1 ; CC(C)(c1ccccc1)c1ccc(N(c2ccc3c(c2)C(C)(C)c2ccccc2-3)c2c3c(cc4ccccc24)C(C)(C)c2ccccc2-3)cc1]

In a flask, 5-bromo-11,11-dimethyl-11H-benzo[8]fluorene (5.3 g, 16.4 mmol), compound 1-7 (6.0 g, 14.9 mmol), tris(dibenzylideneacetone)dipalladium(0) (0.68 g, 0.74 mmol), P(t-bu)₃ (50%, in o-xylene) (0.73 mL, 1.5 mmol), sodium teff-butoxide (2.1 g, 22.3 mmol), and toluene (60 mL) were added, and stirred under reflux at 120° C. for 18 hours. After completion of the reaction, the organic layer was extracted with ethyl acetate, and the residual moisture was removed with magnesium sulfate. The resulting product was dried and separated by column chromatography to obtain compound C-214 (5.1 g, yield: 53%).

### EXAMPLE 10: PREPARATION OF COMPOUND C-215

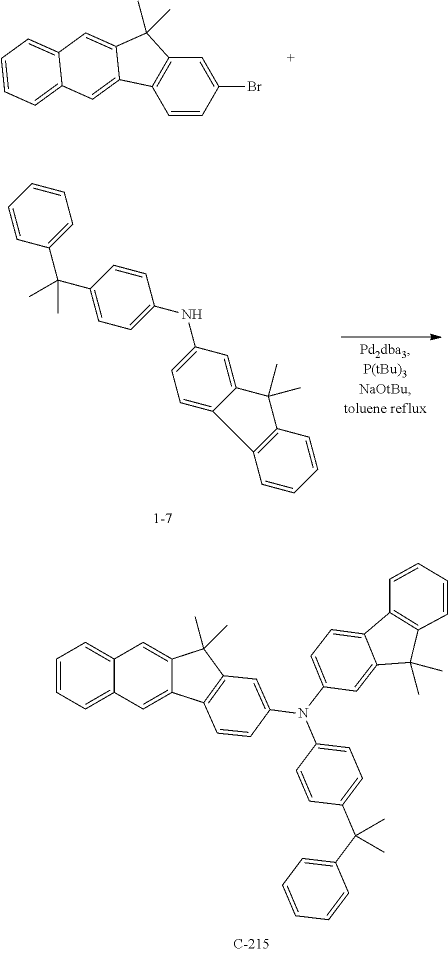 [SMILES: CC1(C)c2cc(Br)ccc2-c2cc3ccccc3cc21 ; CC(C)(c1ccccc1)c1ccc(Nc2ccc3c(c2)C(C)(C)c2ccccc2-3)cc1 ; CC(C)(c1ccccc1)c1ccc(N(c2ccc3c(c2)C(C)(C)c2ccccc2-3)c2ccc3c(c2)C(C)(C)c2cc4ccccc4cc2-3)cc1]

In a flask, 2-bromo-11,11-dimethyl-11H-benzo[B]fluorene (5.3 g, 16.4 mmol), compound 1-7 (6.0 g, 14.9 mmol), tris(dibenzylideneacetone)dipalladium(0) (0.68 g, 0.74 mmol), P(t-bu)₃ (50%, in o-xylene) (0.73 mL, 1.5 mmol), sodium tert-butoxide (2.1 g, 22.3 mmol), and toluene (60 mL) were added, and stirred under reflux at 120° C. for 3 hours. After completion of the reaction, the organic layer was extracted with ethyl acetate, and the residual moisture was removed with magnesium sulfate. The resulting product was dried and separated by column chromatography to obtain compound C-215 (3.2 g, yield: 33%).

### EXAMPLE 11: PREPARATION OF COMPOUND C-216

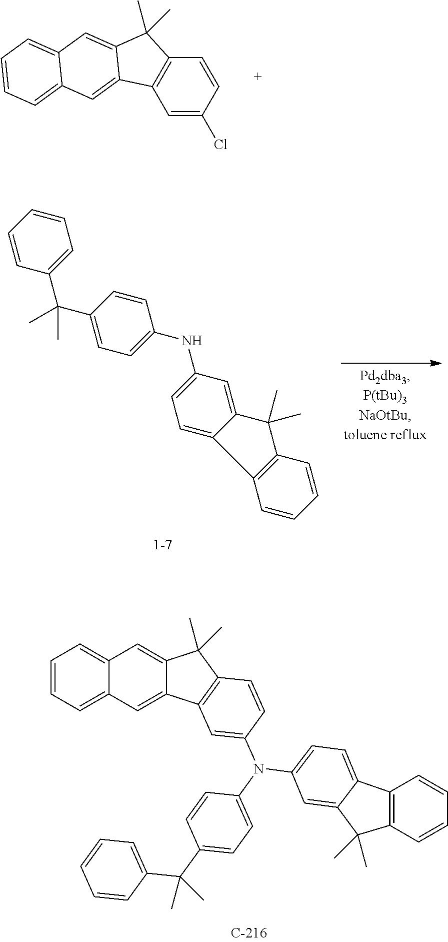 [SMILES: CC1(C)c2ccc(Cl)cc2-c2cc3ccccc3cc21 ; CC(C)(c1ccccc1)c1ccc(Nc2ccc3c(c2)C(C)(C)c2ccccc2-3)cc1 ; CC(C)(c1ccccc1)c1ccc(N(c2ccc3c(c2)-c2cc4ccccc4cc2C3(C)C)c2ccc3c(c2)C(C)(C)c2ccccc2-3)cc1]

In a flask, 3-chloro-11,11-dimethyl-11H-benzo[B]fluorene (4.6 g, 16.4 mmol), compound 1-7 (6.0 g, 14.9 mmol), tris(dibenzylideneacetone)dipalladium(0) (0.68 g, 0.74 mmol), P(t-bu)₃ (50%, in o-xylene) (0.73 mL, 1.5 mmol), sodium tart-butoxide (2.1 g, 22.3 mmol), and toluene (60 mL) were added, and stirred under reflux at 120° C. for 3 hours. After completion of the reaction, the organic layer was extracted with ethyl acetate, and the residual moisture was removed with magnesium sulfate. The resulting product was dried and separated by column chromatography to obtain compound C-216 (5.5 g, yield: 57%).

### EXAMPLE 12: PREPARATION OF COMPOUND C-217

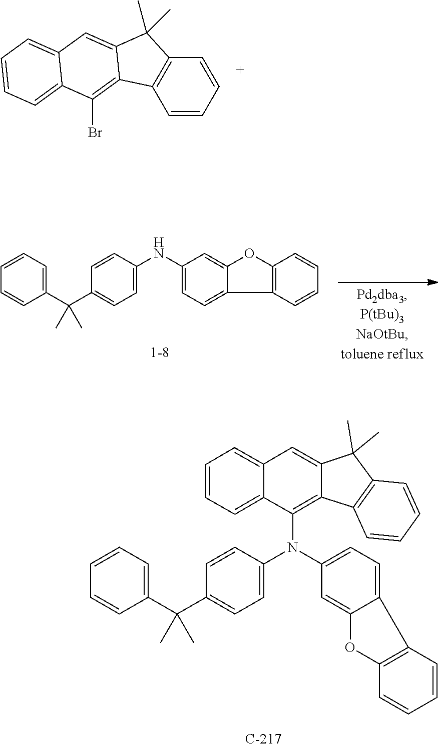 [SMILES: CC1(C)c2ccccc2-c2c1cc1ccccc1c2Br ; CC(C)(c1ccccc1)c1ccc(Nc2ccc3c(c2)oc2ccccc23)cc1 ; CC(C)(c1ccccc1)c1ccc(N(c2ccc3c(c2)oc2ccccc23)c2c3c(cc4ccccc24)C(C)(C)c2ccccc2-3)cc1]

In a flask, 5-bromo-11,11-dimethyl-11H-benzo[B]fluorene (9.4 g, 29.1 mmol), compound 1-8 (10.0 g, 26.5 mmol), tris(dibenzylideneacetone)dipalladium(0) (1.21 g, 1.32 mmol), P(t-bu)₃ (50%, in o-xylene) (1.30 mL, 2.65 mmol), sodium tert-butoxide (3.8 g, 39.7 mmol), and toluene (100 mL) were added, and stirred under reflux at 120° C. for 4 hours. After completion of the reaction, the organic layer was extracted with ethyl acetate, and the residual moisture was removed with magnesium sulfate. The resulting product was dried and separated by column chromatography to obtain compound C-217 (8.7 g, yield: 53%).

### EXAMPLE 13: PREPARATION OF COMPOUND C-218

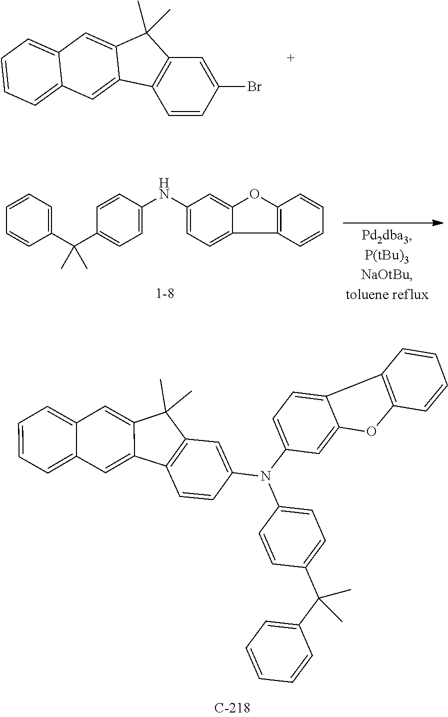 [SMILES: CC1(C)c2cc(Br)ccc2-c2cc3ccccc3cc21 ; CC(C)(c1ccccc1)c1ccc(Nc2ccc3c(c2)oc2ccccc23)cc1 ; CC(C)(c1ccccc1)c1ccc(N(c2ccc3c(c2)C(C)(C)c2cc4ccccc4cc2-3)c2ccc3c(c2)oc2ccccc23)cc1]

In a flask, 2-bromo-11,11-dimethyl-11H-benzo[B]fluorene (5.6 g, 17.8 mmol), compound 1-8 (6.0 g, 15.9 mmol), tris(dibenzylideneacetone)dipalladium(0) (0.73 g, 0.80 mmol), P(t-bu)₃ (50%, in o-xylene) (0.78 mL, 1.59 mmol), sodium tert-butoxide (2.30 g, 23.8 mmol), and toluene (60 mL) were added, and stirred under reflux at 120° C. for 4 hours. After completion of the reaction, the organic layer was extracted with ethyl acetate, and the residual moisture was removed with magnesium sulfate. The resulting product was dried and separated by column chromatography to obtain compound C-218 (4.8 g, yield: 49%).

### EXAMPLE 14: PREPARATION OF COMPOUND C-219

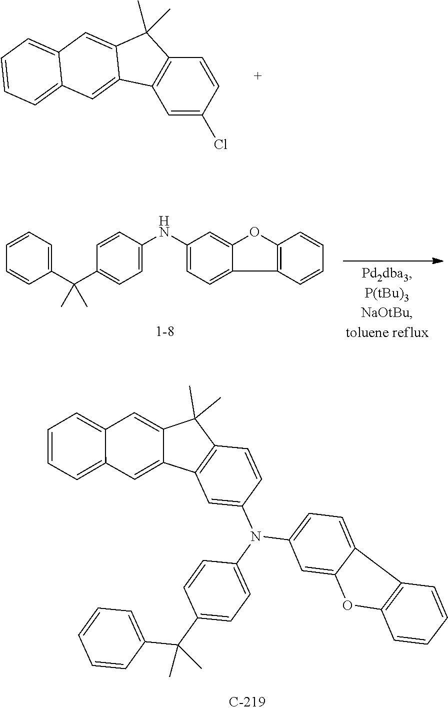 [SMILES: CC1(C)c2ccc(Cl)cc2-c2cc3ccccc3cc21 ; CC(C)(c1ccccc1)c1ccc(Nc2ccc3c(c2)oc2ccccc23)cc1 ; CC(C)(c1ccccc1)c1ccc(N(c2ccc3c(c2)-c2cc4ccccc4cc2C3(C)C)c2ccc3c(c2)oc2ccccc23)cc1]

In a flask, 3-chloro-11,11-dimethyl-11H-benzo[B]fluorene (4.1 g, 14.6 mmol), compound 1-8 (5.0 g, 13.2 mmol), tris(dibenzylideneacetone)dipalladium(0) (0.61 g, 0.66 mmol), P(t-bu),₃ (50%, in o-xylene) (0.65 mL, 1.32 mmol), sodium tert-butoxide (1.91 g, 19.9 mmol), and toluene (50 mL) were added, and stirred under reflux at 120° C. for 4 hours. After completion of the reaction, the organic layer was extracted with ethyl acetate, and the residual moisture was removed with magnesium sulfate. The resulting product was dried and separated by column chromatography to obtain compound C-219 (5.0 g, yield: 61%).

### DEVICE EXAMPLES 1-1 TO 1-9, AND 2-1 TO 2-3: PRODUCING OLEDS COMPRISING THE ORGANIC ELECTROLUMINESCENT COMPOUNDS ACCORDING TO THE PRESENT DISCLOSURE

OLEDs according to the present disclosure were produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 was introduced into a cell of the vacuum vapor deposition apparatus, and compound HT-1 was introduced into another cell of the vacuum vapor deposition apparatus. The two materials were evaporated at different rates, and compound HI-1 was deposited in a doping amount of 3 wt % based on the total amount of compound HI-1 and compound HT-1 to form a hole injection layer having a thickness of 10 nm. Next, compound HT-1 was deposited on the hole injection layer to form a first hole transport layer having a thickness of 90 nm. Then, the compounds shown in Tables 1 and 2 below were deposited to form a second hole transport layer having a thickness of 60 nm. After forming the hole injection layer and the hole transport layers, a light-emitting layer was formed thereon as follows: Compounds shown in Table 1 and 2 below were introduced into two cells of the vacuum vapor deposition apparatus as hosts, and compound D-39 was introduced into another cell as a dopant. The two materials were evaporated at different rates and the dopant was doped in a doping amount of 2 wt % based on the total amount of the hosts and dopant to form a light-emitting layer having a thickness of 40 nm on the second hole transport layer. Next, compound HBL was deposited as an electron buffer material on the light-emitting layer to form an electron buffer layer having a thickness of 5 nm. Next, compounds ETL-1 and EIL-1 were deposited at a weight ratio of 5:5 to form an electron transport layer having a thickness of 30 nm. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, OLEDs were produced.

### DEVICE EXAMPLES 3-1 TO 3-3: PRODUCING OLEDS COMPRISING THE ORGANIC ELECTROLUMINESCENT COMPOUNDS ACCORDING TO THE PRESENT DISCLOSURE

OLEDs were produced in the same manner as in Device Examples 1-1 to 1-9, and 2-1 to 2-3, except for the following: Compound RH-3 and compound RH-4 shown in Table 3 below were introduced into different cells of the vacuum vapor deposition apparatus as hosts, and compound D-39 was introduced into another cell as a dopant. Compound RH-3 and compound RH-4 were evaporated at a rate of 5:5 and the dopant was doped in a doping amount of 2 wt % based on the total amount of the hosts and dopant to form a light-emitting layer having a thickness of 40 nm on the second hole transport layer.

### COMPARATIVE EXAMPLES 1-1 TO 1-3, 2-1 AND 2-2: PRODUCING OLEDS NOT COMPRISING THE ORGANIC ELECTROLUMINESCENT COMPOUNDS ACCORDING TO THE PRESENT DISCLOSURE

OLEDs were produced in the same manner as in Device Examples 1-1 to 1-9, and 2-1 to 2-3, except that the compounds shown in Tables 1 and 2 below were used as the second hole transport layer and the host material.

### COMPARATIVE EXAMPLES 3-1 AND 3-2: PRODUCING OLEDS NOT COMPRISING THE ORGANIC ELECTROLUMINESCENT COMPOUNDS ACCORDING TO THE PRESENT DISCLOSURE

OLEDs were produced in the same manner as in Device Examples 3-1 and 3-2, except that the compound shown in Table 3 below was used in the second hole transport layer.

The driving voltage, current efficiency, and CIE 1931 color coordinate at a luminance of 1,000 nit, and the time taken for luminance to decrease from 100% to 95% at a luminance of 10,000 nit (lifetime: T95) of the OLEDs produced in the Device Examples and the Comparative Examples are provided in Tables 1 to 3 below.

TABLE 1

| | Second | | | | CIE | |
|---|---|---|---|---|---|---|
| | Hole | | Driving | Current | Color | Lifetime |
| | Transport | | Voltage | Efficiency | Coordinate | (T95) |
| | Layer | Host | (V) | (cd/A) | (x, y) | (hr) |
| Device | C-1 | RH-1 | 4.2 | 32.4 | 0.662, 0.338 | 219 |
| Example 1-1 | | | | | | |
| Device | C-41 | RH-1 | 3.3 | 30.9 | 0.662, 0.338 | 175 |
| Example 1-2 | | | | | | |
| Device | C-14 | RH-1 | 2.7 | 29.3 | 0.661, 0.338 | 143 |
| Example 1-3 | | | | | | |
| Device | C-216 | RH-1 | 2.8 | 30.6 | 0.661, 0.339 | 127.7 |
| Example 1-4 | | | | | | |
| Device | C-214 | RH-1 | 3.3 | 31.7 | 0.662, 0.338 | 168.2 |
| Example 1-5 | | | | | | |
| Device | C-215 | RH-1 | 2.7 | 29.2 | 0.661, 0.339 | 121.7 |
| Example 1-6 | | | | | | |
| Device | C-217 | RH-1 | 3.3 | 31.0 | 0.661, 0.338 | 136.6 |
| Example 1-7 | | | | | | |
| Device | C-218 | RH-1 | 2.8 | 29.0 | 0.662, 0.338 | 133.0 |
| Example 1-8 | | | | | | |
| Device | C-219 | RH-1 | 3.4 | 30.2 | 0.662, 0.338 | 123.3 |
| Example 1-9 | | | | | | |
| Comparative | Ref.-1 | RH-1 | 2.9 | 27.5 | 0.662, 0.338 | 118 |
| Example 1-1 | | | | | | |
| Comparative | Ref.-3 | RH-1 | 2.9 | 22.1 | 0.657, 0.342 | 35.6 |
| Example 1-2 | | | | | | |
| Comparative | Ref.-4 | RH-1 | 2.9 | 18.9 | 0.657, 0.343 | 87.1 |
| Example 1-3 | | | | | | |

TABLE 2

| | Second | | | | CIE | |
|---|---|---|---|---|---|---|
| | Hole | | Driving | Current | Color | Lifetime |
| | Transport | | Voltage | Efficiency | Coordinate | (T95) |
| | Layer | Host | (V) | (cd/A) | (x, y) | (hr) |
| Device | C-1 | RH-2 | 4.2 | 31.6 | 0.659, 0.340 | 170 |
| Example 2-1 | | | | | | |
| Device | C-41 | RH-2 | 3.4 | 30.5 | 0.660, 0.340 | 173 |
| Example 2-2 | | | | | | |
| Device | C-14 | RH-2 | 2.8 | 28.2 | 0.658, 0.341 | 94.3 |
| Example 2-3 | | | | | | |
| Comparative | Ref.-1 | RH-2 | 2.9 | 24.4 | 0.655, 0.344 | 48 |
| Example 2-1 | | | | | | |
| Comparative | Ref.-2 | RH-2 | 2.9 | 25.9 | 0.656, 0.343 | 77 |
| Example 2-2 | | | | | | |

TABLE 3

| | Second | | | | CIE | |
|---|---|---|---|---|---|---|
| | Hole | | Driving | Current | Color | Lifetime |
| | Transport | | Voltage | Efficiency | Coordinate | (T95) |
| | Layer | Host | (V) | (cd/A) | (x, y) | (hr) |
| Device | C-1 | RH-3:RH-4 | 4.1 | 33.5 | 0.660, 0.339 | 193 |
| Example 3-1 | | (5:5) | | | | |
| Device | C-41 | RH-3:RH-4 | 3.3 | 31.9 | 0.660, 0.340 | 190 |
| Example 3-2 | | (5:5) | | | | |
| Device | C-14 | RH-3:RH-4 | 2.7 | 30.3 | 0.659, 0.341 | 120 |
| Example 3-3 | | (5:5) | | | | |
| Comparative | Ref.-1 | RH-3:RH-4 | 2.9 | 28.4 | 0.656, 0.343 | 61 |
| Example 3-1 | | (5:5) | | | | |
| Comparative | Ref.-2 | RH-3:RH-4 | 2.8 | 28.6 | 0.658, 0.341 | 78 |
| Example 3-2 | | (5:5) | | | | |

From Tables 1 to 3 above, it can be seen that the OLEDs comprising the compound according to the present disclosure in the second hole transport layer exhibit significantly improved current efficiency and/or lifetime properties, compared to the OLEDs not comprising the compound according to the present disclosure.

Without being limited by theory, the compound according to the present disclosure may comprise a structure in which the aromatic groups are electronically insulated by an alkyl segment such as a methylene group, whereby intramolecular p-orbital overlaps may occur throughout the methyl groups. This electronic interaction is called homoconjugation, and It is due to the close arrangement of the two aromatic rings and the proximity because of the appropriate C—CH₂—C bending angle. The present inventors found that the HOMO (highest occupied molecular orbital) level can be controlled by including the organic electroluminescent compound according to the present disclosure having such a homojunction in the second hole transport layer between the first hole transport layer and the light-emitting layer, and thereby hole injection from the first hole transport layer into the light-emitting layer can be improved and it can be helpful for device stabilization at the interface. Due to these effects, it is understood that the compound represented by formula 1 of the present disclosure may improve the current efficiency and/or lifetime properties of the organic electroluminescent device.

The compounds used in the Device Examples and the Comparative Examples are shown in Table 4 below.

TABLE 4

| | | |
|---|---|---|
| Hole Injection Layer/First Hole Transport Layer | 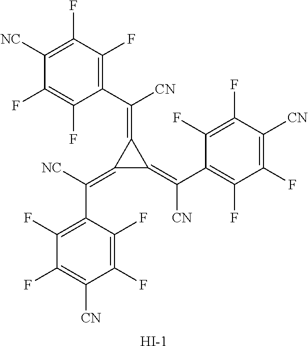 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] | 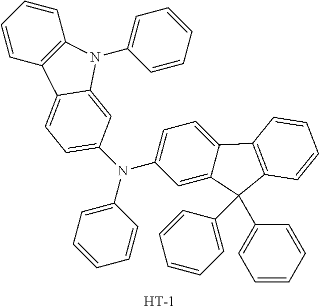 |
| Second Hole Transport Layer | 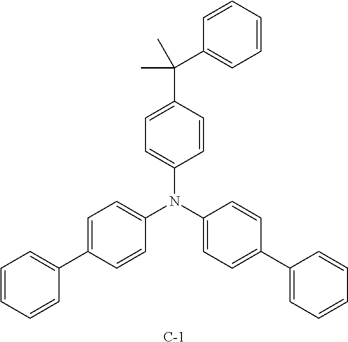 | 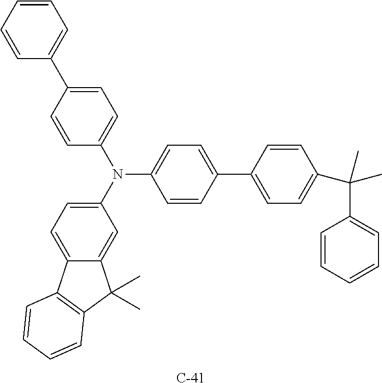 |

| | 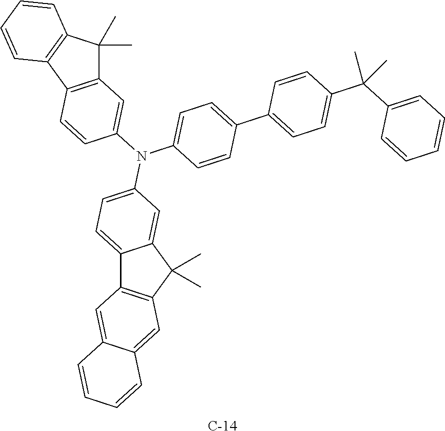 | |
|---|---|---|
| | 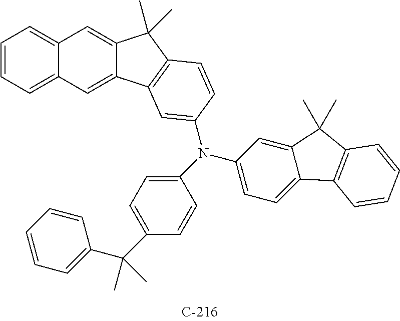 [SMILES: CC(C)(c1ccccc1)c1ccc(N(c2ccc3c(c2)-c2cc4ccccc4cc2C3(C)C)c2ccc3c(c2)C(C)(C)c2ccccc2-3)cc1] | |
| | 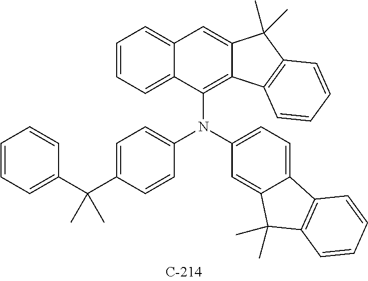 [SMILES: CC(C)(c1ccccc1)c1ccc(N(c2ccc3c(c2)C(C)(C)c2ccccc2-3)c2c3c(cc4ccccc24)C(C)(C)c2ccccc2-3)cc1] | |
| | 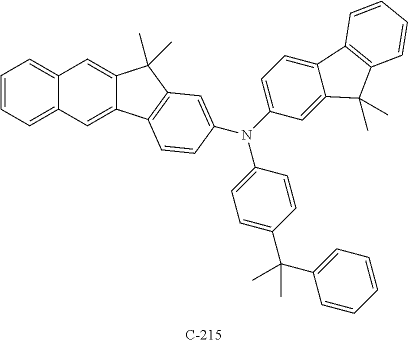 [SMILES: CC(C)(c1ccccc1)c1ccc(N(c2ccc3c(c2)C(C)(C)c2ccccc2-3)c2ccc3c(c2)C(C)(C)c2cc4ccccc4cc2-3)cc1] | |
| | 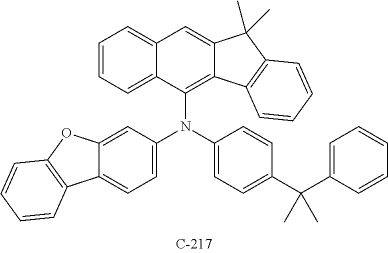 [SMILES: CC(C)(c1ccccc1)c1ccc(N(c2ccc3c(c2)oc2ccccc23)c2c3c(cc4ccccc24)C(C)(C)c2ccccc2-3)cc1] | |
| | 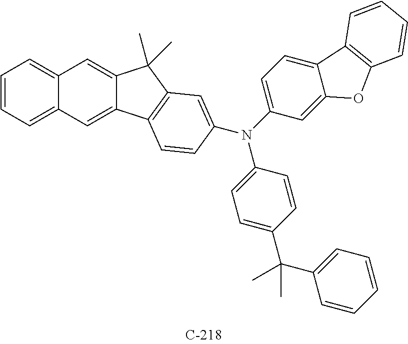 [SMILES: CC(C)(c1ccccc1)c1ccc(N(c2ccc3c(c2)C(C)(C)c2cc4ccccc4cc2-3)c2ccc3c(c2)oc2ccccc23)cc1] | |
| | 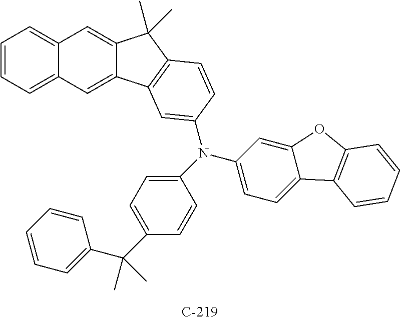 [SMILES: CC(C)(c1ccccc1)c1ccc(N(c2ccc3c(c2)-c2cc4ccccc4cc2C3(C)C)c2ccc3c(c2)oc2ccccc23)cc1] | |
| | 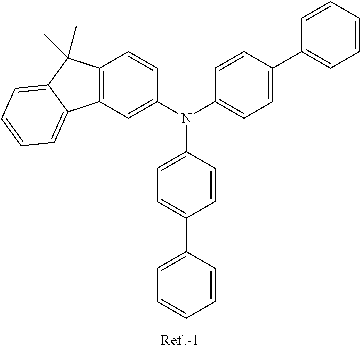 [SMILES: CC1(C)c2ccccc2-c2cc(N(c3ccc(-c4ccccc4)cc3)c3ccc(-c4ccccc4)cc3)ccc21] | |
| | 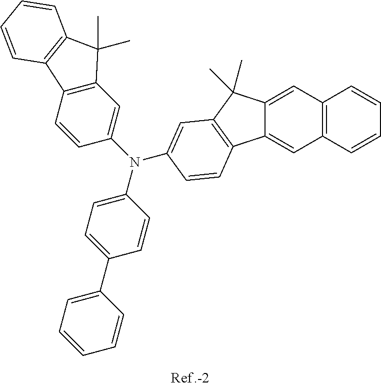 [SMILES: CC1(C)c2ccccc2-c2ccc(N(c3ccc(-c4ccccc4)cc3)c3ccc4c(c3)C(C)(C)c3cc5ccccc5cc3-4)cc21] | |
| | 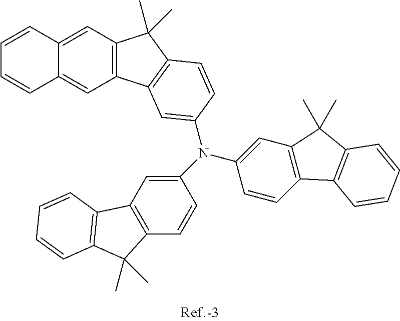 [SMILES: CC1(C)c2ccccc2-c2cc(N(c3ccc4c(c3)-c3cc5ccccc5cc3C4(C)C)c3ccc4c(c3)C(C)(C)c3ccccc3-4)ccc21] | |
| | 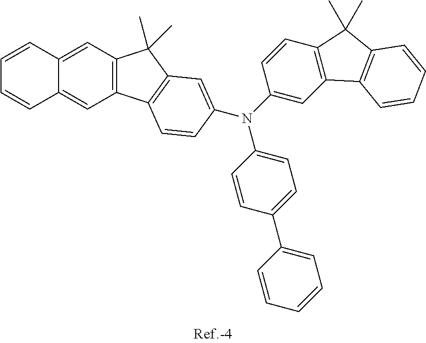 [SMILES: CC1(C)c2ccccc2-c2cc(N(c3ccc(-c4ccccc4)cc3)c3ccc4c(c3)C(C)(C)c3cc5ccccc5cc3-4)ccc21] | |
| Light- Emitting Layer | 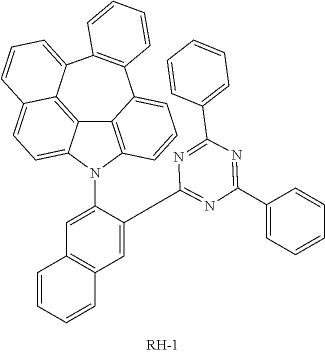 [SMILES: c1ccc(-c2nc(-c3ccccc3)nc(-c3cc4ccccc4cc3-n3c4cccc5c4c4c6c(cccc6ccc43)-c3ccccc3-5)n2)cc1] | 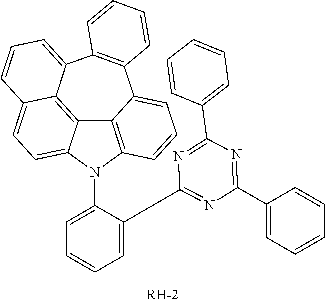 [SMILES: c1ccc(-c2nc(-c3ccccc3)nc(-c3ccccc3-n3c4cccc5c4c4c6c(cccc6ccc43)-c3ccccc3-5)n2)cc1] |
| | 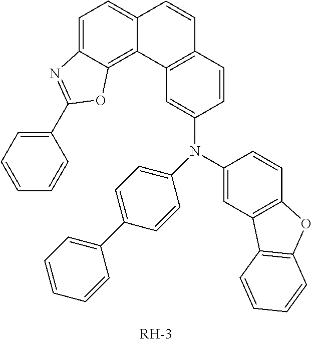 [SMILES: c1ccc(-c2ccc(N(c3ccc4oc5ccccc5c4c3)c3ccc4ccc5ccc6nc(-c7ccccc7)oc6c5c4c3)cc2)cc1] | 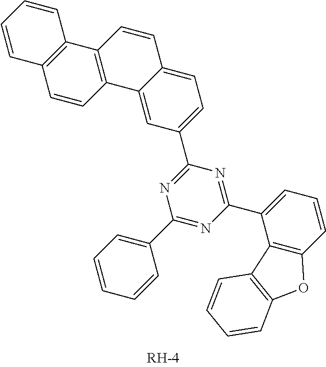 [SMILES: c1ccc(-c2nc(-c3ccc4ccc5c6ccccc6ccc5c4c3)nc(-c3cccc4oc5ccccc5c34)n2)cc1] |
| | 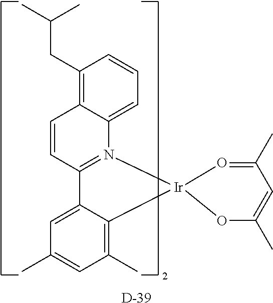 | |
| Electron Buffer Layer | 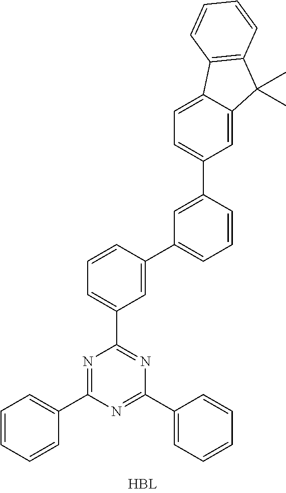 [SMILES: CC1(C)c2ccccc2-c2ccc(-c3cccc(-c4cccc(-c5nc(-c6ccccc6)nc(-c6ccccc6)n5)c4)c3)cc21] | |
| Electron Transport Layer/ Electron Injection Layer | 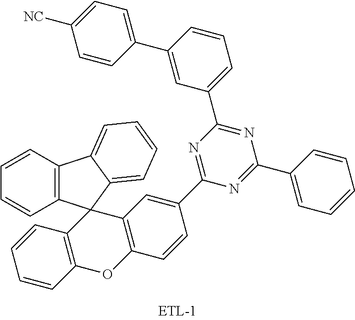 [SMILES: N#Cc1ccc(-c2cccc(-c3nc(-c4ccccc4)nc(-c4ccc5c(c4)C4(c6ccccc6O5)c5ccccc5-c5ccccc54)n3)c2)cc1] | 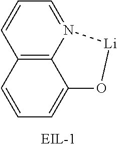 |

1. An organic electroluminescent compound represented by the following formula 1: 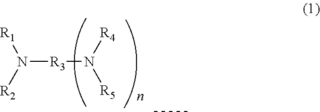
in formula 1,
R₁, R₂, R₄, and R₅, each independently, represent a substituted or unsubstituted (C6-C30)alyl, or a substituted or unsubstituted (3- to 30-membered)heteroaryl;
R₃ represents a substituted or unsubstituted (C6-C30)aryl(ene), or a substituted or unsubstituted (3- to 30-membered)heteroaryl(ene);
with the proviso that when n represents 0, at least one of R₁ to R₃ is substituted with the following formula 1′; and when n represents 1, at least one of R₁ to R₅ is substituted with the following formula 1′; 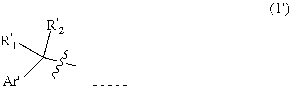
in formula 1′,
R′₁ and R′₂ represent a (C1-C5)alkyl unsubstituted or substituted with deuterium;
Ar′ represents a substituted or unsubstituted (C6-C30)aryl, or a substituted or unsubstituted (3- to 30-membered)heteroaryl, with the proviso that Ar′ does not comprise an amine group; and
n represents an integer of 0 or 1;
with the proviso that formula 1 does not comprise an acridine structure in a spiro form.

2. The organic electroluminescent compound according to claim 1, wherein formula 1 is represented by any one of the following formulas 1-1 to 1-3: 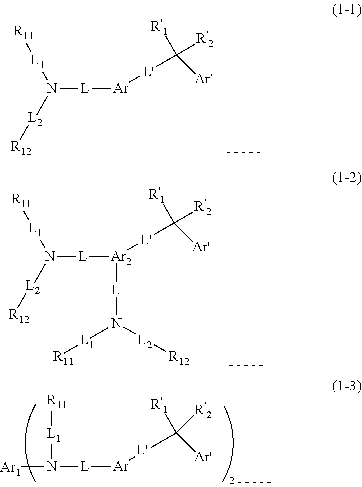
in formulas 1-1 to 1-3,
R₁₁ and R₁₂, each independently, represent a substituted or unsubstituted (C6-C30)aryl, or a substituted or unsubstituted (3- to 30-membered)heteroaryl;
L, L₁, L₂ and L′, each independently, represent a single bond, a substituted or unsubstituted (C6-C30)arylene, or a substituted or unsubstituted (3- to 30-membered)heteroarylene;
Ar and Ar₁, each independently, represent a substituted or unsubstituted (C6-C30)arylene, or a substituted or unsubstituted (3- to 30-membered)heteroarylene;
Ar₂ represents a trivalent group of a substituted or unsubstituted (C6-C30)aryl ring or a substituted or unsubstituted (3- to 30-membered)heteroaryl ring;
when a plurality of R₁₁, R₁₂, L, L₁, L₂, L′, Ar, R′₁, R′₂, and Ar′ are present, each of R₁₁, each of R₁₂, each of L, each of L₁, each of L₂, each of L′, each of Ar, each of R′₁, each of R′₂, and each of Ar′ may be the same as or different from each other; and
R′₁, R′₂ and Ar′ are as defined in claim 1.

3. The organic electroiuminescent compound according to claim 2, wherein formula 1 is represented by any one of the following formulas 1-1-1 to 1-1-6: 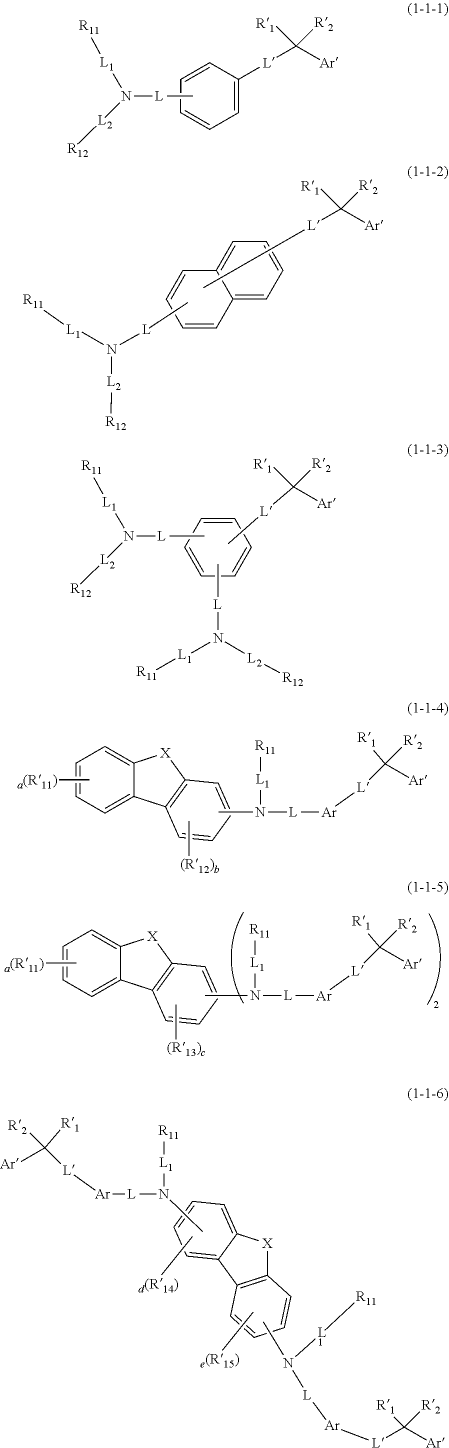
in formulas 1-1-1 to 1-1-6,
X represents —CR′ₐR′_{b}—, —NR′_{c}—, —O—, —S— or —Se—;
R′ₐ to R′_{c}, each independently, represent hydrogen, deuterium, an unsubstituted (C1-C30)alkyl, an unsubstituted (C6-C30)aryl, or an unsubstituted (3- to 30-membered)heteroaryl; or may be linked to an adjacent substituent(s) to form a spiro ring(s);
R′₁₁ to R′₁₅, each independently, represent hydrogen, an unsubstituted (C6-C30)aryl, or an unsubstituted (3- to 30-membered)heteroaryl; or may be linked to an adjacent substituent(s) to form a ring(s);
a represents an integer of 1 to 4; b d, and e, each independently, represent an integer of 1 to 3; and c represents an integer of 1 or 2; where if a to e are each an integer of 2 or more, each of R′₁₁ to each of R′₁₅ may be the same as or different from each other; and
R′₁, R′₂, Ar′, R₁₁, R₁₂, L, L₁, L₂, L′, and Ar are as defined in claim 2.

4. The organic electroluminescent compound according to claim 1, wherein the substituent(s) of the substituted aryl(ene) and the substituted heteroaryl(ene), each independently, are at least one selected from the group consisting of deuterium; a halogen; a cyano; a carboxyl; a nitro, a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of a (C1-C30)alkyl(s) and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di- (C1-C30)alkylamino; a mono- or di- (C2-C30)alkenylamino; a mono- or di- (C6-C30)arylamino unsubstituted or substituted with a (C1-C30)alkyl(s); a mono- or di- (3- to 30-membered)heteroarylamino; a (C1-C30)alkyl(C2-C30)alkenylamino; a (C1-C30)alkyl(C6-C30)aylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl: a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

5. The organic electroluminescent compound according to claim 1, wherein the compound represented by formula 1 is selected from the group consisting of the following compounds:

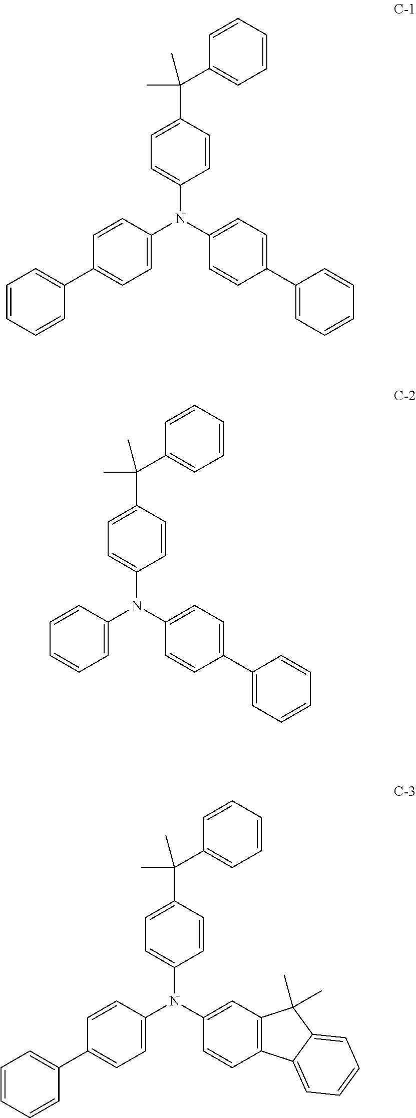 [SMILES: CC(C)(c1ccccc1)c1ccc(N(c2ccc(-c3ccccc3)cc2)c2ccc(-c3ccccc3)cc2)cc1 ; CC(C)(c1ccccc1)c1ccc(N(c2ccccc2)c2ccc(-c3ccccc3)cc2)cc1 ; CC(C)(c1ccccc1)c1ccc(N(c2ccc(-c3ccccc3)cc2)c2ccc3c(c2)C(C)(C)c2ccccc2-3)cc1]

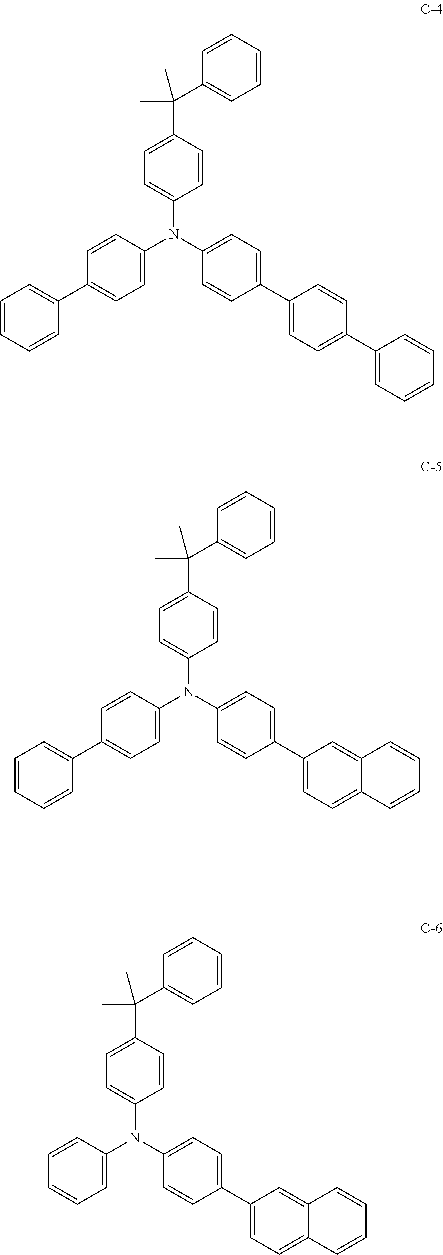 [SMILES: CC(C)(c1ccccc1)c1ccc(N(c2ccc(-c3ccccc3)cc2)c2ccc(-c3ccc(-c4ccccc4)cc3)cc2)cc1 ; CC(C)(c1ccccc1)c1ccc(N(c2ccc(-c3ccccc3)cc2)c2ccc(-c3ccc4ccccc4c3)cc2)cc1 ; CC(C)(c1ccccc1)c1ccc(N(c2ccccc2)c2ccc(-c3ccc4ccccc4c3)cc2)cc1]

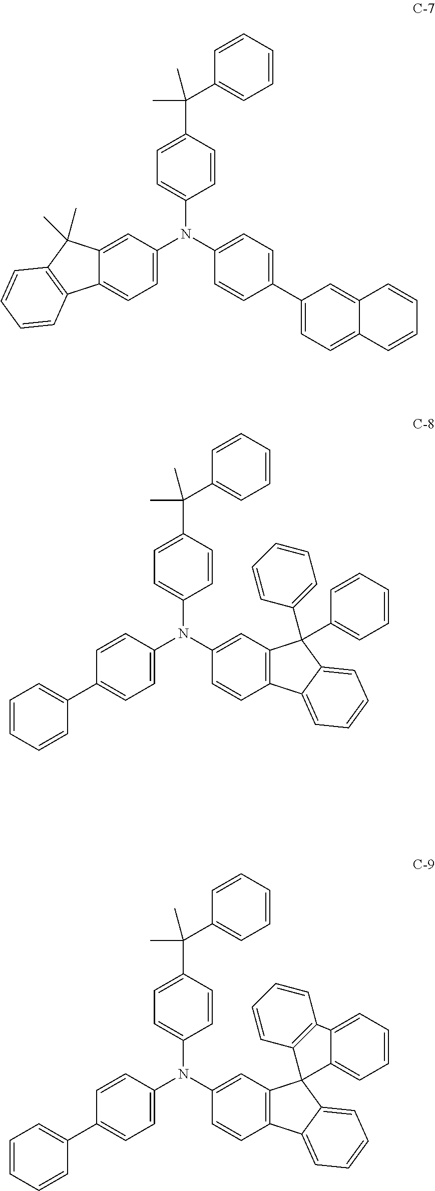 [SMILES: CC(C)(c1ccccc1)c1ccc(N(c2ccc(-c3ccc4ccccc4c3)cc2)c2ccc3c(c2)C(C)(C)c2ccccc2-3)cc1 ; CC(C)(c1ccccc1)c1ccc(N(c2ccc(-c3ccccc3)cc2)c2ccc3c(c2)C(c2ccccc2)(c2ccccc2)c2ccccc2-3)cc1 ; CC(C)(c1ccccc1)c1ccc(N(c2ccc(-c3ccccc3)cc2)c2ccc3c(c2)C2(c4ccccc4-c4ccccc42)c2ccccc2-3)cc1]

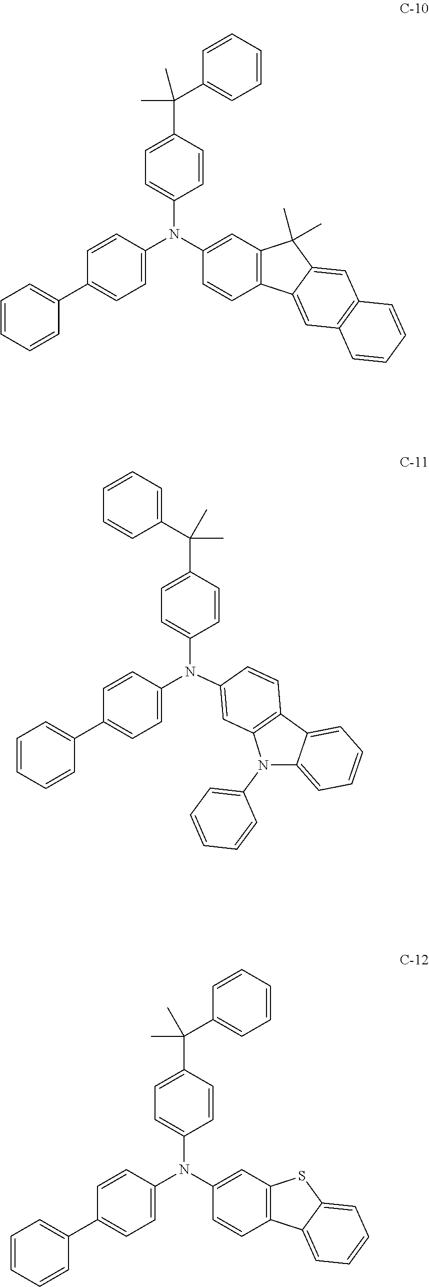 [SMILES: CC(C)(c1ccccc1)c1ccc(N(c2ccc(-c3ccccc3)cc2)c2ccc3c(c2)C(C)(C)c2cc4ccccc4cc2-3)cc1 ; CC(C)(c1ccccc1)c1ccc(N(c2ccc(-c3ccccc3)cc2)c2ccc3c4ccccc4n(-c4ccccc4)c3c2)cc1 ; CC(C)(c1ccccc1)c1ccc(N(c2ccc(-c3ccccc3)cc2)c2ccc3c(c2)sc2ccccc23)cc1]

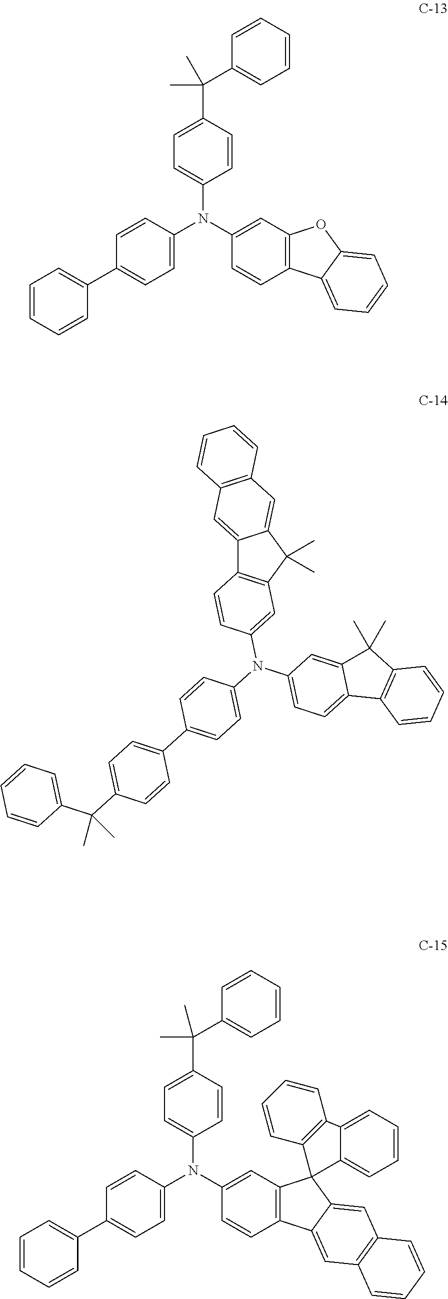 [SMILES: CC(C)(c1ccccc1)c1ccc(N(c2ccc(-c3ccccc3)cc2)c2ccc3c(c2)oc2ccccc23)cc1 ; CC(C)(c1ccccc1)c1ccc(-c2ccc(N(c3ccc4c(c3)C(C)(C)c3ccccc3-4)c3ccc4c(c3)C(C)(C)c3cc5ccccc5cc3-4)cc2)cc1 ; CC(C)(c1ccccc1)c1ccc(N(c2ccc(-c3ccccc3)cc2)c2ccc3c(c2)C2(c4ccccc4-c4ccccc42)c2cc4ccccc4cc2-3)cc1]

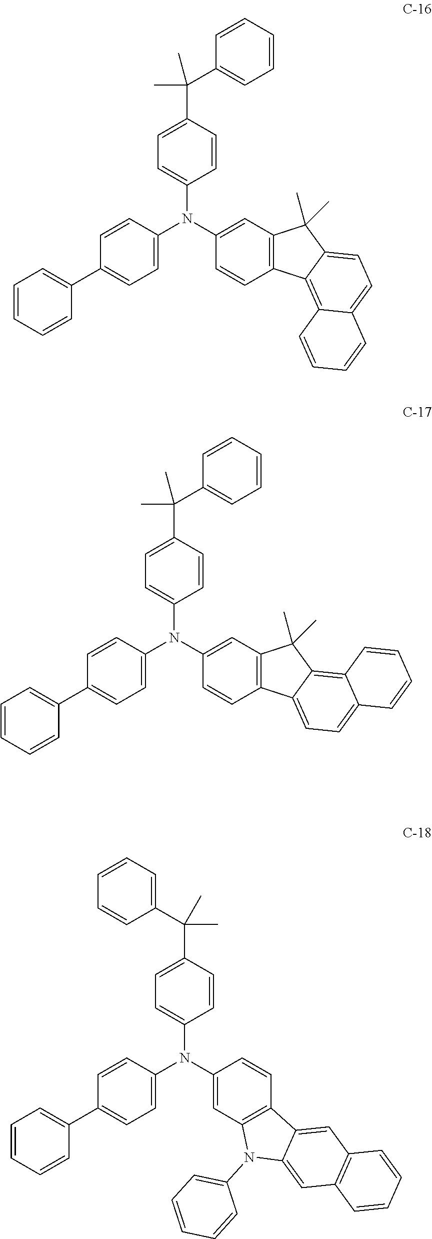 [SMILES: CC(C)(c1ccccc1)c1ccc(N(c2ccc(-c3ccccc3)cc2)c2ccc3c(c2)C(C)(C)c2ccc4ccccc4c2-3)cc1 ; CC(C)(c1ccccc1)c1ccc(N(c2ccc(-c3ccccc3)cc2)c2ccc3c(c2)C(C)(C)c2c-3ccc3ccccc23)cc1 ; CC(C)(c1ccccc1)c1ccc(N(c2ccc(-c3ccccc3)cc2)c2ccc3c4cc5ccccc5cc4n(-c4ccccc4)c3c2)cc1]

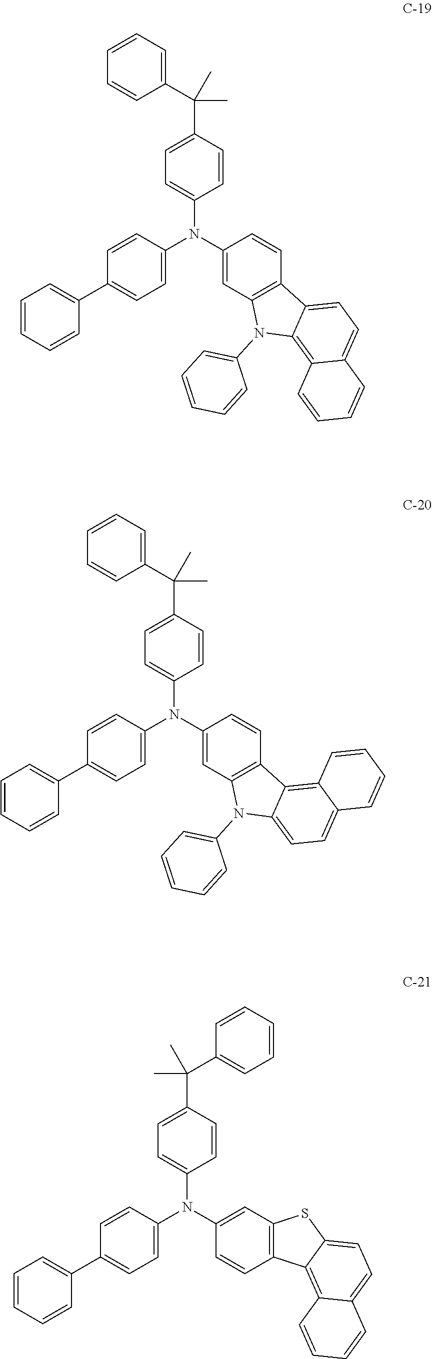 [SMILES: CC(C)(c1ccccc1)c1ccc(N(c2ccc(-c3ccccc3)cc2)c2ccc3c4ccc5ccccc5c4n(-c4ccccc4)c3c2)cc1 ; CC(C)(c1ccccc1)c1ccc(N(c2ccc(-c3ccccc3)cc2)c2ccc3c4c5ccccc5ccc4n(-c4ccccc4)c3c2)cc1 ; CC(C)(c1ccccc1)c1ccc(N(c2ccc(-c3ccccc3)cc2)c2ccc3c(c2)sc2ccc4ccccc4c23)cc1]

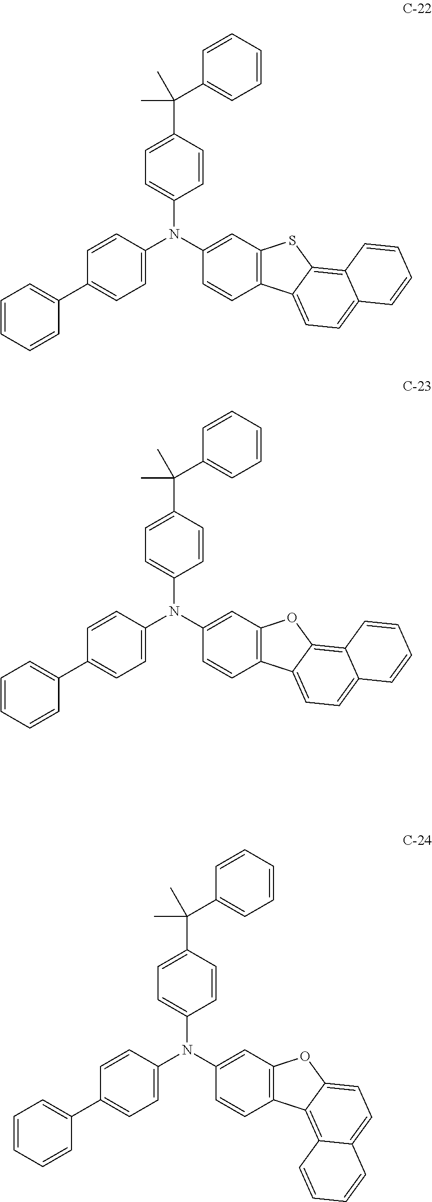 [SMILES: CC(C)(c1ccccc1)c1ccc(N(c2ccc(-c3ccccc3)cc2)c2ccc3c(c2)sc2c4ccccc4ccc32)cc1 ; CC(C)(c1ccccc1)c1ccc(N(c2ccc(-c3ccccc3)cc2)c2ccc3c(c2)oc2c4ccccc4ccc32)cc1 ; CC(C)(c1ccccc1)c1ccc(N(c2ccc(-c3ccccc3)cc2)c2ccc3c(c2)oc2ccc4ccccc4c23)cc1]

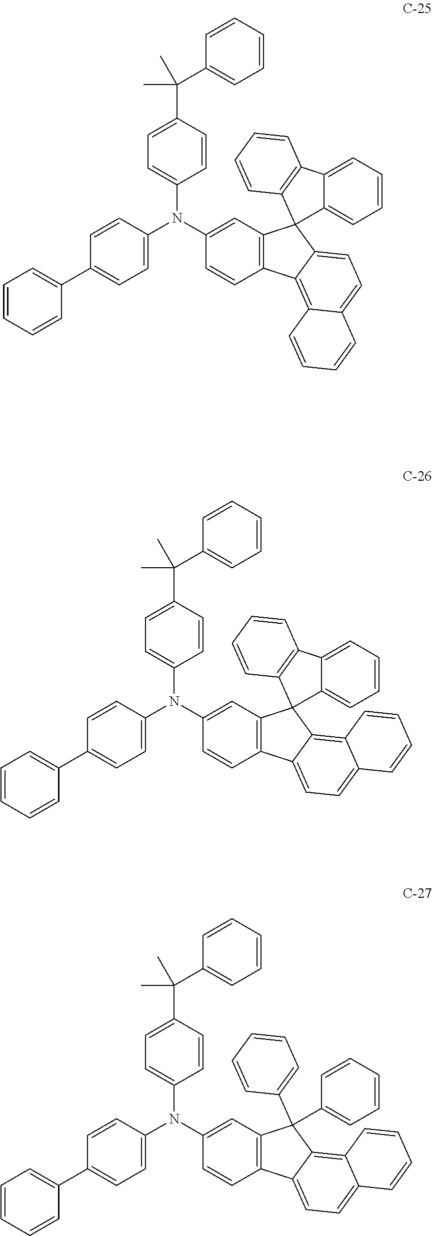 [SMILES: CC(C)(c1ccccc1)c1ccc(N(c2ccc(-c3ccccc3)cc2)c2ccc3c(c2)C2(c4ccccc4-c4ccccc42)c2ccc4ccccc4c2-3)cc1 ; CC(C)(c1ccccc1)c1ccc(N(c2ccc(-c3ccccc3)cc2)c2ccc3c(c2)C2(c4ccccc4-c4ccccc42)c2c-3ccc3ccccc23)cc1 ; CC(C)(c1ccccc1)c1ccc(N(c2ccc(-c3ccccc3)cc2)c2ccc3c(c2)C(c2ccccc2)(c2ccccc2)c2c-3ccc3ccccc23)cc1]

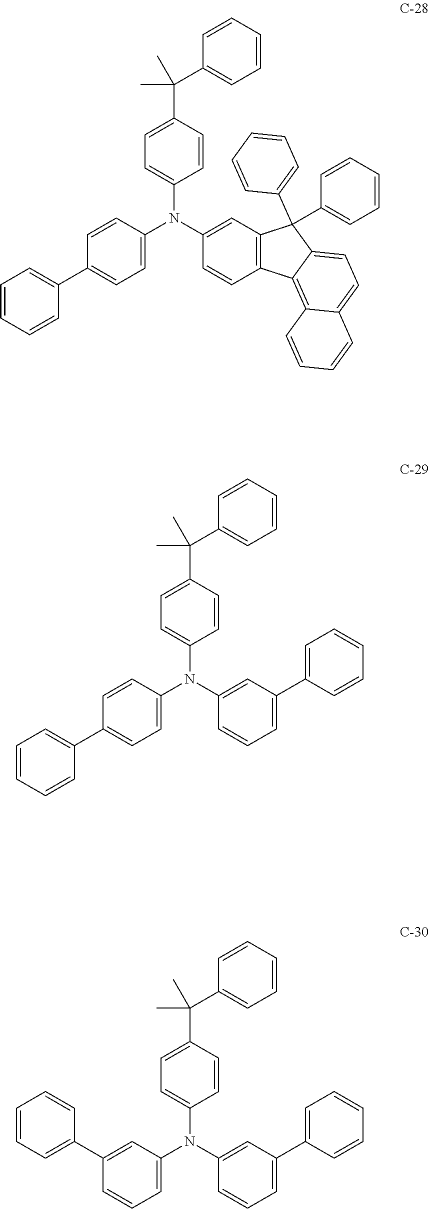 [SMILES: CC(C)(c1ccccc1)c1ccc(N(c2ccc(-c3ccccc3)cc2)c2ccc3c(c2)C(c2ccccc2)(c2ccccc2)c2ccc4ccccc4c2-3)cc1 ; CC(C)(c1ccccc1)c1ccc(N(c2ccc(-c3ccccc3)cc2)c2cccc(-c3ccccc3)c2)cc1 ; CC(C)(c1ccccc1)c1ccc(N(c2cccc(-c3ccccc3)c2)c2cccc(-c3ccccc3)c2)cc1]

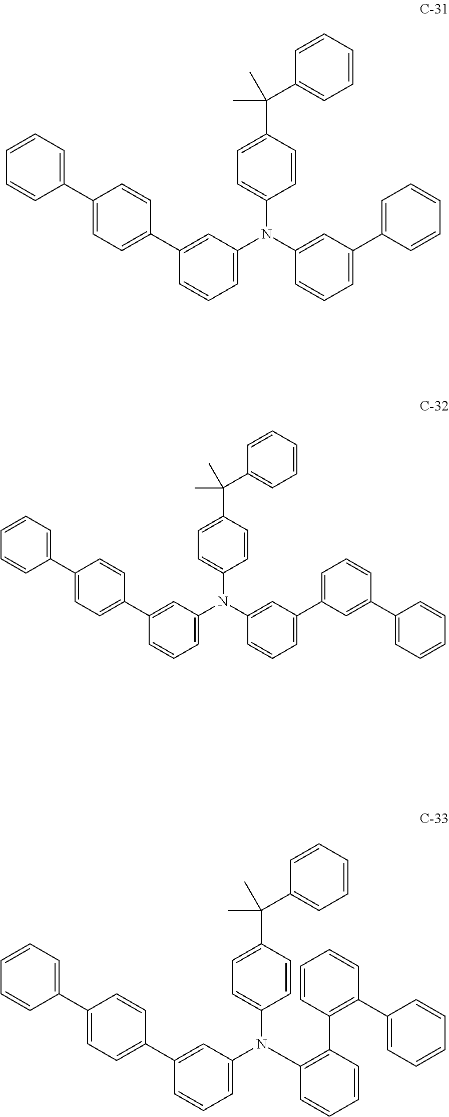 [SMILES: CC(C)(c1ccccc1)c1ccc(N(c2cccc(-c3ccccc3)c2)c2cccc(-c3ccc(-c4ccccc4)cc3)c2)cc1 ; CC(C)(c1ccccc1)c1ccc(N(c2cccc(-c3ccc(-c4ccccc4)cc3)c2)c2cccc(-c3cccc(-c4ccccc4)c3)c2)cc1 ; CC(C)(c1ccccc1)c1ccc(N(c2cccc(-c3ccc(-c4ccccc4)cc3)c2)c2ccccc2-c2ccccc2-c2ccccc2)cc1]

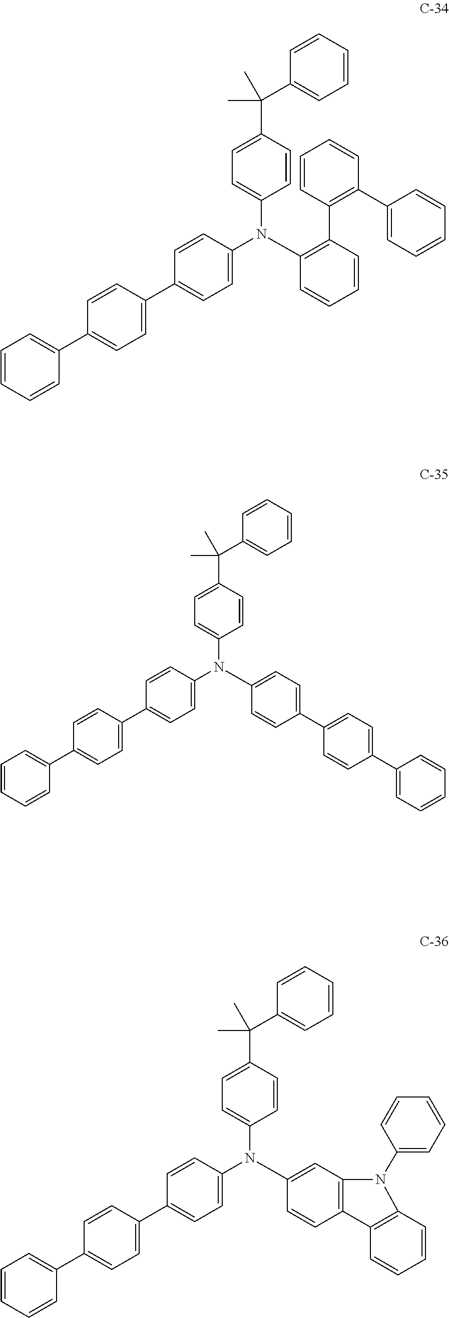 [SMILES: CC(C)(c1ccccc1)c1ccc(N(c2ccc(-c3ccc(-c4ccccc4)cc3)cc2)c2ccccc2-c2ccccc2-c2ccccc2)cc1 ; CC(C)(c1ccccc1)c1ccc(N(c2ccc(-c3ccc(-c4ccccc4)cc3)cc2)c2ccc(-c3ccc(-c4ccccc4)cc3)cc2)cc1 ; CC(C)(c1ccccc1)c1ccc(N(c2ccc(-c3ccc(-c4ccccc4)cc3)cc2)c2ccc3c4ccccc4n(-c4ccccc4)c3c2)cc1]

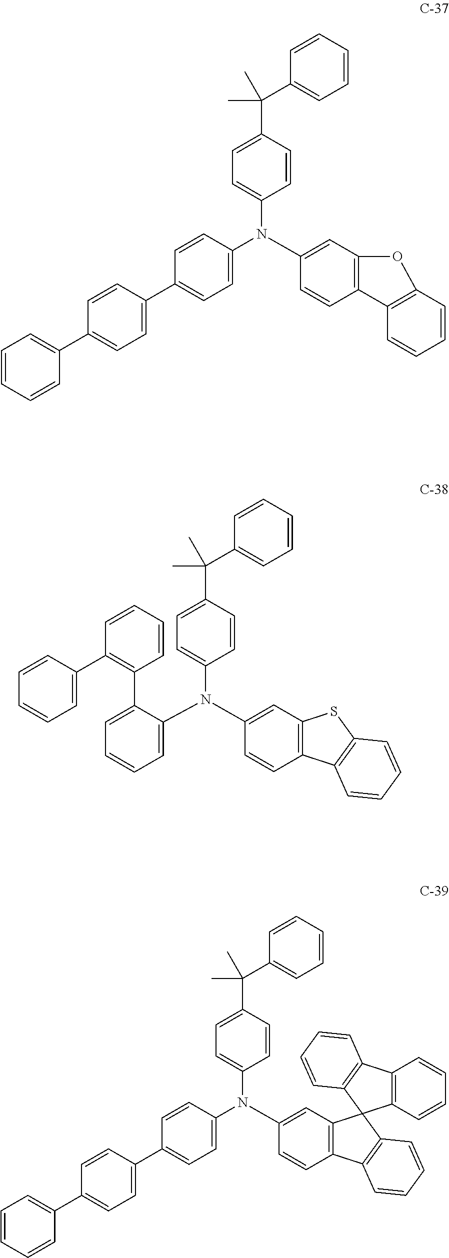 [SMILES: CC(C)(c1ccccc1)c1ccc(N(c2ccc(-c3ccc(-c4ccccc4)cc3)cc2)c2ccc3c(c2)oc2ccccc23)cc1 ; CC(C)(c1ccccc1)c1ccc(N(c2ccc3c(c2)sc2ccccc23)c2ccccc2-c2ccccc2-c2ccccc2)cc1 ; CC(C)(c1ccccc1)c1ccc(N(c2ccc(-c3ccc(-c4ccccc4)cc3)cc2)c2ccc3c(c2)C2(c4ccccc4-c4ccccc42)c2ccccc2-3)cc1]

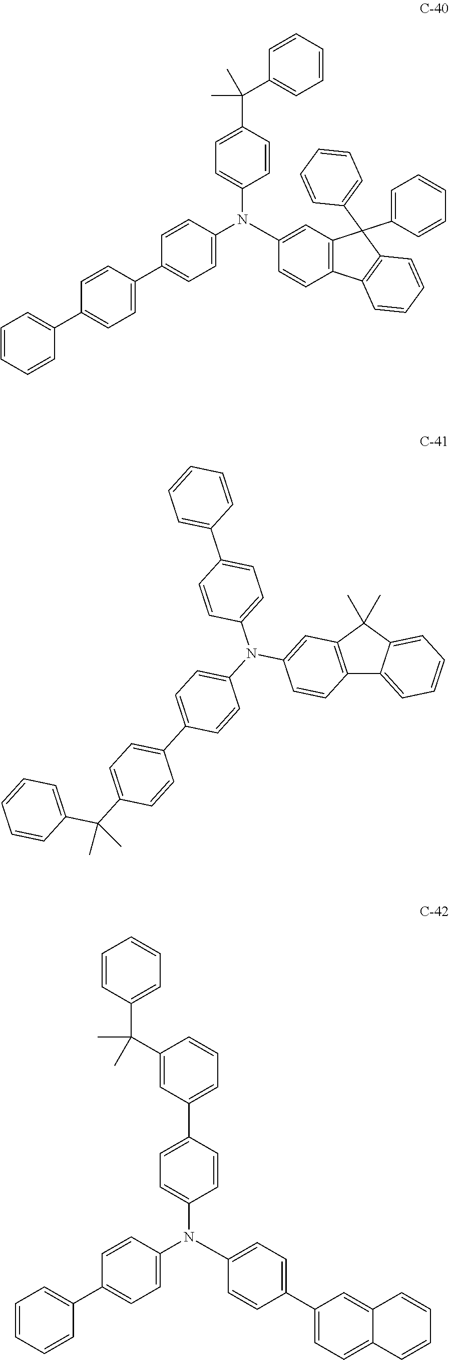 [SMILES: CC(C)(c1ccccc1)c1ccc(N(c2ccc(-c3ccc(-c4ccccc4)cc3)cc2)c2ccc3c(c2)C(c2ccccc2)(c2ccccc2)c2ccccc2-3)cc1 ; CC(C)(c1ccccc1)c1ccc(-c2ccc(N(c3ccc(-c4ccccc4)cc3)c3ccc4c(c3)C(C)(C)c3ccccc3-4)cc2)cc1 ; CC(C)(c1ccccc1)c1cccc(-c2ccc(N(c3ccc(-c4ccccc4)cc3)c3ccc(-c4ccc5ccccc5c4)cc3)cc2)c1]

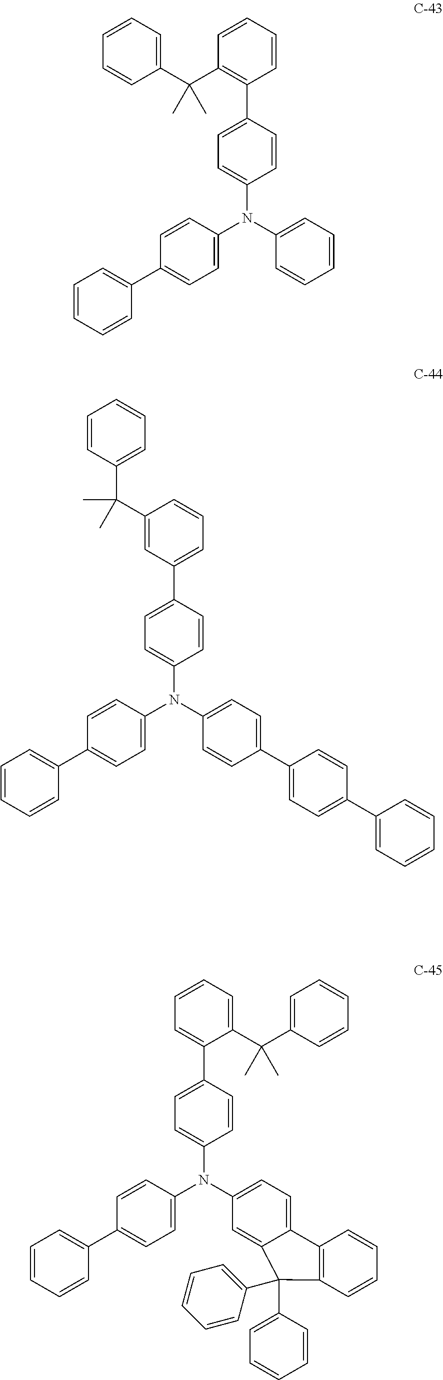 [SMILES: CC(C)(c1ccccc1)c1ccccc1-c1ccc(N(c2ccccc2)c2ccc(-c3ccccc3)cc2)cc1 ; CC(C)(c1ccccc1)c1cccc(-c2ccc(N(c3ccc(-c4ccccc4)cc3)c3ccc(-c4ccc(-c5ccccc5)cc4)cc3)cc2)c1 ; CC(C)(c1ccccc1)c1ccccc1-c1ccc(N(c2ccc(-c3ccccc3)cc2)c2ccc3c(c2)C(c2ccccc2)(c2ccccc2)c2ccccc2-3)cc1]

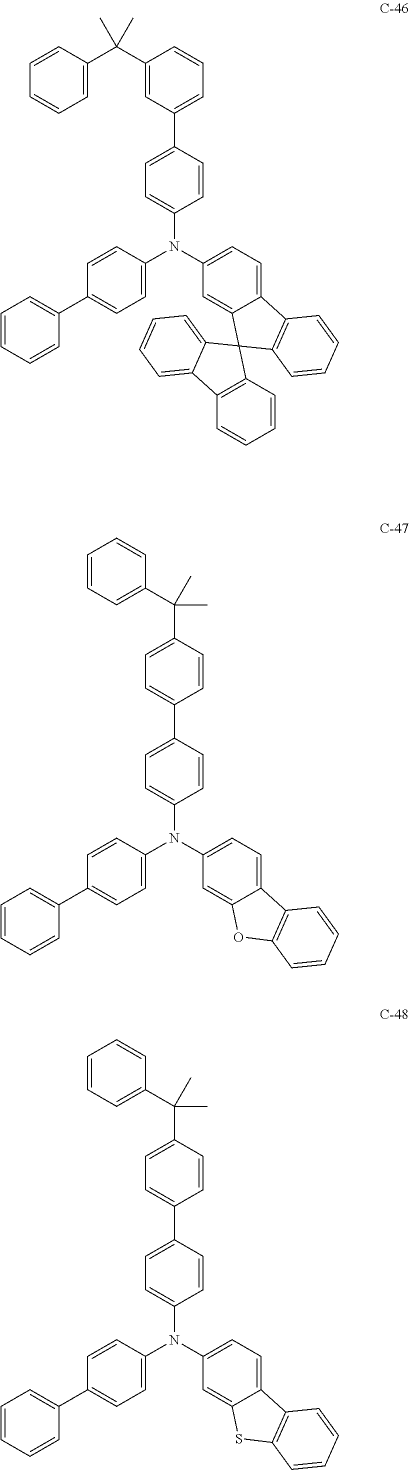 [SMILES: CC(C)(c1ccccc1)c1cccc(-c2ccc(N(c3ccc(-c4ccccc4)cc3)c3ccc4c(c3)C3(c5ccccc5-c5ccccc53)c3ccccc3-4)cc2)c1 ; CC(C)(c1ccccc1)c1ccc(-c2ccc(N(c3ccc(-c4ccccc4)cc3)c3ccc4c(c3)oc3ccccc34)cc2)cc1 ; CC(C)(c1ccccc1)c1ccc(-c2ccc(N(c3ccc(-c4ccccc4)cc3)c3ccc4c(c3)sc3ccccc34)cc2)cc1]

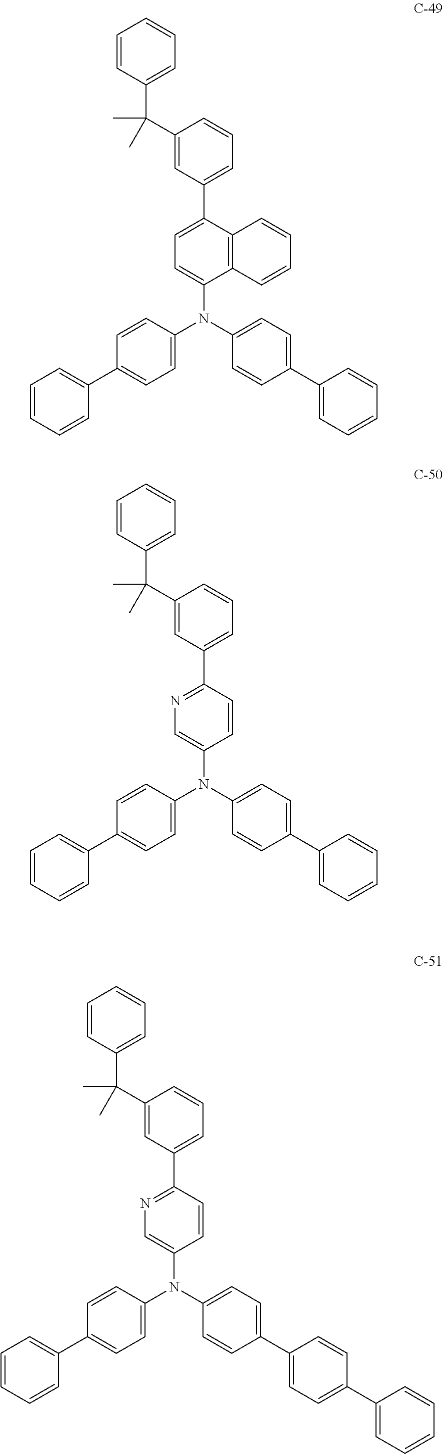 [SMILES: CC(C)(c1ccccc1)c1cccc(-c2ccc(N(c3ccc(-c4ccccc4)cc3)c3ccc(-c4ccccc4)cc3)c3ccccc23)c1 ; CC(C)(c1ccccc1)c1cccc(-c2ccc(N(c3ccc(-c4ccccc4)cc3)c3ccc(-c4ccccc4)cc3)cn2)c1 ; CC(C)(c1ccccc1)c1cccc(-c2ccc(N(c3ccc(-c4ccccc4)cc3)c3ccc(-c4ccc(-c5ccccc5)cc4)cc3)cn2)c1]

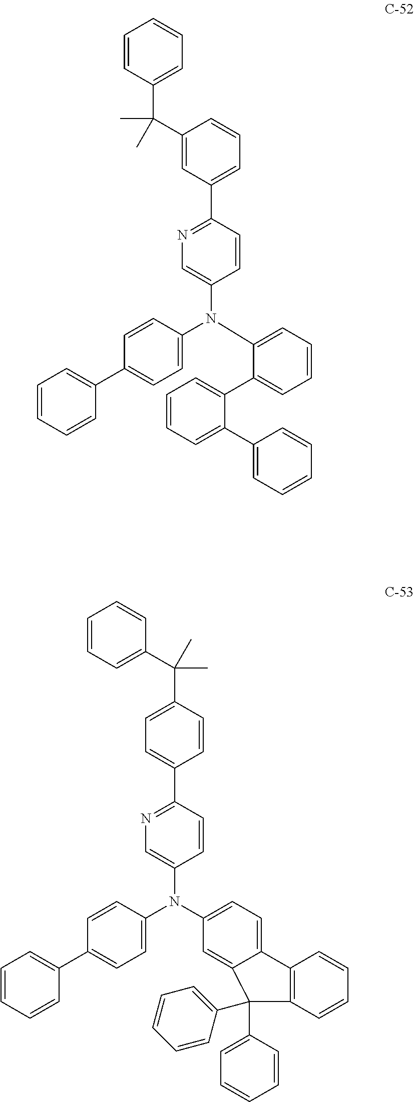 [SMILES: CC(C)(c1ccccc1)c1cccc(-c2ccc(N(c3ccc(-c4ccccc4)cc3)c3ccccc3-c3ccccc3-c3ccccc3)cn2)c1 ; CC(C)(c1ccccc1)c1ccc(-c2ccc(N(c3ccc(-c4ccccc4)cc3)c3ccc4c(c3)C(c3ccccc3)(c3ccccc3)c3ccccc3-4)cn2)cc1]

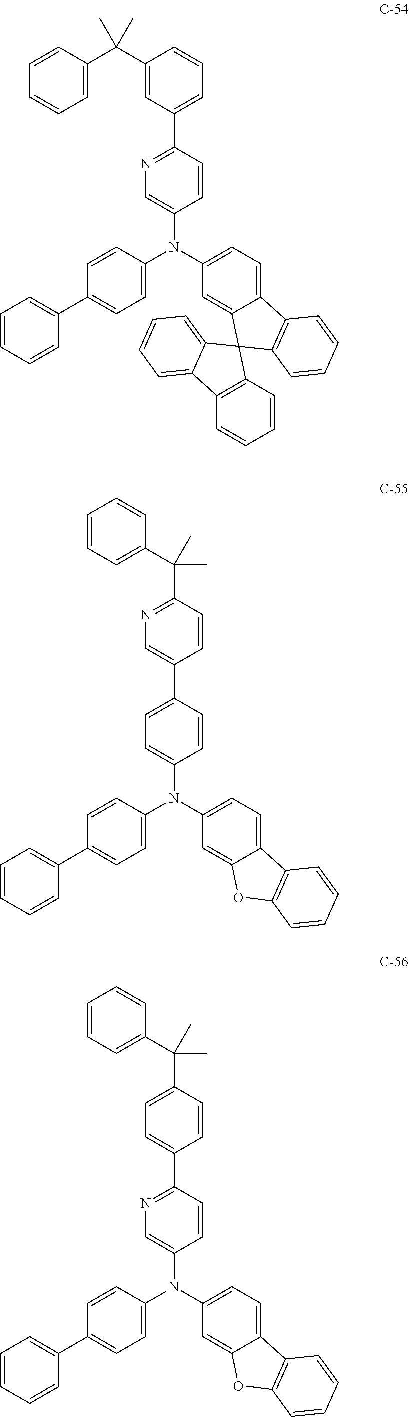 [SMILES: CC(C)(c1ccccc1)c1cccc(-c2ccc(N(c3ccc(-c4ccccc4)cc3)c3ccc4c(c3)C3(c5ccccc5-c5ccccc53)c3ccccc3-4)cn2)c1 ; CC(C)(c1ccccc1)c1ccc(-c2ccc(N(c3ccc(-c4ccccc4)cc3)c3ccc4c(c3)oc3ccccc34)cc2)cn1 ; CC(C)(c1ccccc1)c1ccc(-c2ccc(N(c3ccc(-c4ccccc4)cc3)c3ccc4c(c3)oc3ccccc34)cn2)cc1]

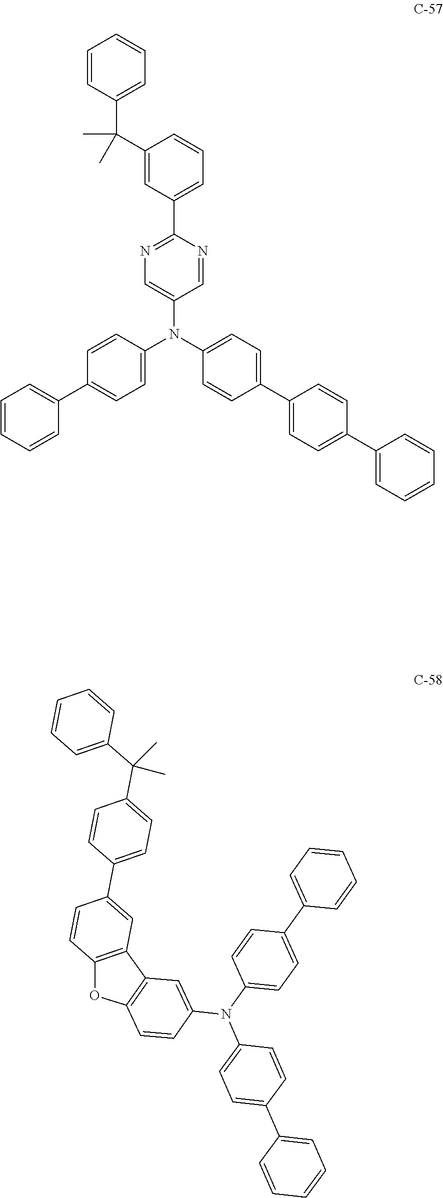 [SMILES: CC(C)(c1ccccc1)c1cccc(-c2ncc(N(c3ccc(-c4ccccc4)cc3)c3ccc(-c4ccc(-c5ccccc5)cc4)cc3)cn2)c1 ; CC(C)(c1ccccc1)c1ccc(-c2ccc3oc4ccc(N(c5ccc(-c6ccccc6)cc5)c5ccc(-c6ccccc6)cc5)cc4c3c2)cc1]

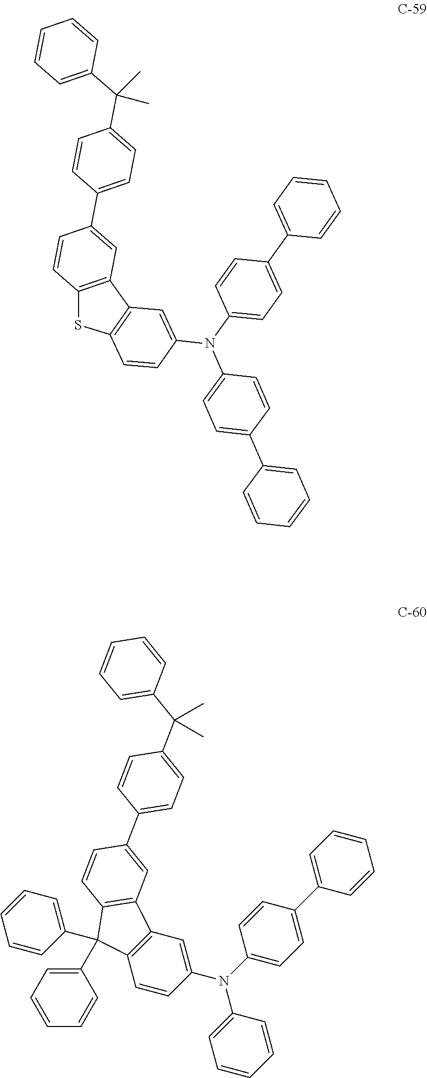 [SMILES: CC(C)(c1ccccc1)c1ccc(-c2ccc3sc4ccc(N(c5ccc(-c6ccccc6)cc5)c5ccc(-c6ccccc6)cc5)cc4c3c2)cc1 ; CC(C)(c1ccccc1)c1ccc(-c2ccc3c(c2)-c2cc(N(c4ccccc4)c4ccc(-c5ccccc5)cc4)ccc2C3(c2ccccc2)c2ccccc2)cc1]

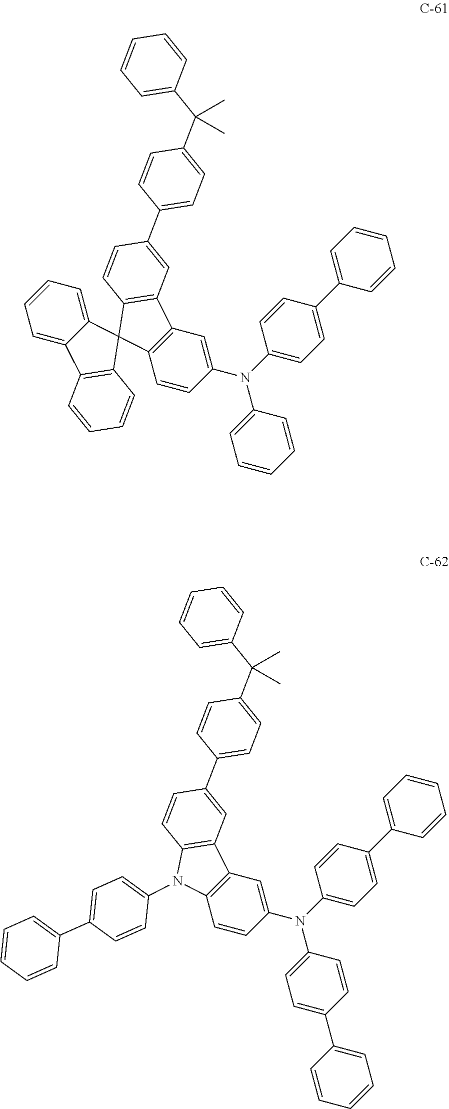 [SMILES: CC(C)(c1ccccc1)c1ccc(-c2ccc3c(c2)-c2cc(N(c4ccccc4)c4ccc(-c5ccccc5)cc4)ccc2C32c3ccccc3-c3ccccc32)cc1 ; CC(C)(c1ccccc1)c1ccc(-c2ccc3c(c2)c2cc(N(c4ccc(-c5ccccc5)cc4)c4ccc(-c5ccccc5)cc4)ccc2n3-c2ccc(-c3ccccc3)cc2)cc1]

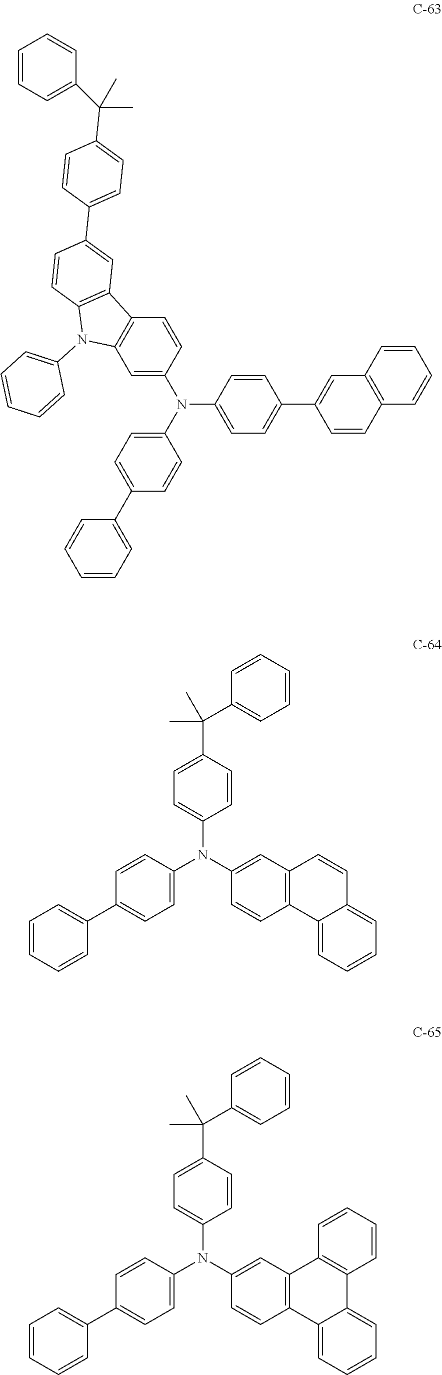 [SMILES: CC(C)(c1ccccc1)c1ccc(-c2ccc3c(c2)c2ccc(N(c4ccc(-c5ccccc5)cc4)c4ccc(-c5ccc6ccccc6c5)cc4)cc2n3-c2ccccc2)cc1 ; CC(C)(c1ccccc1)c1ccc(N(c2ccc(-c3ccccc3)cc2)c2ccc3c(ccc4ccccc43)c2)cc1 ; CC(C)(c1ccccc1)c1ccc(N(c2ccc(-c3ccccc3)cc2)c2ccc3c4ccccc4c4ccccc4c3c2)cc1]

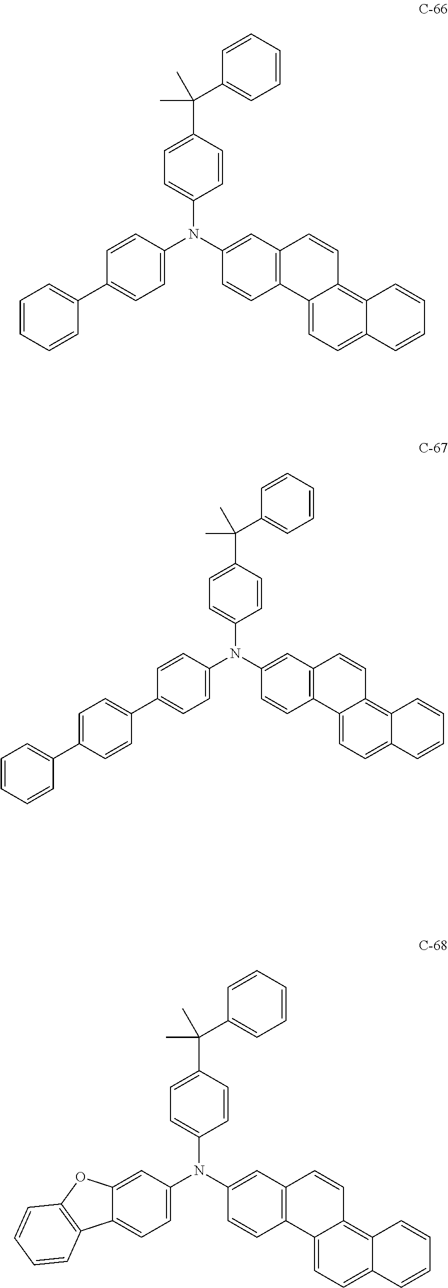 [SMILES: CC(C)(c1ccccc1)c1ccc(N(c2ccc(-c3ccccc3)cc2)c2ccc3c(ccc4c5ccccc5ccc34)c2)cc1 ; CC(C)(c1ccccc1)c1ccc(N(c2ccc(-c3ccc(-c4ccccc4)cc3)cc2)c2ccc3c(ccc4c5ccccc5ccc34)c2)cc1 ; CC(C)(c1ccccc1)c1ccc(N(c2ccc3c(ccc4c5ccccc5ccc34)c2)c2ccc3c(c2)oc2ccccc23)cc1]

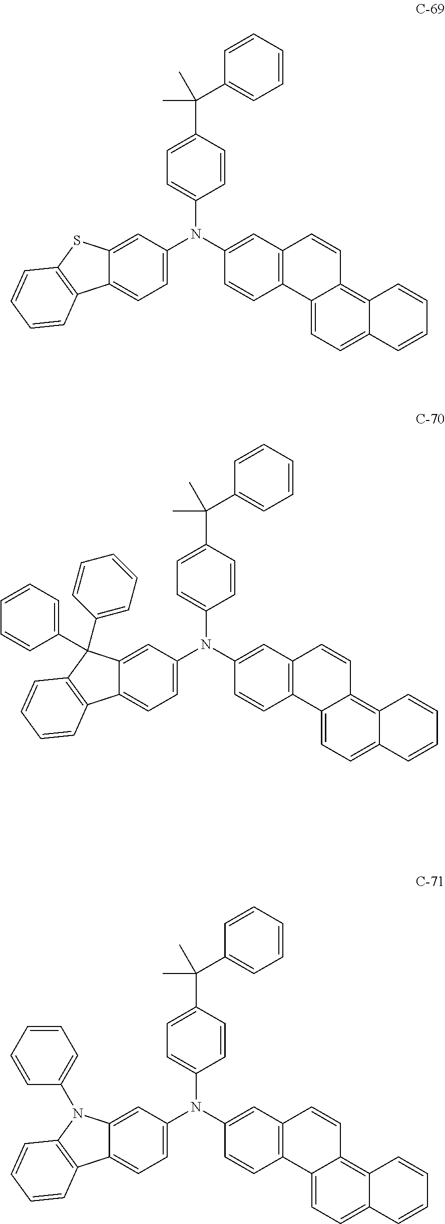 [SMILES: CC(C)(c1ccccc1)c1ccc(N(c2ccc3c(ccc4c5ccccc5ccc34)c2)c2ccc3c(c2)sc2ccccc23)cc1 ; CC(C)(c1ccccc1)c1ccc(N(c2ccc3c(c2)C(c2ccccc2)(c2ccccc2)c2ccccc2-3)c2ccc3c(ccc4c5ccccc5ccc34)c2)cc1 ; CC(C)(c1ccccc1)c1ccc(N(c2ccc3c(ccc4c5ccccc5ccc34)c2)c2ccc3c4ccccc4n(-c4ccccc4)c3c2)cc1]

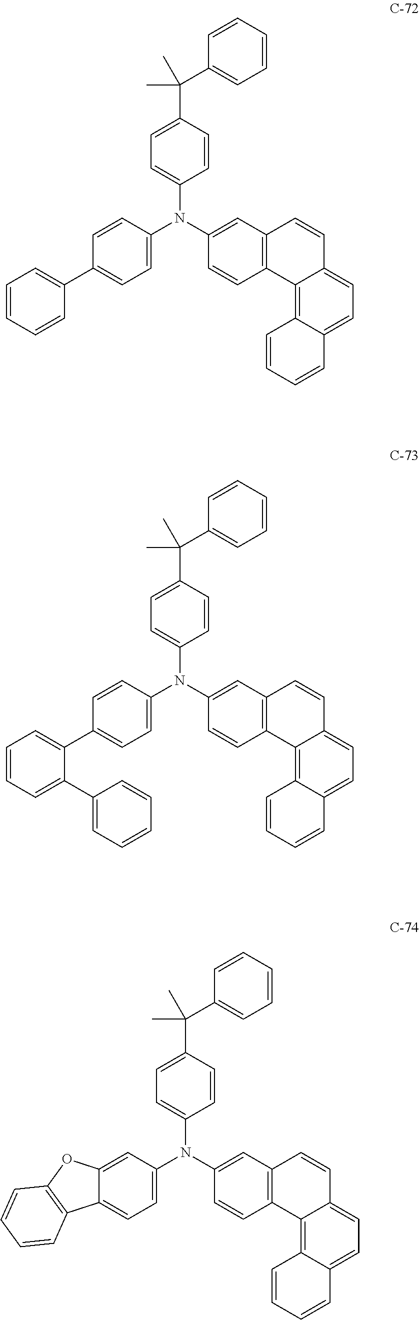 [SMILES: CC(C)(c1ccccc1)c1ccc(N(c2ccc(-c3ccccc3)cc2)c2ccc3c(ccc4ccc5ccccc5c43)c2)cc1 ; CC(C)(c1ccccc1)c1ccc(N(c2ccc(-c3ccccc3-c3ccccc3)cc2)c2ccc3c(ccc4ccc5ccccc5c43)c2)cc1 ; CC(C)(c1ccccc1)c1ccc(N(c2ccc3c(ccc4ccc5ccccc5c43)c2)c2ccc3c(c2)oc2ccccc23)cc1]

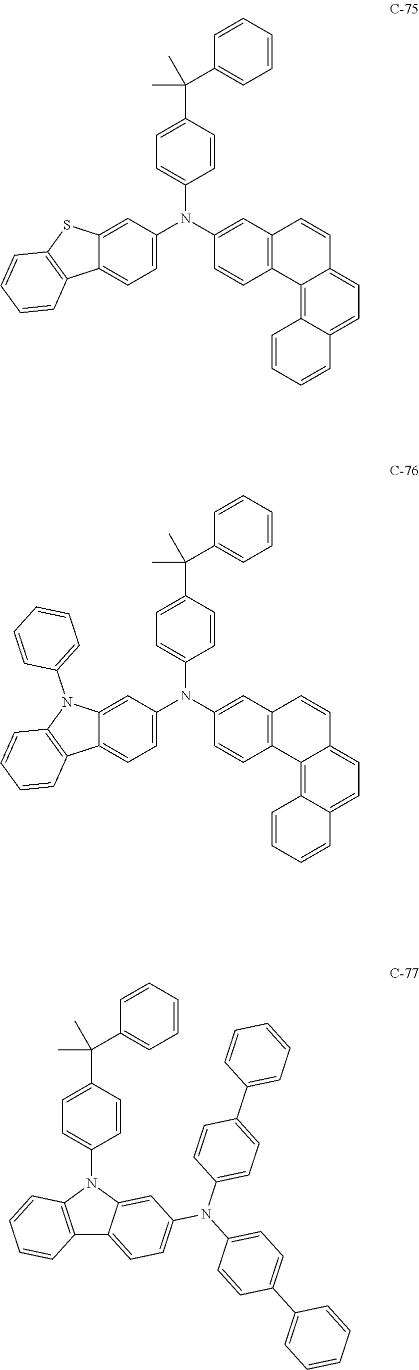 [SMILES: CC(C)(c1ccccc1)c1ccc(N(c2ccc3c(ccc4ccc5ccccc5c43)c2)c2ccc3c(c2)sc2ccccc23)cc1 ; CC(C)(c1ccccc1)c1ccc(N(c2ccc3c(ccc4ccc5ccccc5c43)c2)c2ccc3c4ccccc4n(-c4ccccc4)c3c2)cc1 ; CC(C)(c1ccccc1)c1ccc(-n2c3ccccc3c3ccc(N(c4ccc(-c5ccccc5)cc4)c4ccc(-c5ccccc5)cc4)cc32)cc1]

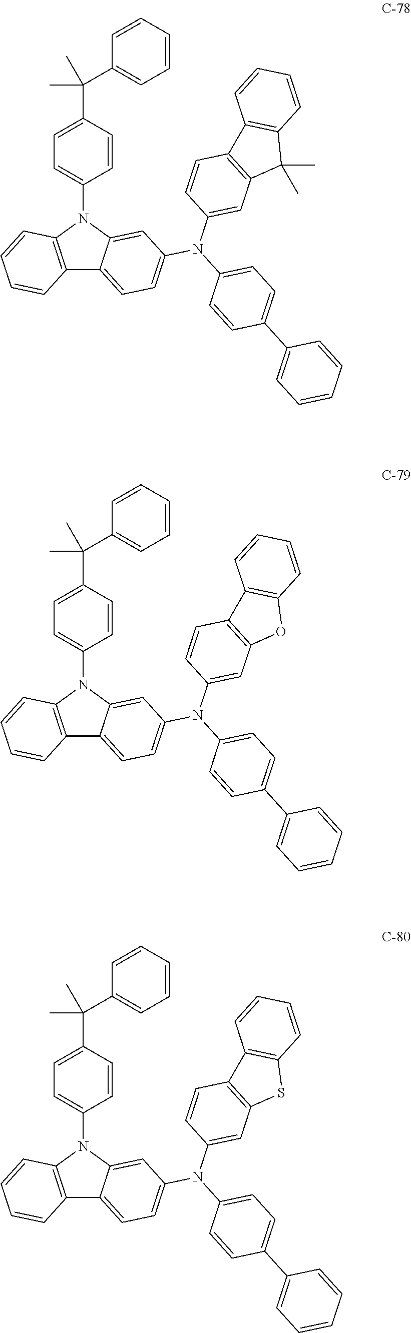 [SMILES: CC(C)(c1ccccc1)c1ccc(-n2c3ccccc3c3ccc(N(c4ccc(-c5ccccc5)cc4)c4ccc5c(c4)C(C)(C)c4ccccc4-5)cc32)cc1 ; CC(C)(c1ccccc1)c1ccc(-n2c3ccccc3c3ccc(N(c4ccc(-c5ccccc5)cc4)c4ccc5c(c4)oc4ccccc45)cc32)cc1 ; CC(C)(c1ccccc1)c1ccc(-n2c3ccccc3c3ccc(N(c4ccc(-c5ccccc5)cc4)c4ccc5c(c4)sc4ccccc45)cc32)cc1]

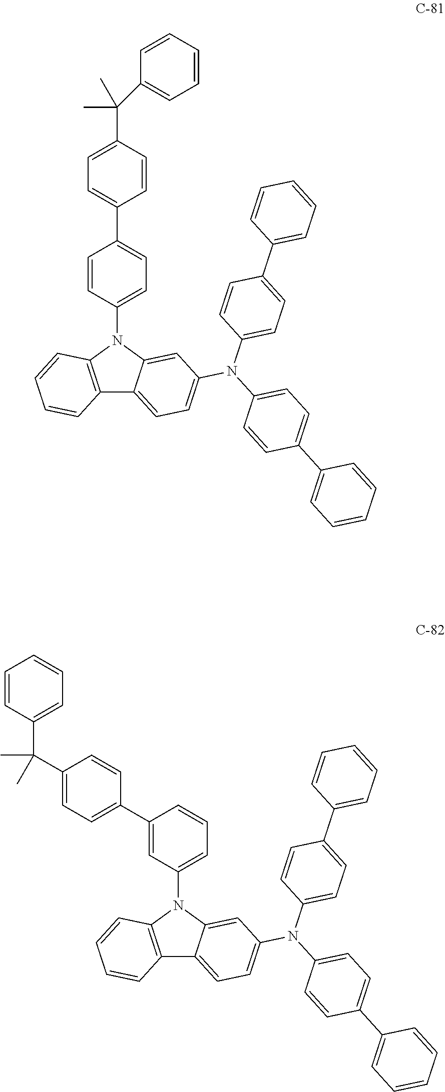 [SMILES: CC(C)(c1ccccc1)c1ccc(-c2ccc(-n3c4ccccc4c4ccc(N(c5ccc(-c6ccccc6)cc5)c5ccc(-c6ccccc6)cc5)cc43)cc2)cc1 ; CC(C)(c1ccccc1)c1ccc(-c2cccc(-n3c4ccccc4c4ccc(N(c5ccc(-c6ccccc6)cc5)c5ccc(-c6ccccc6)cc5)cc43)c2)cc1]

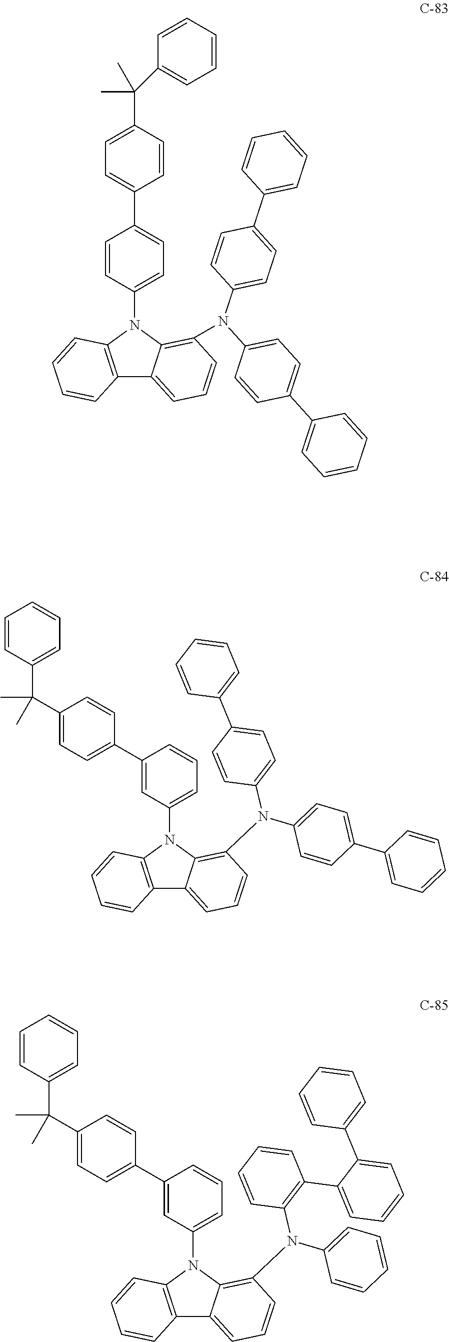 [SMILES: CC(C)(c1ccccc1)c1ccc(-c2ccc(-n3c4ccccc4c4cccc(N(c5ccc(-c6ccccc6)cc5)c5ccc(-c6ccccc6)cc5)c43)cc2)cc1 ; CC(C)(c1ccccc1)c1ccc(-c2cccc(-n3c4ccccc4c4cccc(N(c5ccc(-c6ccccc6)cc5)c5ccc(-c6ccccc6)cc5)c43)c2)cc1 ; CC(C)(c1ccccc1)c1ccc(-c2cccc(-n3c4ccccc4c4cccc(N(c5ccccc5)c5ccccc5-c5ccccc5-c5ccccc5)c43)c2)cc1]

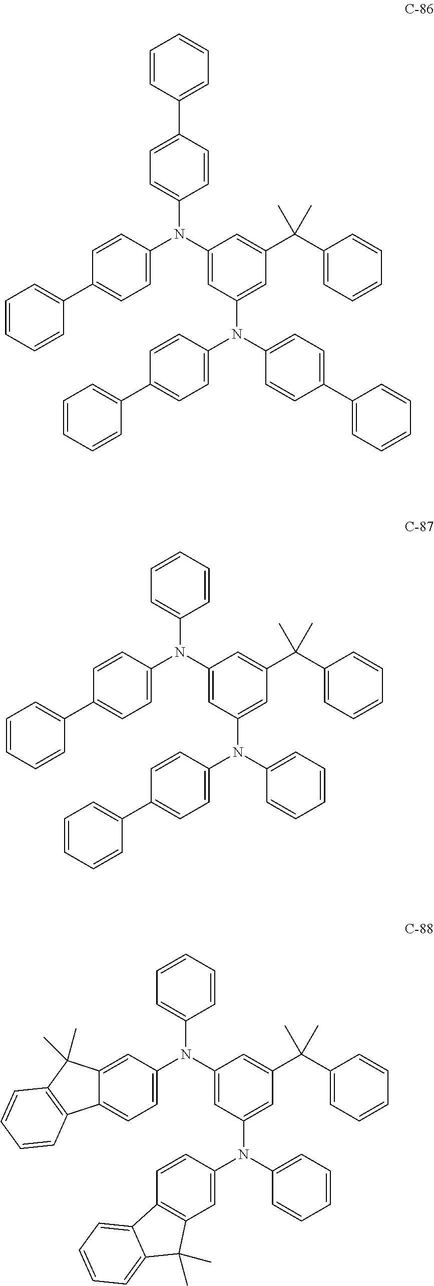 [SMILES: CC(C)(c1ccccc1)c1cc(N(c2ccc(-c3ccccc3)cc2)c2ccc(-c3ccccc3)cc2)cc(N(c2ccc(-c3ccccc3)cc2)c2ccc(-c3ccccc3)cc2)c1 ; CC(C)(c1ccccc1)c1cc(N(c2ccccc2)c2ccc(-c3ccccc3)cc2)cc(N(c2ccccc2)c2ccc(-c3ccccc3)cc2)c1 ; CC(C)(c1ccccc1)c1cc(N(c2ccccc2)c2ccc3c(c2)C(C)(C)c2ccccc2-3)cc(N(c2ccccc2)c2ccc3c(c2)C(C)(C)c2ccccc2-3)c1]

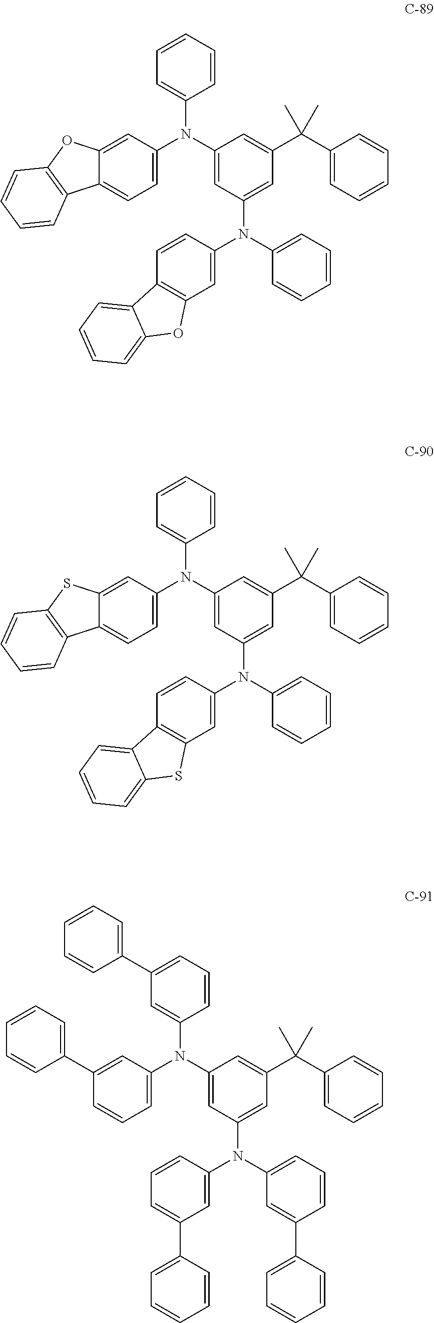 [SMILES: CC(C)(c1ccccc1)c1cc(N(c2ccccc2)c2ccc3c(c2)oc2ccccc23)cc(N(c2ccccc2)c2ccc3c(c2)oc2ccccc23)c1 ; CC(C)(c1ccccc1)c1cc(N(c2ccccc2)c2ccc3c(c2)sc2ccccc23)cc(N(c2ccccc2)c2ccc3c(c2)sc2ccccc23)c1 ; CC(C)(c1ccccc1)c1cc(N(c2cccc(-c3ccccc3)c2)c2cccc(-c3ccccc3)c2)cc(N(c2cccc(-c3ccccc3)c2)c2cccc(-c3ccccc3)c2)c1]

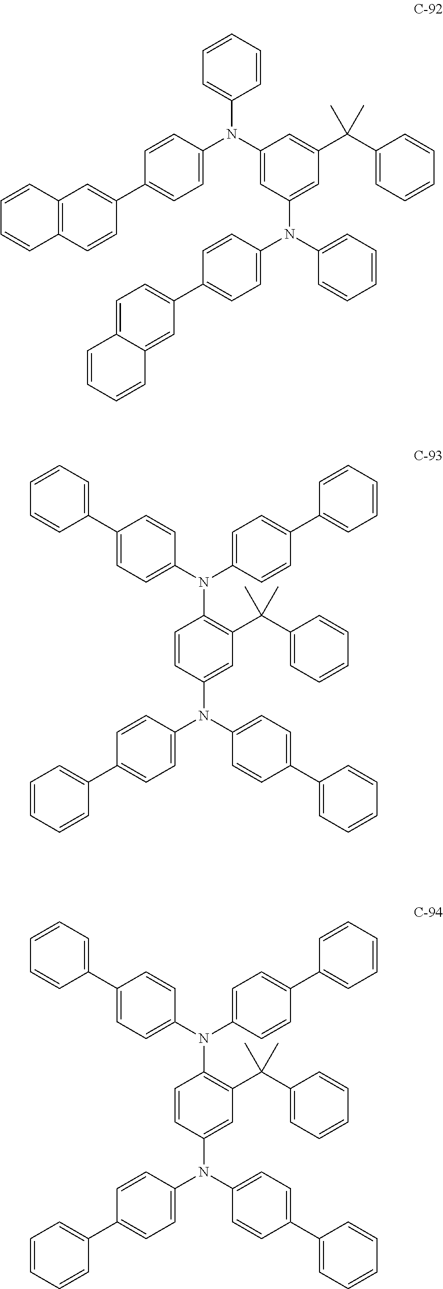 [SMILES: CC(C)(c1ccccc1)c1cc(N(c2ccccc2)c2ccc(-c3ccc4ccccc4c3)cc2)cc(N(c2ccccc2)c2ccc(-c3ccc4ccccc4c3)cc2)c1 ; CC(C)(c1ccccc1)c1cc(N(c2ccc(-c3ccccc3)cc2)c2ccc(-c3ccccc3)cc2)ccc1N(c1ccc(-c2ccccc2)cc1)c1ccc(-c2ccccc2)cc1 ; CC(C)(c1ccccc1)c1cc(N(c2ccc(-c3ccccc3)cc2)c2ccc(-c3ccccc3)cc2)ccc1N(c1ccc(-c2ccccc2)cc1)c1ccc(-c2ccccc2)cc1]

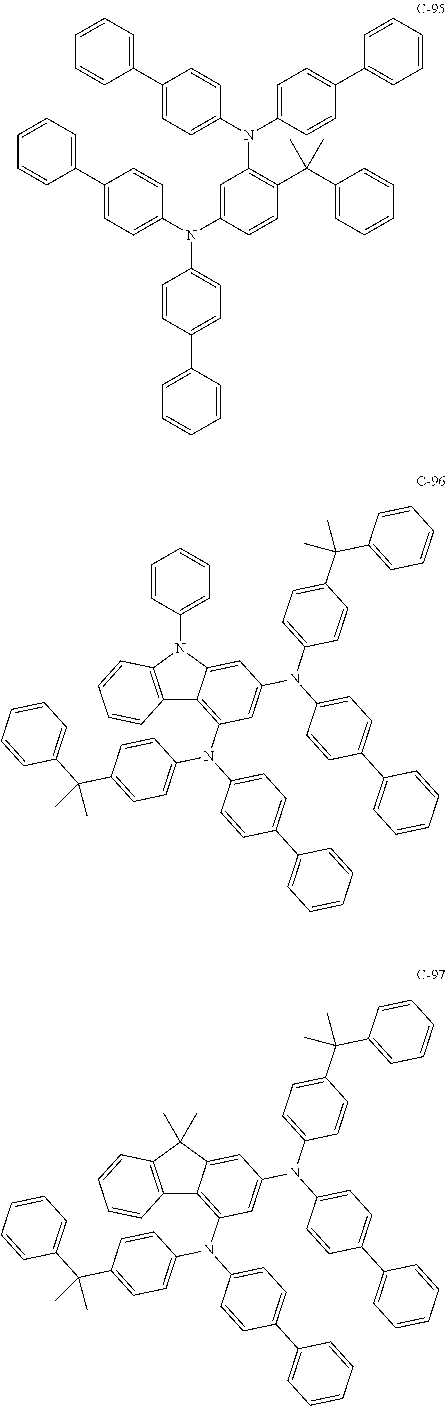 [SMILES: CC(C)(c1ccccc1)c1ccc(N(c2ccc(-c3ccccc3)cc2)c2ccc(-c3ccccc3)cc2)cc1N(c1ccc(-c2ccccc2)cc1)c1ccc(-c2ccccc2)cc1 ; CC(C)(c1ccccc1)c1ccc(N(c2ccc(-c3ccccc3)cc2)c2cc(N(c3ccc(-c4ccccc4)cc3)c3ccc(C(C)(C)c4ccccc4)cc3)c3c4ccccc4n(-c4ccccc4)c3c2)cc1 ; CC(C)(c1ccccc1)c1ccc(N(c2ccc(-c3ccccc3)cc2)c2cc(N(c3ccc(-c4ccccc4)cc3)c3ccc(C(C)(C)c4ccccc4)cc3)c3c(c2)C(C)(C)c2ccccc2-3)cc1]

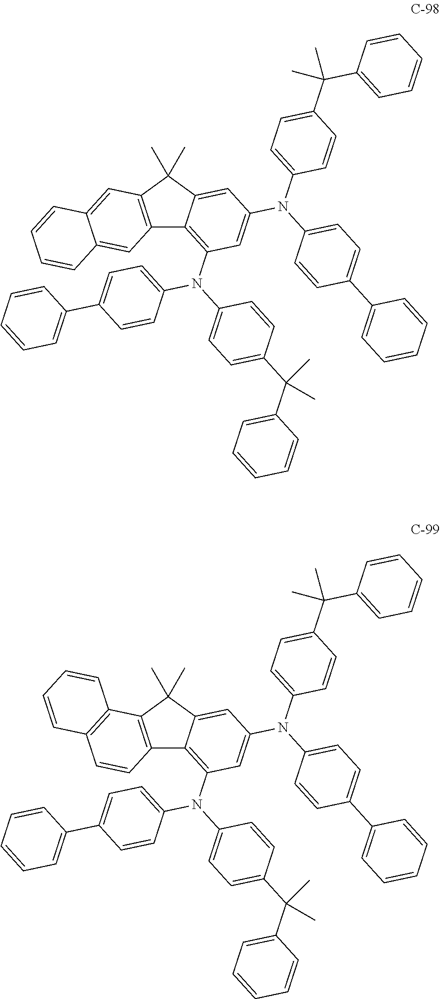 [SMILES: CC(C)(c1ccccc1)c1ccc(N(c2ccc(-c3ccccc3)cc2)c2cc(N(c3ccc(-c4ccccc4)cc3)c3ccc(C(C)(C)c4ccccc4)cc3)c3c(c2)C(C)(C)c2cc4ccccc4cc2-3)cc1 ; CC(C)(c1ccccc1)c1ccc(N(c2ccc(-c3ccccc3)cc2)c2cc(N(c3ccc(-c4ccccc4)cc3)c3ccc(C(C)(C)c4ccccc4)cc3)c3c(c2)C(C)(C)c2c-3ccc3ccccc23)cc1]

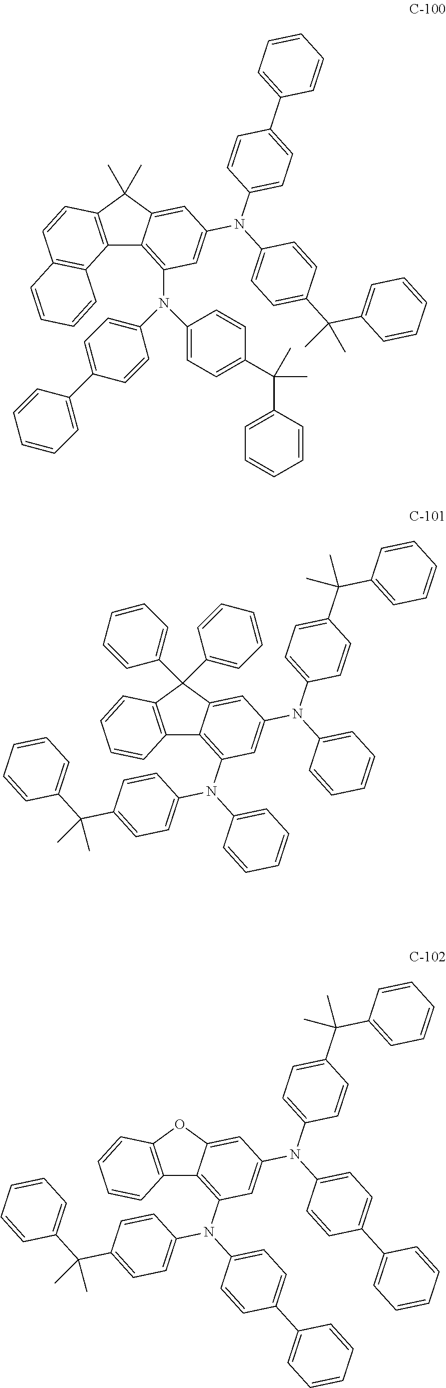 [SMILES: CC(C)(c1ccccc1)c1ccc(N(c2ccc(-c3ccccc3)cc2)c2cc(N(c3ccc(-c4ccccc4)cc3)c3ccc(C(C)(C)c4ccccc4)cc3)c3c(c2)C(C)(C)c2ccc4ccccc4c2-3)cc1 ; CC(C)(c1ccccc1)c1ccc(N(c2ccccc2)c2cc(N(c3ccccc3)c3ccc(C(C)(C)c4ccccc4)cc3)c3c(c2)C(c2ccccc2)(c2ccccc2)c2ccccc2-3)cc1 ; CC(C)(c1ccccc1)c1ccc(N(c2ccc(-c3ccccc3)cc2)c2cc(N(c3ccc(-c4ccccc4)cc3)c3ccc(C(C)(C)c4ccccc4)cc3)c3c(c2)oc2ccccc23)cc1]

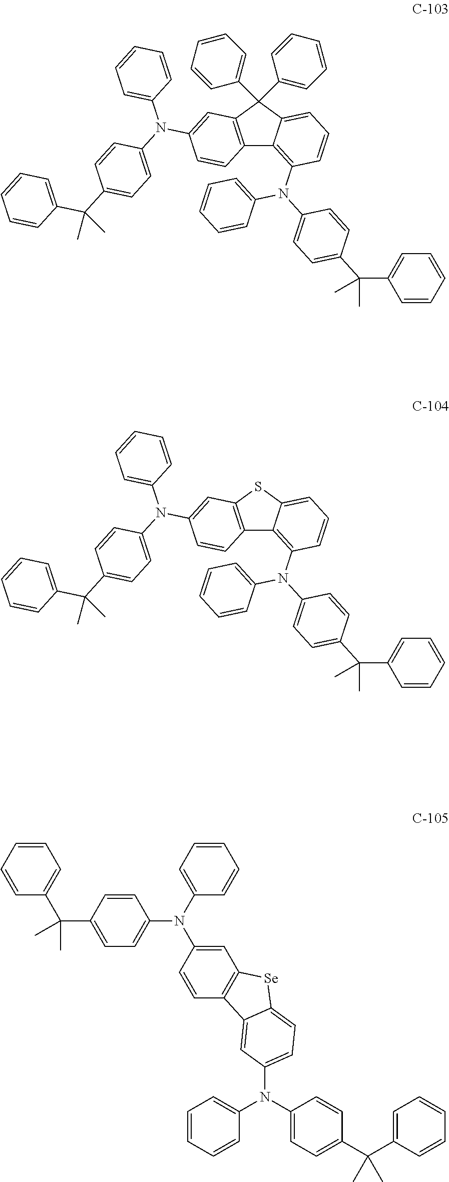 [SMILES: CC(C)(c1ccccc1)c1ccc(N(c2ccccc2)c2ccc3c(c2)C(c2ccccc2)(c2ccccc2)c2cccc(N(c4ccccc4)c4ccc(C(C)(C)c5ccccc5)cc4)c2-3)cc1 ; CC(C)(c1ccccc1)c1ccc(N(c2ccccc2)c2ccc3c(c2)sc2cccc(N(c4ccccc4)c4ccc(C(C)(C)c5ccccc5)cc4)c23)cc1 ; CC(C)(c1ccccc1)c1ccc(N(c2ccccc2)c2ccc3c(c2)[se]c2ccc(N(c4ccccc4)c4ccc(C(C)(C)c5ccccc5)cc4)cc23)cc1]

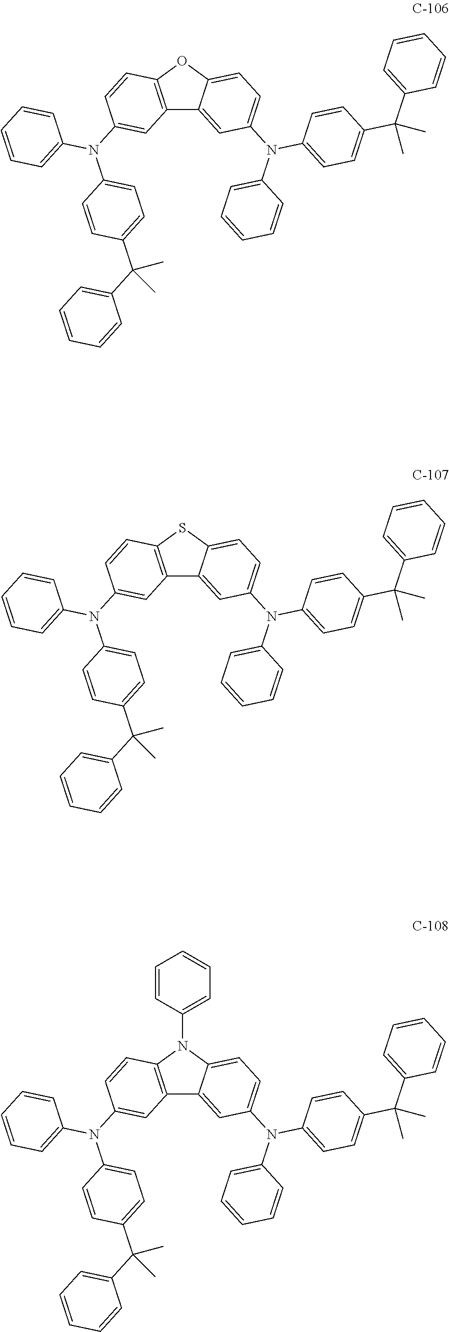 [SMILES: CC(C)(c1ccccc1)c1ccc(N(c2ccccc2)c2ccc3oc4ccc(N(c5ccccc5)c5ccc(C(C)(C)c6ccccc6)cc5)cc4c3c2)cc1 ; CC(C)(c1ccccc1)c1ccc(N(c2ccccc2)c2ccc3sc4ccc(N(c5ccccc5)c5ccc(C(C)(C)c6ccccc6)cc5)cc4c3c2)cc1 ; CC(C)(c1ccccc1)c1ccc(N(c2ccccc2)c2ccc3c(c2)c2cc(N(c4ccccc4)c4ccc(C(C)(C)c5ccccc5)cc4)ccc2n3-c2ccccc2)cc1]

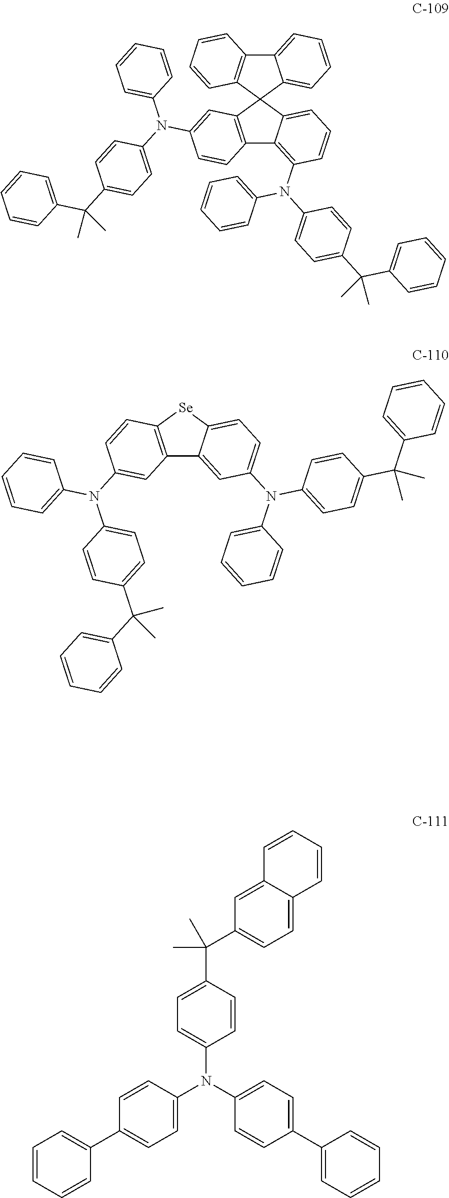 [SMILES: CC(C)(c1ccccc1)c1ccc(N(c2ccccc2)c2ccc3c(c2)C2(c4ccccc4-c4ccccc42)c2cccc(N(c4ccccc4)c4ccc(C(C)(C)c5ccccc5)cc4)c2-3)cc1 ; CC(C)(c1ccccc1)c1ccc(N(c2ccccc2)c2ccc3[se]c4ccc(N(c5ccccc5)c5ccc(C(C)(C)c6ccccc6)cc5)cc4c3c2)cc1 ; CC(C)(c1ccc(N(c2ccc(-c3ccccc3)cc2)c2ccc(-c3ccccc3)cc2)cc1)c1ccc2ccccc2c1]

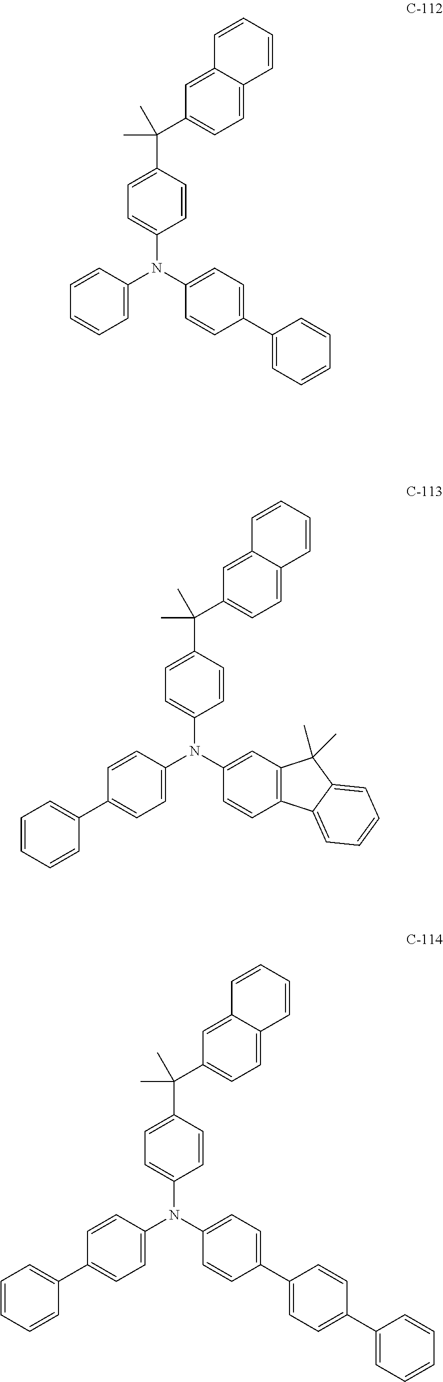 [SMILES: CC(C)(c1ccc(N(c2ccccc2)c2ccc(-c3ccccc3)cc2)cc1)c1ccc2ccccc2c1 ; CC(C)(c1ccc(N(c2ccc(-c3ccccc3)cc2)c2ccc3c(c2)C(C)(C)c2ccccc2-3)cc1)c1ccc2ccccc2c1 ; CC(C)(c1ccc(N(c2ccc(-c3ccccc3)cc2)c2ccc(-c3ccc(-c4ccccc4)cc3)cc2)cc1)c1ccc2ccccc2c1]

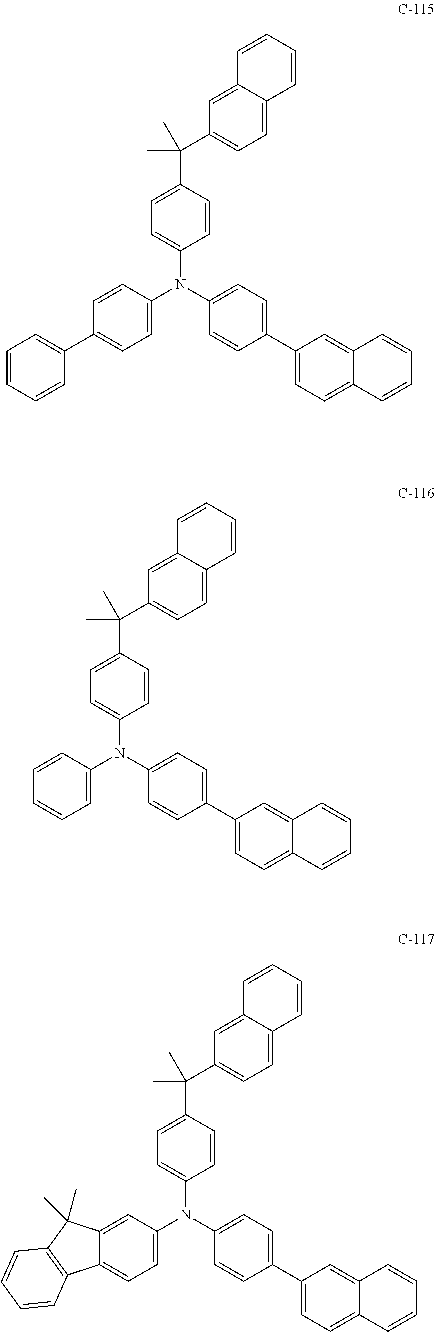 [SMILES: CC(C)(c1ccc(N(c2ccc(-c3ccccc3)cc2)c2ccc(-c3ccc4ccccc4c3)cc2)cc1)c1ccc2ccccc2c1 ; CC(C)(c1ccc(N(c2ccccc2)c2ccc(-c3ccc4ccccc4c3)cc2)cc1)c1ccc2ccccc2c1 ; CC(C)(c1ccc(N(c2ccc(-c3ccc4ccccc4c3)cc2)c2ccc3c(c2)C(C)(C)c2ccccc2-3)cc1)c1ccc2ccccc2c1]

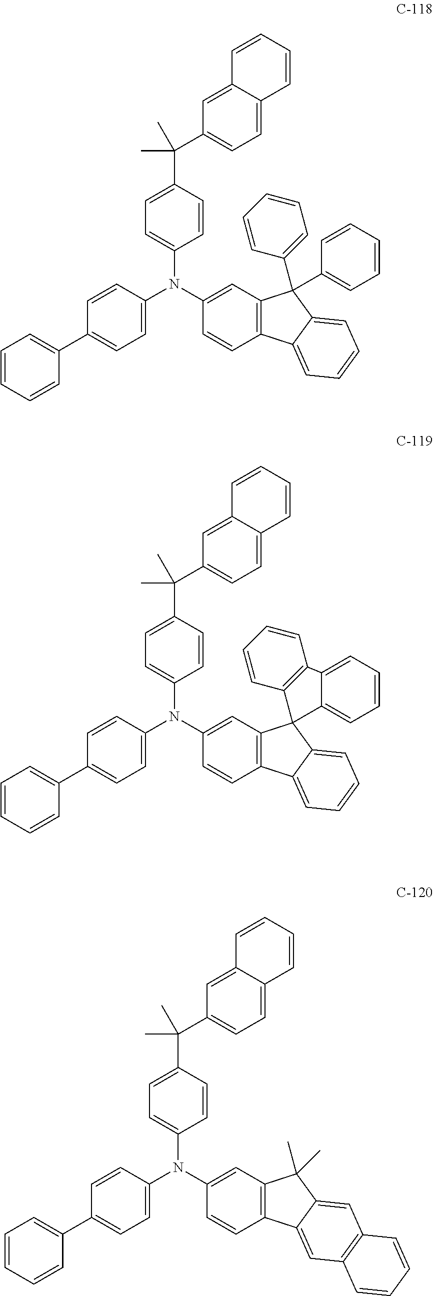 [SMILES: CC(C)(c1ccc(N(c2ccc(-c3ccccc3)cc2)c2ccc3c(c2)C(c2ccccc2)(c2ccccc2)c2ccccc2-3)cc1)c1ccc2ccccc2c1 ; CC(C)(c1ccc(N(c2ccc(-c3ccccc3)cc2)c2ccc3c(c2)C2(c4ccccc4-c4ccccc42)c2ccccc2-3)cc1)c1ccc2ccccc2c1 ; CC(C)(c1ccc(N(c2ccc(-c3ccccc3)cc2)c2ccc3c(c2)C(C)(C)c2cc4ccccc4cc2-3)cc1)c1ccc2ccccc2c1]

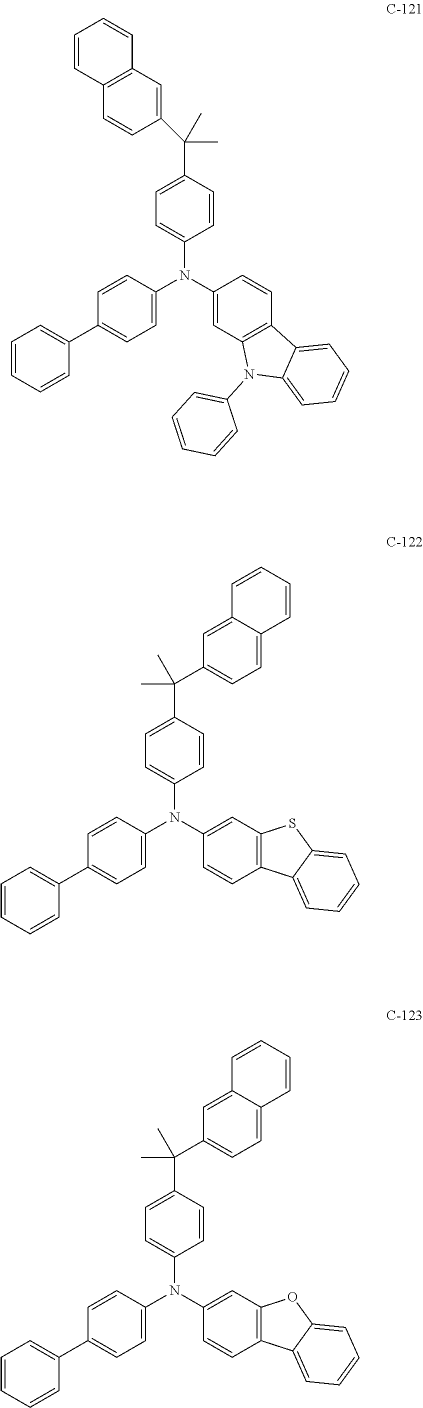 [SMILES: CC(C)(c1ccc(N(c2ccc(-c3ccccc3)cc2)c2ccc3c4ccccc4n(-c4ccccc4)c3c2)cc1)c1ccc2ccccc2c1 ; CC(C)(c1ccc(N(c2ccc(-c3ccccc3)cc2)c2ccc3c(c2)sc2ccccc23)cc1)c1ccc2ccccc2c1 ; CC(C)(c1ccc(N(c2ccc(-c3ccccc3)cc2)c2ccc3c(c2)oc2ccccc23)cc1)c1ccc2ccccc2c1]

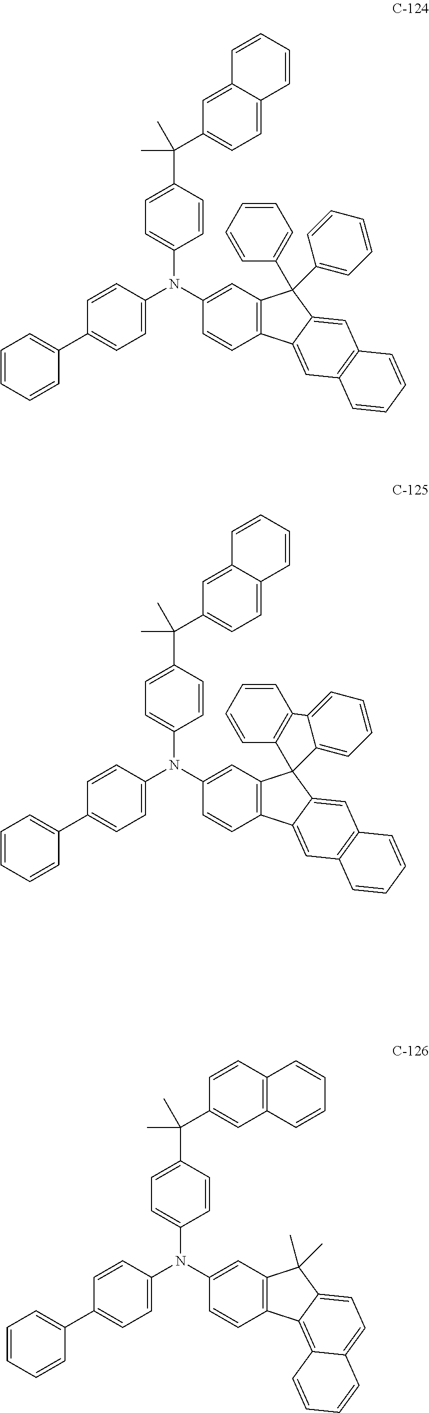 [SMILES: CC(C)(c1ccc(N(c2ccc(-c3ccccc3)cc2)c2ccc3c(c2)C(c2ccccc2)(c2ccccc2)c2cc4ccccc4cc2-3)cc1)c1ccc2ccccc2c1 ; CC(C)(c1ccc(N(c2ccc(-c3ccccc3)cc2)c2ccc3c(c2)C2(c4ccccc4-c4ccccc42)c2cc4ccccc4cc2-3)cc1)c1ccc2ccccc2c1 ; CC(C)(c1ccc(N(c2ccc(-c3ccccc3)cc2)c2ccc3c(c2)C(C)(C)c2ccc4ccccc4c2-3)cc1)c1ccc2ccccc2c1]

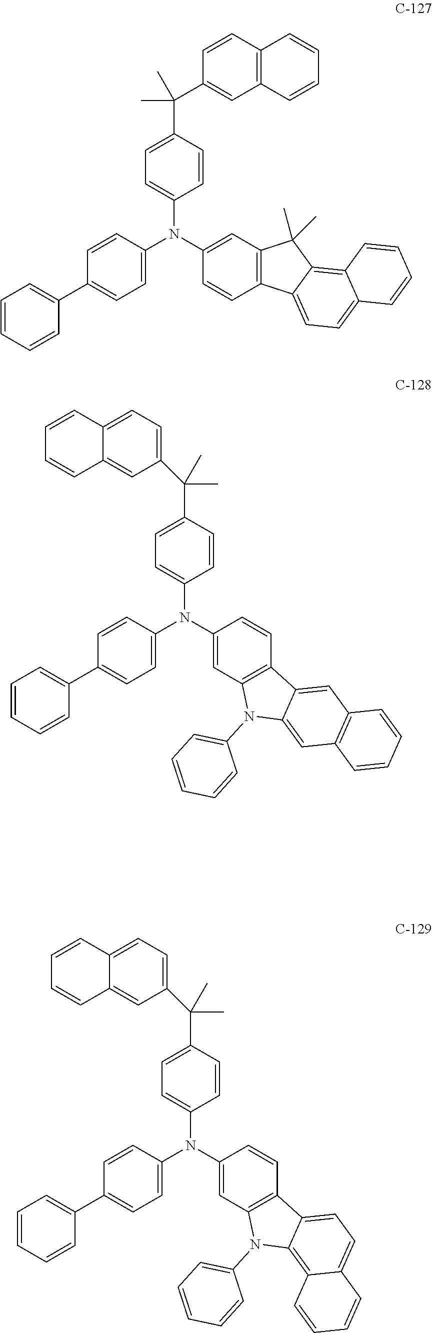 [SMILES: CC(C)(c1ccc(N(c2ccc(-c3ccccc3)cc2)c2ccc3c(c2)C(C)(C)c2c-3ccc3ccccc23)cc1)c1ccc2ccccc2c1 ; CC(C)(c1ccc(N(c2ccc(-c3ccccc3)cc2)c2ccc3c4cc5ccccc5cc4n(-c4ccccc4)c3c2)cc1)c1ccc2ccccc2c1 ; CC(C)(c1ccc(N(c2ccc(-c3ccccc3)cc2)c2ccc3c4ccc5ccccc5c4n(-c4ccccc4)c3c2)cc1)c1ccc2ccccc2c1]

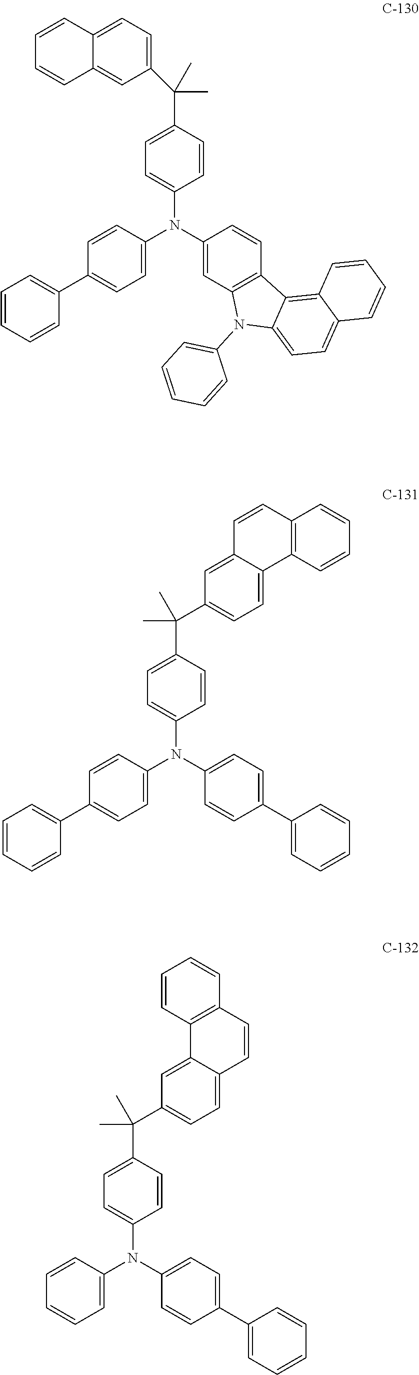 [SMILES: CC(C)(c1ccc(N(c2ccc(-c3ccccc3)cc2)c2ccc3c4c5ccccc5ccc4n(-c4ccccc4)c3c2)cc1)c1ccc2ccccc2c1 ; CC(C)(c1ccc(N(c2ccc(-c3ccccc3)cc2)c2ccc(-c3ccccc3)cc2)cc1)c1ccc2c(ccc3ccccc32)c1 ; CC(C)(c1ccc(N(c2ccccc2)c2ccc(-c3ccccc3)cc2)cc1)c1ccc2ccc3ccccc3c2c1]

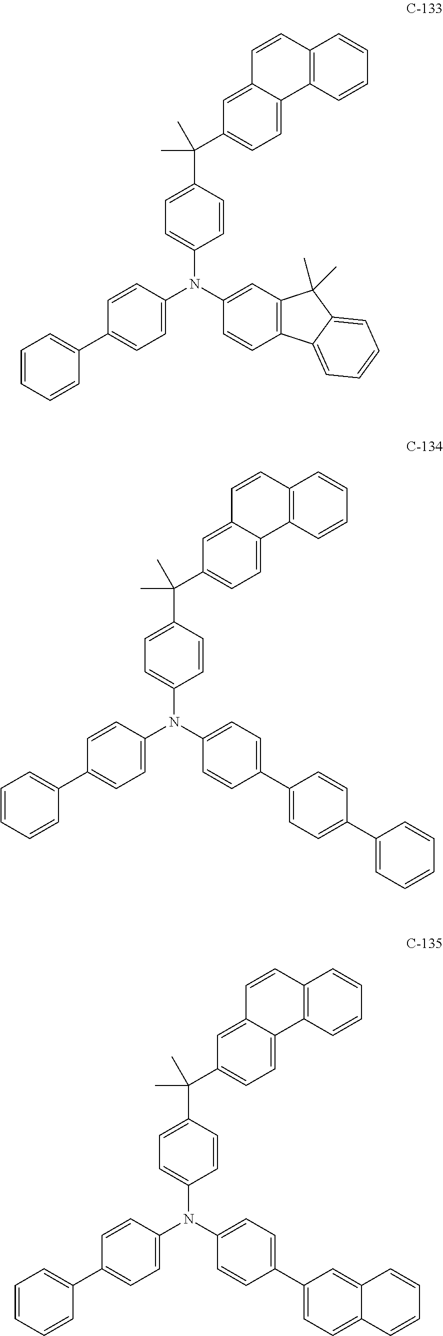 [SMILES: CC(C)(c1ccc(N(c2ccc(-c3ccccc3)cc2)c2ccc3c(c2)C(C)(C)c2ccccc2-3)cc1)c1ccc2c(ccc3ccccc32)c1 ; CC(C)(c1ccc(N(c2ccc(-c3ccccc3)cc2)c2ccc(-c3ccc(-c4ccccc4)cc3)cc2)cc1)c1ccc2c(ccc3ccccc32)c1 ; CC(C)(c1ccc(N(c2ccc(-c3ccccc3)cc2)c2ccc(-c3ccc4ccccc4c3)cc2)cc1)c1ccc2c(ccc3ccccc32)c1]

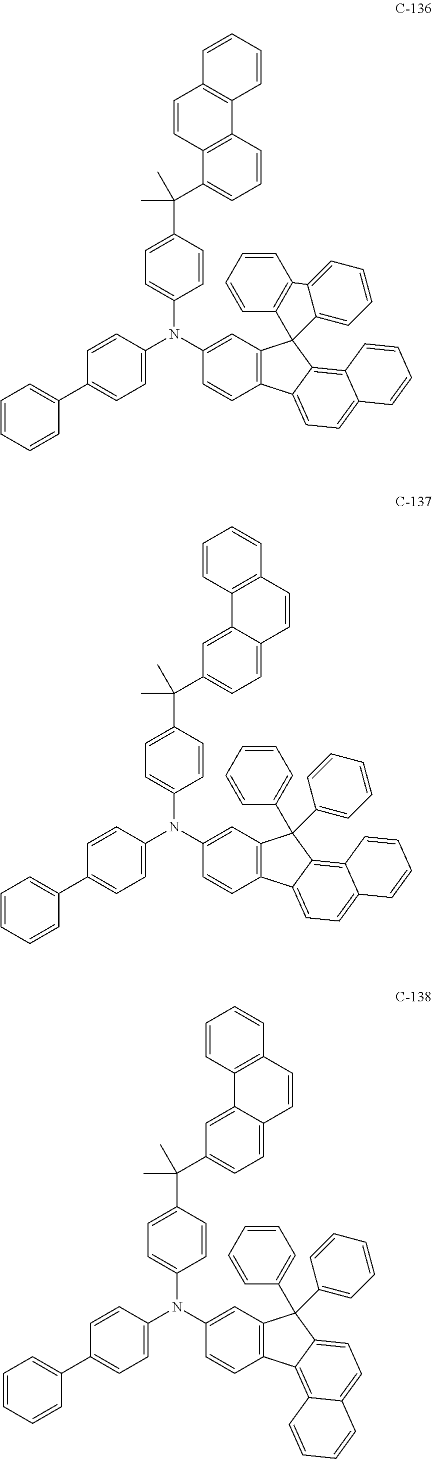 [SMILES: CC(C)(c1ccc(N(c2ccc(-c3ccccc3)cc2)c2ccc3c(c2)C2(c4ccccc4-c4ccccc42)c2c-3ccc3ccccc23)cc1)c1cccc2c1ccc1ccccc12 ; CC(C)(c1ccc(N(c2ccc(-c3ccccc3)cc2)c2ccc3c(c2)C(c2ccccc2)(c2ccccc2)c2c-3ccc3ccccc23)cc1)c1ccc2ccc3ccccc3c2c1 ; CC(C)(c1ccc(N(c2ccc(-c3ccccc3)cc2)c2ccc3c(c2)C(c2ccccc2)(c2ccccc2)c2ccc4ccccc4c2-3)cc1)c1ccc2ccc3ccccc3c2c1]

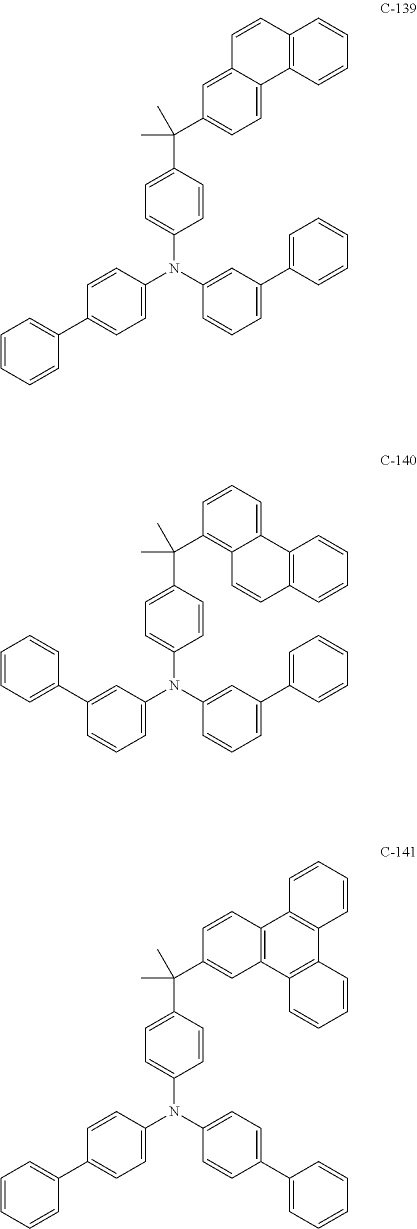 [SMILES: CC(C)(c1ccc(N(c2ccc(-c3ccccc3)cc2)c2cccc(-c3ccccc3)c2)cc1)c1ccc2c(ccc3ccccc32)c1 ; CC(C)(c1ccc(N(c2cccc(-c3ccccc3)c2)c2cccc(-c3ccccc3)c2)cc1)c1cccc2c1ccc1ccccc12 ; CC(C)(c1ccc(N(c2ccc(-c3ccccc3)cc2)c2ccc(-c3ccccc3)cc2)cc1)c1ccc2c3ccccc3c3ccccc3c2c1]

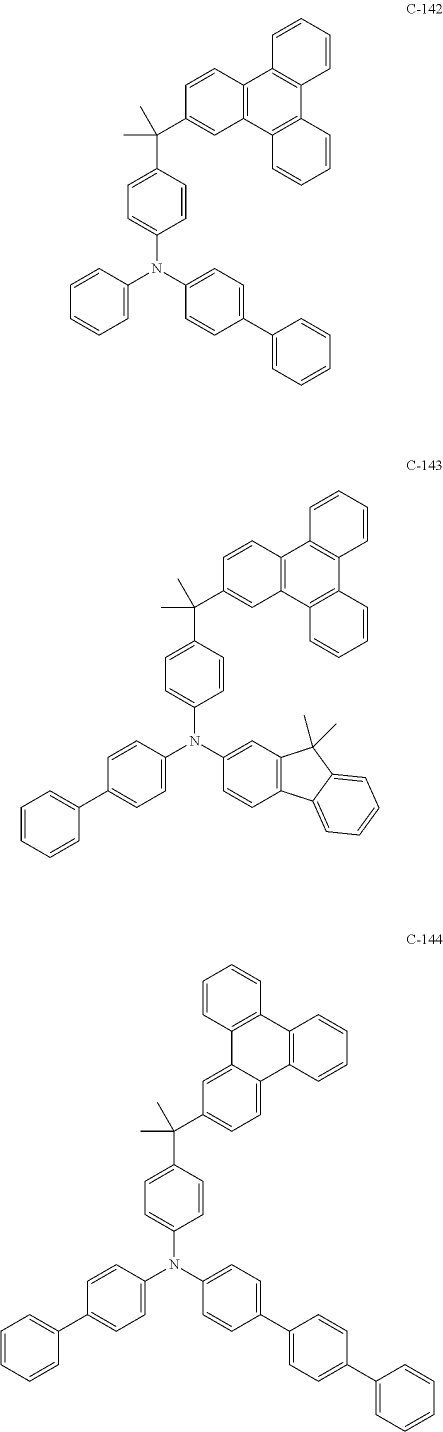 [SMILES: CC(C)(c1ccc(N(c2ccccc2)c2ccc(-c3ccccc3)cc2)cc1)c1ccc2c3ccccc3c3ccccc3c2c1 ; CC(C)(c1ccc(N(c2ccc(-c3ccccc3)cc2)c2ccc3c(c2)C(C)(C)c2ccccc2-3)cc1)c1ccc2c3ccccc3c3ccccc3c2c1 ; CC(C)(c1ccc(N(c2ccc(-c3ccccc3)cc2)c2ccc(-c3ccc(-c4ccccc4)cc3)cc2)cc1)c1ccc2c3ccccc3c3ccccc3c2c1]

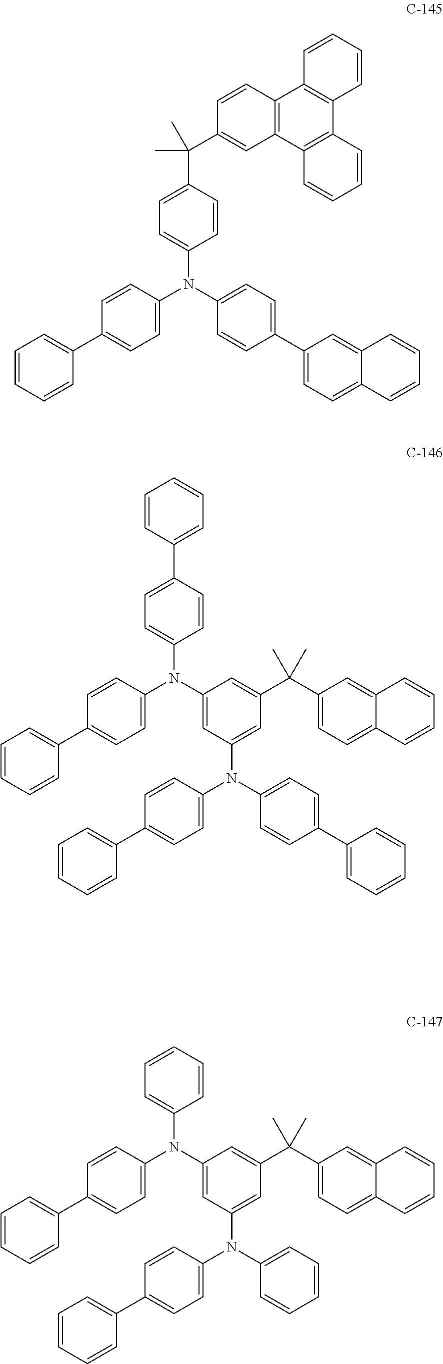 [SMILES: CC(C)(c1ccc(N(c2ccc(-c3ccccc3)cc2)c2ccc(-c3ccc4ccccc4c3)cc2)cc1)c1ccc2c3ccccc3c3ccccc3c2c1 ; CC(C)(c1cc(N(c2ccc(-c3ccccc3)cc2)c2ccc(-c3ccccc3)cc2)cc(N(c2ccc(-c3ccccc3)cc2)c2ccc(-c3ccccc3)cc2)c1)c1ccc2ccccc2c1 ; CC(C)(c1cc(N(c2ccccc2)c2ccc(-c3ccccc3)cc2)cc(N(c2ccccc2)c2ccc(-c3ccccc3)cc2)c1)c1ccc2ccccc2c1]

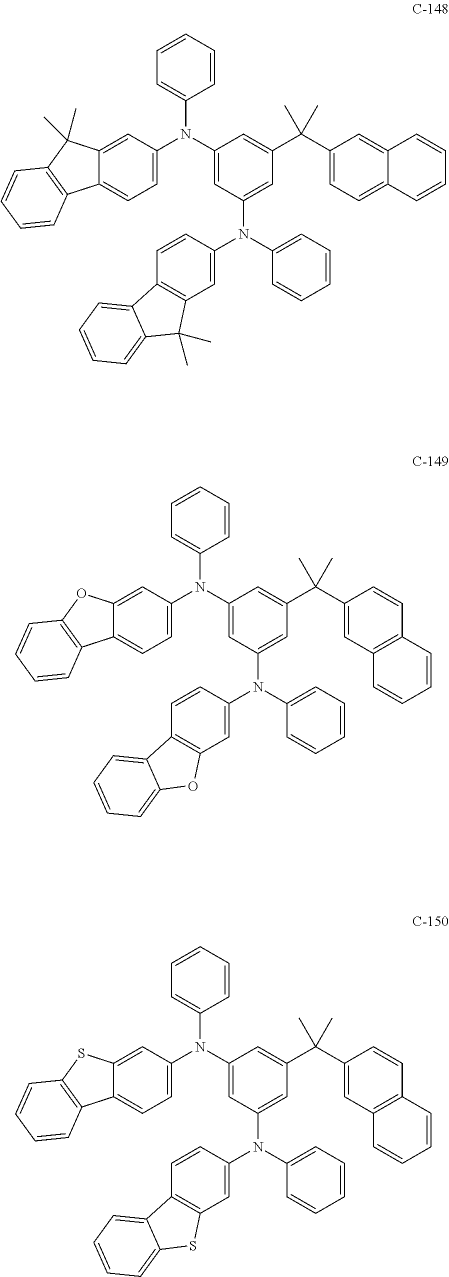 [SMILES: CC(C)(c1cc(N(c2ccccc2)c2ccc3c(c2)C(C)(C)c2ccccc2-3)cc(N(c2ccccc2)c2ccc3c(c2)C(C)(C)c2ccccc2-3)c1)c1ccc2ccccc2c1 ; CC(C)(c1cc(N(c2ccccc2)c2ccc3c(c2)oc2ccccc23)cc(N(c2ccccc2)c2ccc3c(c2)oc2ccccc23)c1)c1ccc2ccccc2c1 ; CC(C)(c1cc(N(c2ccccc2)c2ccc3c(c2)sc2ccccc23)cc(N(c2ccccc2)c2ccc3c(c2)sc2ccccc23)c1)c1ccc2ccccc2c1]

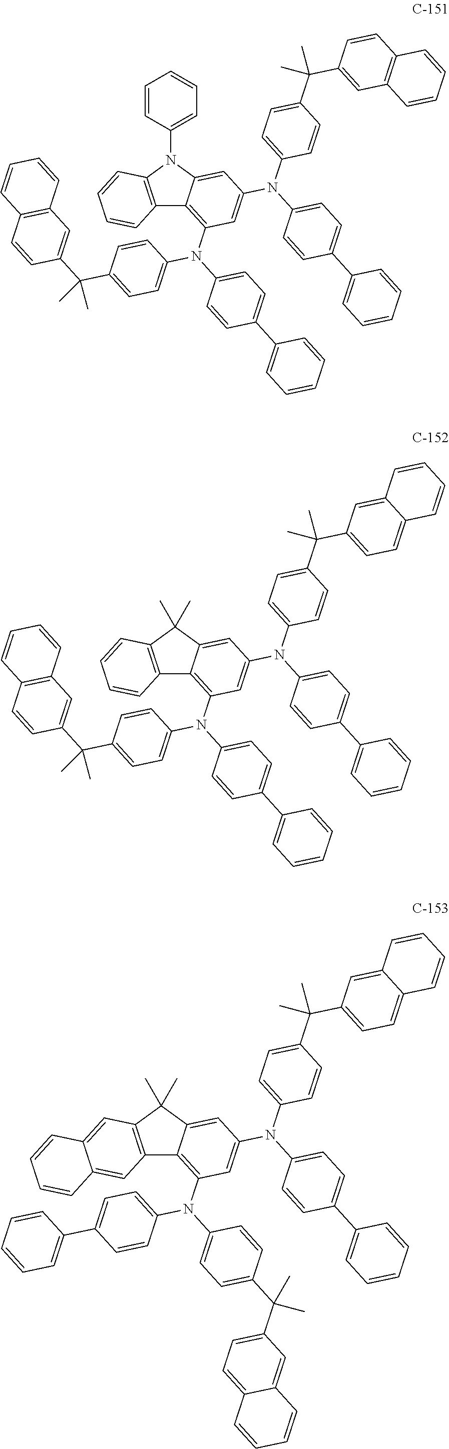 [SMILES: CC(C)(c1ccc(N(c2ccc(-c3ccccc3)cc2)c2cc(N(c3ccc(-c4ccccc4)cc3)c3ccc(C(C)(C)c4ccc5ccccc5c4)cc3)c3c4ccccc4n(-c4ccccc4)c3c2)cc1)c1ccc2ccccc2c1 ; CC(C)(c1ccc(N(c2ccc(-c3ccccc3)cc2)c2cc(N(c3ccc(-c4ccccc4)cc3)c3ccc(C(C)(C)c4ccc5ccccc5c4)cc3)c3c(c2)C(C)(C)c2ccccc2-3)cc1)c1ccc2ccccc2c1 ; CC(C)(c1ccc(N(c2ccc(-c3ccccc3)cc2)c2cc(N(c3ccc(-c4ccccc4)cc3)c3ccc(C(C)(C)c4ccc5ccccc5c4)cc3)c3c(c2)C(C)(C)c2cc4ccccc4cc2-3)cc1)c1ccc2ccccc2c1]

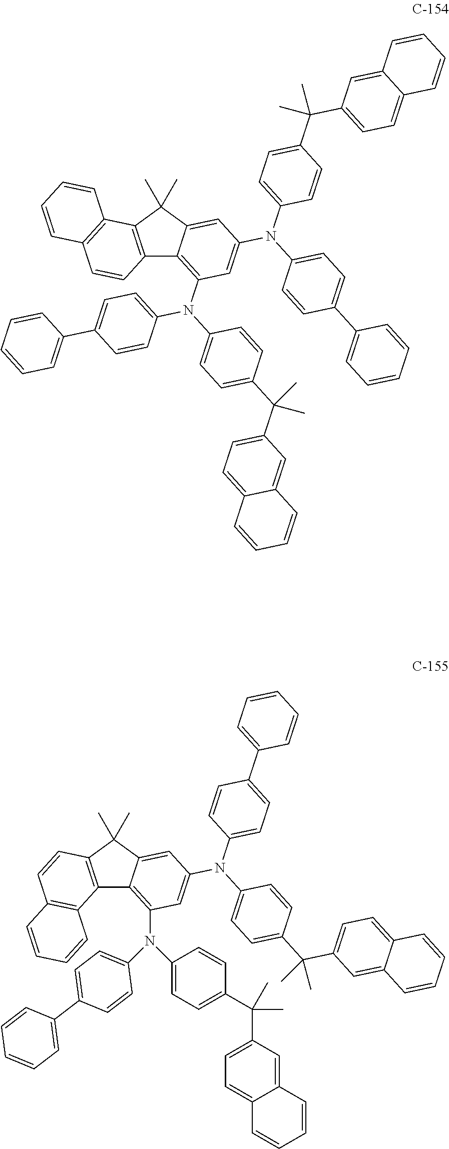 [SMILES: CC(C)(c1ccc(N(c2ccc(-c3ccccc3)cc2)c2cc(N(c3ccc(-c4ccccc4)cc3)c3ccc(C(C)(C)c4ccc5ccccc5c4)cc3)c3c(c2)C(C)(C)c2c-3ccc3ccccc23)cc1)c1ccc2ccccc2c1 ; CC(C)(c1ccc(N(c2ccc(-c3ccccc3)cc2)c2cc(N(c3ccc(-c4ccccc4)cc3)c3ccc(C(C)(C)c4ccc5ccccc5c4)cc3)c3c(c2)C(C)(C)c2ccc4ccccc4c2-3)cc1)c1ccc2ccccc2c1]

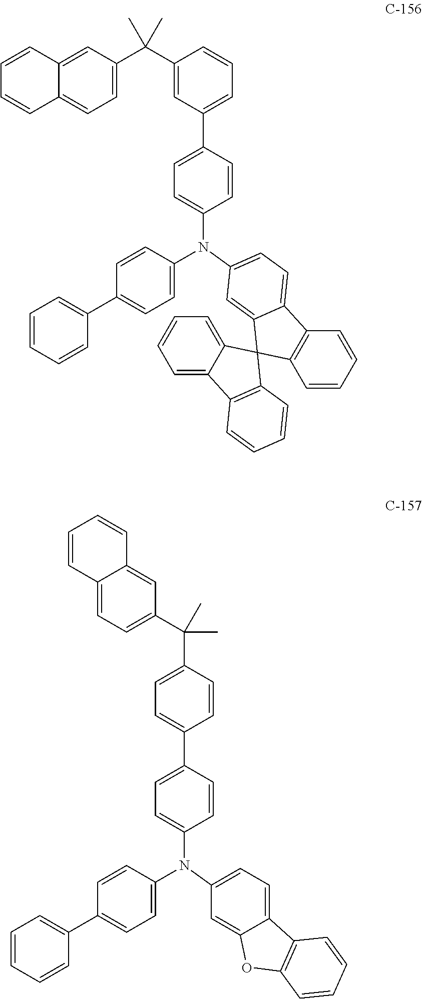 [SMILES: CC(C)(c1cccc(-c2ccc(N(c3ccc(-c4ccccc4)cc3)c3ccc4c(c3)C3(c5ccccc5-c5ccccc53)c3ccccc3-4)cc2)c1)c1ccc2ccccc2c1 ; CC(C)(c1ccc(-c2ccc(N(c3ccc(-c4ccccc4)cc3)c3ccc4c(c3)oc3ccccc34)cc2)cc1)c1ccc2ccccc2c1]

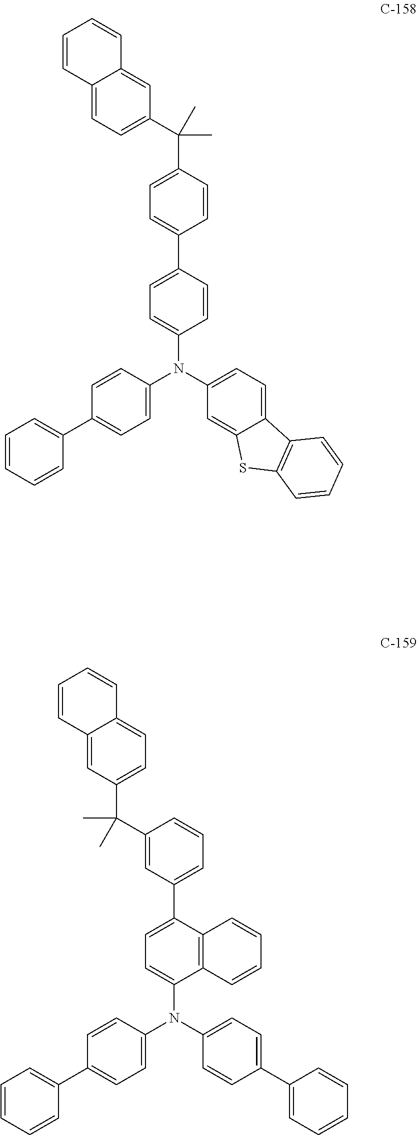 [SMILES: CC(C)(c1ccc(-c2ccc(N(c3ccc(-c4ccccc4)cc3)c3ccc4c(c3)sc3ccccc34)cc2)cc1)c1ccc2ccccc2c1 ; CC(C)(c1cccc(-c2ccc(N(c3ccc(-c4ccccc4)cc3)c3ccc(-c4ccccc4)cc3)c3ccccc23)c1)c1ccc2ccccc2c1]

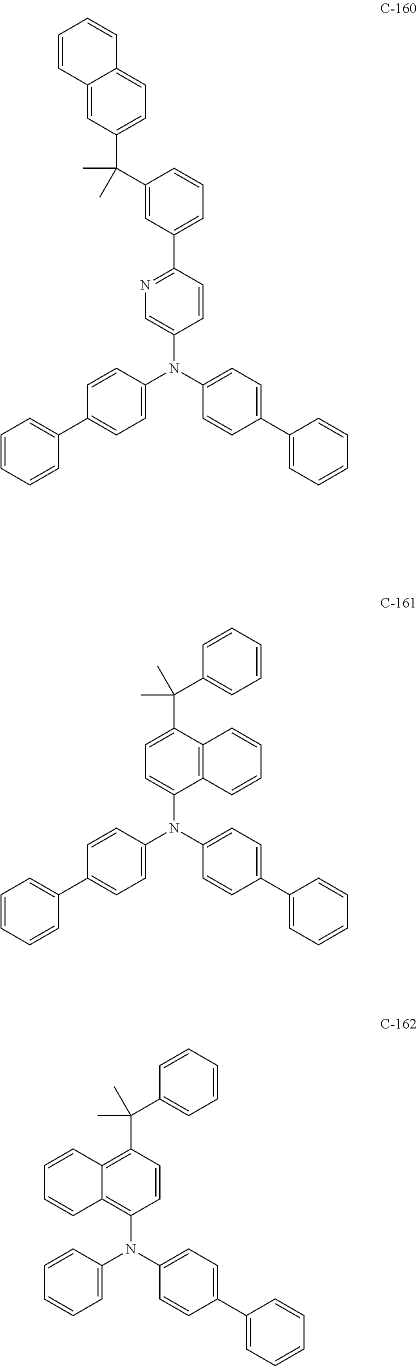 [SMILES: CC(C)(c1cccc(-c2ccc(N(c3ccc(-c4ccccc4)cc3)c3ccc(-c4ccccc4)cc3)cn2)c1)c1ccc2ccccc2c1 ; CC(C)(c1ccccc1)c1ccc(N(c2ccc(-c3ccccc3)cc2)c2ccc(-c3ccccc3)cc2)c2ccccc12 ; CC(C)(c1ccccc1)c1ccc(N(c2ccccc2)c2ccc(-c3ccccc3)cc2)c2ccccc12]

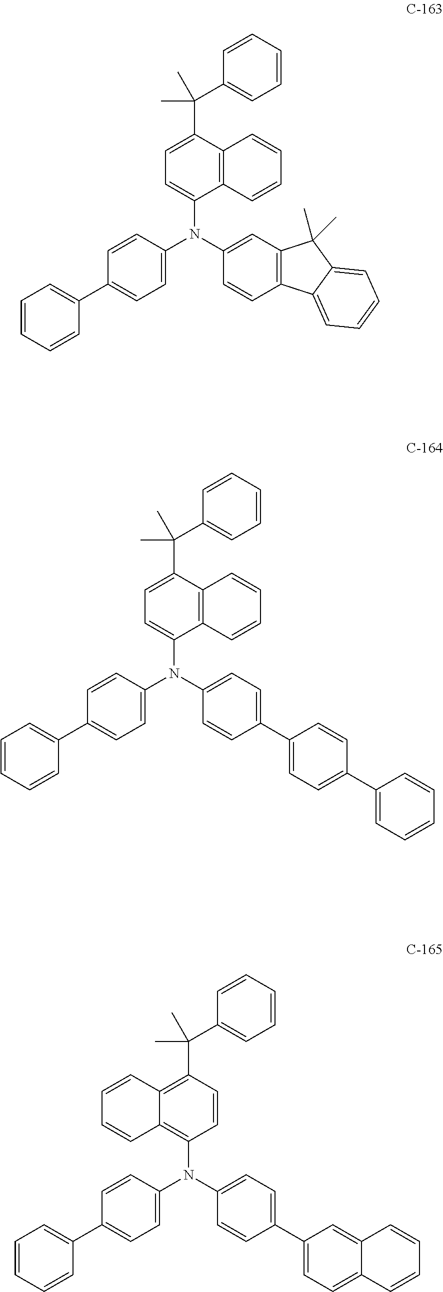 [SMILES: CC1(C)c2ccccc2-c2ccc(N(c3ccc(-c4ccccc4)cc3)c3ccc(C(C)(C)c4ccccc4)c4ccccc34)cc21 ; CC(C)(c1ccccc1)c1ccc(N(c2ccc(-c3ccccc3)cc2)c2ccc(-c3ccc(-c4ccccc4)cc3)cc2)c2ccccc12 ; CC(C)(c1ccccc1)c1ccc(N(c2ccc(-c3ccccc3)cc2)c2ccc(-c3ccc4ccccc4c3)cc2)c2ccccc12]

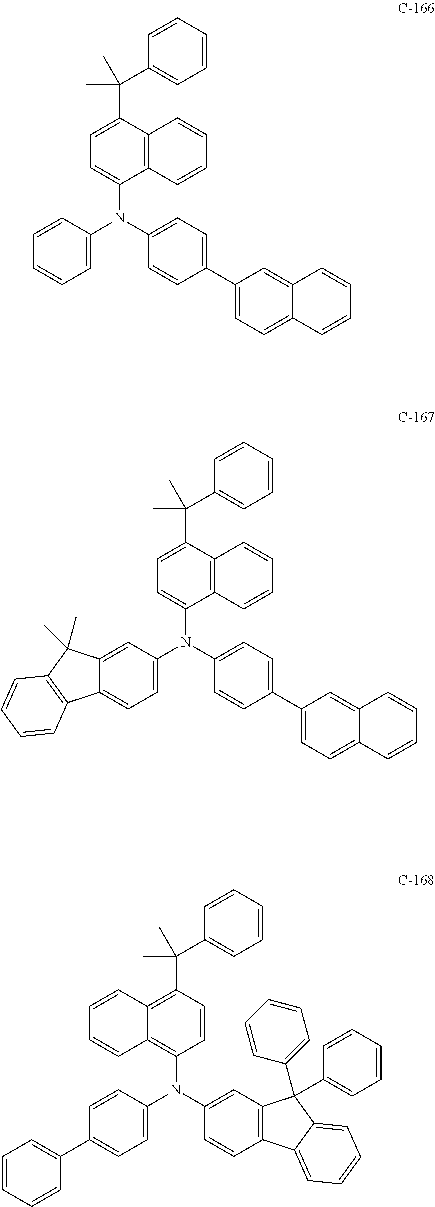 [SMILES: CC(C)(c1ccccc1)c1ccc(N(c2ccccc2)c2ccc(-c3ccc4ccccc4c3)cc2)c2ccccc12 ; CC1(C)c2ccccc2-c2ccc(N(c3ccc(-c4ccc5ccccc5c4)cc3)c3ccc(C(C)(C)c4ccccc4)c4ccccc34)cc21 ; CC(C)(c1ccccc1)c1ccc(N(c2ccc(-c3ccccc3)cc2)c2ccc3c(c2)C(c2ccccc2)(c2ccccc2)c2ccccc2-3)c2ccccc12]

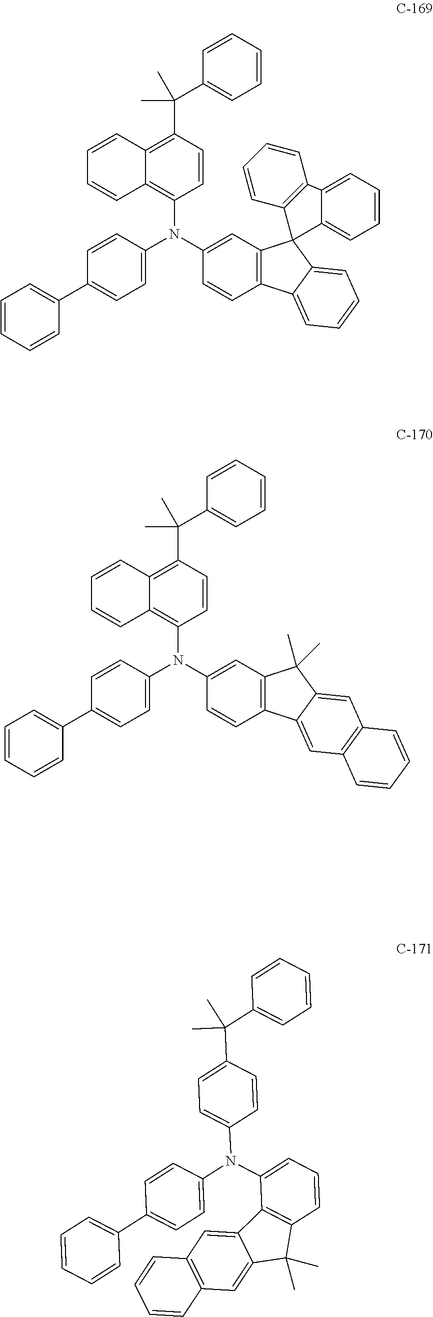 [SMILES: CC(C)(c1ccccc1)c1ccc(N(c2ccc(-c3ccccc3)cc2)c2ccc3c(c2)C2(c4ccccc4-c4ccccc42)c2ccccc2-3)c2ccccc12 ; CC1(C)c2cc(N(c3ccc(-c4ccccc4)cc3)c3ccc(C(C)(C)c4ccccc4)c4ccccc34)ccc2-c2cc3ccccc3cc21 ; CC(C)(c1ccccc1)c1ccc(N(c2ccc(-c3ccccc3)cc2)c2cccc3c2-c2cc4ccccc4cc2C3(C)C)cc1]

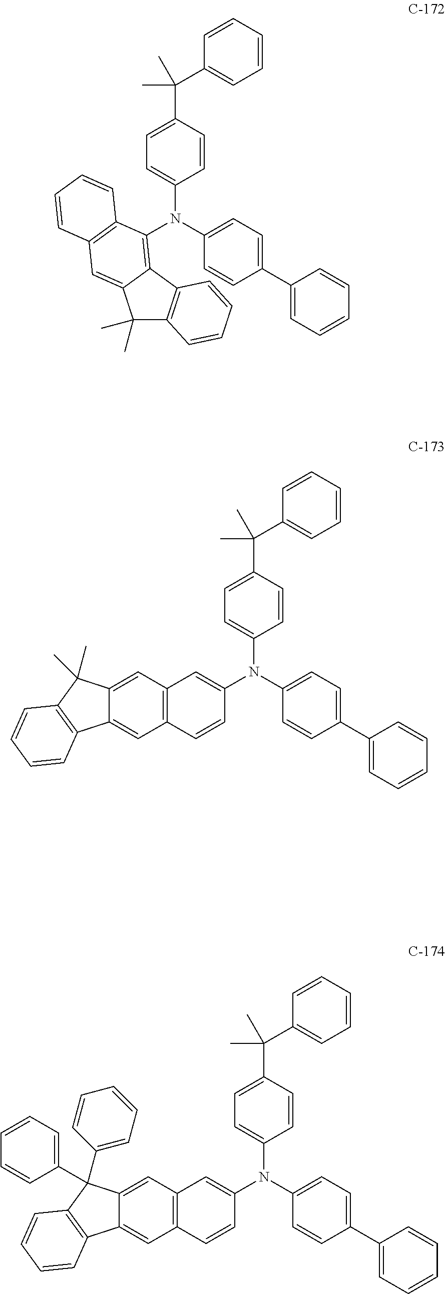 [SMILES: CC(C)(c1ccccc1)c1ccc(N(c2ccc(-c3ccccc3)cc2)c2c3c(cc4ccccc24)C(C)(C)c2ccccc2-3)cc1 ; CC(C)(c1ccccc1)c1ccc(N(c2ccc(-c3ccccc3)cc2)c2ccc3cc4c(cc3c2)C(C)(C)c2ccccc2-4)cc1 ; CC(C)(c1ccccc1)c1ccc(N(c2ccc(-c3ccccc3)cc2)c2ccc3cc4c(cc3c2)C(c2ccccc2)(c2ccccc2)c2ccccc2-4)cc1]

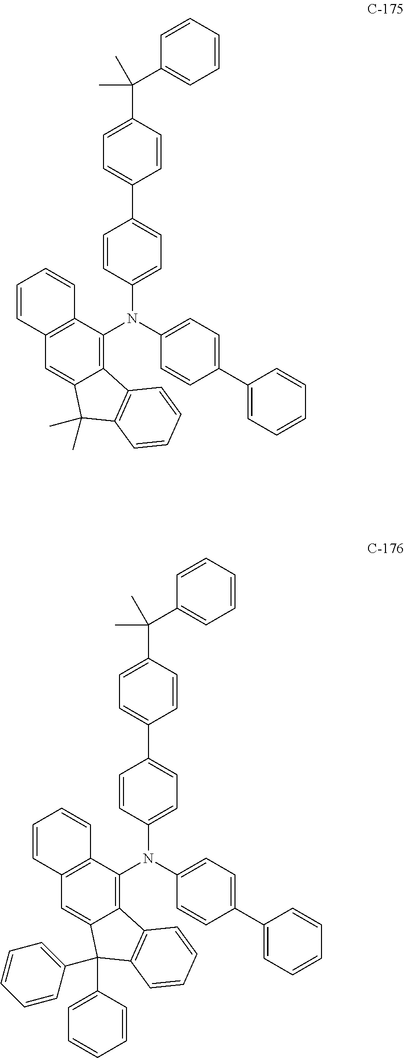 [SMILES: CC(C)(c1ccccc1)c1ccc(-c2ccc(N(c3ccc(-c4ccccc4)cc3)c3c4c(cc5ccccc35)C(C)(C)c3ccccc3-4)cc2)cc1 ; CC(C)(c1ccccc1)c1ccc(-c2ccc(N(c3ccc(-c4ccccc4)cc3)c3c4c(cc5ccccc35)C(c3ccccc3)(c3ccccc3)c3ccccc3-4)cc2)cc1]

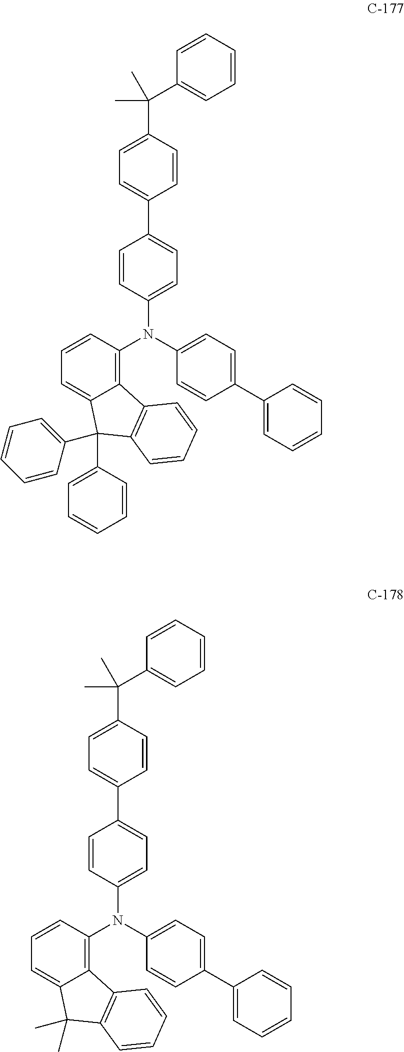 [SMILES: CC(C)(c1ccccc1)c1ccc(-c2ccc(N(c3ccc(-c4ccccc4)cc3)c3cccc4c3-c3ccccc3C4(c3ccccc3)c3ccccc3)cc2)cc1 ; CC(C)(c1ccccc1)c1ccc(-c2ccc(N(c3ccc(-c4ccccc4)cc3)c3cccc4c3-c3ccccc3C4(C)C)cc2)cc1]

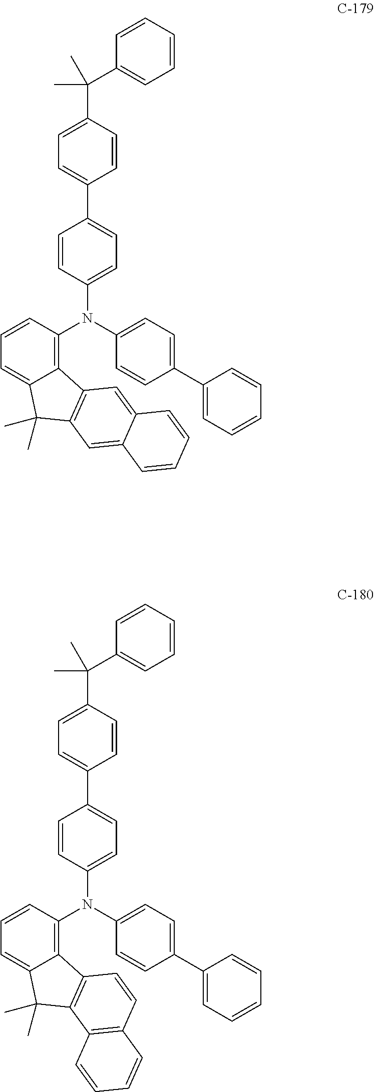 [SMILES: CC(C)(c1ccccc1)c1ccc(-c2ccc(N(c3ccc(-c4ccccc4)cc3)c3cccc4c3-c3cc5ccccc5cc3C4(C)C)cc2)cc1 ; CC(C)(c1ccccc1)c1ccc(-c2ccc(N(c3ccc(-c4ccccc4)cc3)c3cccc4c3-c3ccc5ccccc5c3C4(C)C)cc2)cc1]

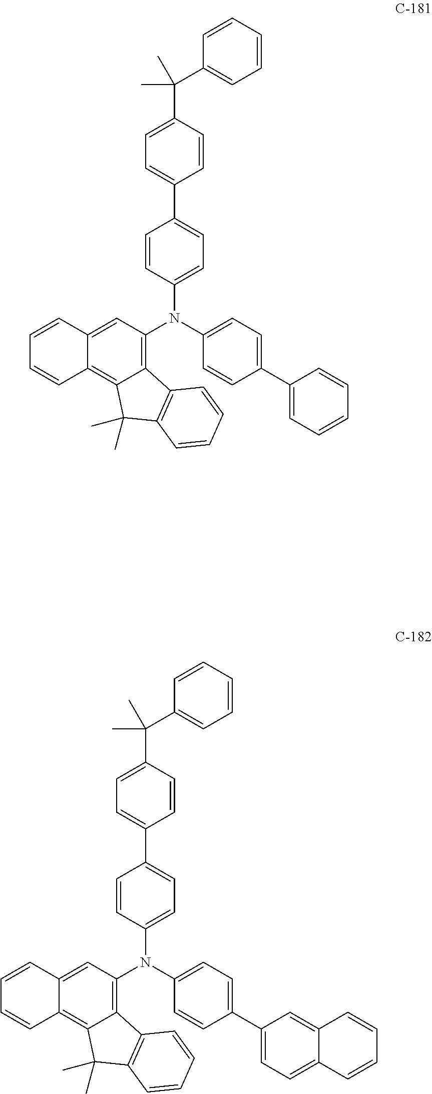 [SMILES: CC(C)(c1ccccc1)c1ccc(-c2ccc(N(c3ccc(-c4ccccc4)cc3)c3cc4ccccc4c4c3-c3ccccc3C4(C)C)cc2)cc1 ; CC(C)(c1ccccc1)c1ccc(-c2ccc(N(c3ccc(-c4ccc5ccccc5c4)cc3)c3cc4ccccc4c4c3-c3ccccc3C4(C)C)cc2)cc1]

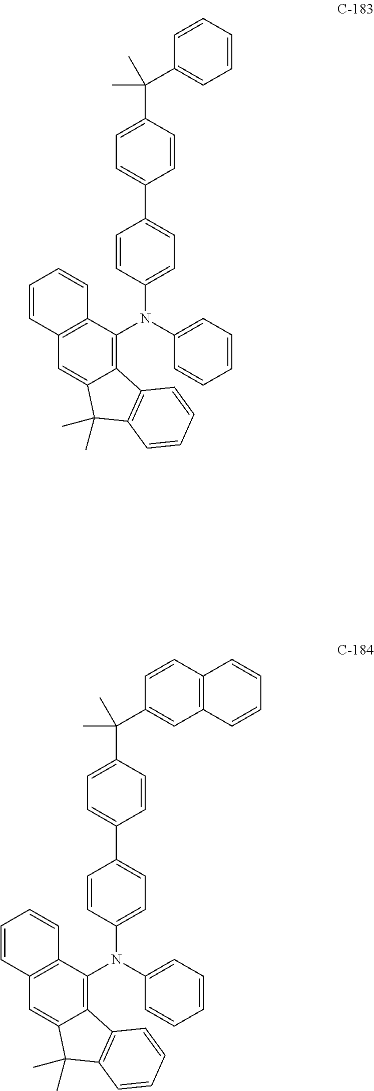 [SMILES: CC(C)(c1ccccc1)c1ccc(-c2ccc(N(c3ccccc3)c3c4c(cc5ccccc35)C(C)(C)c3ccccc3-4)cc2)cc1 ; CC(C)(c1ccc(-c2ccc(N(c3ccccc3)c3c4c(cc5ccccc35)C(C)(C)c3ccccc3-4)cc2)cc1)c1ccc2ccccc2c1]

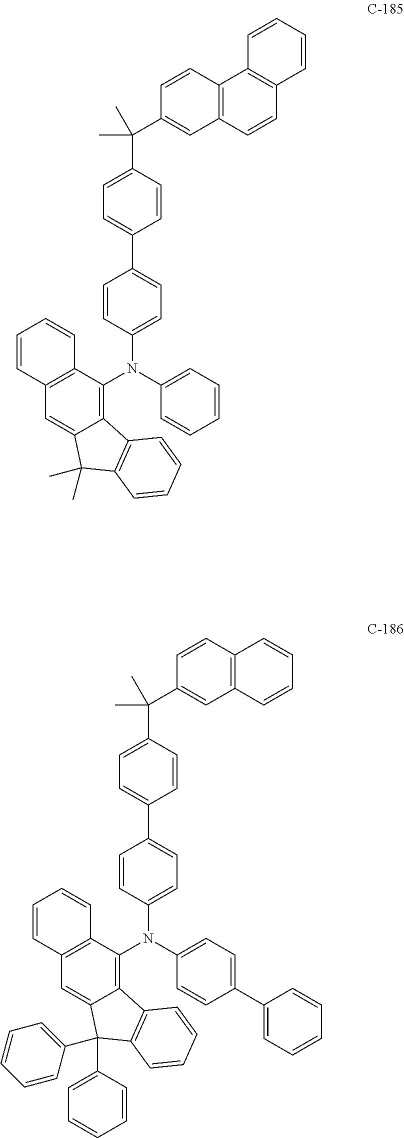 [SMILES: CC(C)(c1ccc(-c2ccc(N(c3ccccc3)c3c4c(cc5ccccc35)C(C)(C)c3ccccc3-4)cc2)cc1)c1ccc2c(ccc3ccccc32)c1 ; CC(C)(c1ccc(-c2ccc(N(c3ccc(-c4ccccc4)cc3)c3c4c(cc5ccccc35)C(c3ccccc3)(c3ccccc3)c3ccccc3-4)cc2)cc1)c1ccc2ccccc2c1]

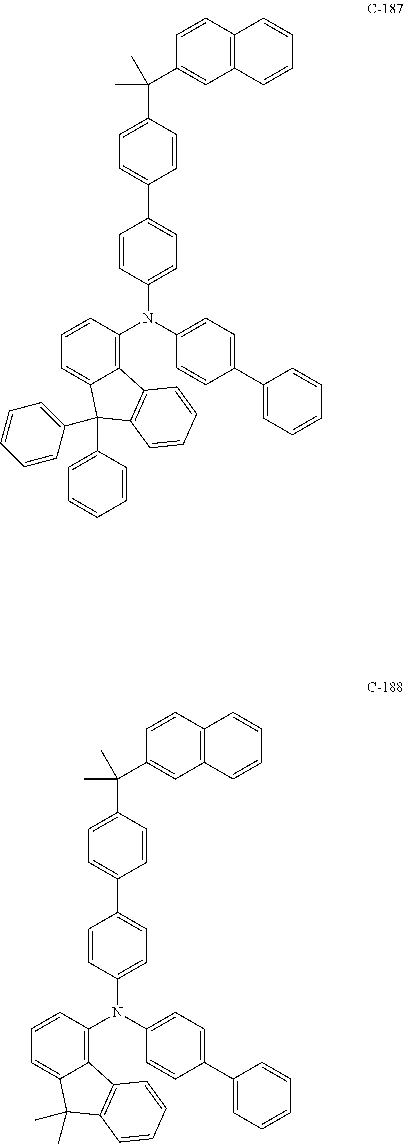 [SMILES: CC(C)(c1ccc(-c2ccc(N(c3ccc(-c4ccccc4)cc3)c3cccc4c3-c3ccccc3C4(c3ccccc3)c3ccccc3)cc2)cc1)c1ccc2ccccc2c1 ; CC(C)(c1ccc(-c2ccc(N(c3ccc(-c4ccccc4)cc3)c3cccc4c3-c3ccccc3C4(C)C)cc2)cc1)c1ccc2ccccc2c1]

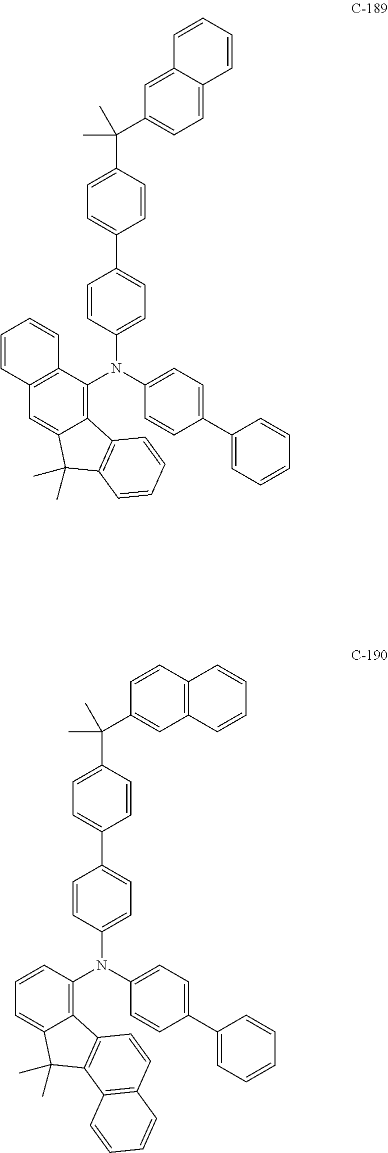 [SMILES: CC(C)(c1ccc(-c2ccc(N(c3ccc(-c4ccccc4)cc3)c3c4c(cc5ccccc35)C(C)(C)c3ccccc3-4)cc2)cc1)c1ccc2ccccc2c1 ; CC(C)(c1ccc(-c2ccc(N(c3ccc(-c4ccccc4)cc3)c3cccc4c3-c3ccc5ccccc5c3C4(C)C)cc2)cc1)c1ccc2ccccc2c1]

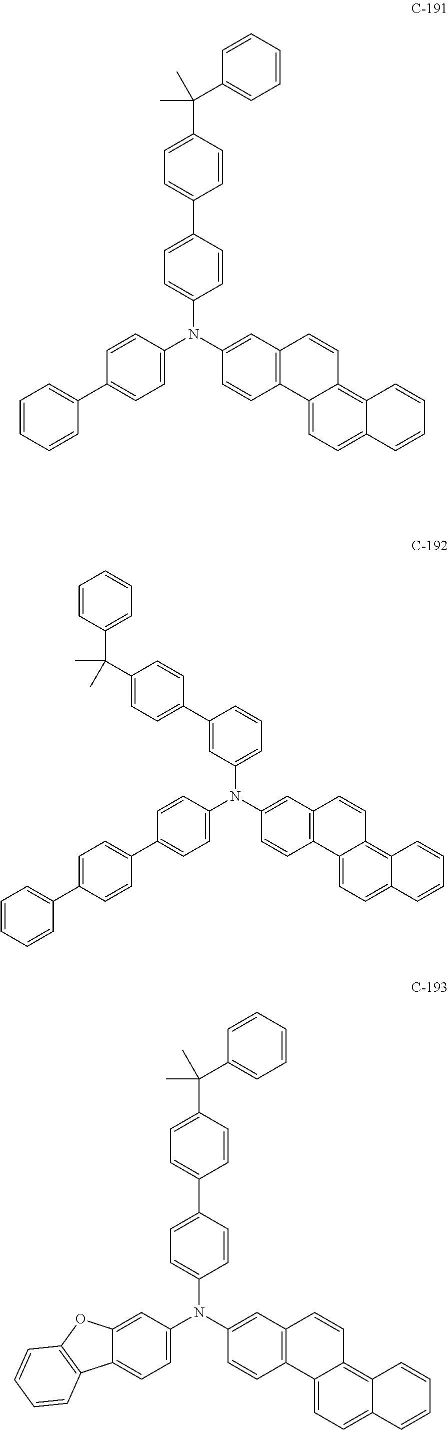 [SMILES: CC(C)(c1ccccc1)c1ccc(-c2ccc(N(c3ccc(-c4ccccc4)cc3)c3ccc4c(ccc5c6ccccc6ccc45)c3)cc2)cc1 ; CC(C)(c1ccccc1)c1ccc(-c2cccc(N(c3ccc(-c4ccc(-c5ccccc5)cc4)cc3)c3ccc4c(ccc5c6ccccc6ccc45)c3)c2)cc1 ; CC(C)(c1ccccc1)c1ccc(-c2ccc(N(c3ccc4c(ccc5c6ccccc6ccc45)c3)c3ccc4c(c3)oc3ccccc34)cc2)cc1]

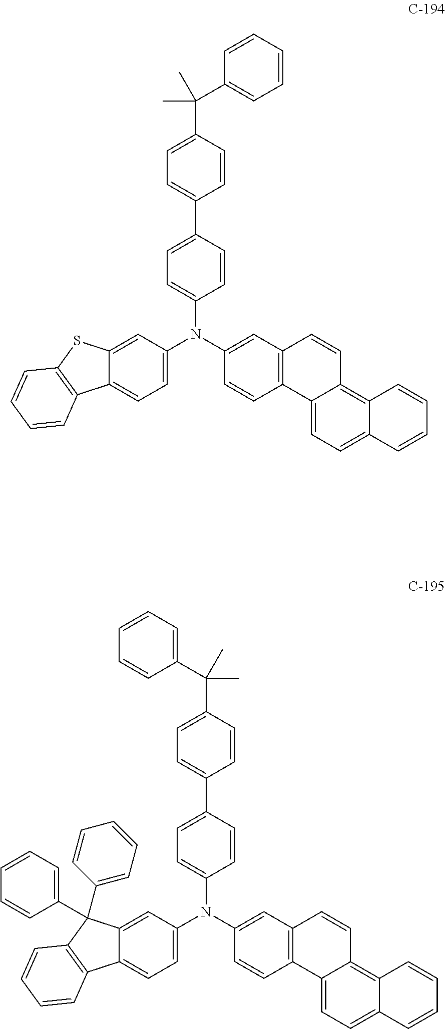 [SMILES: CC(C)(c1ccccc1)c1ccc(-c2ccc(N(c3ccc4c(ccc5c6ccccc6ccc45)c3)c3ccc4c(c3)sc3ccccc34)cc2)cc1 ; CC(C)(c1ccccc1)c1ccc(-c2ccc(N(c3ccc4c(c3)C(c3ccccc3)(c3ccccc3)c3ccccc3-4)c3ccc4c(ccc5c6ccccc6ccc45)c3)cc2)cc1]

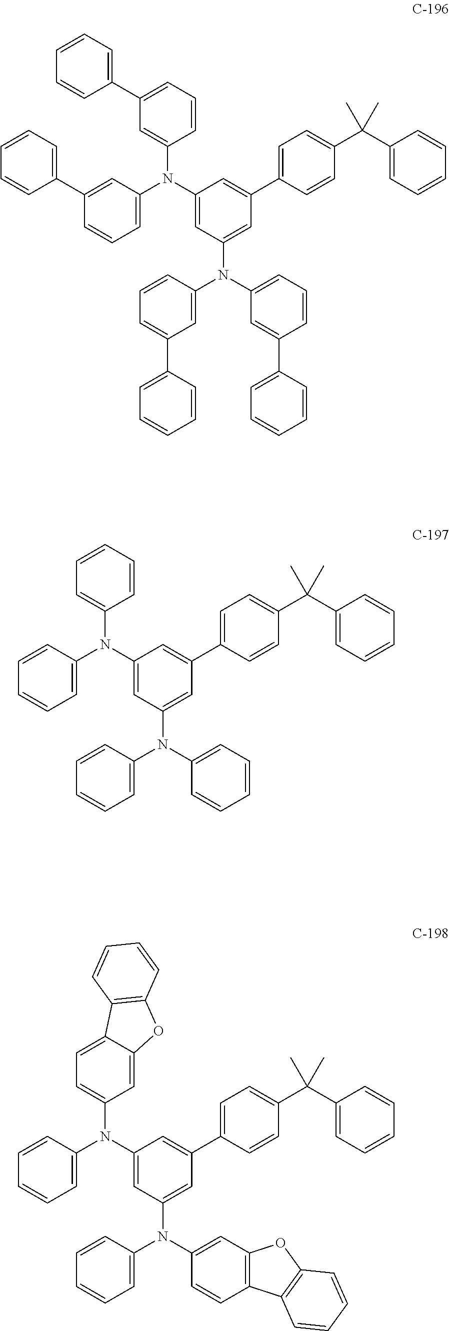 [SMILES: CC(C)(c1ccccc1)c1ccc(-c2cc(N(c3cccc(-c4ccccc4)c3)c3cccc(-c4ccccc4)c3)cc(N(c3cccc(-c4ccccc4)c3)c3cccc(-c4ccccc4)c3)c2)cc1 ; CC(C)(c1ccccc1)c1ccc(-c2cc(N(c3ccccc3)c3ccccc3)cc(N(c3ccccc3)c3ccccc3)c2)cc1 ; CC(C)(c1ccccc1)c1ccc(-c2cc(N(c3ccccc3)c3ccc4c(c3)oc3ccccc34)cc(N(c3ccccc3)c3ccc4c(c3)oc3ccccc34)c2)cc1]

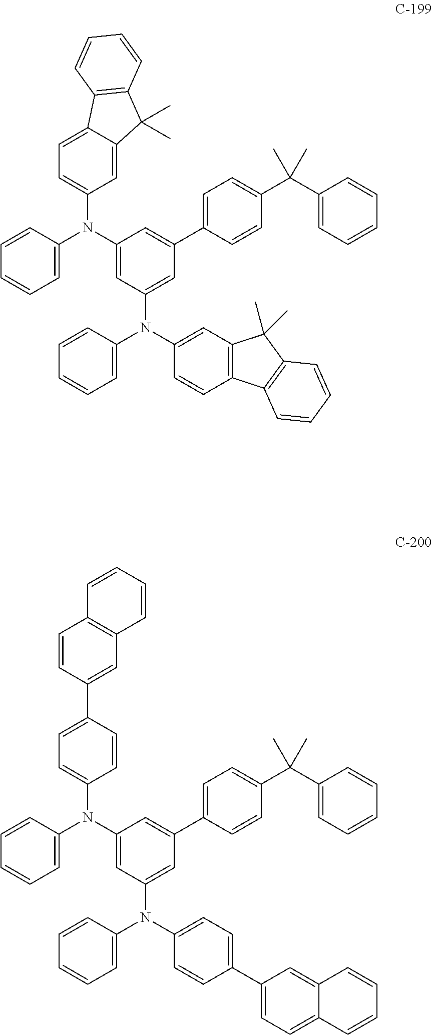 [SMILES: CC(C)(c1ccccc1)c1ccc(-c2cc(N(c3ccccc3)c3ccc4c(c3)C(C)(C)c3ccccc3-4)cc(N(c3ccccc3)c3ccc4c(c3)C(C)(C)c3ccccc3-4)c2)cc1 ; CC(C)(c1ccccc1)c1ccc(-c2cc(N(c3ccccc3)c3ccc(-c4ccc5ccccc5c4)cc3)cc(N(c3ccccc3)c3ccc(-c4ccc5ccccc5c4)cc3)c2)cc1]

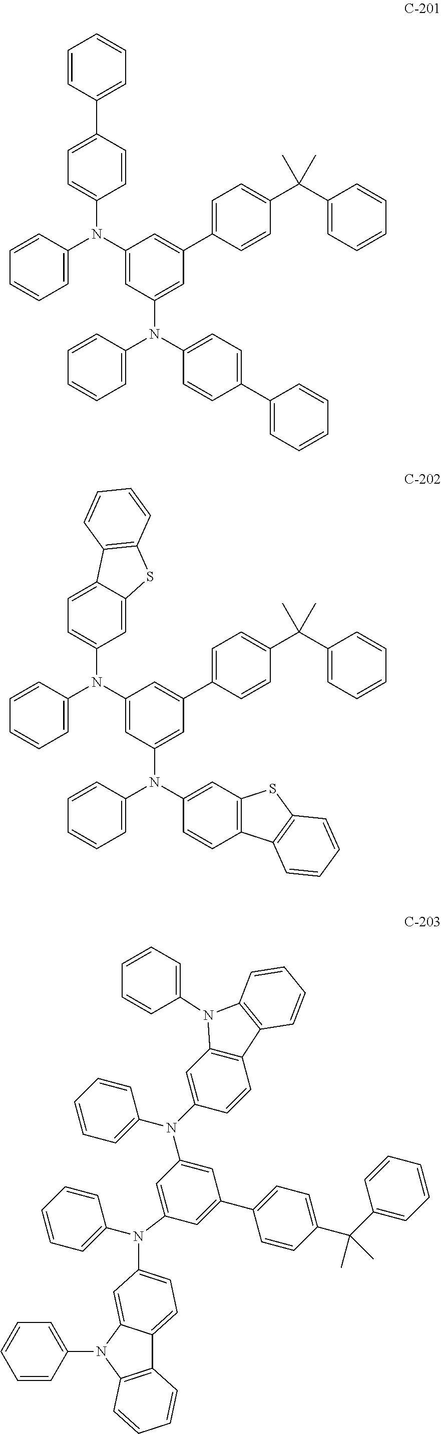 [SMILES: CC(C)(c1ccccc1)c1ccc(-c2cc(N(c3ccccc3)c3ccc(-c4ccccc4)cc3)cc(N(c3ccccc3)c3ccc(-c4ccccc4)cc3)c2)cc1 ; CC(C)(c1ccccc1)c1ccc(-c2cc(N(c3ccccc3)c3ccc4c(c3)sc3ccccc34)cc(N(c3ccccc3)c3ccc4c(c3)sc3ccccc34)c2)cc1 ; CC(C)(c1ccccc1)c1ccc(-c2cc(N(c3ccccc3)c3ccc4c5ccccc5n(-c5ccccc5)c4c3)cc(N(c3ccccc3)c3ccc4c5ccccc5n(-c5ccccc5)c4c3)c2)cc1]

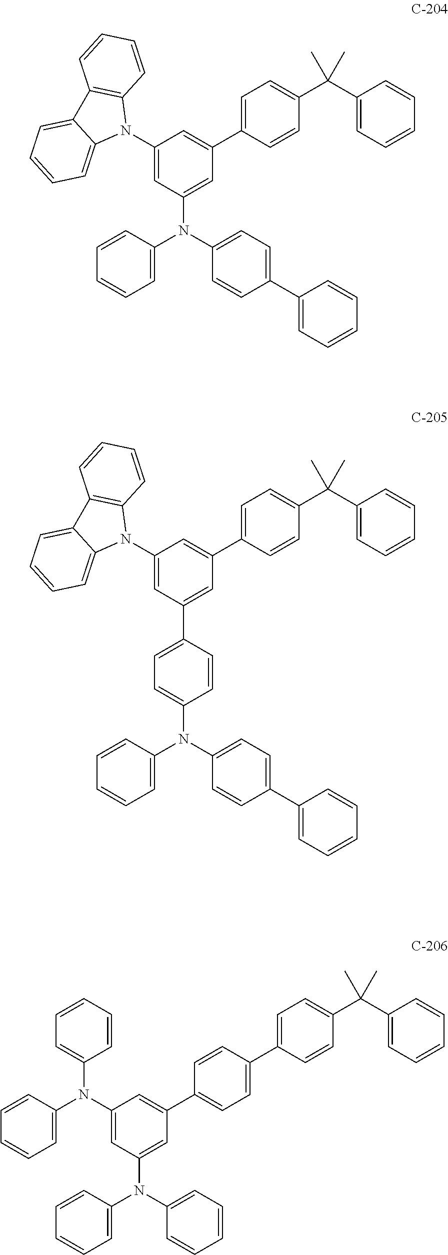 [SMILES: CC(C)(c1ccccc1)c1ccc(-c2cc(N(c3ccccc3)c3ccc(-c4ccccc4)cc3)cc(-n3c4ccccc4c4ccccc43)c2)cc1 ; CC(C)(c1ccccc1)c1ccc(-c2cc(-c3ccc(N(c4ccccc4)c4ccc(-c5ccccc5)cc4)cc3)cc(-n3c4ccccc4c4ccccc43)c2)cc1 ; CC(C)(c1ccccc1)c1ccc(-c2ccc(-c3cc(N(c4ccccc4)c4ccccc4)cc(N(c4ccccc4)c4ccccc4)c3)cc2)cc1]

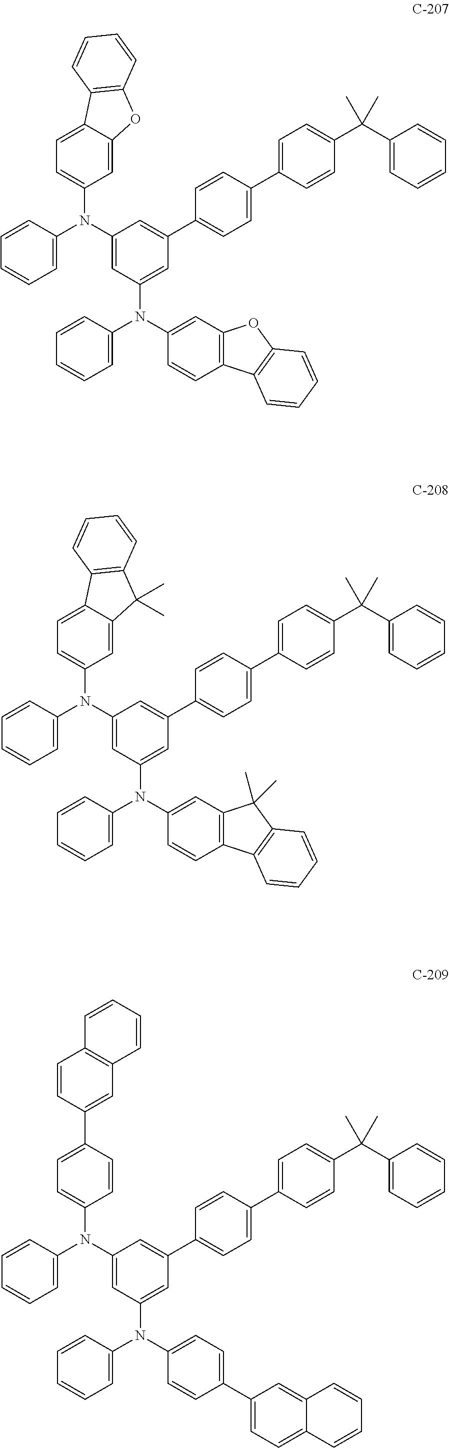 [SMILES: CC(C)(c1ccccc1)c1ccc(-c2ccc(-c3cc(N(c4ccccc4)c4ccc5c(c4)oc4ccccc45)cc(N(c4ccccc4)c4ccc5c(c4)oc4ccccc45)c3)cc2)cc1 ; CC(C)(c1ccccc1)c1ccc(-c2ccc(-c3cc(N(c4ccccc4)c4ccc5c(c4)C(C)(C)c4ccccc4-5)cc(N(c4ccccc4)c4ccc5c(c4)C(C)(C)c4ccccc4-5)c3)cc2)cc1 ; CC(C)(c1ccccc1)c1ccc(-c2ccc(-c3cc(N(c4ccccc4)c4ccc(-c5ccc6ccccc6c5)cc4)cc(N(c4ccccc4)c4ccc(-c5ccc6ccccc6c5)cc4)c3)cc2)cc1]

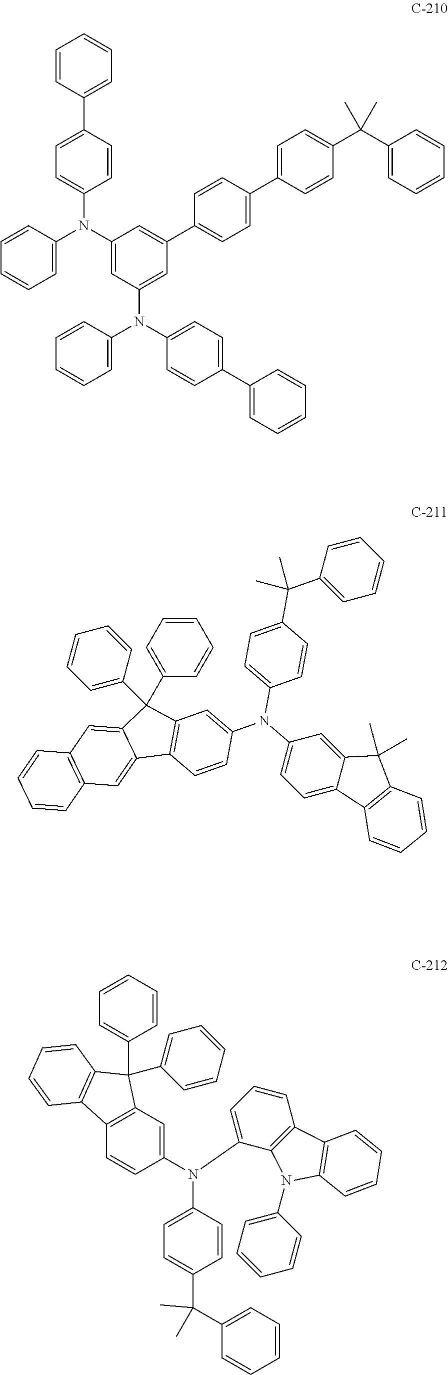 [SMILES: CC(C)(c1ccccc1)c1ccc(-c2ccc(-c3cc(N(c4ccccc4)c4ccc(-c5ccccc5)cc4)cc(N(c4ccccc4)c4ccc(-c5ccccc5)cc4)c3)cc2)cc1 ; CC(C)(c1ccccc1)c1ccc(N(c2ccc3c(c2)C(C)(C)c2ccccc2-3)c2ccc3c(c2)C(c2ccccc2)(c2ccccc2)c2cc4ccccc4cc2-3)cc1 ; CC(C)(c1ccccc1)c1ccc(N(c2ccc3c(c2)C(c2ccccc2)(c2ccccc2)c2ccccc2-3)c2cccc3c4ccccc4n(-c4ccccc4)c23)cc1]

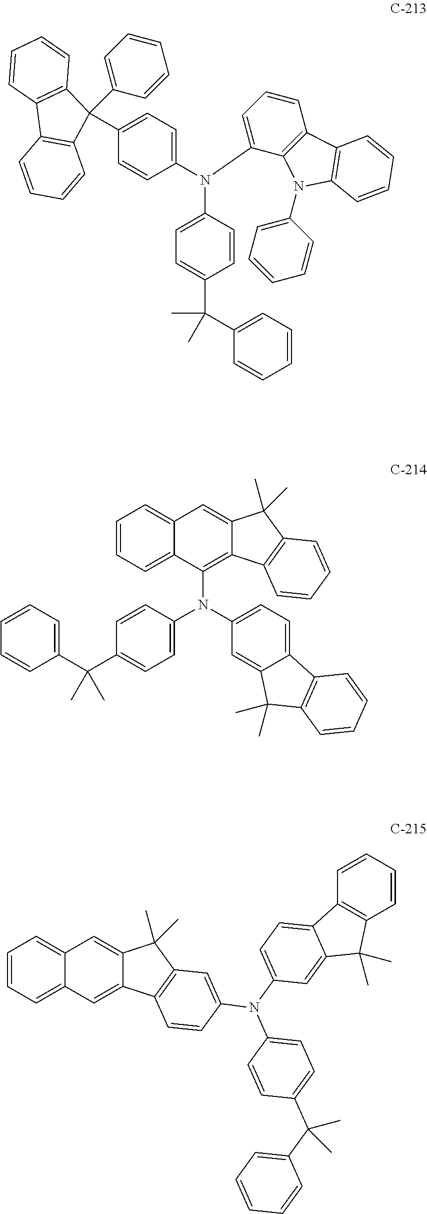 [SMILES: CC(C)(c1ccccc1)c1ccc(N(c2ccc(C3(c4ccccc4)c4ccccc4-c4ccccc43)cc2)c2cccc3c4ccccc4n(-c4ccccc4)c23)cc1 ; CC(C)(c1ccccc1)c1ccc(N(c2ccc3c(c2)C(C)(C)c2ccccc2-3)c2c3c(cc4ccccc24)C(C)(C)c2ccccc2-3)cc1 ; CC(C)(c1ccccc1)c1ccc(N(c2ccc3c(c2)C(C)(C)c2ccccc2-3)c2ccc3c(c2)C(C)(C)c2cc4ccccc4cc2-3)cc1]

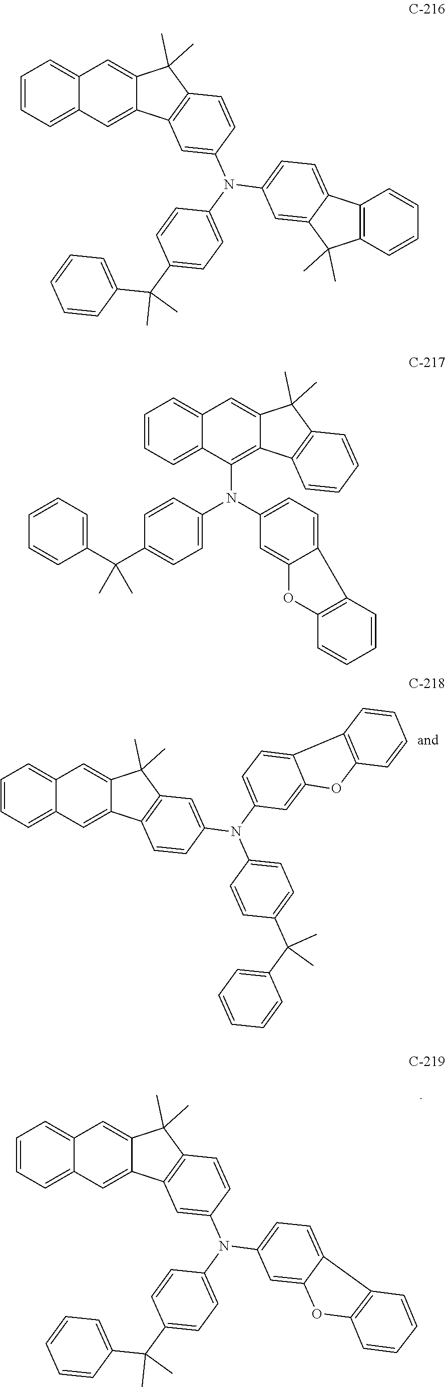 [SMILES: CC(C)(c1ccccc1)c1ccc(N(c2ccc3c(c2)-c2cc4ccccc4cc2C3(C)C)c2ccc3c(c2)C(C)(C)c2ccccc2-3)cc1 ; CC(C)(c1ccccc1)c1ccc(N(c2ccc3c(c2)oc2ccccc23)c2c3c(cc4ccccc24)C(C)(C)c2ccccc2-3)cc1 ; CC(C)(c1ccccc1)c1ccc(N(c2ccc3c(c2)C(C)(C)c2cc4ccccc4cc2-3)c2ccc3c(c2)oc2ccccc23)cc1 ; CC(C)(c1ccccc1)c1ccc(N(c2ccc3c(c2)-c2cc4ccccc4cc2C3(C)C)c2ccc3c(c2)oc2ccccc23)cc1]

6. An organic electroluminescent material comprising the organic electroluminescent compound according to claim 1.

7. An organic electroluminescent material comprising the organic electroluminescent compound according to claim 1 and a compound represented by any one of the following formulas 11 to 13: 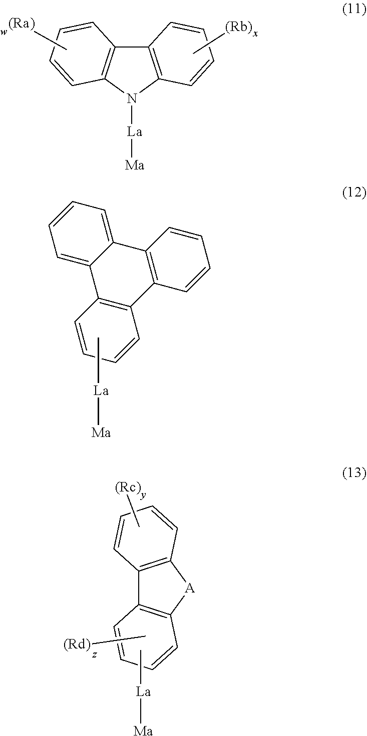
in formulas 11 to 13,
Ma represents a substituted or unsubstituted (C6-C30)aryl, a substituted or unsubstituted mono- or di- (C6-C30)arylamino, or a substituted or unsubstituted (3- to 30-membered)heteroaryl;
La represents a single bond, a substituted or unsubstituted (C6-C30)arylene, or a substituted or unsubstituted (3- to 30-membered)heteroarylene;
A represents S, O, N(Re) or C(Rf)(Rg);
Ra to Rd, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted (3- to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted (C1-C30)alkyl(C6-C30)aylamino, or a substituted or unsubstituted mono- or di- (C6-C30)arylamino; or may be linked to an adjacent substituent(s) to form a substituted or unsubstituted, mono- or polycyclic, (3- to 30-membered) alicyclic or aromatic ring, or the combination thereof, and the formed alicyclic or aromatic ring, or the combination thereof may contain at least one heteroatom selected from N, O, and S;
Re to Rg, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino; or Rf and Rg may be linked to each other to form a substituted or unsubstituted, mono- or polycyclic, (3- to 30-membered) alicyclic or aromatic ring, or the combination thereof, and the formed alicyclic or aromatic ring, or the combination thereof may contain at least one heteroatom selected from N, O, and S;
w to y, each independently, represent an integer of 1 to 4, and z represents an integer of 1 to 3; where if w to z are each an integer of 2 or more, each of Ra to each of Rd may be the same as or different from each other; and
the heteroaryl(ene) contains at least one heteroatom selected from B, N, O, S, Si and P.

8. An organic electroluminescent device comprising the organic electroluminescent compound according to claim 1.

9. The organic electroluminescent device according to claim 8, wherein the organic electroluminescent compound is comprised in at least one layer of a light-emitting layer, a hole transport layer, and a hole auxiliary layer.
